SEQUENCE LISTING (140 sequences)

SEQ ID NO: 1 - protein, 512 aa, Homo sapiens
MTAPWVALALLWGSLCAGSGRGEAETRECIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPTAPTLLTVLAYSLLPIGGLSLIVLLAFWMYRHRKPPYGHVDIHEDPGPPPPSPLVGLKPLQLLEIKARGRFGCVWKAQLMNDFVAVKIFPLQDKQSWQSEREIFSTPGMKHENLLQFIAAEKRGSNLEVELWLITAFHDKGSLTDYLKGNIITWNELCHVAETMSRGLSYLHEDVPWCRGEGHKPSIAHRDFKSKNVLLKSDLTAVLADFGLAVRFEPGKPPGDTHGQVGTRRYMAPEVLEGAINFQRDAFLRIDMYAMGLVLWELVSRCKAADGPVDEYMLPFEEEIGQHPSLEELQEVVVHKKMRPTIKDHWLKHPGLAQLCVTIEECWDHDAEARLSAGCVEERVSLIRRSVNGTTSDCLVSLVTSVTNVDLPPKESSI

SEQ ID NO: 2 - protein, 115 aa, Homo sapiens
GRGEAETRECIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPTAPT

SEQ ID NO: 3 - protein, 100 aa, Homo sapiens
GRGEAETRECIYYNANWELERTNQSGLERCEGEQDKRLHCYASWRNSSGTIELVKKGCWLDDFNCYDRQECVATEENPQVYFCCCEGNFCNERFTHLPEA

SEQ ID NO: 4 - protein, 512 aa, Homo sapiens
MTAPWVALALLWGSLCAGSGRGEAETRECIYYNANWELERTNQSGLERCEGEQDKRLHCYASWANSSGTIELVKKGCWLDDFNCYDRQECVATEENPQVYFCCCEGNFCNERFTHLPEAGGPEVTYEPPPTAPTLLTVLAYSLLPIGGLSLIVLLAFWMYRHRKPPYGHVDIHEDPGPPPPSPLVGLKPLQLLEIKARGRFGCVWKAQLMNDFVAVKIFPLQDKQSWQSEREIFSTPGMKHENLLQFIAAEKRGSNLEVELWLITAFHDKGSLTDYLKGNIITWNELCHVAETMSRGLSYLHEDVPWCRGEGHKPSIAHRDFKSKNVLLKSDLTAVLADFGLAVRFEPGKPPGDTHGQVGTRRYMAPEVLEGAINFQRDAFLRIDMYAMGLVLWELVSRCKAADGPVDEYMLPFEEEIGQHPSLEELQEVVVHKKMRPTIKDHWLKHPGLAQLCVTIEECWDHDAEARLSAGCVEERVSLIRRSVNGTTSDCLVSLVTSVTNVDLPPKESSI

SEQ ID NO: 5 - protein, 115 aa, Homo sapiens
GRGEAETRECIYYNANWELERTNQSGLERCEGEQDKRLHCYASWANSSGTIELVKKGCWLDDFNCYDRQECVATEENPQVYFCCCEGNFCNERFTHLPEAGGPEVTYEPPPTAPT

SEQ ID NO: 6 - protein, 100 aa, Homo sapiens
GRGEAETRECIYYNANWELERTNQSGLERCEGEQDKRLHCYASWANSSGTIELVKKGCWLDDFNCYDRQECVATEENPQVYFCCCEGNFCNERFTHLPEA

SEQ ID NO: 7 - DNA, 1536 bp, Homo sapiens
atgacggcgccctgggtggccctcgccctcctctggggatcgctgtgcgccggctctgggcgtggggaggctgagacacgggagtgcatctactacaacgccaactgggagctggagcgcaccaaccagagcggcctggagcgctgcgaaggcgagcaggacaagcggctgcactgctacgcctcctggcgcaacagctctggcaccatcgagctcgtgaagaagggctgctggctagatgacttcaactgctacgataggcaggagtgtgtggccactgaggagaacccccaggtgtacttctgctgctgtgaaggcaacttctgcaacgaacgcttcactcatttgccagaggctgggggcccggaagtcacgtacgagccacccccgacagcccccaccctgctcacggtgctggcctactcactgctgcccatcgggggcctttccctcatcgtcctgctggccttttggatgtaccggcatcgcaagcccccctacggtcatgtggacatccatgaggaccctgggcctccaccaccatcccctctggtgggcctgaagccactgcagctgctggagatcaaggctcgggggcgctttggctgtgtctggaaggcccagctcatgaatgactttgtagctgtcaagatcttcccactccaggacaagcagtcgtggcagagtgaacgggagatcttcagcacacctggcatgaagcacgagaacctgctacagttcattgctgccgagaagcgaggctccaacctcgaagtagagctgtggctcatcacggccttccatgacaagggctccctcacggattacctcaaggggaacatcatcacatggaacgaactgtgtcatgtagcagagacgatgtcacgaggcctctcatacctgcatgaggatgtgccctggtgccgtggcgagggccacaagccgtctattgcccacagggactttaaaagtaagaatgtattgctgaagagcgacctcacagccgtgctggctgactttggcttggctgttcgatttgagccagggaaacctccaggggacacccacggacaggtaggcacgagacggtacatggctcctgaggtgctcgagggagccatcaacttccagagagatgccttcctgcgcattgacatgtatgccatggggttggtgctgtgggagcttgtgtctcgctgcaaggctgcagacggacccgtggatgagtacatgctgccctttgaggaagagattggccagcacccttcgttggaggagctgcaggaggtggtggtgcacaagaagatgaggcccaccattaaagatcactggttgaaacacccgggcctggcccagctttgtgtgaccatcgaggagtgctgggaccatgatgcagaggctcgcttgtccgcgggctgtgtggaggagcgggtgtccctgattcggaggtcggtcaacggcactacctcggactgtctcgtttccctggtgacctctgtcaccaatgtggacctgccccctaaagagtcaagcatc

SEQ ID NO: 8 - DNA, 345 bp, Homo sapiens
gggcgtggggaggctgagacacgggagtgcatctactacaacgccaactgggagctggagcgcaccaaccagagcggcctggagcgctgcgaaggcgagcaggacaagcggctgcactgctacgcctcctggcgcaacagctctggcaccatcgagctcgtgaagaagggctgctggctagatgacttcaactgctacgataggcaggagtgtgtggccactgaggagaacccccaggtgtacttctgctgctgtgaaggcaacttctgcaacgaacgcttcactcatttgccagaggctgggggcccggaagtcacgtacgagccacccccgacagcccccacc

SEQ ID NO: 9 - protein, 513 aa, Homo sapiens
MGAAAKLAFAVFLISCSSGAILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEMEVTQPTSNPVTPKPPYYNILLYSLVPLMLIAGIVICAFWVYRHHKMAYPPVLVPTQDPGPPPPSPLLGLKPLQLLEVKARGRFGCVWKAQLLNEYVAVKIFPIQDKQSWQNEYEVYSLPGMKHENILQFIGAEKRGTSVDVDLWLITAFHEKGSLSDFLKANVVSWNELCHIAETMARGLAYLHEDIPGLKDGHKPAISHRDIKSKNVLLKNNLTACIADFGLALKFEAGKSAGDTHGQVGTRRYMAPEVLEGAINFQRDAFLRIDMYAMGLVLWELASRCTAADGPVDEYMLPFEEEIGQHPSLEDMQEVVVHKKKRPVLRDYWQKHAGMAMLCETIEECWDHDAEARLSAGCVGERITQMQRLTNIITTEDIVTVVTMVTNVDFPPKESSL

SEQ ID NO: 10 - protein, 115 aa, Homo sapiens
ILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEMEVTQPTSNPVTPKPP

SEQ ID NO: 11 - protein, 100 aa, Homo sapiens
ILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEM

SEQ ID NO: 12 - DNA, 1539 bp, Homo sapiens
atgggagctgctgcaaagttggcgtttgccgtctttcttatctcctgttcttcaggtgctatacttggtagatcagaaactcaggagtgtcttttctttaatgctaattgggaaaaagacagaaccaatcaaactggtgttgaaccgtgttatggtgacaaagataaacggcggcattgttttgctacctggaagaatatttctggttccattgaaatagtgaaacaaggttgttggctggatgatatcaactgctatgacaggactgattgtgtagaaaaaaaagacagccctgaagtatatttttgttgctgtgagggcaatatgtgtaatgaaaagttttcttattttccggagatggaagtcacacagcccacttcaaatccagttacacctaagccaccctattacaacatcctgctctattccttggtgccacttatgttaattgcggggattgtcatttgtgcattttgggtgtacaggcatcacaagatggcctaccctcctgtacttgttccaactcaagacccaggaccacccccaccttctccattactaggtttgaaaccactgcagttattagaagtgaaagcaaggggaagatttggttgtgtctggaaagcccagttgcttaacgaatatgtggctgtcaaaatatttccaatacaggacaaacagtcatggcaaaatgaatacgaagtctacagtttgcctggaatgaagcatgagaacatattacagttcattggtgcagaaaaacgaggcaccagtgttgatgtggatctttggctgatcacagcatttcatgaaaagggttcactatcagactttcttaaggctaatgtggtctcttggaatgaactgtgtcatattgcagaaaccatggctagaggattggcatatttacatgaggatatacctggcctaaaagatggccacaaacctgccatatctcacagggacatcaaaagtaaaaatgtgctgttgaaaaacaacctgacagcttgcattgctgactttgggttggccttaaaatttgaggctggcaagtctgcaggcgatacccatggacaggttggtacccggaggtacatggctccagaggtattagagggtgctataaacttccaaagggatgcatttttgaggatagatatgtatgccatgggattagtcctatgggaactggcttctcgctgtactgctgcagatggacctgtagatgaatacatgttgccatttgaggaggaaattggccagcatccatctcttgaagacatgcaggaagttgttgtgcataaaaaaaagaggcctgttttaagagattattggcagaaacatgctggaatggcaatgctctgtgaaaccattgaagaatgttgggatcacgacgcagaagccaggttatcagctggatgtgtaggtgaaagaattacccagatgcagagactaacaaatattattaccacagaggacattgtaacagtggtcacaatggtgacaaatgttgactttcctcccaaagaatctagtcta

SEQ ID NO: 13 - DNA, 345 bp, Homo sapiens
atacttggtagatcagaaactcaggagtgtcttttctttaatgctaattgggaaaaagacagaaccaatcaaactggtgttgaaccgtgttatggtgacaaagataaacggcggcattgttttgctacctggaagaatatttctggttccattgaaatagtgaaacaaggttgttggctggatgatatcaactgctatgacaggactgattgtgtagaaaaaaaagacagccctgaagtatatttttgttgctgtgagggcaatatgtgtaatgaaaagttttcttattttccggagatggaagtcacacagcccacttcaaatccagttacacctaagccaccc

SEQ ID NO: 14 - protein, 1038 aa, Homo sapiens
MTSSLQRPWRVPWLPWTILLVSTAAASQNQERLCAFKDPYQQDLGIGESRISHENGTILCSKGSTCYGLWEKSKGDINLVKQGCWSHIGDPQECHYEECVVTTTPPSIQNGTYRFCCCSTDLCNVNFTENFPPPDTTPLSPPHSFNRDETIIIALASVSVLAVLIVALCFGYRMLTGDRKQGLHSMNMMEAAASEPSLDLDNLKLLELIGRGRYGAVYKGSLDERPVAVKVFSFANRQNFINEKNIYRVPLMEHDNIARFIVGDERVTADGRMEYLLVMEYYPNGSLCKYLSLHTSDWVSSCRLAHSVTRGLAYLHTELPRGDHYKPAISHRDLNSRNVLVKNDGTCVISDFGLSMRLTGNRLVRPGEEDNAAISEVGTIRYMAPEVLEGAVNLRDCESALKQVDMYALGLIYWEIFMRCTDLFPGESVPEYQMAFQTEVGNHPTFEDMQVLVSREKQRPKFPEAWKENSLAVRSLKETIEDCWDQDAEARLTAQCAEERMAELMMIWERNKSVSPTVNPMSTAMQNERNLSHNRRVPKIGPYPDYSSSSYIEDSIHHTDSIVKNISSEHSMSSTPLTIGEKNRNSINYERQQAQARIPSPETSVTSLSTNTTTTNTTGLTPSTGMTTISEMPYPDETNLHTTNVAQSIGPTPVCLQLTEEDLETNKLDPKEVDKNLKESSDENLMEHSLKQFSGPDPLSSTSSSLLYPLIKLAVEATGQQDFTQTANGQACLIPDVLPTQIYPLPKQQNLPKRPTSLPLNTKNSTKEPRLKFGSKHKSNLKQVETGVAKMNTINAAEPHVVTVTMNGVAGRNHSVNSHAATTQYANGTVLSGQTTNIVTHRAQEMLQNQFIGEDTRLNINSSPDEHEPLLRREQQAGHDEGVLDRLVDRRERPLEGGRTNSNNNNSNPCSEQDVLAQGVPSTAADPGPSKPRRAQRPNSLDLSATNVLDGSSIQIGESTQDGKSGSGEKIKKRVKTPYSLKRWRPSTWVISTESLDCEVNNNGSNRAVHSKSSTAVYLAEGGTATTMVSKDIGMNCL

SEQ ID NO: 15 - protein, 124 aa, Homo sapiens [sequence omitted]

SEQ ID NO: 16 - DNA, 3114 bp, Homo sapiens
atgacttcctcgctgcagcggccctggcgggtgccctggctaccatggaccatcctgctggtcagcactgcggctgcttcgcagaatcaagaacggctatgtgcgtttaaagatccgtatcagcaagaccttgggataggtgagagtagaatctctcatgaaaatgggacaatattatgctcgaaaggtagcacctgctatggcctttgggagaaatcaaaaggggacataaatcttgtaaaacaaggatgttggtctcacattggagatccccaagagtgtcactatgaagaatgtgtagtaactaccactcctccctcaattcagaatggaacataccgtttctgctgttgtagcacagatttatgtaatgtcaactttactgagaattttccacctcctgacacaacaccactcagtccacctcattcatttaaccgagatgagacaataatcattgctttggcatcagtctctgtattagctgttttgatagttgccttatgctttggatacagaatgttgacaggagaccgtaaacaaggtcttcacagtatgaacatgatggaggcagcagcatccgaaccctctcttgatctagataatctgaaactgttggagctgattggccgaggtcgatatggagcagtatataaaggctccttggatgagcgtccagttgctgtaaaagtgttttcctttgcaaaccgtcagaattttatcaacgaaaagaacatttacagagtgcctttgatggaacatgacaacattgcccgctttatagttggagatgagagagtcactgcagatggacgcatggaatatttgcttgtgatggagtactatcccaatggatctttatgcaagtatttaagtctccacacaagtgactgggtaagctcttgccgtcttgctcattctgttactagaggactggcttatcttcacacagaattaccacgaggagatcattataaacctgcaatttcccatcgagatttaaacagcagaaatgtcctagtgaaaaatgatggaacctgtgttattagtgactttggactgtccatgaggctgactggaaatagactggtgcgcccaggggaggaagataatgcagccataagcgaggttggcactatcagatatatggcaccagaagtgctagaaggagctgtgaacttgagggactgtgaatcagctttgaaacaagtagacatgtatgctcttggactaatctattgggagatatttatgagatgtacagacctcttcccaggggaatccgtaccagagtaccagatggcttttcagacagaggttggaaaccatcccacttttgaggatatgcaggttctcgtgtctagggaaaaacagagacccaagttcccagaagcctggaaagaaaatagcctggcagtgaggtcactcaaggagacaatcgaagactgttgggaccaggatgcagaggctcggcttactgcacagtgtgctgaggaaaggatggctgaacttatgatgatttgggaaagaaacaaatctgtgagcccaacagtcaatccaatgtctactgctatgcagaatgaacgcaacctgtcacataataggcgtgtgccaaaaattggtccttatccagattattcttcctcctcatacattgaagactctatccatcatactgacagcatcgtgaagaatatttcctctgagcattctatgtccagcacacctttgactataggggaaaaaaaccgaaattcaattaactatgaacgacagcaagcacaagctcgaatccccagccctgaaacaagtgtcaccagcctctccaccaacacaacaaccacaaacaccacaggactcacgccaagtactggcatgactactatatctgagatgccatacccagatgaaacaaatctgcataccacaaatgttgcacagtcaattgggccaacccctgtctgcttacagctgacagaagaagacttggaaaccaacaagctagacccaaaagaagttgataagaacctcaaggaaagctctgatgagaatctcatggagcactctcttaaacagttcagtggcccagacccactgagcagtactagttctagcttgctttacccactcataaaacttgcagtagaagcaactggacagcaggacttcacacagactgcaaatggccaagcatgtttgattcctgatgttctgcctactcagatctatcctctccccaagcagcagaaccttcccaagagacctactagtttgcctttgaacaccaaaaattcaacaaaagagccccggctaaaatttggcagcaagcacaaatcaaacttgaaacaagtcgaaactggagttgccaagatgaatacaatcaatgcagcagaacctcatgtggtgacagtcaccatgaatggtgtggcaggtagaaaccacagtgttaactcccatgctgccacaacccaatatgccaatgggacagtactatctggccaaacaaccaacatagtgacacatagggcccaagaaatgttgcagaatcagtttattggtgaggacacccggctgaatattaattccagtcctgatgagcatgagcctttactgagacgagagcaacaagctggccatgatgaaggtgttctggatcgtcttgtggacaggagggaacggccactagaaggtggccgaactaattccaataacaacaacagcaatccatgttcagaacaagatgttcttgcacagggtgttccaagcacagcagcagatcctgggccatcaaagcccagaagagcacagaggcctaattctctggatctttcagccacaaatgtcctggatggcagcagtatacagataggtgagtcaacacaagatggcaaatcaggatcaggtgaaaagatcaagaaacgtgtgaaaactccctattctcttaagcggtggcgcccctccacctgggtcatctccactgaatcgctggactgtgaagtcaacaataatggcagtaacagggcagttcattccaaatccagcactgctgtttaccttgcagaaggaggcactgctacaaccatggtgtctaaagatataggaatgaactgtctg

SEQ ID NO: 17 - DNA, 372 bp, Homo sapiens
tcgcagaatcaagaacggctatgtgcgtttaaagatccgtatcagcaagaccttgggataggtgagagtagaatctctcatgaaaatgggacaatattatgctcgaaaggtagcacctgctatggcctttgggagaaatcaaaaggggacataaatcttgtaaaacaaggatgttggtctcacattggagatccccaagagtgtcactatgaagaatgtgtagtaactaccactcctccctcaattcagaatggaacataccgtttctgctgttgtagcacagatttatgtaatgtcaactttactgagaattttccacctcctgacacaacaccactcagtccacctcattcatttaaccgagatgagaca

SEQ ID NO: 18 - protein, 530 aa, Homo sapiens
MTSSLQRPWRVPWLPWTILLVSTAAASQNQERLCAFKDPYQQDLGIGESRISHENGTILCSKGSTCYGLWEKSKGDINLVKQGCWSHIGDPQECHYEECVVTTTPPSIQNGTYRFCCCSTDLCNVNFTENFPPPDTTPLSPPHSFNRDETIIIALASVSVLAVLIVALCFGYRMLTGDRKQGLHSMNMMEAAASEPSLDLDNLKLLELIGRGRYGAVYKGSLDERPVAVKVFSFANRQNFINEKNIYRVPLMEHDNIARFIVGDERVTADGRMEYLLVMEYYPNGSLCKYLSLHTSDWVSSCRLAHSVTRGLAYLHTELPRGDHYKPAISHRDLNSRNVLVKNDGTCVISDFGLSMRLTGNRLVRPGEEDNAAISEVGTIRYMAPEVLEGAVNLRDCESALKQVDMYALGLIYWEIFMRCTDLFPGESVPEYQMAFQTEVGNHPTFEDMQVLVSREKQRPKFPEAWKENSLAVRSLKETIEDCWDQDAEARLTAQCAEERMAELMMIWERNKSVSPTVNPMSTAMQNERR

SEQ ID NO: 19 - DNA, 1590 bp, Homo sapiens
atgacttcctcgctgcagcggccctggcgggtgccctggctaccatggaccatcctgctggtcagcactgcggctgcttcgcagaatcaagaacggctatgtgcgtttaaagatccgtatcagcaagaccttgggataggtgagagtagaatctctcatgaaaatgggacaatattatgctcgaaaggtagcacctgctatggcctttgggagaaatcaaaaggggacataaatcttgtaaaacaaggatgttggtctcacattggagatccccaagagtgtcactatgaagaatgtgtagtaactaccactcctccctcaattcagaatggaacataccgtttctgctgttgtagcacagatttatgtaatgtcaactttactgagaattttccacctcctgacacaacaccactcagtccacctcattcatttaaccgagatgagacaataatcattgctttggcatcagtctctgtattagctgttttgatagttgccttatgctttggatacagaatgttgacaggagaccgtaaacaaggtcttcacagtatgaacatgatggaggcagcagcatccgaaccctctcttgatctagataatctgaaactgttggagctgattggccgaggtcgatatggagcagtatataaaggctccttggatgagcgtccagttgctgtaaaagtgttttcctttgcaaaccgtcagaattttatcaacgaaaagaacatttacagagtgcctttgatggaacatgacaacattgcccgctttatagttggagatgagagagtcactgcagatggacgcatggaatatttgcttgtgatggagtactatcccaatggatctttatgcaagtatttaagtctccacacaagtgactgggtaagctcttgccgtcttgctcattctgttactagaggactggcttatcttcacacagaattaccacgaggagatcattataaacctgcaatttcccatcgagatttaaacagcagaaatgtcctagtgaaaaatgatggaacctgtgttattagtgactttggactgtccatgaggctgactggaaatagactggtgcgcccaggggaggaagataatgcagccataagcgaggttggcactatcagatatatggcaccagaagtgctagaaggagctgtgaacttgagggactgtgaatcagctttgaaacaagtagacatgtatgctcttggactaatctattgggagatatttatgagatgtacagacctcttcccaggggaatccgtaccagagtaccagatggcttttcagacagaggttggaaaccatcccacttttgaggatatgcaggttctcgtgtctagggaaaaacagagacccaagttcccagaagcctggaaagaaaatagcctggcagtgaggtcactcaaggagacaatcgaagactgttgggaccaggatgcagaggctcggcttactgcacagtgtgctgaggaaaggatggctgaacttatgatgatttgggaaagaaacaaatctgtgagcccaacagtcaatccaatgtctactgctatgcagaatgaacgtagg

SEQ ID NO: 20 - protein, 503 aa, Homo sapiens
MTLGSPRKGLLMLLMALVTQGDPVKPSRGPLVTCTCESPHCKGPTCRGAWCTVVLVREEGRHPQEHRGCGNLHRELCRGRPTEFVNHYCCDSHLCNHNVSLVLEATQPPSEQPGTDGQLALILGPVLALLALVALGVLGLWHVRRRQEKQRGLHSELGESSLILKASEQGDSMLGDLLDSDCTTGSGSGLPFLVQRTVARQVALVECVGKGRYGEVWRGLWHGESVAVKIFSSRDEQSWFRETEIYNTVLLRHDNILGFIASDMTSRNSSTQLWLITHYHEHGSLYDFLQRQTLEPHLALRLAVSAACGLAHLHVEIFGTQGKPAIAHRDFKSRNVLVKSNLQCCIADLGLAVMHSQGSDYLDIGNNPRVGTKRYMAPEVLDEQIRTDCFESYKWTDIWAFGLVLWEIARRTIVNGIVEDYRPPFYDVVPNDPSFEDMKKVVCVDQQTPTIPNRLAADPVLSGLAQMMRECWYPNPSARLTALRIKKTLQKISNSPEKPKVIQ

SEQ ID NO: 21 - protein, 97 aa, Homo sapiens
DPVKPSRGPLVTCTCESPHCKGPTCRGAWCTVVLVREEGRHPQEHRGCGNLHRELCRGRPTEFVNHYCCDSHLCNHNVSLVLEATQPPSEQPGTDGQ

SEQ ID NO: 22 - DNA, 1509 bp, Homo sapiens
atgaccttgggctcccccaggaaaggccttctgatgctgctgatggccttggtgacccagggagaccctgtgaagccgtctcggggcccgctggtgacctgcacgtgtgagagcccacattgcaaggggcctacctgccggggggcctggtgcacagtagtgctggtgcgggaggaggggaggcacccccaggaacatcggggctgcgggaacttgcacagggagctctgcagggggcgccccaccgagttcgtcaaccactactgctgcgacagccacctctgcaaccacaacgtgtccctggtgctggaggccacccaacctccttcggagcagccgggaacagatggccagctggccctgatcctgggccccgtgctggccttgctggccctggtggccctgggtgtcctgggcctgtggcatgtccgacggaggcaggagaagcagcgtggcctgcacagcgagctgggagagtccagtctcatcctgaaagcatctgagcagggcgacagcatgttgggggacctcctggacagtgactgcaccacagggagtggctcagggctccccttcctggtgcagaggacagtggcacggcaggttgccttggtggagtgtgtgggaaaaggccgctatggcgaagtgtggcggggcttgtggcacggtgagagtgtggccgtcaagatcttctcctcgagggatgaacagtcctggttccgggagactgagatctataacacagtgttgctcagacacgacaacatcctaggcttcatcgcctcagacatgacctcccgcaactcgagcacgcagctgtggctcatcacgcactaccacgagcacggctccctctacgactttctgcagagacagacgctggagccccatctggctctgaggctagctgtgtccgcggcatgcggcctggcgcacctgcacgtggagatcttcggtacacagggcaaaccagccattgcccaccgcgacttcaagagccgcaatgtgctggtcaagagcaacctgcagtgttgcatcgccgacctgggcctggctgtgatgcactcacagggcagcgattacctggacatcggcaacaacccgagagtgggcaccaagcggtacatggcacccgaggtgctggacgagcagatccgcacggactgctttgagtcctacaagtggactgacatctgggcctttggcctggtgctgtgggagattgcccgccggaccatcgtgaatggcatcgtggaggactatagaccacccttctatgatgtggtgcccaatgaccccagctttgaggacatgaagaaggtggtgtgtgtggatcagcagacccccaccatccctaaccggctggctgcagacccggtcctctcaggcctagctcagatgatgcgggagtgctggtacccaaacccctctgcccgactcaccgcgctgcggatcaagaagacactacaaaaaattagcaacagtccagagaagcctaaagtgattcaa

SEQ ID NO: 23 - DNA, 291 bp, Homo sapiens
gaccctgtgaagccgtctcggggcccgctggtgacctgcacgtgtgagagcccacattgcaaggggcctacctgccggggggcctggtgcacagtagtgctggtgcgggaggaggggaggcacccccaggaacatcggggctgcgggaacttgcacagggagctctgcagggggcgccccaccgagttcgtcaaccactactgctgcgacagccacctctgcaaccacaacgtgtccctggtgctggaggccacccaacctccttcggagcagccgggaacagatggccag

SEQ ID NO: 24 - protein, 658 aa, Homo sapiens
MDRGTLPLAVALLLASCSLSPTSLAETVHCDLQPVGPERGEVTYTTSQVSKGCVAQAPNAILEVHVLFLEFPTGPSQLELTLQASKQNGTWPREVLLVLSVNSSVFLHLQALGIPLHLAYNSSLVTFQEPPGVNTTELPSFPKTQILEWAAERGPITSAAELNDPQSILLRLGQAQGSLSFCMLEASQDMGRTLEWRPRTPALVRGCHLEGVAGHKEAHILRVLPGHSAGPRTVTVKVELSCAPGDLDAVLILQGPPYVSWLIDANHNMQIWTTGEYSFKIFPEKNIRGFKLPDTPQGLLGEARMLNASIVASFVELPLASIVSLHASSCGGRLQTSPAPIQTTPPKDTCSPELLMSLIQTKCADDAMTLVLKKELVAHLKCTITGLTFWDPSCEAEDRGDKFVLRSAYSSCGMQVSASMISNEAVVNILSSSSPQRKKVHCLNMDSLSFQLGLYLSPHFLQASNTIEPGQQSFVQVRVSPSVSEFLLQLDSCHLDLGPEGGTVELIQGRAAKGNCVSLLSPSPEGDPRFSFLLHFYTVPIPKTGTLSCTVALRPKTGSQDQEVHRTVFMRLNIISPDLSGCTSKGLVLPAVLGITFGAFLIGALLTAALWYIYSHTRSPSKREPVVAVAAPASSESSSTNHSIGSTQSTPCSTSSMA

SEQ ID NO: 25 - DNA, 1974 bp, Homo sapiens
atggaccgcggcacgctccctctggctgttgccctgctgctggccagctgcagcctcagccccacaagtcttgcagaaacagtccattgtgaccttcagcctgtgggccccgagaggggcgaggtgacatataccactagccaggtctcgaagggctgcgtggctcaggcccccaatgccatccttgaagtccatgtcctcttcctggagttcccaacgggcccgtcacagctggagctgactctccaggcatccaagcaaaatggcacctggccccgagaggtgcttctggtcctcagtgtaaacagcagtgtcttcctgcatctccaggccctgggaatcccactgcacttggcctacaattccagcctggtcaccttccaagagcccccgggggtcaacaccacagagctgccatccttccccaagacccagatccttgagtgggcagctgagaggggccccatcacctctgctgctgagctgaatgacccccagagcatcctcctccgactgggccaagcccaggggtcactgtccttctgcatgctggaagccagccaggacatgggccgcacgctcgagtggcggccgcgtactccagccttggtccggggctgccacttggaaggcgtggccggccacaaggaggcgcacatcctgagggtcctgccgggccactcggccgggccccggacggtgacggtgaaggtggaactgagctgcgcacccggggatctcgatgccgtcctcatcctgcagggtcccccctacgtgtcctggctcatcgacgccaaccacaacatgcagatctggaccactggagaatactccttcaagatctttccagagaaaaacattcgtggcttcaagctcccagacacacctcaaggcctcctgggggaggcccggatgctcaatgccagcattgtggcatccttcgtggagctaccgctggccagcattgtctcacttcatgcctccagctgcggtggtaggctgcagacctcacccgcaccgatccagaccactcctcccaaggacacttgtagcccggagctgctcatgtccttgatccagacaaagtgtgccgacgacgccatgaccctggtactaaagaaagagcttgttgcgcatttgaagtgcaccatcacgggcctgaccttctgggaccccagctgtgaggcagaggacaggggtgacaagtttgtcttgcgcagtgcttactccagctgtggcatgcaggtgtcagcaagtatgatcagcaatgaggcggtggtcaatatcctgtcgagctcatcaccacagcggaaaaaggtgcactgcctcaacatggacagcctctctttccagctgggcctctacctcagcccacacttcctccaggcctccaacaccatcgagccggggcagcagagctttgtgcaggtcagagtgtccccatccgtctccgagttcctgctccagttagacagctgccacctggacttggggcctgagggaggcaccgtggaactcatccagggccgggcggccaagggcaactgtgtgagcctgctgtccccaagccccgagggtgacccgcgcttcagcttcctcctccacttctacacagtacccatacccaaaaccggcaccctcagctgcacggtagccctgcgtcccaagaccgggtctcaagaccaggaagtccataggactgtcttcatgcgcttgaacatcatcagccctgacctgtctggttgcacaagcaaaggcctcgtcctgcccgccgtgctgggcatcacctttggtgccttcctcatcggggccctgctcactgctgcactctggtacatctactcgcacacgcgttcccccagcaagcgggagcccgtggtggcggtggctgccccggcctcctcggagagcagcagcaccaaccacagcatcgggagcacccagagcaccccctgctccaccagcagcatggca

SEQ ID NO: 26 - protein, 625 aa, Homo sapiens
MDRGTLPLAVALLLASCSLSPTSLAETVHCDLQPVGPERGEVTYTTSQVSKGCVAQAPNAILEVHVLFLEFPTGPSQLELTLQASKQNGTWPREVLLVLSVNSSVFLHLQALGIPLHLAYNSSLVTFQEPPGVNTTELPSFPKTQILEWAAERGPITSAAELNDPQSILLRLGQAQGSLSFCMLEASQDMGRTLEWRPRTPALVRGCHLEGVAGHKEAHILRVLPGHSAGPRTVTVKVELSCAPGDLDAVLILQGPPYVSWLIDANHNMQIWTTGEYSFKIFPEKNIRGFKLPDTPQGLLGEARMLNASIVASFVELPLASIVSLHASSCGGRLQTSPAPIQTTPPKDTCSPELLMSLIQTKCADDAMTLVLKKELVAHLKCTITGLTFWDPSCEAEDRGDKFVLRSAYSSCGMQVSASMISNEAVVNILSSSSPQRKKVHCLNMDSLSFQLGLYLSPHFLQASNTIEPGQQSFVQVRVSPSVSEFLLQLDSCHLDLGPEGGTVELIQGRAAKGNCVSLLSPSPEGDPRFSFLLHFYTVPIPKTGTLSCTVALRPKTGSQDQEVHRTVFMRLNIISPDLSGCTSKGLVLPAVLGITFGAFLIGALLTAALWYIYSHTREYPRPPQ

SEQ ID NO: 27 - DNA, 1875 bp, Homo sapiens
atggaccgcggcacgctccctctggctgttgccctgctgctggccagctgcagcctcagccccacaagtcttgcagaaacagtccattgtgaccttcagcctgtgggccccgagaggggcgaggtgacatataccactagccaggtctcgaagggctgcgtggctcaggcccccaatgccatccttgaagtccatgtcctcttcctggagttcccaacgggcccgtcacagctggagctgactctccaggcatccaagcaaaatggcacctggccccgagaggtgcttctggtcctcagtgtaaacagcagtgtcttcctgcatctccaggccctgggaatcccactgcacttggcctacaattccagcctggtcaccttccaagagcccccgggggtcaacaccacagagctgccatccttccccaagacccagatccttgagtgggcagctgagaggggccccatcacctctgctgctgagctgaatgacccccagagcatcctcctccgactgggccaagcccaggggtcactgtccttctgcatgctggaagccagccaggacatgggccgcacgctcgagtggcggccgcgtactccagccttggtccggggctgccacttggaaggcgtggccggccacaaggaggcgcacatcctgagggtcctgccgggccactcggccgggccccggacggtgacggtgaaggtggaactgagctgcgcacccggggatctcgatgccgtcctcatcctgcagggtcccccctacgtgtcctggctcatcgacgccaaccacaacatgcagatctggaccactggagaatactccttcaagatctttccagagaaaaacattcgtggcttcaagctcccagacacacctcaaggcctcctgggggaggcccggatgctcaatgccagcattgtggcatccttcgtggagctaccgctggccagcattgtctcacttcatgcctccagctgcggtggtaggctgcagacctcacccgcaccgatccagaccactcctcccaaggacacttgtagcccggagctgctcatgtccttgatccagacaaagtgtgccgacgacgccatgaccctggtactaaagaaagagcttgttgcgcatttgaagtgcaccatcacgggcctgaccttctgggaccccagctgtgaggcagaggacaggggtgacaagtttgtcttgcgcagtgcttactccagctgtggcatgcaggtgtcagcaagtatgatcagcaatgaggcggtggtcaatatcctgtcgagctcatcaccacagcggaaaaaggtgcactgcctcaacatggacagcctctctttccagctgggcctctacctcagcccacacttcctccaggcctccaacaccatcgagccggggcagcagagctttgtgcaggtcagagtgtccccatccgtctccgagttcctgctccagttagacagctgccacctggacttggggcctgagggaggcaccgtggaactcatccagggccgggcggccaagggcaactgtgtgagcctgctgtccccaagccccgagggtgacccgcgcttcagcttcctcctccacttctacacagtacccatacccaaaaccggcaccctcagctgcacggtagccctgcgtcccaagaccgggtctcaagaccaggaagtccataggactgtcttcatgcgcttgaacatcatcagccctgacctgtctggttgcacaagcaaaggcctcgtcctgcccgccgtgctgggcatcacctttggtgccttcctcatcggggccctgctcactgctgcactctggtacatctactcgcacacgcgtgagtaccccaggcccccacag

SEQ ID NO: 28 - protein, 582 aa, Artificial Sequence
  Description of Artificial Sequence: Synthetic polypeptide
ETVHCDLQPVGPERDEVTYTTSQVSKGCVAQAPNAILEVHVLFLEFPTGPSQLELTLQASKQNGTWPREVLLVLSVNSSVFLHLQALGIPLHLAYNSSLVTFQEPPGVNTTELPSFPKTQILEWAAERGPITSAAELNDPQSILLRLGQAQGSLSFCMLEASQDMGRTLEWRPRTPALVRGCHLEGVAGHKEAHILRVLPGHSAGPRTVTVKVELSCAPGDLDAVLILQGPPYVSWLIDANHNMQIWTTGEYSFKIFPEKNIRGFKLPDTPQGLLGEARMLNASIVASFVELPLASIVSLHASSCGGRLQTSPAPIQTTPPKDTCSPELLMSLIQTKCADDAMTLVLKKELVATGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 29 - protein, 569 aa, Artificial Sequence
  Description of Artificial Sequence: Synthetic polypeptide
ETVHCDLQPVGPERDEVTYTTSQVSKGCVAQAPNAILEVHVLFLEFPTGPSQLELTLQASKQNGTWPREVLLVLSVNSSVFLHLQALGIPLHLAYNSSLVTFQEPPGVNTTELPSFPKTQILEWAAERGPITSAAELNDPQSILLRLGQAQGSLSFCMLEASQDMGRTLEWRPRTPALVRGCHLEGVAGHKEAHILRVLPGHSAGPRTVTVKVELSCAPGDLDAVLILQGPPYVSWLIDANHNMQIWTTGEYSFKIFPEKNIRGFKLPDTPQGLLGEARMLNASIVASFVELPLASIVSLHASSCGGRLQTSPAPIQTTPPKDTCSPELLMSLITGGGPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 30 - protein, 563 aa, Artificial Sequence
  Description of Artificial Sequence: Synthetic polypeptide
ETVHCDLQPVGPERDEVTYTTSQVSKGCVAQAPNAILEVHVLFLEFPTGPSQLELTLQASKQNGTWPREVLLVLSVNSSVFLHLQALGIPLHLAYNSSLVTFQEPPGVNTTELPSFPKTQILEWAAERGPITSAAELNDPQSILLRLGQAQGSLSFCMLEASQDMGRTLEWRPRTPALVRGCHLEGVAGHKEAHILRVLPGHSAGPRTVTVKVELSCAPGDLDAVLILQGPPYVSWLIDANHNMQIWTTGEYSFKIFPEKNIRGFKLPDTPQGLLGEARMLNASIVASFVELPLASIVSLHASSCGGRLQTSPAPIQTTPPKDTCSPELLMSLITGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - protein, 550 aa, Artificial Sequence
  Description of Artificial Sequence: Synthetic polypeptide
ETVHCDLQPVGPERDEVTYTTSQVSKGCVAQAPNAILEVHVLFLEFPTGPSQLELTLQASKQNGTWPREVLLVLSVNSSVFLHLQALGIPLHLAYNSSLVTFQEPPGVNTTELPSFPKTQILEWAAERGPITSAAELNDPQSILLRLGQAQGSLSFCMLEASQDMGRTLEWRPRTPALVRGCHLEGVAGHKEAHILRVLPGHSAGPRTVTVKVELSCAPGDLDAVLILQGPPYVSWLIDANHNMQIWTTGEYSFKIFPEKNIRGFKLPDTPQGLLGEARMLNASIVASFVELPLASIVSLHASSCGGRLQTSPAPIQTTPPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 32 - protein, 424 aa, Homo sapiens
MGSLVLTLCALFCLAAYLVSGSPIMNLEQSPLEEDMSLFGDVFSEQDGVDFNTLLQSMKDEFLKTLNLSDIPTQDSAKVDPPEYMLELYNKFATDRTSMPSANIIRSFKNEDLFSQPVSFNGLRKYPLLFNVSIPHHEEVIMAELRLYTLVQRDRMIYDGVDRKITIFEVLESKGDNEGERNMLVLVSGEIYGTNSEWETFDVTDAIRRWQKSGSSTHQLEVHIESKHDEAEDASSGRLEIDTSAQNKHNPLLIVFSDDQSSDKERKEELNEMISHEQLPELDNLGLDSFSSGPGEEALLQMRSNIIYDSTARIRRNAKGNYCKRTPLYIDFKEIGWDSWIIAPPGYEAYECRGVCNYPLAEHLTPTKHAIIQALVHLKNSQKASKACCVPTKLEPISILYLDKGVVTYKFKYEGMAVSECGCR

SEQ ID NO: 33 - DNA, 1272 bp, Homo sapiens
atgggctctctggtcctgacactgtgcgctcttttctgcctggcagcttacttggtttctggcagccccatcatgaacctagagcagtctcctctggaagaagatatgtccctctttggtgatgttttctcagagcaagacggtgtcgactttaacacactgctccagagcatgaaggatgagtttcttaagacactaaacctctctgacatccccacgcaggattcagccaaggtggacccaccagagtacatgttggaactctacaacaaatttgcaacagatcggacctccatgccctctgccaacatcattaggagtttcaagaatgaagatctgttttcccagccggtcagttttaatgggctccgaaaataccccctcctcttcaatgtgtccattcctcaccatgaagaggtcatcatggctgaacttaggctatacacactggtgcaaagggatcgtatgatatacgatggagtagaccggaaaattaccatttttgaagtgctggagagcaaaggggataatgagggagaaagaaacatgctggtcttggtgtctggggagatatatggaaccaacagtgagtgggagacttttgatgtcacagatgccatcagacgttggcaaaagtcaggctcatccacccaccagctggaggtccacattgagagcaaacacgatgaagctgaggatgccagcagtggacggctagaaatagataccagtgcccagaataagcataaccctttgctcatcgtgttttctgatgaccaaagcagtgacaaggagaggaaggaggaactgaatgaaatgatttcccatgagcaacttccagagctggacaacttgggcctggatagcttttccagtggacctggggaagaggctttgttgcagatgagatcaaacatcatctatgactccactgcccgaatcagaaggaacgccaaaggaaactactgtaagaggaccccgctctacatcgacttcaaggagattgggtgggactcctggatcatcgctccgcctggatacgaagcctatgaatgccgtggtgtttgtaactaccccctggcagagcatctcacacccacaaagcatgcaattatccaggccttggtccacctcaagaattcccagaaagcttccaaagcctgctgtgtgcccacaaagctagagcccatctccatcctctatttagacaaaggcgtcgtcacctacaagtttaaatacgaaggcatggccgtctccgaatgtggctgtaga

SEQ ID NO: 34 - protein, 296 aa, Homo sapiens
GSPIMNLEQSPLEEDMSLFGDVFSEQDGVDFNTLLQSMKDEFLKTLNLSDIPTQDSAKVDPPEYMLELYNKFATDRTSMPSANIIRSFKNEDLFSQPVSFNGLRKYPLLFNVSIPHHEEVIMAELRLYTLVQRDRMIYDGVDRKITIFEVLESKGDNEGERNMLVLVSGEIYGTNSEWETFDVTDAIRRWQKSGSSTHQLEVHIESKHDEAEDASSGRLEIDTSAQNKHNPLLIVFSDDQSSDKERKEELNEMISHEQLPELDNLGLDSFSSGPGEEALLQMRSNIIYDSTARIRR

SEQ ID NO: 35 - DNA, 885 bp, Homo sapiens
agccccatcatgaacctagagcagtctcctctggaagaagatatgtccctctttggtgatgttttctcagagcaagacggtgtcgactttaacacactgctccagagcatgaaggatgagtttcttaagacactaaacctctctgacatccccacgcaggattcagccaaggtggacccaccagagtacatgttggaactctacaacaaatttgcaacagatcggacctccatgccctctgccaacatcattaggagtttcaagaatgaagatctgttttcccagccggtcagttttaatgggctccgaaaataccccctcctcttcaatgtgtccattcctcaccatgaagaggtcatcatggctgaacttaggctatacacactggtgcaaagggatcgtatgatatacgatggagtagaccggaaaattaccatttttgaagtgctggagagcaaaggggataatgagggagaaagaaacatgctggtcttggtgtctggggagatatatggaaccaacagtgagtgggagacttttgatgtcacagatgccatcagacgttggcaaaagtcaggctcatccacccaccagctggaggtccacattgagagcaaacacgatgaagctgaggatgccagcagtggacggctagaaatagataccagtgcccagaataagcataaccctttgctcatcgtgttttctgatgaccaaagcagtgacaaggagaggaaggaggaactgaatgaaatgatttcccatgagcaacttccagagctggacaacttgggcctggatagcttttccagtggacctggggaagaggctttgttgcagatgagatcaaacatcatctatgactccactgcccgaatcagaagg

SEQ ID NO: 36 - protein, 225 aa, Homo sapiens
THTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 37 - protein, 223 aa, Homo sapiens
VECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 38 - protein, 232 aa, Homo sapiens
EPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 39 - protein, 279 aa, Homo sapiens
ELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 40 - protein, 229 aa, Homo sapiens
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 41 - protein, 3 aa, Artificial Sequence
  Description of Artificial Sequence: Synthetic peptide
GGG

SEQ ID NO: 42 - protein, 4 aa, Artificial Sequence
  Description of Artificial Sequence: Synthetic peptide
GGGG

SEQ ID NO: 43 - protein, 5 aa, Artificial Sequence
  Description of Artificial Sequence: Synthetic peptide
TGGGG

SEQ ID NO: 44 - protein, 5 aa, Artificial Sequence
  Description of Artificial Sequence: Synthetic peptide
SGGGG

SEQ ID NO: 45 - protein, 4 aa, Artificial Sequence
  Description of Artificial Sequence: Synthetic peptide
TGGG

SEQ ID NO: 46 - protein, 4 aa, Artificial Sequence
  Description of Artificial Sequence: Synthetic peptide
SGGG

SEQ ID NO: 47 - protein, 5 aa, Artificial Sequence
  Description of Artificial Sequence: Synthetic peptide
GGGGS

SEQ ID NO: 48-49 - skipped (2 entries)

SEQ ID NO: 50 - protein, 344 aa, Artificial Sequence
  Description of Artificial Sequence: Synthetic polypeptide
ILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEMEVTQPTSNPVTPKPPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPVPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 51 - protein, 21 aa, Apis sp.
MKFLVNVALVFMVVYISYIYA

SEQ ID NO: 52 - protein, 22 aa, Unknown
  Description of Unknown: Tissue plasminogen activator peptide
MDAMKRGLCCVLLLCGAVFVSP

SEQ ID NO: 53 - protein, 20 aa, Unknown
  Description of Unknown: Native peptide
MGAAAKLAFAVFLISCSSGA

SEQ ID NO: 54 - protein, 369 aa, Artificial Sequence
  Description of Artificial Sequence: Synthetic polypeptide
MDAMKRGLCCVLLLCGAVFVSPGAAILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEMEVTQPTSNPVTPKPPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPVPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 55 - DNA, 1114 bp, Artificial Sequence
  Description of Artificial Sequence: Synthetic polynucleotide
atggatgcaatgaagagagggctctgctgtgtgctgctgctgtgtggagcagtcttcgtttcgcccggcgccgctatacttggtagatcagaaactcaggagtgtctttttttaatgctaattgggaaaaagacagaaccaatcaaactggtgttgaaccgtgttatggtgacaaagataaacggcggcattgttttgctacctggaagaatatttctggttccattgaatagtgaaacaaggttgttggctggatgatatcaactgctatgacaggactgattgtgtagaaaaaaaagacagccctgaagtatatttctgttgctgtgagggcaatatgtgtaatgaaaagttttcttattttccggagatggaagtcacacagcccacttcaaatccagttacacctaagccacccaccggtggtggaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagtccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatgagaattc

SEQ ID NO: 56 - protein, 9 aa, Artificial Sequence
  Description of Artificial Sequence: Synthetic peptide
ILGRSETQE

SEQ ID NO: 57 - protein, 329 aa, Artificial Sequence
  Description of Artificial Sequence: Synthetic polypeptide
ILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEMTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPVPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 58 - protein, 343 aa, Artificial Sequence
  Description of Artificial Sequence: Synthetic polypeptide
GRGEAETRECIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPTAPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPVPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 59 - protein, 20 aa, Unknown
  Description of Unknown: Native peptide
MGAAAKLAFAVFLISCSSGA

SEQ ID NO: 60 - protein, 368 aa, Artificial Sequence
  Description of Artificial Sequence: Synthetic polypeptide
MDAMKRGLCCVLLLCGAVFVSPGASGRGEAETRECIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPTAPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPVPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 61 - DNA, 1107 bp, Artificial Sequence
  Description of Artificial Sequence: Synthetic polynucleotide
atggatgcaatgaagagagggctctgctgtgtgctgctgctgtgtggagcagtcttcgtttcgcccggcgcctctgggcgtggggaggctgagacacgggagtgcatctactacaacgccaactgggagctggagcgcaccaaccagagcggcctggagcgctgcgaaggcgagcaggacaagcggctgcactgctacgcctcctggcgcaacagctctggcaccatcgagctcgtgaagaagggctgctggctagatgacttcaactgctacgataggcaggagtgtgtggccactgaggagaacccccaggtgtacttctgctgctgtgaaggcaacttctgcaacgagcgcttcactcatttgccagaggctgggggcccggaagtcacgtacgagccacccccgacagcccccaccggtggtggaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagtccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 62 - protein, 6 aa, Artificial Sequence
  Description of Artificial Sequence: Synthetic peptide
GRGEAE

SEQ ID NO: 63 - protein, 335 aa, Artificial Sequence
  Description of Artificial Sequence: Synthetic polypeptide
ETRECIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 64 - protein, 343 aa, Artificial Sequence
  Description of Artificial Sequence: Synthetic polypeptide
GRGEAETRECIYYNANWELERTNQSGLERCEGEQDKRLHCYASWRNSSGTIELVKKGCWDDDFNCYDRQECVATEENPQVYFCCCEGNFCNERFTHLPEAGGPEVTYEPPPTAPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPVPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 65 - protein, 115 aa, Artificial Sequence
  Description of Artificial Sequence: Synthetic polypeptide
GRGEAETRECIYYNANWELERTNQSGLERCEGEQDKRLHCYASWRNSSGTIELVKKGCWDDDFNCYDRQECVATEENPQVYFCCCEGNFCNERFTHLPEAGGPEVTYEPPPTAPT

SEQ ID NO: 66 - protein, 368 aa, Artificial Sequence
  Description of Artificial Sequence: Synthetic polypeptide
MDAMKRGLCCVLLLCGAVFVSPGASGRGEAETRECIYYNANWELERTNQSGLERCEGEQDKRLHCYASWRNSSGTIELVKKGCWDDDFNCYDRQECVATEENPQVYFCCCEGNFCNERFTHLPEAGGPEVTYEPPPTAPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 67 - DNA, 1107 bp, Artificial Sequence
  Description of Artificial Sequence: Synthetic polynucleotide
atggatgcaatgaagagagggctctgctgtgtgctgctgctgtgtggagcagtcttcgtttcgcccggcgcctctgggcgtggggaggctgagacacgggagtgcatctactacaacgccaactgggagctggagcgcaccaaccagagcggcctggagcgctgcgaaggcgagcaggacaagcggctgcactgctacgcctcctggcgcaacagctctggcaccatcgagctcgtgaagaagggctgctgggacgatgacttcaactgctacgataggcaggagtgtgtggccactgaggagaacccccaggtgtacttctgctgctgtgaaggcaacttctgcaacgagcgcttcactcatttgccagaggctgggggcccggaagtcacgtacgagccacccccgacagcccccaccggtggtggaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagtccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 68 - protein, 26 aa, Homo sapiens
MTSSLQRPWRVPWLPWTILLVSTAAA

SEQ ID NO: 69 - protein, 376 aa, Artificial Sequence
  Description of Artificial Sequence: Synthetic polypeptide
MDAMKRGLCCVLLLCGAVFVSPGASQNQERLCAFKDPYQQDLGIGESRISHENGTILCSKGSTCYGLWEKSKGDINLVKQGCWSHIGDPQECHYEECVVTTTPPSIQNGTYRFCCCSTDLCNVNFTENFPPPDTTPLSPPHSFNRDETGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 70 - DNA, 1128 bp, Artificial Sequence
  Description of Artificial Sequence: Synthetic polynucleotide
atggatgcaatgaagagagggctctgctgtgtgctgctgctgtgtggagcagtcttcgtttcgcccggcgcctcgcagaatcaagaacgcctatgtgcgtttaaagatccgtatcagcaagaccttgggataggtgagagtagaatctctcatgaaaatgggacaatattatgctcgaaaggtagcacctgctatggcctttgggagaaatcaaaaggggacataaatcttgtaaaacaaggatgttggtctcacattggagatccccaagagtgtcactatgaagaatgtgtagtaactaccactcctccctcaattcagaatggaacataccgtttctgctgttgtagcacagatttatgtaatgtcaactttactgagaattttccacctcctgacacaacaccactcagtccacctcattcatttaaccgagatgagaccggtggtggaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 71 - protein, 352 aa, Artificial Sequence
  Description of Artificial Sequence: Synthetic polypeptide
SQNQERLCAFKDPYQQDLGIGESRISHENGTILCSKGSTCYGLWEKSKGDINLVKQGCWSHIGDPQECHYEECVVTTTPPSIQNGTYRFCCCSTDLCNVNFTENFPPPDTTPLSPPHSFNRDETGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 72 - DNA, 1056 bp, Artificial Sequence
  Description of Artificial Sequence: Synthetic polynucleotide
tcgcagaatcaagaacgcctatgtgcgtttaaagatccgtatcagcaagaccttgggataggtgagagtagaatctctcatgaaaatgggacaatattatgctcgaaaggtagcacctgctatggcctttgggagaaatcaaaaggggacataaatcttgtaaaacaaggatgttggtctcacattggagatccccaagagtgtcactatgaagaatgtgtagtaactaccactcctccctcaattcagaatggaacataccgtttctgctgttgtagcacagatttatgtaatgtcaactttactgagaattttccacctcctgacacaacaccactcagtccacctcattcatttaaccgagatgagaccggtggtggaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 73 - protein, 21 aa, Unknown
  Description of Unknown: Native peptide
MTLGSPRKGLLMLLMALVTQG

SEQ ID NO: 74 - protein, 352 aa, Artificial Sequence
  Description of Artificial Sequence: Synthetic polypeptide
MDAMKRGLCCVLLLCGAVFVSPGADPVKPSRGPLVTCTCESPHCKGPTCRGAWCTVVLVREEGRHPQEHRGCGNLHRELCRGRPTEFVNHYCCDSHLCNHNVSLVLEATQPPSEQPGTDGQLATGGGTHTCPPCPAPEALGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPVPIEKTISKAKGQPREPQVYTLPPSREEMTKNQVSLTCLVKGFYPSDIAVEWESNGQPENNYKTTPPVLDSDGPFFLYSKLTVDKSRWQQGNVFSCSVMHEALHNHYTQKSLSLSPGK

SEQ ID NO: 75 - skipped

SEQ ID NO: 76 - protein, 328 aa, Artificial Sequence
  Description of Artificial Sequence: Synthetic polypeptide
DPVKPSRGPLVTCTCESPHCKGPTCRGAWCTVVLVREEGRHPQEHRGCGNLHRELCRGRPTEFVNHYCCDSHLCNHNVSLVLEATQPPSEQPGTDGQLATGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPVPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 77 - protein, 25 aa, Homo sapiens
MDRGTLPLAVALLLASCSLSPTSLA

SEQ ID NO: 78 - protein, 820 aa, Artificial Sequence [sequence omitted]

SEQ ID NO: 79 - DNA, 2463 bp, Artificial Sequence
  Description of Artificial Sequence: Synthetic polynucleotide
atggatgcaatgaagagagggctctgctgtgtgctgctgctgtgtggagcagtcttcgtttcgcccggcgccgaaacagtccattgtgaccttcagcctgtgggccccgagagggacgaggtgacatataccactagccaggtctcgaagggctgcgtggctcaggcccccaatgccatccttgaagtccatgtcctcttcctggagttcccaacgggcccgtcacagctggagctgactctccaggcatccaagcaaaatggcacctggccccgagaggtgcttctggtcctcagtgtaaacagcagtgtcttcctgcatctccaggccctgggaatcccactgcacttggcctacaattccagcctggtcaccttccaagagcccccgggggtcaacaccacagagctgccatccttccccaagacccagatccttgagtgggcagctgagaggggccccatcacctctgctgctgagctgaatgacccccagagcatcctcctccgactgggccaagcccaggggtcactgtccttctgcatgctggaagccagccaggacatgggccgcacgctcgagtggcggccgcgtactccagccttggtccggggctgccacttggaaggcgtggccggccacaaggaggcgcacatcctgagggtcctgccgggccactcggccgggccccggacggtgacggtgaaggtggaactgagctgcgcacccggggatctcgatgccgtcctcatcctgcagggtcccccctacgtgtcctggctcatcgacgccaaccacaacatgcagatctggaccactggagaatactccttcaagatctttccagagaaaaacattcgtggcttcaagctcccagacacacctcaaggcctcctgggggaggcccggatgctcaatgccagcattgtggcatccttcgtggagctaccgctggccagcattgtctcacttcatgcctccagctgcggtggtaggctgcagacctcacccgcaccgatccagaccactcctcccaaggacacttgtagcccggagctgctcatgtccttgatccagacaaagtgtgccgacgacgccatgaccctggtactaaagaaagagcttgttgcgcatttgaagtgcaccatcacgggcctgaccttctgggaccccagctgtgaggcagaggacaggggtgacaagtttgtcttgcgcagtgcttactccagctgtggcatgcaggtgtcagcaagtatgatcagcaatgaggcggtggtcaatatcctgtcgagctcatcaccacagcggaaaaaggtgcactgcctcaacatggacagcctctctttccagctgggcctctacctcagcccacacttcctccaggcctccaacaccatcgagccggggcagcagagctttgtgcaggtcagagtgtccccatccgtctccgagttcctgctccagttagacagctgccacctggacttggggcctgagggaggcaccgtggaactcatccagggccgggcggccaagggcaactgtgtgagcctgctgtccccaagccccgagggtgacccgcgcttcagcttcctcctccacttctacacagtacccatacccaaaaccggcaccctcagctgcacggtagccctgcgtcccaagaccgggtctcaagaccaggaagtccataggactgtcttcatgcgcttgaacatcatcagccctgacctgtctggttgcacaagcaaaggcaccggtggtggacccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatga

SEQ ID NO: 80 - protein, 814 aa, Artificial Sequence
  Description of Artificial Sequence: Synthetic polypeptide
MDAMKRGLCCVLLLCGAVFVSPGAETVHCDLQPVGPERDEVTYTTSQVSKGCVAQAPNAILEVHVLFLEFPTGPSQLELTLQASKQNGTWPREVLLVLSVNSSVFLHLQALGIPLHLAYNSSLVTFQEPPGVNTTELPSFPKTQILEWAAERGPITSAAELNDPQSILLRLGQAQGSLSFCMLEASQDMGRTLEWRPRTPALVRGCHLEGVAGHKEAHILRVLPGHSAGPRTVTVKVELSCAPGDLDAVLILQGPPYVSWLIDANHNMQIWTTGEYSFKIFPEKNIRGFKLPDTPQGLLGEARMLNASIVASFVELPLASIVSLHASSCGGRLQTSPAPIQTTPPKDTCSPELLMSLIQTKCADDAMTLVLKKELVAHLKCTITGLTFWDPSCEAEDRGDKFVLRSAYSSCGMQVSASMISNEAVVNILSSSSPQRKKVHCLNMDSLSFQLGLYLSPHFLQASNTIEPGQQSFVQVRVSPSVSEFLLQLDSCHLDLGPEGGTVELIQGRAAKGNCVSLLSPSPEGDPRFSFLLHFYTVPIPKTGTLSCTVALRPKTGSQDQEVHRTVFMRLNIISPDLSGCTSKGTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 81 - protein, 21 aa, Homo sapiens
MGSLVLTLCALFCLAAYLVSG

SEQ ID NO: 82 - protein, 547 aa, Artificial Sequence
  Description of Artificial Sequence: Synthetic polypeptide
MDAMKRGLCCVLLLCGAVFVSPGASPIMNLEQSPLEEDMSLFGDVFSEQDGVDFNTLLQSMKDEFLKTLNLSDIPTQDSAKVDPPEYMLELYNKFATDRTSMPSANIIRSFKNEDLFSQPVSFNGLRKYPLLFNVSIPHHEEVIMAELRLYTLVQRDRMIYDGVDRKITIFEVLESKGDNEGERNMLVLVSGEIYGTNSEWETFDVTDAIRRWQKSGSSTHQLEVHIESKHDEAEDASSGRLEIDTSAQNKHNPLLIVFSDDQSSDKERKEELNEMISHEQLPELDNLGLDSFSSGPGEEALLQMRSNIIYDSTARIRTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 83 - DNA, 1644 bp, Artificial Sequence
  Description of Artificial Sequence: Synthetic polynucleotide
atggatgcaatgaagagagggctctgctgtgtgctgctgctgtgtggagcagtcttcgtttcgcccggcgccagccccatcatgaacctagagcagtctcctctggaagaagatatgtccctctttggtgatgttttctcagagcaagacggtgtcgactttaacacactgctccagagcatgaaggatgagtttcttaagacactaaacctctctgacatccccacgcaggattcagccaaggtggacccaccagagtacatgttggaactctacaacaaatttgcaacagatcggacctccatgccctctgccaacatcattaggagtttcaagaatgaagatctgttttcccagccggtcagttttaatgggctccgaaaataccccctcctcttcaatgtgtccattcctcaccatgaagaggtcatcatggctgaacttaggctatacacactggtgcaaagggatcgtatgatatacgatggagtagaccggaaaattaccatttttgaagtgctggagagcaaaggggataatgagggagaaagaaacatgctggtcttggtgtctggggagatatatggaaccaacagtgagtgggagacttttgatgtcacagatgccatcagacgttggcaaaagtcaggctcatccacccaccagctggaggtccacattgagagcaaacacgatgaagctgaggatgccagcagtggacggctagaaatagataccagtgcccagaataagcataaccctttgctcatcgtgttttctgatgaccaaagcagtgacaaggagaggaaggaggaactgaatgaaatgatttcccatgagcaacttccagagctggacaacttgggcctggatagcttttccagtggacctggggaagaggctttgttgcagatgagatcaaacatcatctatgactccactgcccgaatcagaaccggtggtggaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 84 - protein, 523 aa, Artificial Sequence
  Description of Artificial Sequence: Synthetic polypeptide
SPIMNLEQSPLEEDMSLFGDVFSEQDGVDFNTLLQSMKDEFLKTLNLSDIPTQDSAKVDPPEYMLELYNKFATDRTSMPSANIIRSFKNEDLFSQPVSFNGLRKYPLLFNVSIPHHEEVIMAELRLYTLVQRDRMIYDGVDRKITIFEVLESKGDNEGERNMLVLVSGEIYGTNSEWETFDVTDAIRRWQKSGSSTHQLEVHIESKHDEAEDASSGRLEIDTSAQNKHNPLLIVFSDDQSSDKERKEELNEMISHEQLPELDNLGLDSFSSGPGEEALLQMRSNIIYDSTARIRTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 85 - protein, 544 aa, Artificial Sequence
  Description of Artificial Sequence: Synthetic polypeptide
MDAMKRGLCCVLLLCGAVFVSPGASPIMNLEQSPLEEDMSLFGDVFSEQDGVDFNTLLQSMKDEFLKTLNLSDIPTQDSAKVDPPEYMLELYNKFATDRTSMPSANIIRSFKNEDLFSQPVSFNGLRKYPLLFNVSIPHHEEVIMAELRLYTLVQRDRMIYDGVDRKITIFEVLESKGDNEGERNMLVLVSGEIYGTNSEWETFDVTDAIRRWQKSGSSTHQLEVHIESKHDEAEDASSGRLEIDTSAQNKHNPLLIVFSDDQSSDKERKEELNEMISHEQLPELDNLGLDSFSSGPGEEALLQMRSNIIYDSTATGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 86 - DNA, 1635 bp, Artificial Sequence
  Description of Artificial Sequence: Synthetic polynucleotide
atggatgcaatgaagagagggctctgctgtgtgctgctgctgtgtggagcagtcttcgtttcgcccggcgccagccccatcatgaacctagagcagtctcctctggaagaagatatgtccctctttggtgatgttttctcagagcaagacggtgtcgactttaacacactgctccagagcatgaaggatgagtttcttaagacactaaacctctctgacatccccacgcaggattcagccaaggtggacccaccagagtacatgttggaactctacaacaaatttgcaacagatcggacctccatgccctctgccaacatcattaggagtttcaagaatgaagatctgttttcccagccggtcagttttaatgggctccgaaaataccccctcctcttcaatgtgtccattcctcaccatgaagaggtcatcatggctgaacttaggctatacacactggtgcaaagggatcgtatgatatacgatggagtagaccggaaaattaccatttttgaagtgctggagagcaaaggggataatgagggagaaagaaacatgctggtcttggtgtctggggagatatatggaaccaacagtgagtgggagacttttgatgtcacagatgccatcagacgttggcaaaagtcaggctcatccacccaccagctggaggtccacattgagagcaaacacgatgaagctgaggatgccagcagtggacggctagaaatagataccagtgcccagaataagcataaccctttgctcatcgtgttttctgatgaccaaagcagtgacaaggagaggaaggaggaactgaatgaaatgatttcccatgagcaacttccagagctggacaacttgggcctggatagcttttccagtggacctggggaagaggctttgttgcagatgagatcaaacatcatctatgactccactgccaccggtggtggaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 87 - protein, 520 aa, Artificial Sequence
  Description of Artificial Sequence: Synthetic polypeptide
SPIMNLEQSPLEEDMSLFGDVFSEQDGVDFNTLLQSMKDEFLKTLNLSDIPTQDSAKVDPPEYMLELYNKFATDRTSMPSANIIRSFKNEDLFSQPVSFNGLRKYPLLFNVSIPHHEEVIMAELRLYTLVQRDRMIYDGVDRKITIFEVLESKGDNEGERNMLVLVSGEIYGTNSEWETFDVTDAIRRWQKSGSSTHQLEVHIESKHDEAEDASSGRLEIDTSAQNKHNPLLIVFSDDQSSDKERKEELNEMISHEQLPELDNLGLDSFSSGPGEEALLQMRSNIIYDSTATGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 88-99 - skipped (12 entries)

SEQ ID NO: 100 - protein, 150 aa, Rattus sp.
MTAPWAALALLWGSLCAGSGRGEAETRECIYYNANWELERTNQSGLERCEGEQDKRLHCYASWPNSSGTIELVKKGCWLDDFNCYDRQECVATEENPQVYFCCCEGNFCNERFTHLPEPGGPEVTYEPPPTAPTLLTVLAYSLLPIGGLS

SEQ ID NO: 101 - protein, 150 aa, Sus scrofa
MTAPWAALALLWGSLCVGSGRGEAETRECIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPTAPTLLTVLAYSLLPIGGLS

SEQ ID NO: 102 - protein, 150 aa, Mus musculus
MTAPWAALALLWGSLCAGSGRGEAETRECIYYNANWELERTNQSGLERCEGEQDKRLHCYASWRNSSGTIELVKKGCWLDDFNCYDRQECVATEENPQVYFCCCEGNFCNERFTHLPEPGGPEVTYEPPPTAPTLLTVLAYSLLPIGGLS

SEQ ID NO: 103 - protein, 150 aa, Homo sapiens
MTAPWVALALLWGSLCAGSGRGEAETRECIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPTAPTLLTVLAYSLLPIGGLS

SEQ ID NO: 104 - protein, 150 aa, Bos taurus
MTAPWAALALLWGSLCAGSGRGEAETRECIYYNANWELERTNQSGLERCEGERDKRLHCYASWRNSSGTIELVKKGCWLDDFNCYDRQECVATEENPQVYFCCCEGNFCNERFTHLPEAGGPEVTYEPPPTAPTLLTVLAYSLLPVGGLS

SEQ ID NO: 105 - protein, 150 aa, Xenopus sp.
MGASVALTFLLLLATFRAGSGHDEVETRECIYYNANWELEKTNQSGVERLVEGKKDKRLHCYASWRNNSGFIELVKKGCWLDDFNCYDRQECIAKEENPQVFFCCCEGNYCNKKFTHLPEVETFDPKPQPSASVLNILIYSLLPIVGLSM

SEQ ID NO: 106 - protein, 154 aa, Artificial Sequence
  Description of Artificial Sequence: Synthetic consensus sequence
  MOD_RES (8)..(8): Thr, Ala or absent
  MOD_RES (121)..(121): Pro, Ala, Val or Met
MTAPWAAXLALLWGSLCAGSGRGEAETRECIYYNANWELERTNQSGLERLCEGEQDKRLHCYASWRNSSGTIELVKKGCWLDDFNCYDRQECVATEENPQVYFCCCEGNFCNERFTHLPEXGGPEVTYEPKPPTAPTLLTVLAYSLLPIGGLSM

SEQ ID NO: 107 - protein, 115 aa, Homo sapiens; identical to SEQ ID NO: 10

SEQ ID NO: 108 - protein, 115 aa, Ovis aries
ILGRSETQECIFYNANWERDRTNRTGVESCYGDKDKRRHCFATWKNISGSIDIVKQGCWLDDINCYDRTDCIEKKDSPEVYFCCCEGNMCNERFSYFPEMEVTQPTSNPVTPKPP

SEQ ID NO: 109 - protein, 115 aa, Condylura cristata
ILGRSETQECLFFNANWERDRTNQTGVEPCYGDKDKRRHCFATWKNISGSIEIVKQGCWLDDINCYDRTDCIEKKDSPEVYFCCCEGNMCNEKFSYFPEMEVTQPTSNPVTPKAP

SEQ ID NO: 110 - protein, 115 aa, Mus musculus
ILGRSETQECLFFNANWERDRTNQTGVEPCYGDKDKRRHCFATWKNISGSIEIVKQGCWLDDINCYDRTDCIEKKDSPEVYFCCCEGNMCNEKFSYFPEMEVTQPTSNPVTPKPP

SEQ ID NO: 111 - protein, 115 aa, Gallus gallus
ILGRSETQECIYYNANWEKDKTNRSGIEPCYGDKDKRRHCFATWKNISGSIEIVKQGCWLDDINCYDRNDCIEKKDSPEVFFCCCEGNMCNERFFYFPEMEVTQPTSNPVTPKPP

SEQ ID NO: 112 - protein, 115 aa, Bos taurus
ILGRSETQECIFYNANWERDRTNRTGVESCYGDKDKRRHCFATWKNISGSIEIVKQGCWLDDINCYDRTDCIEKKDSPEVYFCCCEGNMCNERFSYFPEMEVTQPTSNPVTPKPP

SEQ ID NO: 113 - protein, 115 aa, Tyto alba; identical to SEQ ID NO: 111

SEQ ID NO: 114 - protein, 115 aa, Myotis davidii
ILGRSETQECIFYNANWERDKTNRTGVELCYGDKDKRRHCFATWKNISGSIEIVKQGCWLDDINCYDRTDCIEKKDSPEVYFCCCEGNMCNERFSYFPEMEVTQPTSNPVTPKPP

SEQ ID NO: 115-121 - skipped (7 entries)

SEQ ID NO: 122 - protein, 150 aa, Homo sapiens
MGAAAKLAFAVFLISCSSGAILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEMEVTQPTSNPVTPKPPYYNILLYSLVPLMLI

SEQ ID NO: 123 - protein, 360 aa, Artificial Sequence
  Description of Artificial Sequence: Synthetic polypeptide
MDAMKRGLCCVLLLCGAVFVSPGAAETRECIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 124 - DNA, 1083 bp, Artificial Sequence
  Description of Artificial Sequence: Synthetic polynucleotide
  CDS (73)..(396)
atggatgcaatgaagagagggctctgctgtgtgctgctgctgtgtggagcagtcttcgtttcgcccggcgccgctgagacacgggagtgcatctactacaacgccaactgggagctggagcgcaccaaccagagcggcctggagcgctgcgaaggcgagcaggacaagcggctgcactgctacgcctcctggcgcaacagctctggcaccatcgagctcgtgaagaagggctgctggctagatgacttcaactgctacgataggcaggagtgtgtggccactgaggagaacccccaggtgtacttctgctgctgtgaaggcaacttctgcaacgagcgcttcactcatttgccagaggctgggggcccggaagtcacgtacgagccacccccgacaggtggtggaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatga

SEQ ID NO: 125 - DNA, 1083 bp, Artificial Sequence
  Description of Artificial Sequence: Synthetic polynucleotide
tcatttacccggggacagggagaggctcttctgcgtgtagtggttgtgcagagcctcatgcatcacggagcatgagaagacgttcccctgctgccacctgctcttgtccacggtgagcttgctatagaggaagaaggagccgtcggagtccagcacgggaggcgtggtcttgtagttgttctccggctgcccattgctctcccactccacggcgatgtcgctgggatagaagcctttgaccaggcaggtcaggctgacctggttcttggtcatctcctcccgggatgggggcagggtgtacacctgtggttctcggggctgccctttggctttggagatggttttctcgatgggggctgggagggctttgttggagaccttgcacttgtactccttgccattcagccagtcctggtgcaggacggtgaggacgctgaccacacggtacgtgctgttgtactgctcctcccgcggctttgtcttggcattatgcacctccacgccgtccacgtaccagttgaacttgacctcagggtcttcgtggctcacgtccaccaccacgcatgtgacctcaggggtccgggagatcatgagggtgtccttgggttttggggggaagaggaagactgacggtccccccaggagttcaggtgctgggcacggtgggcatgtgtgagttccaccacctgtcgggggtggctcgtacgtgacttccgggcccccagcctctggcaaatgagtgaagcgctcgttgcagaagttgccttcacagcagcagaagtacacctgggggttctcctcagtggccacacactcctgcctatcgtagcagttgaagtcatctagccagcagcccttcttcacgagctcgatggtgccagagctgttgcgccaggaggcgtagcagtgcagccgcttgtcctgctcgccttcgcagcgctccaggccgctctggttggtgcgctccagctcccagttggcgttgtagtagatgcactcccgtgtctcagcggcgccgggcgaaacgaagactgctccacacagcagcagcacacagcagagccctctcttcattgcatccat

SEQ ID NO: 126 - DNA, 1083 bp, Artificial Sequence
  Description of Artificial Sequence: Synthetic polynucleotide
  CDS (73)..(396)
atggatgcaatgaagagagggctctgctgtgtgctgctgctgtgtggagcagtcttcgtttcgcccggcgccgccgaaacccgcgaatgtatttattacaatgctaattgggaactcgaacggacgaaccaatccgggctcgaacggtgtgagggggaacaggataaacgcctccattgctatgcgtcgtggaggaactcctccgggacgattgaactggtcaagaaagggtgctggctggacgatttcaattgttatgaccgccaggaatgtgtcgcgaccgaagagaatccgcaggtctatttctgttgttgcgaggggaatttctgtaatgaacggtttacccacctccccgaagccggcgggcccgaggtgacctatgaacccccgcccaccggtggtggaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatga

SEQ ID NO: 127 - DNA, 1083 bp, Artificial Sequence
  Description of Artificial Sequence: Synthetic polynucleotide
tcatttacccggggacagggagaggctcttctgcgtgtagtggttgtgcagagcctcatgcatcacggagcatgagaagacgttcccctgctgccacctgctcttgtccacggtgagcttgctatagaggaagaaggagccgtcggagtccagcacgggaggcgtggtcttgtagttgttctccggctgcccattgctctcccactccacggcgatgtcgctgggatagaagcctttgaccaggcaggtcaggctgacctggttcttggtcatctcctcccgggatgggggcagggtgtacacctgtggttctcggggctgccctttggctttggagatggttttctcgatgggggctgggagggctttgttggagaccttgcacttgtactccttgccattcagccagtcctggtgcaggacggtgaggacgctgaccacacggtacgtgctgttgtactgctcctcccgcggctttgtcttggcattatgcacctccacgccgtccacgtaccagttgaacttgacctcagggtcttcgtggctcacgtccaccaccacgcatgtgacctcaggggtccgggagatcatgagggtgtccttgggttttggggggaagaggaagactgacggtccccccaggagttcaggtgctgggcacggtgggcatgtgtgagttccaccaccggtgggcgggggttcataggtcacctcgggcccgccggcttcggggaggtgggtaaaccgttcattacagaaattcccctcgcaacaacagaaatagacctgcggattctcttcggtcgcgacacattcctggcggtcataacaattgaaatcgtccagccagcaccctttcttgaccagttcaatcgtcccggaggagttcctccacgacgcatagcaatggaggcgtttatcctgttccccctcacaccgttcgagcccggattggttcgtccgttcgagttcccaattagcattgtaataaatacattcgcgggtttcggcggcgccgggcgaaacgaagactgctccacacagcagcagcacacagcagagccctctcttcattgcatccat

SEQ ID NO: 128-130 - skipped (3 entries)

SEQ ID NO: 131 - protein, 360 aa, Artificial Sequence
  Description of Artificial Sequence: Synthetic polypeptide
MDAMKRGLCCVLLLCGAVFVSPGAAETRECIYYNANWELERTNQSGLERCEGEQDKRLHCYASWRNSSGTIELVKKGCWDDDFNCYDRQECVATEENPQVYFCCCEGNFCNERFTHLPEAGGPEVTYEPPPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 132 - protein, 335 aa, Artificial Sequence
  Description of Artificial Sequence: Synthetic polypeptide
ETRECIYYNANWELERTNQSGLERCEGEQDKRLHCYASWRNSSGTIELVKKGCWDDDFNCYDRQECVATEENPQVYFCCCEGNFCNERFTHLPEAGGPEVTYEPPPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 133 - protein, 107 aa, Artificial Sequence
  Description of Artificial Sequence: Synthetic polypeptide
ETRECIYYNANWELERTNQSGLERCEGEQDKRLHCYASWRNSSGTIELVKKGCWDDDFNCYDRQECVATEENPQVYFCCCEGNFCNERFTHLPEAGGPEVTYEPPPT

SEQ ID NO: 134 - DNA, 1083 bp, Artificial Sequence
  Description of Artificial Sequence: Synthetic polynucleotide
  CDS (76)..(396)
atggatgcaatgaagagagggctctgctgtgtgctgctgctgtgtggagcagtcttcgtttcgcccggcgccgctgagacacgggagtgcatctactacaacgccaactgggagctggagcgcaccaaccagagcggcctggagcgctgcgaaggcgagcaggacaagcggctgcactgctacgcctcctggcgcaacagctctggcaccatcgagctcgtgaagaagggctgctgggacgatgacttcaactgctacgataggcaggagtgtgtggccactgaggagaacccccaggtgtacttctgctgctgtgaaggcaacttctgcaacgagcgcttcactcatttgccagaggctgggggcccggaagtcacgtacgagccacccccgacaggtggtggaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatga

SEQ ID NO: 135 - DNA, 1083 bp, Artificial Sequence
  Description of Artificial Sequence: Synthetic polynucleotide
tcatttacccggggacagggagaggctcttctgcgtgtagtggttgtgcagagcctcatgcatcacggagcatgagaagacgttcccctgctgccacctgctcttgtccacggtgagcttgctatagaggaagaaggagccgtcggagtccagcacgggaggcgtggtcttgtagttgttctccggctgcccattgctctcccactccacggcgatgtcgctgggatagaagcctttgaccaggcaggtcaggctgacctggttcttggtcatctcctcccgggatgggggcagggtgtacacctgtggttctcggggctgccctttggctttggagatggttttctcgatgggggctgggagggctttgttggagaccttgcacttgtactccttgccattcagccagtcctggtgcaggacggtgaggacgctgaccacacggtacgtgctgttgtactgctcctcccgcggctttgtcttggcattatgcacctccacgccgtccacgtaccagttgaacttgacctcagggtcttcgtggctcacgtccaccaccacgcatgtgacctcaggggtccgggagatcatgagggtgtccttgggttttggggggaagaggaagactgacggtccccccaggagttcaggtgctgggcacggtgggcatgtgtgagttccaccacctgtcgggggtggctcgtacgtgacttccgggcccccagcctctggcaaatgagtgaagcgctcgttgcagaagttgccttcacagcagcagaagtacacctgggggttctcctcagtggccacacactcctgcctatcgtagcagttgaagtcatcgtcccagcagcccttcttcacgagctcgatggtgccagagctgttgcgccaggaggcgtagcagtgcagccgcttgtcctgctcgccttcgcagcgctccaggccgctctggttggtgcgctccagctcccagttggcgttgtagtagatgcactcccgtgtctcagcggcgccgggcgaaacgaagactgctccacacagcagcagcacacagcagagccctctcttcattgcatccat

SEQ ID NO: 136 - DNA, 1083 bp, Artificial Sequence
  Description of Artificial Sequence: Synthetic polynucleotide
  CDS (76)..(396)
atggatgcaatgaagagagggctctgctgtgtgctgctgctgtgtggagcagtcttcgtttcgcccggcgccgccgaaacccgcgaatgtatttattacaatgctaattgggaactcgaacggacgaaccaatccgggctcgaacggtgtgagggggaacaggataaacgcctccattgctatgcgtcgtggaggaactcctccgggacgattgaactggtcaagaaagggtgctgggacgacgatttcaattgttatgaccgccaggaatgtgtcgcgaccgaagagaatccgcaggtctatttctgttgttgcgaggggaatttctgtaatgaacggtttacccacctccccgaagccggcgggcccgaggtgacctatgaacccccgcccaccggtggtggaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatga

SEQ ID NO: 137 - DNA, 1083 bp, Artificial Sequence
  Description of Artificial Sequence: Synthetic polynucleotide
tcatttacccggggacagggagaggctcttctgcgtgtagtggttgtgcagagcctcatgcatcacggagcatgagaagacgttcccctgctgccacctgctcttgtccacggtgagcttgctatagaggaagaaggagccgtcggagtccagcacgggaggcgtggtcttgtagttgttctccggctgcccattgctctcccactccacggcgatgtcgctgggatagaagcctttgaccaggcaggtcaggctgacctggttcttggtcatctcctcccgggatgggggcagggtgtacacctgtggttctcggggctgccctttggctttggagatggttttctcgatgggggctgggagggctttgttggagaccttgcacttgtactccttgccattcagccagtcctggtgcaggacggtgaggacgctgaccacacggtacgtgctgttgtactgctcctcccgcggctttgtcttggcattatgcacctccacgccgtccacgtaccagttgaacttgacctcagggtcttcgtggctcacgtccaccaccacgcatgtgacctcaggggtccgggagatcatgagggtgtccttgggttttggggggaagaggaagactgacggtccccccaggagttcaggtgctgggcacggtgggcatgtgtgagttccaccaccggtgggcgggggttcataggtcacctcgggcccgccggcttcggggaggtgggtaaaccgttcattacagaaattcccctcgcaacaacagaaatagacctgcggattctcttcggtcgcgacacattcctggcggtcataacaattgaaatcgtcgtcccagcaccctttcttgaccagttcaatcgtcccggaggagttcctccacgacgcatagcaatggaggcgtttatcctgttccccctcacaccgttcgagcccggattggttcgtccgttcgagttcccaattagcattgtaataaatacattcgcgggtttcggcggcgccgggcgaaacgaagactgctccacacagcagcagcacacagcagagccctctcttcattgcatccat

SEQ ID NO: 138 - protein, 4 aa, Homo sapiens
RIRR

SEQ ID NO: 139 - protein, 6 aa, Artificial Sequence
  Description of Artificial Sequence: Synthetic 6xHis tag
HHHHHH

SEQ ID NO: 140 - protein, 108 aa, Artificial Sequence
  Description of Artificial Sequence: Synthetic polypeptide
AETRECIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPT

(19) United States
(12) Patent Application Publication
(10) Pub. No.: US 2020/0087367 A1
(43) Pub. Date: Mar. 19, 2020

(54) COMPOSITIONS AND METHODS FOR TREATING HEART FAILURE

(71) Applicant: Acceleron Pharma Inc., Cambridge, MA (US)

(72) Inventors: Gang Li, Sudbury, MA (US); Asya Grinberg, Lexington, MA (US); Dianne Sako, Medford, MA (US)

(21) Appl. No.: 16/482,883

(22) PCT Filed: Feb. 5, 2018
(86) PCT No.: PCT/US2018/016794

§ 371 Date: Aug. 1, 2019

Related U.S. Application Data
(60) Provisional application No. 62/455,266, filed on Feb. 6, 2017.

(51) Int. Cl. C07K 14/51; C07K 14/71; C07K 14/705; C07K 16/22; A61P 9/04

(52) U.S. Cl. CPC C07K 14/51; C07K 14/71; A61K 45/06; C07K 16/22; A61P 9/04; C07K 14/70596

(57) ABSTRACT

In certain aspects, the present disclosure relates to methods of treating, preventing, and reducing the severity or progression of heart failure or one or more complications of heart failure. For example, the disclosure provides various BMP antagonists, such as ActRIIA polypeptides, ActRIIB polypeptides, BMPRII polypeptides, ALKI polypeptides, endoglin polypeptides, BMP 10 propeptide proteins and Fc fusion proteins thereof as well as anti-BMP9 antibodies, for treating, preventing and reducing the severity or progression of heart failure or one or more complications of heart failure.

### CROSS-REFERENCE TO RELATED APPLICATIONS

This application claims the benefit of priority from U.S. Provisional Application No. 62/455,266, filed Feb. 6, 2017. The specification of the foregoing application is incorporated herein by reference in its entirety.

### BACKGROUND OF THE INVENTION

It has been estimated that over five million people suffer from heart failure in the United States alone. Statistics from the American Heart Association also suggest that new cases of heart failure are diagnosed at a rate of about 550,000 each year. Of the newly diagnosed patients fifty percent are likely to die within five years from the initial diagnosis. Of course, these numbers do not account for the number of patients in other countries who also suffer from heart failure. Given these numbers, it is clear that heart failure is a significant human crisis.

Heart failure is a condition that is characterized, in part, by a reduced ability of the heart to circulate blood through the body. Typically, an underlying disease, such as high blood pressure (e.g., hypertension), clogged arteries (e.g., coronary artery disease), heart defect (e.g., cardiomyopathy, or valvular heart disease) or some other problem (e.g., diabetes, hyperthyroidism, or alcohol abuse) will lead to a decrease in circulation over time. As the heart works less efficiently, its capacity to circulate blood decreases and the body's requirements for oxygen are not met. The cardiac muscle tends to enlarge as the heart works harder over time to compensate for the decrease in efficiency.

Treatments typically include the use of a number of different pharmaceutical agents including, for example, angiotensin-converting (ACE) enzyme inhibitors, diuretics, beta-blockers, and surgical procedures. Although these treatments can improve some symptoms associated with heart failure, they are imperfect as many are associated with various side-effect and have limited efficacy on treating multiple manifestations of heart failure. The only permanent treatment for heart failure is heart transplant. Consequently, there is a need for additional therapeutics for the treatment of heart failure.

### SUMMARY OF THE INVENTION

In part, the data presented herein demonstrates that BMP antagonists (inhibitors) can be used to treat heart failure. For example, it was shown that a soluble BMP10 propeptide (BMP10pro) polypeptide can be used to prevent or reduce the severity of cardiac hypertrophy, cardiac remodeling, and cardiac fibrosis as well as improve cardiac function in a transverse aortic constriction (TAC) heart failure model. Moreover, BMP10pro treatment increased survival time of heart failure patients. In additional studies, the BMP10pro polypeptide was shown to prevent or reduce the severity of cardiac hypertrophy, cardiac remodeling, and cardiac fibrosis in a myocardial infarction (MI) heart failure model as well as increase survival time in these patient. Binding studies demonstrated that BMP10pro polypeptides have high affinity and can antagonize activity of BMP10. In addition, data of the disclosure show that BMP10pro polypeptides bind with high affinity to BMP9, BMP6, and BMP3b, and to a lesser extent BMP5. Furthermore, the experiments described herein demonstrate than a soluble endoglin polypeptide may be used to treat heart failure. For example, treatment with an endoglin polypeptide reduced the severity of cardiac hypertrophy, reduced cardiac function, and cardiac fibrosis in a TAC heart failure model as well as reducing the severity of cardiac hypertrophy, cardiac remodeling, reduced cardiac function, and cardiac fibrosis in a MI heart failure model. In addition, treatment with the endoglin polypeptide increased survival time of patient in both the TAC and MI heart failure models. In addition, data of the disclosure show that endoglin polypeptides bind with high affinity to BMP9 and BMP10. Thus, the disclosure establishes that antagonists of BMP signaling (e.g., signaling by one or more of BMP10, BMP9, BMP6, BMP3b, and BMP5) may be used to treat heart failure. While BMP10pro and endoglin polypeptides may affect heart failure through a mechanism other than BMP antagonism, the disclosure nonetheless demonstrates that desirable therapeutic agents may be selected on the basis of BMP signaling antagonism activity. Therefore, in some embodiments, the disclosure provides method for using various BMP signaling antagonists for treating heart failure including, for example, antagonists that inhibit one or more BMP ligands, particularly one or more of BMP10, BMP9, BMP6, BMP3b and BMP5; antagonists that inhibit one or more BMP-interacting type I-, type II-, or co-receptor (e.g., ALK1, ActRIIA, ActRIIB, BMPRII, and endoglin); and antagonists that inhibit one or more downstream signaling components (e.g., Smad proteins such as Smads 2 and 3). As used herein, such signaling antagonists are collectively referred to as “BMP antagonists” or “BMP inhibitors”. Accordingly, the disclosure provides in part, BMP antagonists compositions and methods for treating heart failure, particularly preventing or reducing the severity of one or more complications of heart failure (e.g., hypertrophy, cardiac remodeling, fibrosis, reduced cardiac function) as well as reducing the risk of death from one or more cardiac complications (events). BMP antagonists to be used in accordance with the methods and uses of the disclosure include, for example, ligand traps (e.g., soluble ActRIIA, ActRIIB, ALK1, and endoglin polypeptides), antibody antagonists, small molecule antagonists, and nucleotide antagonists. Optionally, BMP antagonists may be used in combination with one or more supportive therapies and/or additional active agents for treating heart failure.

In certain aspects, the disclosure relates to methods of reducing the risk of death (increasing survival) of a patient having heart failure comprising administering to a patient in need thereof an effective amount of a BMP antagonist. In some embodiments, the risk of death of a patient is from any cause (all-cause mortality). In some embodiments, the risk of death of a patient is from a cardiovascular event (complication). In some embodiments, the cardiovascular event comprises one or more of myocardial infarction, stroke, angina, arrhythmia, fluid retention, and progression of heart failure [e.g., class progression as categorized by the New York Heart Association (NYHA) or stage progression as categorized by American College of Cardiology/American Heart Association working group (AAC)]. In some embodiments, the BMP antagonist is administered to the patient after myocardial infarction. In some embodiments, the patient has left ventricular systolic dysfunction. In some embodiments, the disclosure relates to methods of reducing the risk of death of a patient having heart failure comprising administering to a patient in need thereof an effective amount of a BMP antagonist, wherein the BMP antagonist is administered after myocardial infarction. In some embodiments, the disclosure relates to methods of reducing the risk of death of a patient having heart failure comprising administering to a patient in need thereof an effective amount of a BMP antagonist, wherein the BMP antagonist is administered after myocardial infarction and the patient has left ventricular systolic dysfunction. In some embodiments, the patient has ≤40% ejection fraction. In some embodiments, the patient has ≤35% ejection fraction. In some embodiments, the disclosure relates to methods of reducing the risk of death of a patient having heart failure comprising administering to a patient in need thereof an effective amount of a BMP antagonist, wherein the BMP antagonist is administered after myocardial infarction and the patient has left ventricular systolic dysfunction with ≤40% ejection fraction (e.g., ≤35% ejection fraction). In some embodiments, the patient has one or more types of heart failure selected from the group consisting of: heart failure due to left ventricular dysfunction, heart failure with normal ejection fraction, acute heart failure, chronic heart failure, congestive heart failure, congenital heart failure, compensated heart failure, decompensated heart failure, diastolic heart failure, systolic heart failure, right-side heart (ventricle) failure, left-side heart (ventricle) failure, forward heart failure, backward heart failure, high output heart failure, low output heart failure, and myocardial edema. In some embodiments, the patient has one or more conditions selected from the group consisting of: systemic hypertension, pulmonary hypertension, diabetes, kidney (renal) failure (e.g., acute or chronic renal failure), coronary artery disease, hypertension, left ventricular dysfunction, heart valve disease, congenital heart defects, acute ischemic injury, reperfusion injury, cardiac remodeling pericardium disorders, myocardium disorders, great vessel disorders, and endocardium disorders. In some embodiments, the patient has at least class I heart failure (class I, class II, class III, or class IV) in accordance with the New York Heart Association (NYHA) functional classification. In some embodiments, the patient has at least stage A heart failure (stage A, stage B, stage C, or stage D) in accordance with the AAC functional classification. In some embodiments, the patient is further administered one or more additional active agents or supportive therapies for treating, preventing, or reducing the severity of heart failure or one or more complications of heart failure [e.g., adrenergic blockers (alpha- and beta-blockers), centrally acting alpha-agonists, angiotensin-converting enzyme (ACE) inhibitors, angiotensin receptor blockers, calcium channel blockers, positive inotropes, vasodilators, benzodiazepines, renin inhibitors, antithrombotic agents, diuretics, pacemaker, implantable cardiac defibrillator, cardiac contractility modulation, cardiac resynchronization therapy, ventricular assist device, biventricular cardiac resynchronization therapy, and heart transplant].

In certain aspects, the disclosure relates to methods of reducing the risk of hospitalization of a patient having heart failure comprising administering to a patient in need thereof an effective amount of a BMP antagonist. In some embodiments, the risk of hospitalization of a patient is from any cause (all-cause mortality). In some embodiments, the hospitalization of death of a patient is from a cardiovascular event (complication). In some embodiments, the cardiovascular event comprises one or more of myocardial infarction, stroke, angina, arrhythmia, fluid retention, and progression of heart failure [e.g., class progression as categorized by NYHA or stage progression as categorized by AAC]. In some embodiments, the BMP antagonist is administered to the patient after myocardial infarction. In some embodiments, the patient has left ventricular systolic dysfunction. In some embodiments, the disclosure relates to methods of reducing the risk of hospitalization of a patient having heart failure comprising administering to a patient in need thereof an effective amount of a BMP antagonist, wherein the BMP antagonist is administered after myocardial infarction. In some embodiments, the disclosure relates to methods of reducing the risk of hospitalization of a patient having heart failure comprising administering to a patient in need thereof an effective amount of a BMP antagonist, wherein the BMP antagonist is administered after myocardial infarction and the patient has left ventricular systolic dysfunction. In some embodiments, the patient has ≤40% ejection fraction. In some embodiments, the patient has ≤35% ejection fraction. In some embodiments, the disclosure relates to methods of reducing the risk of hospitalization of a patient having heart failure comprising administering to a patient in need thereof an effective amount of a BMP antagonist, wherein the BMP antagonist is administered after myocardial infarction and the patient has left ventricular systolic dysfunction with ≤40% ejection fraction (e.g., ≤35% ejection fraction). In some embodiments, the patient has one or more types of heart failure selected from the group consisting of: heart failure due to left ventricular dysfunction, heart failure with normal ejection fraction, acute heart failure, chronic heart failure, congestive heart failure, congenital heart failure, compensated heart failure, decompensated heart failure, diastolic heart failure, systolic heart failure, right-side heart (ventricle) failure, left-side heart (ventricle) failure, forward heart failure, backward heart failure, high output heart failure, low output heart failure, and myocardial edema. In some embodiments, the patient has one or more conditions selected from the group consisting of: systemic hypertension, pulmonary hypertension, diabetes, kidney (renal) failure (e.g., acute or chronic renal failure), coronary artery disease, hypertension, left ventricular dysfunction, heart valve disease, congenital heart defects, acute ischemic injury, reperfusion injury, cardiac remodeling pericardium disorders, myocardium disorders, great vessel disorders, and endocardium disorders. In some embodiments, the patient has class I heart failure in accordance with the New York Heart Association (NYHA) functional classification. In some embodiments, the patient has at least class I heart failure (class I, class II, class III, or class IV) in accordance with the New York Heart Association (NYHA) functional classification. In some embodiments, the patient has at least stage A heart failure (stage A, stage B, stage C, or stage D) in accordance with the American College of Cardiology/American Heart Association working group (AAC) functional classification. In some embodiments, the patient is further administered one or more additional active agents or supportive therapies for treating, preventing, or reducing the severity of heart failure or one or more complications of heart failure [e.g., adrenergic blockers (alpha- and beta-blockers), centrally acting alpha-agonists, angiotensin-converting enzyme (ACE) inhibitors, angiotensin receptor blockers, calcium channel blockers, positive inotropes, vasodilators, benzodiazepines, renin inhibitors, antithrombotic agents, diuretics, pacemaker, implantable cardiac defibrillator, cardiac contractility modulation, cardiac resynchronization therapy, ventricular assist device, biventricular cardiac resynchronization therapy, and heart transplant].

In certain aspects, the disclosure relates to methods of improving or reducing (delaying) progression of heart failure in a patient, comprising administering to a patient in need thereof an effective amount of a BMP antagonist. In some embodiments, the patient has class I heart failure in accordance with the New York Heart Association (NYHA) functional classification. In some embodiments, the patient has class II heart failure in accordance with the New York Heart Association (NYHA) functional classification. In some embodiments, the patient has class III heart failure in accordance with the New York Heart Association (NYHA) functional classification. In some embodiments, the patient has class IV heart failure in accordance with the New York Heart Association (NYHA) functional classification. In some embodiments, the patient has class II or III heart failure in accordance with the New York Heart Association (NYHA) functional classification. In some embodiments, the patient has class III or IV heart failure in accordance with the New York Heart Association (NYHA) functional classification. In some embodiments, the patient has class II, III, or IV heart failure in accordance with the New York Heart Association (NYHA) functional classification. In some embodiments, the method improves the patient's heart failure score in accordance with the NYHA functional classification system by at least one class (e.g., improvement from class IV to class III heart failure, from class IV to class II heart failure, from class IV to class I heart failure, from stage III to stage II heart failure, from stage III to stage I heart failure, or from class II to class I heart failure). In some embodiments, the method reduces progression of the patient's heart failure score in accordance with the NYHA functional classification system by at least one class (e.g., prevents or delays progression from class I to class II heart failure, delays progression from class I to class III heart failure, delays progression from class I to class IV heart failure, delays progression from class II to class III heart failure, delays progression from class II to class IV heart failure, or delays progression from class III to class IV heart failure. In some embodiments, the patient has stage A heart failure in accordance with the American College of Cardiology/American Heart Association working group (AAC) functional classification. In some embodiments, the patient has stage B heart failure in accordance with the American College of Cardiology/American Heart Association working group (AAC) functional classification. In some embodiments, the patient has stage C heart failure in accordance with the American College of Cardiology/American Heart Association working group (AAC) functional classification. In some embodiments, the patient has stage D heart failure in accordance with the American College of Cardiology/American Heart Association working group (AAC) functional classification. In some embodiments, the patient has stage B or C heart failure in accordance with the American College of Cardiology/American Heart Association working group (AAC) functional classification. In some embodiments, the patient has stage C or D heart failure in accordance with the American College of Cardiology/American Heart Association working group (AAC) functional classification. In some embodiments, the patient has stage B, C, or D heart failure in accordance with the American College of Cardiology/American Heart Association working group (AAC) functional classification. In some embodiments, the method improves the patient's heart failure score in accordance with the ACC functional classification system by at least one stage (e.g., improvement from stage D to stage C heart failure, from stage D to stage B heart failure, from stage D to stage A heart failure, from stage C to stage B heart failure, from stage C to stage A heart failure, or from stage B to stage A heart failure). In some embodiments, the method reduces progression of the patient's heart failure score in accordance with the ACC functional classification system by at least one stage (e.g., prevents or delays progression from stage A to stage B heart failure, delays progression from stage A to stage C heart failure, delays progression from stage A to stage D heart failure, delays progression from stage B to stage C heart failure, delays progression from stage B to stage D heart failure, or delays progression from stage C to stage D heart failure. In some embodiments, the patient previously had a myocardial infarction. In some embodiments, the patient has left ventricular systolic dysfunction. In some embodiments, the patient has ≤40% ejection fraction. In some embodiments, the patient has ≤35% ejection fraction. In some embodiments, the patient has one or more types of heart failure selected from the group consisting of: heart failure due to left ventricular dysfunction, heart failure with normal ejection fraction, acute heart failure, chronic heart failure, congestive heart failure, congenital heart failure, compensated heart failure, decompensated heart failure, diastolic heart failure, systolic heart failure, right-side heart (ventricle) failure, left-side heart (ventricle) failure, forward heart failure, backward heart failure, high output heart failure, low output heart failure, and myocardial edema. In some embodiments, the patient has one or more conditions selected from the group consisting of: systemic hypertension, pulmonary hypertension, diabetes, kidney (renal) failure (e.g., acute or chronic renal failure), coronary artery disease, hypertension, left ventricular dysfunction, heart valve disease, congenital heart defects, acute ischemic injury, reperfusion injury, cardiac remodeling pericardium disorders, myocardium disorders, great vessel disorders, and endocardium disorders. In some embodiments, the patient is further administered one or more additional active agents or supportive therapies for treating, preventing, or reducing the severity of heart failure or one or more complications of heart failure [e.g., adrenergic blockers (alpha- and beta-blockers), centrally acting alpha-agonists, angiotensin-converting enzyme (ACE) inhibitors, angiotensin receptor blockers, calcium channel blockers, positive inotropes, vasodilators, benzodiazepines, renin inhibitors, antithrombotic agents, diuretics, pacemaker, implantable cardiac defibrillator, cardiac contractility modulation, cardiac resynchronization therapy, ventricular assist device, biventricular cardiac resynchronization therapy, and heart transplant].

In some embodiments, the disclosure relates to methods of reducing incidence of cardiovascular events (complications) in a patient comprising administering to a patient in need thereof an effective amount of a BMP antagonist. In some embodiments, cardiovascular event is one or more of myocardial infarction, stroke, angina, arrhythmia, fluid retention, progression of heart failure. In some embodiments, the cardiovascular event is one or more of dyspnea, orthopnea, paroxysmal nocturnal dyspnea, fatigue, fluid retention, pulmonary congestion, edema, peripheral edema, angina, hypertension, arrhythmia, ventricular arrhythmia, cardiomyopathy, cardiac hypertrophy, reduced renal blood flow, renal insufficiency, myocardial infarct, cardiac remodeling, cardiac fibrosis, cardiac hypertension, cardiac wall stress, cardiac inflammation, cardiac pressure overload, cardiac volume overload, stroke, cardiac chamber dilation, increase in ventricular sphericity, interstitial fibrosis, perivascular fibrosis, cardiomyocyte hypertrophy, cardiac asthma, nocturia, ascities, congestive hepatopathy, coagulopathy, acute ischemic injury, reperfusion injury, impairment of left ventricle function, and impairment of right ventricle function. In some embodiments, the cardiovascular event would result in patient hospitalization. The determination of whether a patient should be hospitalized due to a cardiovascular event can be determined by one of skill in the art (e.g., a physician, particularly an emergency physician and cardiologists). In some embodiments, the patient has at least class I heart failure (class I, class II, class III, or class IV) in accordance with the New York Heart Association (NYHA) functional classification. In some embodiments, the patient has at least stage A heart failure (stage A, stage B, stage C, or stage D) in accordance with the American College of Cardiology/American Heart Association working group (AAC) functional classification. In some embodiments, the patient has cardiac fibrosis. In some embodiments, the patient has cardiac hypertrophy. In some embodiments, the patient has cardiac remodeling. In some embodiments, the patient has cardiac dysfunction (e.g., ≤40% or ≤35% ejection fraction). In some embodiments, the patient is hypertensive. In some embodiments, the patient is further administered one or more additional active agents or supportive therapies for treating, preventing, or reducing the severity of heart failure or one or more complications of heart failure [e.g., adrenergic blockers (alpha- and beta-blockers), centrally acting alpha-agonists, angiotensin-converting enzyme (ACE) inhibitors, angiotensin receptor blockers, calcium channel blockers, positive inotropes, vasodilators, benzodiazepines, renin inhibitors, antithrombotic agents, diuretics, pacemaker, implantable cardiac defibrillator, cardiac contractility modulation, cardiac resynchronization therapy, ventricular assist device, biventricular cardiac resynchronization therapy, and heart transplant].

In some embodiments, the disclosure relates to methods of treating, preventing, or reducing the severity of cardiac fibrosis in a patient, comprising administering to a patient in need thereof an effective amount of a BMP antagonist. In some embodiments, the patient has heart failure. In some embodiments, the patient has one or more types of heart failure selected from the group consisting of: heart failure due to left ventricular dysfunction, heart failure with normal ejection fraction, acute heart failure, chronic heart failure, congestive heart failure, congenital heart failure, compensated heart failure, decompensated heart failure, diastolic heart failure, systolic heart failure, right-side heart (ventricle) failure, left-side heart (ventricle) failure, forward heart failure, backward heart failure, high output heart failure, low output heart failure, and myocardial edema. In some embodiments, the patient has at least class I heart failure (class I, class II, class III, or class IV) in accordance with the New York Heart Association (NYHA) functional classification. In some embodiments, the patient has at least stage A heart failure (stage A, stage B, stage C, or stage D) in accordance with the American College of Cardiology/American Heart Association working group (AAC) functional classification. In some embodiments, the patient previously had a myocardial infarction. In some embodiments, the patient has left ventricular systolic dysfunction. In some embodiments, the patient previously had a myocardial infarction. In some embodiments, the patient has left ventricular systolic dysfunction. In some embodiments, the patient has ≤40% ejection fraction. In some embodiments, the patient has ≤35% ejection fraction). In some embodiments, the patient is further administered one or more additional active agents or supportive therapies for treating, preventing, or reducing the severity of heart failure or one or more complications of heart failure [e.g., adrenergic blockers (alpha- and beta-blockers), centrally acting alpha-agonists, angiotensin-converting enzyme (ACE) inhibitors, angiotensin receptor blockers, calcium channel blockers, positive inotropes, vasodilators, benzodiazepines, renin inhibitors, antithrombotic agents, diuretics, pacemaker, implantable cardiac defibrillator, cardiac contractility modulation, cardiac resynchronization therapy, ventricular assist device, biventricular cardiac resynchronization therapy, and heart transplant].

In some embodiments, the disclosure relates to methods of treating, preventing, or reducing the severity of cardiac hypertrophy in a patient comprising administering to a patient in need thereof an effective amount of a BMP antagonist. In some embodiments, the cardiac hypertrophy is concentric hypertrophy. In some embodiments, the cardiac hypertrophy is eccentric hypertrophy. In some embodiments, the patient has both concentric hypertrophy and eccentric hypertrophy. In some embodiments, the patient has heart failure. In some embodiments, the patient has one or more types of heart failure selected from the group consisting of: heart failure due to left ventricular dysfunction, heart failure with normal ejection fraction, acute heart failure, chronic heart failure, congestive heart failure, congenital heart failure, compensated heart failure, decompensated heart failure, diastolic heart failure, systolic heart failure, right-side heart (ventricle) failure, left-side heart (ventricle) failure, forward heart failure, backward heart failure, high output heart failure, low output heart failure, and myocardial edema. In some embodiments, the patient has at least class I heart failure (class I, class II, class III, or class IV) in accordance with the New York Heart Association (NYHA) functional classification. In some embodiments, the patient has at least stage A heart failure (stage A, stage B, stage C, or stage D) in accordance with the American College of Cardiology/American Heart Association working group (AAC) functional classification. In some embodiments, the patient previously had a myocardial infarction. In some embodiments, the patient has left ventricular systolic dysfunction. In some embodiments, the patient previously had a myocardial infarction. In some embodiments, the patient has left ventricular systolic dysfunction. In some embodiments, the patient has ≤40% ejection fraction. In some embodiments, the patient has ≤35% ejection fraction). In some embodiments, the patient is further administered one or more additional active agents or supportive therapies for treating, preventing, or reducing the severity of heart failure or one or more complications of heart failure [e.g., adrenergic blockers (alpha- and beta-blockers), centrally acting alpha-agonists, angiotensin-converting enzyme (ACE) inhibitors, angiotensin receptor blockers, calcium channel blockers, positive inotropes, vasodilators, benzodiazepines, renin inhibitors, antithrombotic agents, diuretics, pacemaker, implantable cardiac defibrillator, cardiac contractility modulation, cardiac resynchronization therapy, ventricular assist device, biventricular cardiac resynchronization therapy, and heart transplant.

In some embodiments, the disclosure relates to a method of treating, preventing, or reducing the severity of cardiac remodeling in a patient comprising administering to a patient in need thereof an effective amount of a BMP antagonist. In some embodiments, the cardiac remodeling is ventricle remodeling. In some embodiments, the cardiac remodeling is ventricular dilation. In some embodiments, the method decreases interventricular septal remodeling. In some embodiments, the method decreases interventricular septal end diastole. In some embodiments, the method decreases posterior wall remodeling. In some embodiments, the method decreases posterior wall end diastole. In some embodiments, the patient has heart failure. In some embodiments, the patient has one or more types of heart failure selected from the group consisting of: heart failure due to left ventricular dysfunction, heart failure with normal ejection fraction, acute heart failure, chronic heart failure, congestive heart failure, congenital heart failure, compensated heart failure, decompensated heart failure, diastolic heart failure, systolic heart failure, right-side heart (ventricle) failure, left-side heart (ventricle) failure, forward heart failure, backward heart failure, high output heart failure, low output heart failure, and myocardial edema. In some embodiments, the patient has at least class I heart failure (class I, class II, class III, or class IV) in accordance with the New York Heart Association (NYHA) functional classification. In some embodiments, the patient has at least stage A heart failure (stage A, stage B, stage C, or stage D) in accordance with the American College of Cardiology/American Heart Association working group (AAC) functional classification. In some embodiments, the patient previously had a myocardial infarction. In some embodiments, the patient has left ventricular systolic dysfunction. In some embodiments, the patient previously had a myocardial infarction. In some embodiments, the patient has left ventricular systolic dysfunction. In some embodiments, the patient has ≤40% ejection fraction. In some embodiments, the patient has ≤35% ejection fraction). In some embodiments, the patient is further administered one or more additional active agents or supportive therapies for treating, preventing, or reducing the severity of heart failure or one or more complications of heart failure [e.g., adrenergic blockers (alpha- and beta-blockers), centrally acting alpha-agonists, angiotensin-converting enzyme (ACE) inhibitors, angiotensin receptor blockers, calcium channel blockers, positive inotropes, vasodilators, benzodiazepines, renin inhibitors, antithrombotic agents, diuretics, pacemaker, implantable cardiac defibrillator, cardiac contractility modulation, cardiac resynchronization therapy, ventricular assist device, biventricular cardiac resynchronization therapy, and heart transplant].

In some embodiments, the disclosure relates to methods of treating, preventing, or reducing the severity of cardiac dysfunction in a patient, comprising administering to a patient in need thereof an effective amount of a BMP antagonist. In some embodiments, the method increases cardiac ejection fraction. In some embodiments, the patient has ≤40% ejection fraction. In some embodiments, the patient has ≤35% ejection fraction). In some embodiments, the method increases cardiac ejection fraction by at least 5% (e.g., at least 5%, 6%, 7%, 8%, 9%, 10%, 15%, 20%, 25%, 30%, 35%, 40%, 50%, 60%, 65% or more). In some embodiments, the method decreases isovolumic relaxation time. In some embodiments, the method decreases isovolumic relaxation time by at least 2 ms (e.g., at least 2, 3, 4, 5, 6, 7, 8, 9, 10, 11, 12, 13, 14, 15, 16, 17, 18, 19, 20 or more ms). In some embodiment, wherein the method increases fractional shorting. In some embodiments, the method increase fractional shorting by at least 5% (e.g., at least 5%, 6%, 7%, 8%, 9%, 10%, 15%, 20%, 25%, 30%, 35%, 40%, 50%, or more). In some embodiments, the patient has heart failure. In some embodiments, the patient has one or more types of heart failure selected from the group consisting of: heart failure due to left ventricular dysfunction, heart failure with normal ejection fraction, acute heart failure, chronic heart failure, congestive heart failure, congenital heart failure, compensated heart failure, decompensated heart failure, diastolic heart failure, systolic heart failure, right-side heart (ventricle) failure, left-side heart (ventricle) failure, forward heart failure, backward heart failure, high output heart failure, low output heart failure, and myocardial edema. In some embodiments, the patient has at least class I heart failure (class I, class II, class III, or class IV) in accordance with the New York Heart Association (NYHA) functional classification. In some embodiments, the patient has at least stage A heart failure (stage A, stage B, stage C, or stage D) in accordance with the American College of Cardiology/American Heart Association working group (AAC) functional classification. In some embodiments, the patient previously had a myocardial infarction. In some embodiments, the patient has left ventricular systolic dysfunction. In some embodiments, the patient previously had a myocardial infarction. In some embodiments, the patient has left ventricular systolic dysfunction. In some embodiments, the patient is further administered one or more additional active agents or supportive therapies for treating, preventing, or reducing the severity of heart failure or one or more complications of heart failure [e.g., adrenergic blockers (alpha- and beta-blockers), centrally acting alpha-agonists, angiotensin-converting enzyme (ACE) inhibitors, angiotensin receptor blockers, calcium channel blockers, positive inotropes, vasodilators, benzodiazepines, renin inhibitors, antithrombotic agents, diuretics, pacemaker, implantable cardiac defibrillator, cardiac contractility modulation, cardiac resynchronization therapy, ventricular assist device, biventricular cardiac resynchronization therapy, and heart transplant].

In some embodiments, the disclosure relates to a method of treating, preventing, or reducing the severity of hypertension in a patient, comprising administering to a patient in need thereof an effective amount of a BMP antagonist. In some embodiments, the method reduces the patient's blood pressure. In some embodiments, the method reduces systolic blood pressure. In some embodiments, the method reduces systolic blood pressure by at least 4 mm Hg (e.g., at least 4, 5, 6, 7, 8, 9, 10, 11, 12, 13, 14, 15, 16, 17, 18, 19, 20, or more mm Hg). In some embodiments, the method reduces diastolic blood pressure. In some embodiments, the method reduces diastolic blood pressure by at least 2 mm Hg (e.g., at least 2, 3, 4, 5, 6, 7, 8, 9, 10, 11, 12, 13, 14, 15, 16, 17, 18, 19, 20, or more mm Hg). In some embodiments, the method the blood pressure is measured as resting blood pressure. In some embodiments, the method the blood pressure is measured as ambulatory blood pressure. In some embodiments, the patient has heart failure. In some embodiments, the patient has one or more types of heart failure selected from the group consisting of: heart failure due to left ventricular dysfunction, heart failure with normal ejection fraction, acute heart failure, chronic heart failure, congestive heart failure, congenital heart failure, compensated heart failure, decompensated heart failure, diastolic heart failure, systolic heart failure, right-side heart (ventricle) failure, left-side heart (ventricle) failure, forward heart failure, backward heart failure, high output heart failure, low output heart failure, and myocardial edema. In some embodiments, the patient has at least class I heart failure (class I, class II, class III, or class IV) in accordance with the New York Heart Association (NYHA) functional classification. In some embodiments, the patient has at least stage A heart failure (stage A, stage B, stage C, or stage D) in accordance with the American College of Cardiology/American Heart Association working group (AAC) functional classification. In some embodiments, the patient previously had a myocardial infarction. In some embodiments, the patient has left ventricular systolic dysfunction. In some embodiments, the patient previously had a myocardial infarction. In some embodiments, the patient has left ventricular systolic dysfunction. In some embodiments, the patient is further administered one or more additional active agents or supportive therapies for treating, preventing, or reducing the severity of heart failure or one or more complications of heart failure [e.g., adrenergic blockers (alpha- and beta-blockers), centrally acting alpha-agonists, angiotensin-converting enzyme (ACE) inhibitors, angiotensin receptor blockers, calcium channel blockers, positive inotropes, vasodilators, benzodiazepines, renin inhibitors, antithrombotic agents, diuretics, pacemaker, implantable cardiac defibrillator, cardiac contractility modulation, cardiac resynchronization therapy, ventricular assist device, biventricular cardiac resynchronization therapy, and heart transplant.

In some embodiments, the disclosure relates to methods of treating, preventing, or reducing the severity of heart disease or one or more complications of heart disease, comprising administering to a patient in need thereof an effective amount of a BMP antagonist. In some embodiments, the one or more complication of heart disease is one or more of dyspnea, orthopnea, paroxysmal nocturnal dyspnea, fatigue, fluid retention, pulmonary congestion, edema, peripheral edema, angina, hypertension, arrhythmia, ventricular arrhythmia, cardiomyopathy, cardiac hypertrophy, reduced renal blood flow, renal insufficiency, myocardial infarct, cardiac remodeling, cardiac fibrosis, cardiac hypertension, cardiac wall stress, cardiac inflammation, cardiac pressure overload, cardiac volume overload, stroke, cardiac chamber dilation, increase in ventricular sphericity, interstitial fibrosis, perivascular fibrosis, cardiomyocyte hypertrophy, cardiac asthma, nocturia, ascities, congestive hepatopathy, coagulopathy, acute ischemic injury, reperfusion injury, impairment of left ventricle function, and impairment of right ventricle function. In some embodiments, the complication is cardiac fibrosis. In some embodiments, the complication is cardiac hypertrophy. In some embodiments, the complication is cardiac remodeling. In some embodiments, the complication is cardiac dysfunction (e.g., ≤40% or ≤35% ejection fraction). In some embodiments, the complication is hypertension. In some embodiments, the patient has one or more types of heart failure selected from the group consisting of: heart failure due to left ventricular dysfunction, heart failure with normal ejection fraction, acute heart failure, chronic heart failure, congestive heart failure, congenital heart failure, compensated heart failure, decompensated heart failure, diastolic heart failure, systolic heart failure, right-side heart (ventricle) failure, left-side heart (ventricle) failure, forward heart failure, backward heart failure, high output heart failure, low output heart failure, and myocardial edema. In some embodiments, the patient has at least class I heart failure (class I, class II, class III, or class IV) in accordance with the New York Heart Association (NYHA) functional classification. In some embodiments, the patient has at least stage A heart failure (stage A, stage B, stage C, or stage D) in accordance with the American College of Cardiology/American Heart Association working group (AAC) functional classification. In some embodiments, the patient previously had a myocardial infarction. In some embodiments, the patient has left ventricular systolic dysfunction. In some embodiments, the patient previously had a myocardial infarction. In some embodiments, the patient has left ventricular systolic dysfunction. In some embodiments, the patient has ≤40% ejection fraction. In some embodiments, the patient has ≤35% ejection fraction). In some embodiments, the patient is further administered one or more additional active agents or supportive therapies for treating, preventing, or reducing the severity of heart failure or one or more complications of heart failure [e.g., adrenergic blockers (alpha- and beta-blockers), centrally acting alpha-agonists, angiotensin-converting enzyme (ACE) inhibitors, angiotensin receptor blockers, calcium channel blockers, positive inotropes, vasodilators, benzodiazepines, renin inhibitors, antithrombotic agents, diuretics, pacemaker, implantable cardiac defibrillator, cardiac contractility modulation, cardiac resynchronization therapy, ventricular assist device, biventricular cardiac resynchronization therapy, and heart transplant.

In certain aspects, a BMP antagonist to be used in accordance with methods and uses described herein is an agent that inhibits BMP10 (a BMP10 antagonist). Effects on BMP10 inhibition may be determined, for example, using a cell-based assay including those described herein (e.g., Smad signaling reporter assay). Such cell-based assays may be used to determine the inhibitory effects of other BMP antagonists including those described herein. Therefore, in some embodiments, a BMP10 antagonist may bind to BMP10. Ligand binding activity may be determined, for example, using a binding affinity assay including such as those described herein. Such ligand-binding assays may be used to determine the binding affinity of other BMP antagonists including those described herein. In some embodiments, a BMP10 antagonist binds to BMP10 with a K_{D} of at least 1×10⁻⁸ M (e.g., at least at least 1×10⁻⁹ M, at least 1×10⁻¹⁰ M, at least 1×10⁻¹¹ M, or at least 1×10⁻¹² M). In some embodiments, a BMP10 antagonist further inhibits the activity of BMP9. In some embodiments, the BMP10 antagonist further inhibits one or more of BMP6, BMP3b, and BMP5. Therefore, in some embodiments, a BMP10 antagonist may bind to one or more of BMP9, BMP6, BMP3b, and BMP5. Examples of BMP10 antagonists are described herein and include, e.g., ligand traps (e.g., soluble, ligand-binding domain of type I-, type II-, or co-receptors of the TGFβ receptor superfamily), antibodies, small molecules, and polynucleotides. In some embodiments, a BMP10 antagonist may further inhibit one or more type I-, type II-, or co-receptor of the TGFβ superfamily and/or signaling mediator (e.g., Smads)

In certain aspects, a BMP antagonist to be used in accordance with methods and uses described herein is an agent that inhibits BMP9 (a BMP9 antagonist). Therefore, in some embodiments, a BMP9 antagonist may bind to BMP9. In some embodiments, a BMP9 antagonist binds to BMP9 with a K_{D} of at least 1×10⁻⁸ M (e.g., at least at least 1×10⁻⁹ M, at least 1×10⁻¹⁰ M, at least 1×10⁻¹¹ M, or at least 1×10⁻¹² M). In some embodiments, a BMP9 antagonist further inhibits the activity of BMP10. In some embodiments, the BMP9 antagonist further inhibits one or more of BMP6, BMP3b, and BMP5. Therefore, in some embodiments, a BMP9 antagonist may bind to one or more of BMP10, BMP6, BMP3b, and BMP5. Examples of BMP9 antagonists are described herein and include, e.g., ligand traps (e.g., soluble, ligand-binding domain of type I-, type II-, or co-receptors of the TGFβ receptor superfamily), antibodies, small molecules, and polynucleotides. In some embodiments, a BMP9 antagonist may further inhibit one or more type I-, type II-, or co-receptor of the TGFβ superfamily and/or signaling mediator (e.g., Smads).

In certain aspects, a BMP antagonist to be used in accordance with methods and uses described herein is an agent that inhibits BMP6 (a BMP6 antagonist). Therefore, in some embodiments, a BMP6 antagonist may bind to BMP6. In some embodiments, a BMP6 antagonist binds to BMP6 with a K_{D} of at least 1×10⁻⁸ M (e.g., at least at least 1×10⁻⁹ M, at least 1×10⁻¹⁰ M, at least 1×10⁻¹¹ M, or at least 1×10⁻¹² M). In some embodiments, a BMP6 antagonist further inhibits the activity of BMP10 and/or BMP9. In some embodiments, the BMP6 antagonist further inhibits BMP3b and/or BMP5. Therefore, in some embodiments, a BMP6 antagonist may bind to one or more of BMP10, BMP9, BMP3b, and BMP5. Examples of BMP6 antagonists are described herein and include, e.g., ligand traps (e.g., soluble, ligand-binding domain of type I-, type II-, or co-receptors of the TGFβ receptor superfamily), antibodies, small molecules, and polynucleotides. In some embodiments, a BMP6 antagonist may further inhibit one or more type I-, type II-, or co-receptor of the TGFβ superfamily and/or signaling mediator (e.g., Smads).

In certain aspects, a BMP antagonist to be used in accordance with methods and uses described herein is an agent that inhibits BMP3b (a BMP3b antagonist). Therefore, in some embodiments, a BMP3b antagonist may bind to BMP3b. In some embodiments, a BMP3b antagonist binds to BMP3b with a K_{D} of at least 1×10⁻⁸ M (e.g., at least at least 1×10⁻⁹ M, at least 1×10⁻¹⁰ M, at least 1×10⁻¹¹ M, or at least 1×10⁻¹² M). In some embodiments, a BMP3b antagonist further inhibits the activity of BMP10 and/or BMP9. In some embodiments, the BMP3b antagonist further inhibits BMP6 and/or BMP5. Therefore, in some embodiments, a BMP3b antagonist may bind to one or more of BMP10, BMP9, BMP6, and BMP5. Examples of BMP3b antagonists are described herein and include, e.g., ligand traps (e.g., soluble, ligand-binding domain of type I-, type II-, or co-receptors of the TGFβ receptor superfamily), antibodies, small molecules, and polynucleotides. In some embodiments, a BMP3b antagonist may further inhibit one or more type I-, type II-, or co-receptor of the TGFβ superfamily and/or signaling mediator (e.g., Smads).

In certain aspects, a BMP antagonist to be used in accordance with methods and uses described herein is an agent that inhibits BMP5 (a BMP5 antagonist). Therefore, in some embodiments, a BMP5 antagonist may bind to BMP5. In some embodiments, a BMP5 antagonist binds to BMP5 with a K_{D} of at least 1×10⁻⁸ M (e.g., at least at least 1×10⁻⁹ M, at least 1×10⁻¹⁰ M, at least 1×10⁻¹¹ M, or at least 1×10⁻¹² M). In some embodiments, a BMP5 antagonist further inhibits the activity of BMP10 and/or BMP9. In some embodiments, the BMP5 antagonist further inhibits BMP6 and/or BMP5. Therefore, in some embodiments, a BMP5 antagonist may bind to one or more of BMP10, BMP9, BMP6, and BMP3b. Examples of BMP5 antagonists are described herein and include, e.g., ligand traps (e.g., soluble, ligand-binding domain of type I-, type II-, or co-receptors of the TGFβ receptor superfamily), antibodies, small molecules, and polynucleotides. In some embodiments, a BMP5 antagonist may further inhibit one or more type I-, type II-, or co-receptor of the TGFβ superfamily and/or signaling mediator (e.g., Smads).

In certain aspects, a BMP antagonist to be used in accordance with methods and uses described herein is an agent that inhibits one or more receptors or signaling mediators of one or more of BMP10, BMP9, BMP6, BMP3b, and BMP5. For example, in some embodiments, a BMP antagonist may inhibit ActRIIA. In some embodiments, a BMP antagonist may inhibit ActRIIB In some embodiments, a BMP antagonist may inhibit ActRIIA and ActRIIB In some embodiments, a BMP antagonist may inhibit BMPRII. In some embodiments, a BMP antagonist may inhibit ALK1. In some embodiments, a BMP antagonist may inhibit endoglin. In some embodiments, a BMP antagonist may inhibit one or more Smad proteins (e.g., Smad 2 and/or 3). Therefore, in some embodiments, a BMP antagonist may bind to one or more of ActRIIA, ActRIIB, BMPRII, endoglin, and Smad proteins. In some embodiments, a BMP antagonist binds to one or more of ActRIIA, ActRIIB, BMPRII, endoglin, and Smad proteins with a K_{D} of at least 1×10⁻⁸ M (e.g., at least at least 1×10⁻⁹ M, at least 1×10⁻¹⁰ M, at least 1×10⁻¹¹ M, or at least 1×10⁻¹² M). Examples of ActRIIA, ActRIIB, BMPRII, endoglin, and Smad protein antagonists are described herein and include, e.g., antibodies, small molecules, and polynucleotides.

In certain aspects, a BMP antagonist of the disclosure is an ActRII polypeptide. The term “ActRII polypeptide” collectively refers to naturally occurring ActRIIA and ActRIIB polypeptides as well as truncations and variants thereof such as those described herein. Preferably ActRII polypeptides comprise a ligand-binding domain of an ActRII polypeptide or modified (variant) form thereof. For example, in some embodiments, an ActRIIA polypeptide may comprise an extracellular domain of ActRIIA. Similarly, an ActRIIB polypeptide may comprise an extracellular domain of ActRIIB Preferably, ActRII polypeptides to be used in accordance with the methods and uses described herein are soluble polypeptides. In some embodiments, an ActRIIA polypeptide comprises an amino acid sequence that is at least 70%, 75% 80%, 85%, 90%, 91%, 92%, 93%, 94%, 95%, 96%, 97%, 98%, 99%, or 100% identical to the amino acid sequence of SEQ ID NO: 10. In some embodiments, an ActRIIA polypeptide comprises an amino acid sequence that is at least 70%, 75% 80%, 85%, 90%, 91%, 92%, 93%, 94%, 95%, 96%, 97%, 98%, 99%, or 100% identical to the amino acid sequence of SEQ ID NO: 11. In some embodiments, an ActRIIA polypeptide comprises an amino acid sequence that is at least 70%, 75% 80%, 85%, 90%, 91%, 92%, 93%, 94%, 95%, 96%, 97%, 98%, 99%, or 100% identical to the sequence of amino acid 30-110 of SEQ ID NO: 9. In some embodiments, an ActRIIA polypeptide comprises an amino acid sequence that is at least 70%, 75% 80%, 85%, 90%, 91%, 92%, 93%, 94%, 95%, 96%, 97%, 98%, 99%, or 100% identical to the amino acid sequence of SEQ ID NO: 50. In some embodiments, an ActRIIA polypeptide comprises an amino acid sequence that is at least 70%, 75% 80%, 85%, 90%, 91%, 92%, 93%, 94%, 95%, 96%, 97%, 98%, 99%, or 100% identical to the amino acid sequence of SEQ ID NO: 54. In some embodiments, an ActRIIA polypeptide comprises an amino acid sequence that is at least 70%, 75% 80%, 85%, 90%, 91%, 92%, 93%, 94%, 95%, 96%, 97%, 98%, 99%, or 100% identical to the amino acid sequence of SEQ ID NO: 57. In some embodiments, an ActRIIB polypeptide comprises an amino acid sequence that is at least 70%, 75% 80%, 85%, 90%, 91%, 92%, 93%, 94%, 95%, 96%, 97%, 98%, 99%, or 100% identical to amino acids 29-109 of SEQ ID NO: 1. In some embodiments, an ActRIIB polypeptide comprises an amino acid sequence that is at least 70%, 75% 80%, 85%, 90%, 91%, 92%, 93%, 94%, 95%, 96%, 97%, 98%, 99%, or 100% identical to amino acids 25-131 of SEQ ID NO: 1. In some embodiments, an ActRIIB polypeptide comprises an amino acid sequence that is at least 70%, 75% 80%, 85%, 90%, 91%, 92%, 93%, 94%, 95%, 96%, 97%, 98%, 99%, or 100% identical to the amino acid sequence of SEQ ID NO: 2. In some embodiments, an ActRIIB polypeptide comprises an amino acid sequence that is at least 70%, 75% 80%, 85%, 90%, 91%, 92%, 93%, 94%, 95%, 96%, 97%, 98%, 99%, or 100% identical to the amino acid sequence of SEQ ID NO: 3. In some embodiments, an ActRIIB polypeptide comprises an amino acid sequence that is at least 70%, 75% 80%, 85%, 90%, 91%, 92%, 93%, 94%, 95%, 96%, 97%, 98%, 99%, or 100% identical to the amino acid sequence of SEQ ID NO: 5. In some embodiments, an ActRIIB polypeptide comprises an amino acid sequence that is at least 70%, 75% 80%, 85%, 90%, 91%, 92%, 93%, 94%, 95%, 96%, 97%, 98%, 99%, or 100% identical to the amino acid sequence of SEQ ID NO: 6. In some embodiments, an ActRIIB polypeptide comprises an amino acid sequence that is at least 70%, 75% 80%, 85%, 90%, 91%, 92%, 93%, 94%, 95%, 96%, 97%, 98%, 99%, or 100% identical to the amino acid sequence of SEQ ID NO: 65. In some embodiments, an ActRIIB polypeptide comprises an amino acid sequence that is at least 70%, 75% 80%, 85%, 90%, 91%, 92%, 93%, 94%, 95%, 96%, 97%, 98%, 99%, or 100% identical to the amino acid sequence of SEQ ID NO: 133. In some embodiments, an ActRIIB polypeptide comprises an amino acid sequence that is at least 70%, 75% 80%, 85%, 90%, 91%, 92%, 93%, 94%, 95%, 96%, 97%, 98%, 99%, or 100% identical to the amino acid sequence of SEQ ID NO: 58. In some embodiments, an ActRIIB polypeptide comprises an amino acid sequence that is at least 70%, 75% 80%, 85%, 90%, 91%, 92%, 93%, 94%, 95%, 96%, 97%, 98%, 99%, or 100% identical to the amino acid sequence of SEQ ID NO: 60. In some embodiments, an ActRIIB polypeptide comprises an amino acid sequence that is at least 70%, 75% 80%, 85%, 90%, 91%, 92%, 93%, 94%, 95%, 96%, 97%, 98%, 99%, or 100% identical to the amino acid sequence of SEQ ID NO: 63. In some embodiments, an ActRIIB polypeptide comprises an amino acid sequence that is at least 70%, 75% 80%, 85%, 90%, 91%, 92%, 93%, 94%, 95%, 96%, 97%, 98%, 99%, or 100% identical to the amino acid sequence of SEQ ID NO: 64. In some embodiments, an ActRIIB polypeptide comprises an amino acid sequence that is at least 70%, 75% 80%, 85%, 90%, 91%, 92%, 93%, 94%, 95%, 96%, 97%, 98%, 99%, or 100% identical to the amino acid sequence of SEQ ID NO: 66. In some embodiments, an ActRIIB polypeptide comprises an amino acid sequence that is at least 70%, 75% 80%, 85%, 90%, 91%, 92%, 93%, 94%, 95%, 96%, 97%, 98%, 99%, or 100% identical to the amino acid sequence of SEQ ID NO: 123. In some embodiments, an ActRIIB polypeptide comprises an amino acid sequence that is at least 70%, 75% 80%, 85%, 90%, 91%, 92%, 93%, 94%, 95%, 96%, 97%, 98%, 99%, or 100% identical to the amino acid sequence of SEQ ID NO: 131. In some embodiments, an ActRIIB polypeptide comprises an amino acid sequence that is at least 70%, 75% 80%, 85%, 90%, 91%, 92%, 93%, 94%, 95%, 96%, 97%, 98%, 99%, or 100% identical to the amino acid sequence of SEQ ID NO: 132. In some embodiments, ActRIIB polypeptides do not comprise an acidic amino acid at position 79 with respect to SEQ ID NO: 1 (e.g., an artificially acidic amino acid or naturally occurring acidic amino acid such as D or E).

In certain aspects, a BMP antagonist of the disclosure is a BMPRII polypeptide. The term BMPRII polypeptide collectively refers to naturally occurring polypeptides as well as truncations and variants thereof such as those described herein. Preferably BMPRII polypeptides comprise a ligand-binding domain of a BMPRII polypeptide or modified (variant) form thereof. For example, in some embodiments, a BMPRII polypeptide may comprise an extracellular domain of BMPRII. Preferably, BMPRII polypeptides to be used in accordance with the methods and uses described herein are soluble polypeptides. In some embodiments, a BMPRII polypeptide may comprise an amino acid sequence that is at least 70%, 75% 80%, 85%, 90%, 91%, 92%, 93%, 94%, 95%, 96%, 97%, 98%, 99%, or 100% identical to amino acids 27-150 of SEQ ID NO: 14. In some embodiments, a BMPRII polypeptide may comprise an amino acid sequence that is at least 70%, 75% 80%, 85%, 90%, 91%, 92%, 93%, 94%, 95%, 96%, 97%, 98%, 99%, or 100% identical to amino acids 34-123 of SEQ ID NO: 14. In some embodiments, a BMPRII polypeptide may comprise an amino acid sequence that is at least 70%, 75% 80%, 85%, 90%, 91%, 92%, 93%, 94%, 95%, 96%, 97%, 98%, 99%, or 100% identical to the amino acid sequence of SEQ ID NO: 15. In some embodiments, a BMPRII polypeptide may comprise an amino acid sequence that is at least 70%, 75% 80%, 85%, 90%, 91%, 92%, 93%, 94%, 95%, 96%, 97%, 98%, 99%, or 100% identical to the amino acid sequence of SEQ ID NO: 69. In some embodiments, a BMPRII polypeptide may comprise an amino acid sequence that is at least 70%, 75% 80%, 85%, 90%, 91%, 92%, 93%, 94%, 95%, 96%, 97%, 98%, 99%, or 100% identical to the amino acid sequence of SEQ ID NO: 71.

In certain aspects, a BMP antagonist of the disclosure is an ALK1 polypeptide. The term ALK1 polypeptide collectively refers to naturally occurring polypeptides as well as truncations and variants thereof such as those described herein. Preferably ALK1 polypeptides comprise a ligand-binding domain of an ALK1 polypeptide or modified (variant) form thereof. For example, in some embodiments, an ALK1 polypeptide may comprise an extracellular domain of ALK1. Preferably, ALK1 polypeptides to be used in accordance with the methods and uses described herein are soluble polypeptides. In some embodiments, an ALK1 polypeptide may comprise an amino acid sequence that is at least 70%, 75% 80%, 85%, 90%, 91%, 92%, 93%, 94%, 95%, 96%, 97%, 98%, 99%, or 100% identical to amino acids 22-118 of SEQ ID NO: 20. In some embodiments, an ALK1 polypeptide may comprise an amino acid sequence that is at least 70%, 75% 80%, 85%, 90%, 91%, 92%, 93%, 94%, 95%, 96%, 97%, 98%, 99%, or 100% identical to amino acids 34-95 of SEQ ID NO: 20. In some embodiments, an ALK1 polypeptide may comprise an amino acid sequence that is at least 70%, 75% 80%, 85%, 90%, 91%, 92%, 93%, 94%, 95%, 96%, 97%, 98%, 99%, or 100% identical to the amino acid sequence of SEQ ID NO: 21. In some embodiments, an ALK1 polypeptide may comprise an amino acid sequence that is at least 70%, 75% 80%, 85%, 90%, 91%, 92%, 93%, 94%, 95%, 96%, 97%, 98%, 99%, or 100% identical to the amino acid sequence of SEQ ID NO: 74. In some embodiments, an ALK1 polypeptide may comprise an amino acid sequence that is at least 70%, 75% 80%, 85%, 90%, 91%, 92%, 93%, 94%, 95%, 96%, 97%, 98%, 99%, or 100% identical to the amino acid sequence of SEQ ID NO: 76.

In certain aspects, a BMP antagonist of the disclosure is an endoglin polypeptide. The term endoglin polypeptide collectively refers to naturally occurring polypeptides as well as truncations and variants thereof such as those described herein. Preferably endoglin polypeptides comprise a ligand-binding domain of an endoglin polypeptide or modified (variant) form thereof. For example, in some embodiments, an endoglin polypeptide may comprise an extracellular domain of endoglin. Preferably, endoglin polypeptides to be used in accordance with the methods and uses described herein are soluble polypeptides. In some embodiments, an endoglin polypeptide may comprise an amino acid sequence that is at least 70%, 75% 80%, 85%, 90%, 91%, 92%, 93%, 94%, 95%, 96%, 97%, 98%, 99%, or 100% identical to amino acids 26-378 of SEQ ID NO: 24. In some embodiments, an endoglin polypeptide may comprise an amino acid sequence that is at least 70%, 75% 80%, 85%, 90%, 91%, 92%, 93%, 94%, 95%, 96%, 97%, 98%, 99%, or 100% identical to amino acids 42-333 of SEQ ID NO: 24. In some embodiments, an endoglin polypeptide may comprise an amino acid sequence that is at least 70%, 75% 80%, 85%, 90%, 91%, 92%, 93%, 94%, 95%, 96%, 97%, 98%, 99%, or 100% identical to amino acids 26-346 of SEQ ID NO: 24. In some embodiments, an endoglin polypeptide may comprise an amino acid sequence that is at least 70%, 75% 80%, 85%, 90%, 91%, 92%, 93%, 94%, 95%, 96%, 97%, 98%, 99%, or 100% identical to amino acids 27-581 of SEQ ID NO: 24. In some embodiments, an endoglin polypeptide may comprise an amino acid sequence that is at least 70%, 75% 80%, 85%, 90%, 91%, 92%, 93%, 94%, 95%, 96%, 97%, 98%, 99%, or 100% identical to amino acids 26-359 of SEQ ID NO: 24. In some embodiments, an endoglin polypeptide may comprise an amino acid sequence that is at least 70%, 75% 80%, 85%, 90%, 91%, 92%, 93%, 94%, 95%, 96%, 97%, 98%, 99%, or 100% identical to the amino acid sequence of SEQ ID NO: 78. In some embodiments, an endoglin polypeptide may comprise an amino acid sequence that is at least 70%, 75% 80%, 85%, 90%, 91%, 92%, 93%, 94%, 95%, 96%, 97%, 98%, 99%, or 100% identical to the amino acid sequence of SEQ ID NO: 80. In some embodiments, an endoglin polypeptide may comprise an amino acid sequence that is at least 70%, 75% 80%, 85%, 90%, 91%, 92%, 93%, 94%, 95%, 96%, 97%, 98%, 99%, or 100% identical to the amino acid sequence of SEQ ID NO: 28. In some embodiments, an endoglin polypeptide may comprise an amino acid sequence that is at least 70%, 75% 80%, 85%, 90%, 91%, 92%, 93%, 94%, 95%, 96%, 97%, 98%, 99%, or 100% identical to the amino acid sequence of SEQ ID NO: 29. In some embodiments, an endoglin polypeptide may comprise an amino acid sequence that is at least 70%, 75% 80%, 85%, 90%, 91%, 92%, 93%, 94%, 95%, 96%, 97%, 98%, 99%, or 100% identical to the amino acid sequence of SEQ ID NO: 30. In some embodiments, an endoglin polypeptide may comprise an amino acid sequence that is at least 70%, 75% 80%, 85%, 90%, 91%, 92%, 93%, 94%, 95%, 96%, 97%, 98%, 99%, or 100% identical to the amino acid sequence of SEQ ID NO: 31. In some embodiments, endoglin polypeptides do not comprise a sequence consisting of amino acids 379-430 of SEQ ID NO: 24. In some embodiments, endoglin polypeptides do not comprise more than 50 consecutive amino acids from a sequence consisting of amino acids 379-586 of SEQ ID NO: 24.

In certain aspects, a BMP antagonist of the disclosure is a BMP10 propeptide (BMP10pro) polypeptide. The term BMP10pro polypeptide collectively refers to naturally occurring propeptide polypeptides as well as truncations and variants thereof such as those described herein. Preferably BMP10pro polypeptides comprise a ligand-binding domain of a BMP10 propeptide polypeptide or modified (variant) form thereof. Preferably, BMP10pro polypeptides to be used in accordance with the methods and uses described herein are soluble polypeptides. In some embodiments, a BMP10pro polypeptide may comprise an amino acid sequence that is at least 70%, 75%, 80%, 85%, 90%, 91%, 92%, 93%, 94%, 95%, 96%, 97%, 98%, 99%, or 100% identical to a sequence that begins at a position corresponding to any one of amino acids 1-6 of SEQ ID NO: 34 and ends at a position corresponding any one of amino acids 291-295 of SEQ ID NO: 34. In some embodiments, a BMP10pro polypeptide may comprise an amino acid sequence that is at least 70%, 75%, 80%, 85%, 90%, 91%, 92%, 93%, 94%, 95%, 96%, 97%, 98%, 99%, or 100% identical to a sequence that begins at a position corresponding to any one of amino acids 1-6 of SEQ ID NO: 34 and ends at a position corresponding any one of amino acids 291-294 of SEQ ID NO: 34, wherein the polypeptide does not comprise the sequence of amino acids RIRR. In some embodiments, a BMP10pro polypeptide may comprise an amino acid sequence that is at least 70%, 75%, 80%, 85%, 90%, 91%, 92%, 93%, 94%, 95%, 96%, 97%, 98%, 99%, or 100% identical to amino acids 1-291 of SEQ ID NO: 34. In some embodiments, a BMP10pro polypeptide may comprise an amino acid sequence that is at least 70%, 75%, 80%, 85%, 90%, 91%, 92%, 93%, 94%, 95%, 96%, 97%, 98%, 99%, or 100% identical to amino acids 1-291 of SEQ ID NO: 34, wherein the polypeptide does not comprise the sequence of amino acids RIRR. In some embodiments, a BMP10pro polypeptide may comprise an amino acid sequence that is at least 70%, 75%, 80%, 85%, 90%, 91%, 92%, 93%, 94%, 95%, 96%, 97%, 98%, 99%, or 100% identical to amino acids 1-294 of SEQ ID NO: 34. In some embodiments, a BMP10pro polypeptide may comprise an amino acid sequence that is at least 70%, 75%, 80%, 85%, 90%, 91%, 92%, 93%, 94%, 95%, 96%, 97%, 98%, 99%, or 100% identical to amino acids 1-294 of SEQ ID NO: 34, wherein the polypeptide does not comprise the sequence of amino acids RIRR. In some embodiments, a BMP10pro polypeptide may comprise an amino acid sequence that is at least 70%, 75%, 80%, 85%, 90%, 91%, 92%, 93%, 94%, 95%, 96%, 97%, 98%, 99%, or 100% identical to a sequence that begins at a position corresponding to any one of amino acids 1-6 of SEQ ID NO: 34 and ends at a position corresponding any one of amino acids 291-291 of SEQ ID NO: 34, wherein the C-terminus of the polypeptide is not R295 of SEQ ID NO: 34. In some embodiments, a BMP10pro polypeptide may comprise an amino acid sequence that is at least 70%, 75%, 80%, 85%, 90%, 91%, 92%, 93%, 94%, 95%, 96%, 97%, 98%, 99%, or 100% identical to a sequence that begins at a position corresponding to any one of amino acids 1-6 of SEQ ID NO: 34 and ends at a position corresponding any one of amino acids 291-294 of SEQ ID NO: 34, wherein the C-terminus of the polypeptide is not R295 of SEQ ID NO: 34. In some embodiments, a BMP10pro polypeptide may comprise an amino acid sequence that is at least 70%, 75%, 80%, 85%, 90%, 91%, 92%, 93%, 94%, 95%, 96%, 97%, 98%, 99%, or 100% identical to amino acids 1-291 of SEQ ID NO: 34, wherein the C-terminus of the polypeptide is not R295 of SEQ ID NO: 34. In some embodiments, a BMP10pro polypeptide may comprise an amino acid sequence that is at least 70%, 75%, 80%, 85%, 90%, 91%, 92%, 93%, 94%, 95%, 96%, 97%, 98%, 99%, or 100% identical to amino acids 1-294 of SEQ ID NO: 34, wherein the C-terminus of the polypeptide is not R295 of SEQ ID NO: 34. In some embodiments, a BMP10pro polypeptide may comprise an amino acid sequence that is at least 70%, 75% 80%, 85%, 90%, 91%, 92%, 93%, 94%, 95%, 96%, 97%, 98%, 99%, or 100% identical to the amino acid sequence of SEQ ID NO: 82. In some embodiments, a BMP10pro polypeptide may comprise an amino acid sequence that is at least 70%, 75% 80%, 85%, 90%, 91%, 92%, 93%, 94%, 95%, 96%, 97%, 98%, 99%, or 100% identical to the amino acid sequence of SEQ ID NO: 84. In some embodiments, a BMP10pro polypeptide may comprise an amino acid sequence that is at least 70%, 75% 80%, 85%, 90%, 91%, 92%, 93%, 94%, 95%, 96%, 97%, 98%, 99%, or 100% identical to the amino acid sequence of SEQ ID NO: 85. In some embodiments, a BMP10pro polypeptide may comprise an amino acid sequence that is at least 70%, 75% 80%, 85%, 90%, 91%, 92%, 93%, 94%, 95%, 96%, 97%, 98%, 99%, or 100% identical to the amino acid sequence of SEQ ID NO: 87.

In certain aspects, BMP10pro polypeptides, ActRII polypeptides, BMPRII polypeptides, ALK1 polypeptides, and endoglin polypeptides, including variants thereof, may be fusion proteins. For example, in some embodiments, a BMP10pro polypeptide, ActRII polypeptide, BMPRII polypeptide, ALK1 polypeptide, or endoglin polypeptide may be a fusion protein comprising a BMP10pro polypeptide, ActRII polypeptide, BMPRII polypeptide, ALK1 polypeptide, or endoglin polypeptide domain and one or more heterologous (non-BMP10pro, non-ActRII, non-BMPRII, non-ALK1, or non-endoglin) polypeptide domains. In some embodiments, a BMP10pro polypeptide, ActRII polypeptide, BMPRII polypeptide, ALK1 polypeptide, or endoglin polypeptide may be a fusion protein that has, as one domain, an amino acid sequence derived from a BMP10pro polypeptide, ActRII polypeptide, BMPRII polypeptide, ALK1 polypeptide, or endoglin polypeptide (e.g., a ligand-binding domain of a BMP propeptide, ActRII receptor, BMPRII receptor, ALK1 receptor, or endoglin receptor or a variant thereof) and one or more heterologous domains that provide a desirable property, such as improved pharmacokinetics, easier purification, targeting to particular tissues, etc. For example, a domain of a fusion protein may enhance one or more of in vivo stability, in vivo half-life, uptake/administration, tissue localization or distribution, formation of protein complexes, multimerization of the fusion protein, and/or purification. Optionally, a BMP10pro polypeptide, ActRII polypeptide, BMPRII polypeptide, ALK1 polypeptide, or endoglin polypeptide domain of a fusion protein is connected directly (fused) to one or more heterologous polypeptide domains, or an intervening sequence, such as a linker, may be positioned between the amino acid sequence of the BMP10pro polypeptide, ActRII polypeptide, BMPRII polypeptide, ALK1 polypeptide, or endoglin polypeptide and the amino acid sequence of the one or more heterologous domains. In certain embodiments, a BMP10pro, ActRII, BMPRII, ALK1, or endoglin polypeptide fusion comprises a linker positioned between the heterologous domain and the BMP10pro domain, ActRII domain, BMPRII domain, ALK1 domain, or endoglin domain. The linker may correspond to the roughly 4-15 amino acid unstructured region at the C-terminal end of the BMP10pro domain, ActRII domain, BMPRII domain, ALK1 domain, or endoglin domain, or it may be an artificial sequence of between 3 and 15, 20, 30, 50 or more amino acids that are relatively free of secondary structure. A linker may be rich in glycine and proline residues and may, for example, contain repeating sequences of threonine/serine and glycines. Examples of linkers include, but are not limited to, the sequences TGGG (SEQ ID NO: 45), SGGG (SEQ ID NO: 46), TGGGG (SEQ ID NO: 43), SGGGG (SEQ ID NO: 44), GGGGS (SEQ ID NO: 47), GGGG (SEQ ID NO: 42), and GGG (SEQ ID NO: 41). In some embodiments, BMP10pro, ActRII, BMPRII, ALK1, and endoglin fusion proteins may comprise a constant domain of an immunoglobulin, including, for example, the Fc portion of an immunoglobulin. For example, an amino acid sequence that is derived from an Fc domain of an IgG (IgG1, IgG2, IgG3, or IgG4), IgA (IgA1 or IgA2), IgE, or IgM immunoglobulin. For example, am Fc portion of an immunoglobulin domain may comprise, consist essentially of, or consist of an amino acid sequence that is at least 70%, 75%, 80%, 85%, 90%, 91%, 92%, 93%, 94%, 95%, 96%, 97%, 98%, 99% or 100% identical to any one of SEQ ID NOs: 36-40. Such immunoglobulin domains may comprise one or more amino acid modifications (e.g., deletions, additions, and/or substitutions) that confer an altered Fc activity, e.g., decrease of one or more Fc effector functions. In some embodiments, a BMP10pro, ActRII, BMPRII, ALK1, or endoglin fusion protein comprises an amino acid sequence as set forth in the formula A-B-C. For example, the B portion is an N- and C-terminally truncated BMP10pro polypeptide as described herein. The A and C portions may be independently zero, one, or more than one amino acids, and both A and C portions are heterologous to B. The A and/or C portions may be attached to the B portion via a linker sequence. In certain embodiments, a BMP10pro, ActRII, BMPRII, ALK1, or endoglin fusion protein comprises a leader sequence. The leader sequence may be a native BMP10pro, ActRII, BMPRII, ALK1, or endoglin leader sequence or a heterologous leader sequence. In certain embodiments, the leader sequence is a tissue plasminogen activator (TPA) leader sequence.

A BMP10pro, ActRII, BMPRII, ALK1, or endoglin polypeptide, including variants thereof, may comprise a purification subsequence, such as an epitope tag, a FLAG tag, a polyhistidine sequence, and a GST fusion. Optionally, a BMP10pro, ActRII, BMPRII, ALK1, or endoglin polypeptide includes one or more modified amino acid residues selected from: a glycosylated amino acid, a PEGylated amino acid, a farnesylated amino acid, an acetylated amino acid, a biotinylated amino acid, an amino acid conjugated to a lipid moiety, and an amino acid conjugated to an organic derivatizing agent. BMP10pro, ActRII, ALK1, and endoglin polypeptides may comprise at least one N-linked sugar, and may include two, three or more N-linked sugars. Such polypeptides may also comprise O-linked sugars. In general, it is preferable that BMP10pro, ActRII, BMPRII, ALK1, and endoglin polypeptides be expressed in a mammalian cell line that mediates suitably natural glycosylation of the polypeptide so as to diminish the likelihood of an unfavorable immune response in a patient. BMP10pro, ActRII, BMPRII, ALK1, and endoglin polypeptides may be produced in a variety of cell lines that glycosylate the protein in a manner that is suitable for patient use, including engineered insect or yeast cells, and mammalian cells such as COS cells, CHO cells, HEK cells and NSO cells. In some embodiments, a BMP10pro, ActRII, BMPRII, ALK1, or endoglin polypeptide is glycosylated and has a glycosylation pattern obtainable from a Chinese hamster ovary cell line. In some embodiments, BMP10pro, ActRII, BMPRII, ALK1, and endoglin polypeptides of the disclosure exhibit a serum half-life of at least 4, 6, 12, 24, 36, 48, or 72 hours in a mammal (e.g., a mouse or a human). Optionally, BMP10pro, ActRII, BMPRII, ALK1, and endoglin polypeptides may exhibit a serum half-life of at least 6, 8, 10, 12, 14, 20, 25, or 30 days in a mammal (e.g., a mouse or a human).

In certain aspects, a BMP antagonist to be used in accordance with the teachings of the disclosure is an antibody or combination of antibodies. In some embodiments, the antibody or combination of antibodies binds to at least BMP10. In some embodiments, the antibody or combination of antibodies that binds to BMP10 further binds to one or more of BMP9, BMP6, BMP3b, BMP5, ActRII, BMPRII, ALK1, and endoglin. In some embodiments, the antibody or combination of antibodies binds to at least BMP9. In some embodiments, the antibody or combination of antibodies that binds to BMP9 further binds to one or more of BMP10, BMP6, BMP3b, BMP5, ActRII, BMPRII, ALK1, and endoglin. In some embodiments, the antibody or combination of antibodies binds to at least BMP6. In some embodiments, the antibody or combination of antibodies that binds to BMP6 further binds to one or more of BMP9, BMP10, BMP3b, BMP5, ActRII, BMPRII, ALK1, and endoglin. In some embodiments, the antibody or combination of antibodies binds to at least BMP3b. In some embodiments, the antibody or combination of antibodies that binds to BMP10 further binds to one or more of BMP9, BMP6, BMP10, BMP5, ActRII, BMPRII, ALK1, and endoglin. In some embodiments, the antibody or combination of antibodies binds to at least BMP5. In some embodiments, the antibody or combination of antibodies that binds to BMP5 further binds to one or more of BMP9, BMP6, BMP3b, BMP10, ActRII, BMPRII, ALK1, and endoglin. In some embodiments, the antibody or combination of antibodies binds to at least ActRII. In some embodiments, the antibody or combination of antibodies that binds to ActRII further binds to one or more of BMP10, BMP9, BMP6, BMP3b, BMP5, BMPRII, ALK1, and endoglin. In some embodiments, the antibody or combination of antibodies binds to at least BMPRII. In some embodiments, the antibody or combination of antibodies that binds to BMPRII further binds to one or more of BMP10, BMP9, BMP6, BMP3b, BMP5, ActRII, ALK1, and endoglin. In some embodiments, the antibody or combination of antibodies binds to at least ALK1. In some embodiments, the antibody or combination of antibodies that binds to ALK1 further binds to one or more of BMP10, BMP9, BMP6, BMP3b, BMP5, ActRII, BMPRII, and endoglin. In some embodiments, the antibody or combination of antibodies binds to at least endoglin. In some embodiments, the antibody or combination of antibodies that binds to endoglin further binds to one or more of BMP10, BMP9, BMP6, BMP3b, BMP5, ActRII, BMPRII, and ALK1. In some embodiments, the antibody or combination of antibodies binds to at least BMP10 and BMP9. In some embodiments, the antibody or combination of antibodies that binds to BMP10 and BMP9 further binds to one or more of BMP6, BMP3b, BMP5, ActRII, BMPRII, ALK1, and endoglin. In certain preferred embodiments, antibodies or combinations of antibodies disclosed herein inhibit activity of one or more of BMP10, BMP9, BMP6, BMP3b, BMP5, ActRII, BMPRII, ALK1, and endoglin. In certain preferred embodiments, a BMP10 antibody binds to the mature BMP10 protein. In certain preferred embodiments, a BMP10 antibody binds to the mature BMP10 protein competitively with a BMP10 propeptide.

In certain aspects, a BMP antagonist to be used in accordance with the teachings of the disclosure is a small molecule or combination of small molecules. In some embodiments, a small molecule or combination of small molecules inhibits at least BMP10 activity. In some embodiments, a small molecule or combination of small molecules that inhibits BMP10 activity further inhibits the activity of one or more of BMP9, BMP6, BMP3b, BMP5, ActRII, BMPRII, ALK1, endoglin, and Smad proteins (e.g., Smads 2 and/or 3). In some embodiments, a small molecule or combination of small molecules inhibits at least BMP9 activity. In some embodiments, a small molecule or combination of small molecules that inhibits BMP9 activity further inhibits the activity of one or more of BMP10, BMP6, BMP3b, BMP5, ActRII, BMPRII, ALK1, endoglin, and Smad proteins (e.g., Smads 2 and/or 3). In some embodiments, a small molecule or combination of small molecules inhibits at least BMP6 activity. In some embodiments, a small molecule or combination of small molecules that inhibits BMP6 activity further inhibits the activity of one or more of BMP10, BMP9, BMP3b, BMP5, ActRII, BMPRII, ALK1, endoglin, and Smad proteins (e.g., Smads 2 and/or 3). In some embodiments, a small molecule or combination of small molecules inhibits at least BMP3b activity. In some embodiments, a small molecule or combination of small molecules that inhibits BMP3b activity further inhibits the activity of one or more of BMP10, BMP9, BMP6, BMP5, ActRII, BMPRII, ALK1, endoglin, and Smad proteins (e.g., Smads 2 and/or 3). In some embodiments, a small molecule or combination of small molecules inhibits at least BMP5 activity. In some embodiments, a small molecule or combination of small molecules that inhibits BMP5 activity further inhibits the activity of one or more of BMP10, BMP9, BMP6, BMP3b, ActRII, BMPRII, ALK1, endoglin, and Smad proteins (e.g., Smads 2 and/or 3). In some embodiments, a small molecule or combination of small molecules inhibits at least ActRII activity. In some embodiments, a small molecule or combination of small molecules that inhibits ActRII activity further inhibits the activity of one or more of BMP10, BMP9, BMP6, BMP3b, BMP5, BMPRII, ALK1, endoglin, and Smad proteins (e.g., Smads 2 and/or 3). In some embodiments, a small molecule or combination of small molecules inhibits at least BMPRII activity. In some embodiments, a small molecule or combination of small molecules that inhibits BMPRII activity further inhibits the activity of one or more of BMP10, BMP9, BMP6, BMP3b, BMP5, ActRII, ALK1, endoglin, and Smad proteins (e.g., Smads 2 and/or 3). In some embodiments, a small molecule or combination of small molecules inhibits at least ALK1 activity. In some embodiments, a small molecule or combination of small molecules that inhibits ALK1 activity further inhibits the activity of one or more of BMP10, BMP9, BMP6, BMP3b, BMP5, ActRII, BMPRII, endoglin, and Smad proteins (e.g., Smads 2 and/or 3). In some embodiments, a small molecule or combination of small molecules inhibits at least endoglin activity. In some embodiments, a small molecule or combination of small molecules that inhibits endoglin activity further inhibits the activity of one or more of BMP10, BMP9, BMP6, BMP3b, BMP5, ActRII, BMPRII, ALK1, and Smad proteins (e.g., Smads 2 and/or 3). In some embodiments, a small molecule or combination of small molecules inhibits at least one or more Smads (e.g., Smads 2 and/or 3) activity. In some embodiments, a small molecule or combination of small molecules that inhibits one or more Smads activity further inhibits the activity of one or more of BMP10, BMP9, BMP6, BMP3b, BMP5, ActRII, BMPRII, ALK1, and endoglin. In some embodiments, a small molecule or combination of small molecules inhibits at least BMP10 and BMP9 activity. In some embodiments, a small molecule or combination of small molecules that inhibits BMP10 and BMP9 activity further inhibits the activity of one or more of BMP6, BMP3b, BMP5, ActRII, BMPRII, ALK1, endoglin, and Smad proteins (e.g., Smads 2 and/or 3).

In certain aspects, a BMP antagonist to be used in accordance with the teachings of the disclosure is a nucleotide or combination of nucleotides. In some embodiments, a nucleotide or combination of nucleotides inhibits at least BMP10 activity. In some embodiments, a nucleotide or combination of nucleotides that inhibits BMP10 activity further inhibits the activity of one or more of BMP9, BMP6, BMP3b, BMP5, ActRII, BMPRII, ALK1, endoglin, and Smad proteins (e.g., Smads 2 and/or 3). In some embodiments, a nucleotide or combination of nucleotides inhibits at least BMP9 activity. In some embodiments, a nucleotide or combination of nucleotides that inhibits BMP9 activity further inhibits the activity of one or more of BMP10, BMP6, BMP3b, BMP5, ActRII, BMPRII, ALK1, endoglin, and Smad proteins (e.g., Smads 2 and/or 3). In some embodiments, a nucleotide or combination of nucleotides inhibits at least BMP6 activity. In some embodiments, a nucleotide or combination of nucleotides that inhibits BMP6 activity further inhibits the activity of one or more of BMP10, BMP9, BMP3b, BMP5, ActRII, BMPRII, ALK1, endoglin, and Smad proteins (e.g., Smads 2 and/or 3). In some embodiments, a nucleotide or combination of nucleotides inhibits at least BMP3b activity. In some embodiments, a nucleotide or combination of nucleotides that inhibits BMP3b activity further inhibits the activity of one or more of BMP10, BMP9, BMP6, BMP5, ActRII, BMPRII, ALK1, endoglin, and Smad proteins (e.g., Smads 2 and/or 3). In some embodiments, a nucleotide or combination of nucleotides inhibits at least BMP5 activity. In some embodiments, a nucleotide or combination of nucleotides that inhibits BMP5 activity further inhibits the activity of one or more of BMP10, BMP9, BMP6, BMP3b, ActRII, BMPRII, ALK1, endoglin, and Smad proteins (e.g., Smads 2 and/or 3). In some embodiments, a nucleotide or combination of nucleotides inhibits at least ActRII activity. In some embodiments, a nucleotide or combination of nucleotides that inhibits ActRII activity further inhibits the activity of one or more of BMP10, BMP9, BMP6, BMP3b, BMP5, BMPRII, ALK1, endoglin, and Smad proteins (e.g., Smads 2 and/or 3). In some embodiments, a nucleotide or combination of nucleotides inhibits at least BMPRII activity. In some embodiments, a nucleotide or combination of nucleotides that inhibits BMPRII activity further inhibits the activity of one or more of BMP10, BMP9, BMP6, BMP3b, BMP5, ActRII, ALK1, endoglin, and Smad proteins (e.g., Smads 2 and/or 3). In some embodiments, a nucleotide or combination of nucleotides inhibits at least ALK1 activity. In some embodiments, a nucleotide or combination of nucleotides that inhibits ALK1 activity further inhibits the activity of one or more of BMP10, BMP9, BMP6, BMP3b, BMP5, ActRII, BMPRII, endoglin, and Smad proteins (e.g., Smads 2 and/or 3). In some embodiments, a nucleotide or combination of nucleotides inhibits at least endoglin activity. In some embodiments, a nucleotide or combination of nucleotides that inhibits endoglin activity further inhibits the activity of one or more of BMP10, BMP9, BMP6, BMP3b, BMP5, ActRII, BMPRII, ALK1, and Smad proteins (e.g., Smads 2 and/or 3). In some embodiments, a nucleotide or combination of nucleotides inhibits at least one or more Smads (e.g., Smads 2 and/or 3) activity. In some embodiments, a nucleotide or combination of nucleotides that inhibits one or more Smads activity further inhibits the activity of one or more of BMP10, BMP9, BMP6, BMP3b, BMP5, ActRII, BMPRII, ALK1, and endoglin. In some embodiments, a nucleotide or combination of nucleotides inhibits at least BMP10 and BMP9 activity. In some embodiments, a nucleotide or combination of nucleotides that inhibits BMP10 and BMP9 activity further inhibits the activity of one or more of BMP6, BMP3b, BMP5, ActRII, BMPRII, ALK1, endoglin, and Smad proteins (e.g., Smads 2 and/or 3).

In certain aspects, the present disclosure provides to BMP10 propeptides. As demonstrated by the examples herein, BMP10 propeptides have been generated that that bind to and antagonize activity of a mature BMP10 polypeptide. It was further discovered that these BMP10 propeptides bind to other BMP proteins, particularly BMP9, BMP6, and BMP3b and to a lesser extent BMP5. Therefore, BMP propeptides may antagonize other members of the BMP family and therefore may be useful in the treatment of additional disorder or conditions associated with these other BMP proteins (e.g., BMP9-, BMP6, BMP3b, and BMP6-associated disorders or conditions. Moreover, a C-terminally truncated BMP10 propeptide variant, which lacks the four C-terminal amino acids of the propeptide domain, was surprisingly found to have increased BMP10 antagonizing activity compared to a longer length BMP10 propeptide variant. Therefore, BMP10 propeptides can tolerate C-terminal truncations of 1, 2, 3, or 4 amino acids without losing BMP10 antagonizing activity. In addition, BMP10 propeptide variants lacking the four C-terminal amino acids may have increased BMP10 antagonizing activity and therefore be useful in certain experimental and clinical situations where such increased BMP10 antagonism is desirable. The disclosure further provides nucleic acid sequence encoding BMP10 propeptides, pharmaceutical compositions and kits comprising BMP10 propeptides and methods of manufacturing BMP10 propeptides.

In certain aspects, the disclosure provides a BMP10 propeptide (BMP10pro) polypeptide comprising an amino acid sequence that is at least 70%, 75%, 80%, 85%, 90%, 91%, 92%, 93%, 94%, 95%, 96%, 97%, 98%, 99%, or 100% identical to a sequence that begins at a position corresponding to any one of amino acids 1-6 of SEQ ID NO: 34 and ends at a position corresponding any one of amino acids 292-295 of SEQ ID NO: 34. In some embodiments, BMP10pro polypeptides do not comprise the sequence of amino acids RIRR. In some embodiments, C-terminus of a BMP10pro polypeptide is not R296 of SEQ ID NO: 34. In some embodiments, BMP10pro polypeptides comprise an amino acid sequence that is at least 70%, 75%, 80%, 85%, 90%, 91%, 92%, 93%, 94%, 95%, 96%, 97%, 98%, 99%, or 100% identical to amino acids 1-292 of SEQ ID NO: 34. In some embodiments, BMP10pro polypeptides comprise an amino acid sequence that is at least 70%, 75%, 80%, 85%, 90%, 91%, 92%, 93%, 94%, 95%, 96%, 97%, 98%, 99%, or 100% identical to amino acids 1-292 of SEQ ID NO: 34 wherein polypeptide does not comprise the sequence of amino acids RIRR. In some embodiments, BMP10pro polypeptides comprise an amino acid sequence that is at least 70%, 75%, 80%, 85%, 90%, 91%, 92%, 93%, 94%, 95%, 96%, 97%, 98%, 99%, or 100% identical to amino acids 1-292 of SEQ ID NO: 34 wherein C-terminus of the polypeptide is not R296 of SEQ ID NO: 34. In some embodiments, BMP10pro polypeptides comprise an amino acid sequence that is at least 70%, 75%, 80%, 85%, 90%, 91%, 92%, 93%, 94%, 95%, 96%, 97%, 98%, 99%, or 100% identical to amino acids 1-295 of SEQ ID NO: 34. In some embodiments, BMP10pro polypeptides comprise an amino acid sequence that is at least 70%, 75%, 80%, 85%, 90%, 91%, 92%, 93%, 94%, 95%, 96%, 97%, 98%, 99%, or 100% identical to amino acids 1-295 of SEQ ID NO: 34 wherein polypeptide does not comprise the sequence of amino acids RIRR.

In some embodiments, BMP10pro polypeptides comprise an amino acid sequence that is at least 70%, 75%, 80%, 85%, 90%, 91%, 92%, 93%, 94%, 95%, 96%, 97%, 98%, 99%, or 100% identical to amino acids 1-295 of SEQ ID NO: 34 wherein C-terminus of the polypeptide is not R296 of SEQ ID NO: 34.

As previously discussed, the disclosure provides BMP10pro polypeptides that are fusion proteins comprising a BMP10pro polypeptide domain and one or more heterologous (non-BMP10pro polypeptide domains. For example, BMP10pro polypeptides are Fc fusion proteins comprising a BMP10pro polypeptide domain and an immunoglobulin Fc domain. Optionally, a BMP10pro polypeptide domain of a fusion protein is connected directly (fused) to one or more heterologous polypeptide domains, or an intervening sequence, such as a linker, may be positioned between the amino acid sequence of the BMP10pro polypeptide and the amino acid sequence of the one or more heterologous domains. In certain embodiments, a BMP10pro polypeptide fusion comprises a linker positioned between the heterologous domain and the BMP10pro domain. The linker may correspond to the roughly 4-15 amino acid unstructured region at the C-terminal end of the BMP10pro domain, or it may be an artificial sequence of between 3 and 15, 20, 30, 50 or more amino acids that are relatively free of secondary structure. A linker may be rich in glycine and proline residues and may, for example, contain repeating sequences of threonine/serine and glycines. Examples of linkers include, but are not limited to, the sequences TGGG (SEQ ID NO: 45), SGGG (SEQ ID NO: 46), TGGGG (SEQ ID NO: 43), SGGGG (SEQ ID NO: 44), GGGGS (SEQ ID NO: 47), GGGG (SEQ ID NO: 42), and GGG (SEQ ID NO: 41). In some embodiments, BMP10pro endoglin fusion proteins may comprise a constant domain of an immunoglobulin, including, for example, the Fc portion of an immunoglobulin. For example, an amino acid sequence that is derived from an Fc domain of an IgG (IgG1, IgG2, IgG3, or IgG4), IgA (IgA1 or IgA2), IgE, or IgM immunoglobulin. For example, am Fc portion of an immunoglobulin domain may comprise of an amino acid sequence that is at least 70%, 75%, 80%, 85%, 90%, 91%, 92%, 93%, 94%, 95%, 96%, 97%, 98%, 99% or 100% identical to any one of SEQ ID NOs: 36-40. Such immunoglobulin domains may comprise one or more amino acid modifications (e.g., deletions, additions, and/or substitutions) that confer an altered Fc activity, e.g., decrease of one or more Fc effector functions. In some embodiments, a BMP10pro fusion protein comprises an amino acid sequence as set forth in the formula A-B-C. For example, the B portion is an N- and C-terminally truncated BMP10pro polypeptide as described herein. The A and C portions may be independently zero, one, or more than one amino acids, and both A and C portions are heterologous to B. The A and/or C portions may be attached to the B portion via a linker sequence. In certain embodiments, a BMP10pro fusion protein comprises a leader sequence. The leader sequence may be a native BMP10pro leader sequence or a heterologous leader sequence. In certain embodiments, the leader sequence is a tissue plasminogen activator (TPA) leader sequence.

In certain aspects, the disclosure provides BMP10pro polypeptides that are Fc fusion proteins comprising a BMP10pro polypeptide domain and an immunoglobulin Fc domain. In certain aspects, the disclosure provides a BMP10pro-Fc fusion protein comprising an amino acid sequence that is at least 70%, 75%, 80%, 85%, 90%, 91%, 92%, 93%, 94%, 95%, 96%, 97%, 98%, 99%, or 100% identical to a sequence that begins at a position corresponding to any one of amino acids 1-6 of SEQ ID NO: 34 and ends at a position corresponding any one of amino acids 292-295 of SEQ ID NO: 34. In some embodiments, BMP10pro-Fc fusion proteins do not comprise the sequence of amino acids RIRR. In some embodiments, C-terminus of a BMP10pro domain of a BMP10pro-Fc fusion protein is not R296 of SEQ ID NO: 34. In some embodiments, a BMP10pro-Fc fusion protein comprises an amino acid sequence that is at least 70%, 75%, 80%, 85%, 90%, 91%, 92%, 93%, 94%, 95%, 96%, 97%, 98%, 99%, or 100% identical to amino acids 1-292 of SEQ ID NO: 34. In some embodiments, a BMP10pro-Fc fusion protein comprises an amino acid sequence that is at least 70%, 75%, 80%, 85%, 90%, 91%, 92%, 93%, 94%, 95%, 96%, 97%, 98%, 99%, or 100% identical to amino acids 1-292 of SEQ ID NO: 34 wherein polypeptide does not comprise the sequence of amino acids RIRR. In some embodiments, a BMP10pro-Fc fusion protein comprises an amino acid sequence that is at least 70%, 75%, 80%, 85%, 90%, 91%, 92%, 93%, 94%, 95%, 96%, 97%, 98%, 99%, or 100% identical to amino acids 1-292 of SEQ ID NO: 34 wherein C-terminus of the BMP10pro domain is not R296 of SEQ ID NO: 34. In some embodiments, a BMP10pro-Fc fusion protein comprises an amino acid sequence that is at least 70%, 75%, 80%, 85%, 90%, 91%, 92%, 93%, 94%, 95%, 96%, 97%, 98%, 99%, or 100% identical to amino acids 1-295 of SEQ ID NO: 34. In some embodiments, a BMP10pro-Fc fusion protein comprises an amino acid sequence that is at least 70%, 75%, 80%, 85%, 90%, 91%, 92%, 93%, 94%, 95%, 96%, 97%, 98%, 99%, or 100% identical to amino acids 1-295 of SEQ ID NO: 34 wherein BMP10pro domain does not comprise the sequence of amino acids RIRR. In some embodiments, a BMP10pro-Fc fusion protein comprises an amino acid sequence that is at least 70%, 75%, 80%, 85%, 90%, 91%, 92%, 93%, 94%, 95%, 96%, 97%, 98%, 99%, or 100% identical to amino acids 1-295 of SEQ ID NO: 34 wherein C-terminus of the BMP10pro domain is not R296 of SEQ ID NO: 34. In some embodiments, a BMP10pro-Fc fusion protein comprises an amino acid sequence that is at least 70%, 75% 80%, 85%, 90%, 91%, 92%, 93%, 94%, 95%, 96%, 97%, 98%, 99%, or 100% identical to the amino acid sequence of SEQ ID NO: 82. In some embodiments, a BMP10pro-Fc fusion protein comprises an amino acid sequence that is at least 70%, 75% 80%, 85%, 90%, 91%, 92%, 93%, 94%, 95%, 96%, 97%, 98%, 99%, or 100% identical to the amino acid sequence of SEQ ID NO: 82 wherein the fusion protein does not comprise the sequence of amino acids RIRR. In some embodiments, a BMP10pro-Fc fusion protein comprises an amino acid sequence that is at least 70%, 75% 80%, 85%, 90%, 91%, 92%, 93%, 94%, 95%, 96%, 97%, 98%, 99%, or 100% identical to the amino acid sequence of SEQ ID NO: 82 wherein the C-terminus of the BMP10pro domain is not R296 of SEQ ID NO: 34. In some embodiments, a BMP10pro-Fc fusion protein comprises an amino acid sequence that is at least 70%, 75% 80%, 85%, 90%, 91%, 92%, 93%, 94%, 95%, 96%, 97%, 98%, 99%, or 100% identical to the amino acid sequence of SEQ ID NO: 87. In some embodiments, a BMP10pro-Fc fusion protein comprises an amino acid sequence that is at least 70%, 75% 80%, 85%, 90%, 91%, 92%, 93%, 94%, 95%, 96%, 97%, 98%, 99%, or 100% identical to the amino acid sequence of SEQ ID NO: 87 wherein the fusion protein does not comprise the sequence of amino acids RIRR. In some embodiments, a BMP10pro-Fc fusion protein comprises an amino acid sequence that is at least 70%, 75% 80%, 85%, 90%, 91%, 92%, 93%, 94%, 95%, 96%, 97%, 98%, 99%, or 100% identical to the amino acid sequence of SEQ ID NO: 87 wherein the C-terminus of the BMP10pro domain is not R296 of SEQ ID NO: 34.

A BMP10pro polypeptides, including variants thereof, may comprise a purification subsequence, such as an epitope tag, a FLAG tag, a polyhistidine sequence, and a GST fusion. Optionally, a BMP10pro polypeptide includes one or more modified amino acid residues selected from: a glycosylated amino acid, a PEGylated amino acid, a farnesylated amino acid, an acetylated amino acid, a biotinylated amino acid, an amino acid conjugated to a lipid moiety, and an amino acid conjugated to an organic derivatizing agent. BMP10pro polypeptides may comprise at least one N-linked sugar, and may include two, three or more N-linked sugars. Such polypeptides may also comprise O-linked sugars. In general, it is preferable that BMP10pro polypeptides be expressed in a mammalian cell line that mediates suitably natural glycosylation of the polypeptide so as to diminish the likelihood of an unfavorable immune response in a patient. BMP10pro polypeptides may be produced in a variety of cell lines that glycosylate the protein in a manner that is suitable for patient use, including engineered insect or yeast cells, and mammalian cells such as COS cells, CHO cells, HEK cells and NSO cells. In some embodiments, a BMP10pro polypeptide is glycosylated and has a glycosylation pattern obtainable from a Chinese hamster ovary cell line. In some embodiments, BMP10pro polypeptides exhibit a serum half-life of at least 4, 6, 12, 24, 36, 48, or 72 hours in a mammal (e.g., a mouse or a human). Optionally, BMP10pro may exhibit a serum half-life of at least 6, 8, 10, 12, 14, 20, 25, or 30 days in a mammal (e.g., a mouse or a human).

In certain aspects, the disclosure provides nucleic acids encoding a BMP10 propeptide that do not encode a complete, translatable mature portion of a BMP10. An isolated and/or recombinant polynucleotide may comprise a coding sequence for a BMP10 propeptide, such as described above. An isolated nucleic acid may include a sequence coding for a BMP10 propeptide and a sequence that would code for part or all of a mature portion, but for a stop codon positioned within the mature portion or positioned between the propeptide and the mature portion. In some embodiments, the disclosure provides a nucleic acid sequence that is at least 70%, 75% 80%, 85%, 90%, 91%, 92%, 93%, 94%, 95%, 96%, 97%, 98%, 99%, or 100% identical to the nucleic acid sequence of SEQ ID NO: 83. In some embodiments, the disclosure provides a nucleic acid sequence that is at least 70%, 75% 80%, 85%, 90%, 91%, 92%, 93%, 94%, 95%, 96%, 97%, 98%, 99%, or 100% identical to the nucleic acid sequence of SEQ ID NO: 86. Nucleic acids disclosed herein may be operably linked to a promoter for expression, and the disclosure provides vectors comprising such polynucleotides as well as cells transformed with such polynucleotides. Preferably the cell is a mammalian cell such as a CHO cell.

In certain aspects, the disclosure provides methods for making a BMP10 propeptide. Such a method may include expressing any of the propeptide encoding nucleic acids disclosed herein in a suitable cell, such as a Chinese hamster ovary (CHO) cell. Such a method may comprise culturing a cell under conditions suitable for expression of the propeptide wherein the cell comprises a BMP10 propeptide expression construct. The method may further comprise a step of recovering the propeptide expressed BMP10 propeptide. BMP10 propeptides may be recovered as crude, partially purified or highly purified fractions using any of the well-known techniques for obtaining protein from cell cultures.

In certain aspects, the disclosure provides a use of a BMP10 propeptide for making a medicament for preventing, treating, or reducing the severity of heart failure or one or more complications of heart failure as well as for other cardiac-related uses described herein. In certain aspects, the disclosure provides a BMP10 propeptide for use preventing, treating, or reducing the severity of heart failure or one or more complications of heart failure as well as for other cardiac-related uses described herein.

In certain aspects, the disclosure provides methods for identifying an agent that may be used for treating a heart failure or one or more complications of heart failure. A method may comprise: a) identifying a test agent that binds a mature BMP10 polypeptide competitively with a BMP10 propeptide; and b) evaluating the effect of the agent on a heart disorder. A test agent may be, for example, a variant BMP10 propeptide, an antibody, or a small molecule.

In certain aspects, the disclosure provides pharmaceutical preparations (compositions) comprising a BMP10pro polypeptide and a pharmaceutically acceptable carrier. A pharmaceutical preparation comprising a BMP10pro polypeptide may also comprise one or more additional active agents such as a compound that is used to treat or prevent a disorder or condition as described herein [e.g., heart failure or one or more complications of heart failure]. In some embodiments A pharmaceutical preparation comprising a BMP10pro polypeptide will be pyrogen-free (e.g., pyrogen free to the extent required by regulations governing the quality of products for therapeutic use).

### BRIEF DESCRIPTION OF THE DRAWINGS

FIG. 1 shows an alignment of extracellular domains of human ActRIIB and human ActRIIA with the residues that are deduced herein, based on composite analysis of multiple ActRIIB and ActRIIA crystal structures, to directly contact ligand indicated with boxes.

FIG. 2 shows a multiple sequence alignment of various vertebrate ActRIIB proteins (SEQ ID NOs: 100-105) and human ActRIIA (SEQ ID NO: 122) as well as a consensus ActRII sequence derived from the alignment (SEQ ID NO: 106).

FIG. 3 shows a multiple sequence alignment of various vertebrate ActRIIA proteins and human ActRIIA (SEQ ID NOs: 107-114).

FIG. 4 shows multiple sequence alignment of Fc domains from human IgG isotypes using Clustal 2.1. Hinge regions are indicated by dotted underline. Double underline indicates examples of positions engineered in IgG1 Fc to promote asymmetric chain pairing and the corresponding positions with respect to other isotypes IgG2, IgG3 and IgG4.

FIG. 5 shows the full, unprocessed amino acid sequence for ActRIIB(25-131)-hFc (SEQ ID NO: 123). The TPA leader (residues 1-22) and double-truncated ActRIIB extracellular domain (residues 24-131, using numbering based on the native sequence in SEQ ID NO: 1) are each underlined. Highlighted is the glutamate revealed by sequencing to be the N-terminal amino acid of the mature fusion protein, which is at position 25 relative to SEQ ID NO: 1.

FIG. 6 shows a nucleotide sequence encoding ActRIIB(25-131)-hFc (the coding strand is shown at top, SEQ ID NO: 124, and the complement shown at bottom 3′-5′, SEQ ID NO: 125). Sequences encoding the TPA leader (nucleotides 1-66) and ActRIIB extracellular domain (nucleotides 73-396) are underlined. The corresponding amino acid sequence for ActRIIB(25-131) is also shown.

FIG. 7 shows an alternative nucleotide sequence encoding ActRIIB(25-131)-hFc (the coding strand is shown at top, SEQ ID NO: 126, and the complement shown at bottom 3′-5′, SEQ ID NO: 127). This sequence confers a greater level of protein expression in initial transformants, making cell line development a more rapid process. Sequences encoding the TPA leader (nucleotides 1-66) and ActRIIB extracellular domain (nucleotides 73-396) are underlined, and substitutions in the wild type nucleotide sequence of the ECD (see FIG. 6) are highlighted. The corresponding amino acid sequence for ActRIIB(25-131) is also shown.

FIG. 8 shows the full amino acid sequence for the truncated GDF trap ActRIIB(L79D 25-131)-hFc (SEQ ID NO: 131), including the TPA leader (double underline), truncated ActRIIB extracellular domain (residues 25-131 in SEQ ID NO:1; single underline), and hFc domain. The aspartate substituted at position 79 in the native sequence is double underlined and highlighted, as is the glutamate revealed by sequencing to be the N-terminal residue in the mature fusion protein.

FIG. 9 shows the amino acid sequence for the truncated GDF trap ActRIIB(L79D 25-131)-hFc without a leader (SEQ ID NO: 132). The truncated ActRIIB extracellular domain (residues 25-131 in SEQ ID NO: 1) is underlined. The aspartate substituted at position 79 in the native sequence is double underlined and highlighted, as is the glutamate revealed by sequencing to be the N-terminal residue in the mature fusion protein.

FIG. 10 shows the amino acid sequence for the truncated GDF trap ActRIIB(L79D 25-131) without the leader, hFc domain, and linker (SEQ ID NO: 133). The aspartate substituted at position 79 in the native sequence is underlined and highlighted, as is the glutamate revealed by sequencing to be the N-terminal residue in the mature fusion protein.

FIG. 11 shows a nucleotide sequence encoding ActRIIB(L79D 25-131)-hFc. SEQ ID NO: 134 corresponds to the sense strand, and SEQ ID NO: 135 corresponds to the antisense strand. The TPA leader (nucleotides 1-66) is double underlined, and the truncated ActRIIB extracellular domain (nucleotides 76-396) is single underlined. The amino acid sequence for the ActRIIB extracellular domain (residues 25-131 in SEQ ID NO: 1) is also shown.

FIG. 12 shows an alternative nucleotide sequence encoding ActRIIB(L79D 25-131)-hFc. SEQ ID NO: 136 corresponds to the sense strand, and SEQ ID NO: 137 corresponds to the antisense strand. The TPA leader (nucleotides 1-66) is double underlined, the truncated ActRIIB extracellular domain (nucleotides 76-396) is underlined, and substitutions in the wild-type nucleotide sequence of the extracellular domain are double underlined and highlighted. The amino acid sequence for the ActRIIB extracellular domain (residues 25-131 in SEQ ID NO: 1) is also shown.

FIG. 13 shows the domain structure of hENG-Fc fusion constructs. Full-length ENG extracellular domain (residues 26-586 in top structure) consists of an orphan domain and N-terminal and C-terminal zona pellucida (ZP) domains. Below it are shown structures of selected truncated variants and whether they exhibit high-affinity binding (+/−) to BMP-9 and BMP-10 in an SPR-based assay.

FIG. 14 shows the effects of BMP10pro(22-312)-Fc on cardiac hypertrophy in a mouse TAC model. Heart weight versus body weight (HW/BW; mg/g) were compared from sham, TAC-PBS (vehicle control), and TAC-BMP10pro(22-312)-Fc treated animals. TAC-PBS control animals displayed significant cardiac hypertrophy compared to sham operated animals. BMP10pro(22-312)-Fc treatment inhibited cardiac hypertrophy in this TAC model of heart failure. (*) denotes one way ANOVA followed by Tukey.

FIG. 15 shows the effects of BMP10pro(22-312)-Fc on interventricular septal thickness at end diastole in a mouse TAC model. M-mode echocardiogram was acquired to measure interventricular septal thickness at end diastole in sham, TAC-PBS (vehicle control), and TAC-BMP10pro(22-312)-Fc treated animals. TAC-PBS mice displayed increased interventricular septal end diastole (LVSd mm) compared to sham operated animals. BMP10pro(22-312)-Fc treatment significantly decreased interventricular septal end diastole in this TAC model of heart failure. (*) and (#) denote one way ANOVA followed by Tukey.

FIG. 16 shows the effects of BMP10pro(22-312)-Fc on left ventricular posterior wall thickness at end diastole in a mouse TAC model. M-mode echocardiogram was acquired to measure left ventricular posterior wall thickness at end diastole in sham, TAC-PBS (vehicle control), and TAC-BMP10pro(22-312)-Fc treated animals. TAC-PBS mice displayed increased left ventricular posterior wall end diastole (LVPTd mm) compared to sham operated animals. BMP10pro(22-312)-Fc treatment significantly decreased left ventricular posterior wall end diastole in this TAC model of heart failure. (*) and (#) denote one way ANOVA followed by Tukey.

FIG. 17 shows the effects of BMP10pro(22-312)-Fc on factional shortening in a mouse TAC model. M-mode echocardiogram was acquired to measure left ventricle end diastolic diameter and left ventricle end systolic diameter in sham, TAC-PBS (vehicle control), and TAC-BMP10pro(22-312)-Fc treated animals. Fractional shortening (FS) was calculated from the end diastolic diameter (EDD) and end systolic diameter (ESD) using the following equation: FS=100%×[(EDD−ESD)/EDD]. TAC-PBS mice displayed ˜20% decreased fractional shortening compared to sham operated animal. BMP10pro(22-312)-Fc treatment significantly increased fractional shortening diastole in this TAC model of heart failure. (*) denotes one way ANOVA followed by Tukey.

FIG. 18 shows the effects of BMP10pro(22-312)-Fc on ejection fraction in a mouse TAC model. M-mode echocardiogram was acquired to measure end diastolic volume (EDD) and end systolic volume (ESD) in sham, TAC-PBS (vehicle control), and TAC-BMP10pro(22-312)-Fc treated animals. Ejection fractional (EF) was calculated using the following equation: EF %=(EDV−ESV)/EDV. TAC-PBS mice displayed decreased ejection fractional compared to sham operated animal. BMP10pro(22-312)-Fc treatment significantly increased ejection fractional in this TAC model of heart failure. (*) denotes one way ANOVA followed by Tukey.

FIG. 19 shows the effects of BMP10pro(22-312)-Fc on isovolumetric relaxation time in a mouse TAC model. M-mode echocardiogram was acquired to measure isovolumetric relaxation time (IVRT ms) in sham, TAC-PBS (vehicle control), and TAC-BMP10pro(22-312)-Fc treated animals. TAC+PBS mice displayed increased IVRT compared to sham operated animal. BMP10pro(22-312)-Fc treatment significantly decreased IVRT in this TAC model of heart failure. (*) and (#) denotes one way ANOVA followed by Tukey.

FIG. 20 shows the effects of BMP10pro(22-312)-Fc on cardiac fibrosis in a mouse TAC model. Hearts from sham, TAC-PBS (vehicle control), and TAC-BMP10pro(22-312)-Fc treated animals were removed, fixed in 10% formalin, and then sectioned for Masson's trichrome stain to assess fibrosis. TAC-PBS mice displayed increased cardiac fibrosis compared to sham operated animal. BMP10pro(22-312)-Fc treatment significantly decreased cardiac fibrosis in this TAC model of heart failure. (*) and (#) denotes one way ANOVA followed by Tukey.

FIG. 21 shows a nucleic acid sequence encoding a human BMP10 precursor protein, designated as SEQ ID NO: 33.

FIG. 22 shows a nucleic acid sequence encoding a human BMP10 propeptide domain protein, designated as SEQ ID NO: 35.

FIG. 23 shows the effects of hENG(27-581)-mFc on cardiac hypertrophy in a mouse TAC model. Heart weight versus body weight (HW/BW; mg/g) were compared from sham, TAC-PBS (vehicle control), and TAC-hENG(27-581)-mFc treated animals. TAC-PBS control animals displayed significant cardiac hypertrophy compared to sham operated animals. hENG(27-581)-mFc treatment inhibited cardiac hypertrophy in this TAC model of heart failure. (*) denotes one way ANOVA followed by Tukey.

FIG. 24 shows the effects of hENG(27-581)-mFc on factional shortening in a mouse TAC model. M-mode echocardiogram was acquired to measure left ventricle end diastolic diameter and left ventricle end systolic diameter in sham, TAC-PBS (vehicle control), and TAC-hENG(26-5861)-mFc treated animals. Fractional shortening (FS) was calculated from the end diastolic diameter (EDD) and end systolic diameter (ESD) using the following equation: FS=100%×[(EDD−ESD)/EDD]. TAC-PBS mice displayed ˜20% decreased fractional shortening compared to sham operated animal. hENG(267-581)-mFc treatment significantly increased fractional shortening diastole in this TAC model of heart failure. (*) denotes one way ANOVA followed by Tukey.

FIG. 25 shows the effects of hENG(27-581)-mFc on ejection fraction in a mouse TAC model. M-mode echocardiogram was acquired to measure end diastolic volume (EDD) and end systolic volume (ESD) in sham, TAC-PBS (vehicle control), and TAC-hENG(27-581)-mFc treated animals. Ejection fractional (EF) was calculated using the following equation: EF %=(EDV-ESV)/EDV. TAC-PBS mice displayed decreased ejection fractional compared to sham operated animal. hENG(27-581)-mFc treatment significantly increased ejection fractional in this TAC model of heart failure. (*) denotes one way ANOVA followed by Tukey.

FIG. 26 shows the effects of hENG(27-581)-mFc on cardiac fibrosis in a mouse TAC model. Hearts from sham, TAC-PBS (vehicle control), and TAC-hENG(27-581)-mFc treated animals were removed, fixed in 10% formalin, and then sectioned for Masson's trichrome stain to assess fibrosis. TAC-PBS mice displayed increased cardiac fibrosis compared to sham operated animal. hENG(27-581)-mFc treatment significantly decreased cardiac fibrosis in this TAC model of heart failure. (*) denotes one way ANOVA followed by Tukey.

FIG. 27 shows the effects of BMP10pro(22-312)-Fc or hENG(26-5861)-mFc on cardiac hypertrophy in a mouse MI model. Heart weight versus body weight (HW/BW; mg/g) were compared from MI-PBS (vehicle control), MI-BMP10pro(22-312)-Fc and TAC-hENG(27-581)-mFc treated animals. MI-PBS control animals displayed significant cardiac hypertrophy compared to sham operated animals. hENG(27-581)-mFc or BMP10pro(22-312)-Fc treatment inhibited cardiac hypertrophy in this MI model of heart failure. (*) denotes one way ANOVA followed by Tukey.

FIG. 28 shows the effects of BMP10pro(22-312)-Fc or hENG(27-581)-mFc on left ventricular dilation at end systole in a mouse MI model. M-mode echocardiogram was acquired to measure left ventricle dilation at end systole in sham, MI-PBS (vehicle control), MI-hENG(27-581)-mFc and MI-BMP10pro(22-312)-Fc treated animals. MI-PBS mice displayed increased left ventricle end systolic diameter (LVESD mm) compared to sham operated animals. BMP10pro(22-312)-Fc or hENG(27-581)-mFc treatment significantly decreased left ventricle end systolic diameter in this MI model of heart failure. (*) denote one way ANOVA followed by Tukey.

FIG. 29 shows the effects of BMP10pro(22-312)-Fc or hENG(27-581)-mFc on left ventricular dilation at end diastole in a mouse MI model. M-mode echocardiogram was acquired to measure left ventricle dilation at end diastole in sham, MI-PBS (vehicle control), MI-hENG(27-581)-mFc and MI-BM10Ppro(22-312)-Fc treated animals. MI-PBS mice displayed increased left ventricle end diastolic diameter (LVEDD mm) compared to sham operated animals. BMP10pro(22-312)-Fc or hENG(27-581)-mFc treatment significantly decreased left ventricle end diastolic diameter in this MI model of heart failure. (*) denote one way ANOVA followed by Tukey.

FIG. 30 shows the effects of BMP10pro(22-312)-Fc or hENG(27-581)-mFc on cardiac fibrosis in a mouse MI model. Hearts from MI-PBS (vehicle control), MI-BMP10pro(22-312)-Fc and MI-hENG(27-581)-mFc treated animals were removed, fixed in 10% formalin, and then sectioned for Masson's trichrome stain to assess fibrosis. MI-PBS mice displayed increased cardiac fibrosis compared to sham operated animal. BMP10pro(22-312)-Fc or hENG(27-581)-mFc treatment significantly decreased cardiac fibrosis in this MI model of heart failure. (*) denotes one way ANOVA followed by Tukey.

FIG. 31 shows the effects of BMP10pro(22-312)-Fc or hENG(27-581)-mFc on factional shortening in a mouse MI model. M-mode echocardiogram was acquired to measure left ventricle end diastolic diameter and left ventricle end systolic diameter in sham, MI-PBS (vehicle control), MI-BMP10pro(22-312)-Fc or and MI-hENG(27-581)-mFc treated animals. Fractional shortening (FS) was calculated from the end diastolic diameter (EDD) and end systolic diameter (ESD) using the following equation: FS=100%×[(EDD−ESD)/EDD]. TAC-PBS mice displayed ˜20% decreased fractional shortening compared to sham operated animal. hENG(27-581)-mFc treatment significantly increased fractional shortening diastole in this MI model of heart failure. (*) denotes one way ANOVA followed by Tukey.

FIG. 32 shows the effects of BMP10pro(22-312)-Fc or hENG(27-581)-mFc on ejection fraction in a mouse MI model. M-mode echocardiogram was acquired to measure end diastolic volume (EDD) and end systolic volume (ESD) in sham, MI-PBS (vehicle control), MI-BMP10pro(22-312)-Fc and MI-hENG(27-581)-mFc treated animals. Ejection fractional (EF) was calculated using the following equation: EF %=(EDV-ESV)/EDV. MI-PB S mice displayed decreased ejection fractional compared to sham operated animal. hENG(27-581)-mFc treatment significantly increased ejection fractional in this MI model of heart failure. (*) denotes one way ANOVA followed by Tukey.

### DETAIL DESCRIPTION OF THE INVENTION

### 1. Overview

The TGFβ superfamily is comprised of over 30 secreted factors including TGFβs, activins, nodals, bone morphogenetic proteins (BMPs), growth and differentiation factors (GDFs), and anti-Mullerian hormone (AMH) [Weiss et al. (2013) Developmental Biology, 2(1): 47-63]. Members of the superfamily, which are found in both vertebrates and invertebrates, are ubiquitously expressed in diverse tissues and function during the earliest stages of development throughout the lifetime of an animal. Indeed, TGFβ superfamily proteins are key mediators of stem cell self-renewal, gastrulation, differentiation, organ morphogenesis, and adult tissue homeostasis. Consistent with this ubiquitous activity, aberrant TGFβ superfamily signaling is associated with a wide range of human pathologies.

Ligands of the TGFβ superfamily share the same dimeric structure in which the central 3½ turn helix of one monomer packs against the concave surface formed by the beta-strands of the other monomer. The majority of TGFβ family members are further stabilized by an intermolecular disulfide bond. This disulfide bonds traverses through a ring formed by two other disulfide bonds generating what has been termed a ‘cysteine knot’ motif [Lin et al. (2006) Reproduction 132: 179-190; and Hinck et al. (2012) FEBS Letters 586: 1860-1870].

TGFβ superfamily signaling is mediated by heteromeric complexes of type I and type II serine/threonine kinase receptors, which phosphorylate and activate downstream SMAD proteins (e.g., SMAD proteins 1, 2, 3, 5, and 8) upon ligand stimulation [Massagué (2000) Nat. Rev. Mol. Cell Biol. 1:169-178]. These type I and type II receptors are transmembrane proteins, composed of a ligand-binding extracellular domain with cysteine-rich region, a transmembrane domain, and a cytoplasmic domain with predicted serine/threonine kinase specificity. In general, type I receptors mediate intracellular signaling while the type II receptors are required for binding TGF-beta superfamily ligands. Type I and II receptors form a stable complex after ligand binding, resulting in phosphorylation of type I receptors by type II receptors.

The TGFβ family can be divided into two phylogenetic branches based on the type I receptors they bind and the Smad proteins they activate. One is the more recently evolved branch, which includes, e.g., the TGFβs, activins, GDF8, GDF9, GDF11, BMP3 and nodal, which signal through type I receptors that activate Smads 2 and 3 [Hinck (2012) FEBS Letters 586:1860-1870]. The other branch comprises the more distantly related proteins of the superfamily and includes, e.g., BMP2, BMP4, BMP5, BMP6, BMP7, BMP8a, BMP8b, BMP9, BMP10, GDF1, GDF5, GDF6, and GDF7, which signal through Smads 1, 5, and 8.

Activins are members of the TGFβ superfamily and were initially discovered as regulators of secretion of follicle-stimulating hormone, but subsequently various reproductive and non-reproductive roles have been characterized. There are three principal activin forms (A, B, and AB) that are homo/heterodimers of two closely related β subunits (β_{A}β_{A}, β_{B}β_{B}, and β_{A}β_{B}, respectively). The human genome also encodes an activin C and an activin E, which are primarily expressed in the liver, and heterodimeric forms containing β_{C} or β_{E} are also known. In the TGF-beta superfamily, activins are unique and multifunctional factors that can stimulate hormone production in ovarian and placental cells, support neuronal cell survival, influence cell-cycle progress positively or negatively depending on cell type, and induce mesodermal differentiation at least in amphibian embryos [DePaolo et al. (1991) Proc Soc Ep Biol Med. 198:500-512; Dyson et al. (1997) Curr Biol. 7:81-84; and Woodruff (1998) Biochem Pharmacol. 55:953-963]. In several tissues, activin signaling is antagonized by its related heterodimer, inhibin. For example, in the regulation of follicle-stimulating hormone (FSH) secretion from the pituitary, activin promotes FSH synthesis and secretion, while inhibin reduces FSH synthesis and secretion. Other proteins that may regulate activin bioactivity and/or bind to activin include follistatin (FS), follistatin-related protein (FSRP, also known as FLRG or FSTL3), and α₂-macroglobulin.

The BMPs and GDFs together form a family of cysteine-knot cytokines sharing the characteristic fold of the TGFβ superfamily [Rider et al. (2010) Biochem J., 429(1):1-12]. This family includes, for example, BMP2, BMP4, BMP6, BMP7, BMP2a, BMP3, BMP3b (also known as GDF10), BMP4, BMP5, BMP6, BMP7, BMP8, BMP8a, BMP8b, BMP9 (also known as GDF2), BMP10, BMP11 (also known as GDF11), BMP12 (also known as GDF7), BMP13 (also known as GDF6), BMP14 (also known as GDF5), BMP15, GDF1, GDF3 (also known as VGR2), GDF8 (also known as myostatin), GDF9, GDF15, and decapentaplegic. Besides the ability to induce bone formation, which gave the BMPs their name, the BMP/GDFs display morphogenetic activities in the development of a wide range of tissues. BMP/GDF homo- and hetero-dimers interact with combinations of type I and type II receptor dimers to produce multiple possible signaling complexes, leading to the activation of one of two competing sets of SMAD transcription factors. BMP/GDFs have highly specific and localized functions. These are regulated in a number of ways, including the developmental restriction of BMP/GDF expression and through the secretion of several specific BMP antagonist proteins that bind with high affinity to the cytokines. Curiously, a number of these antagonists resemble TGFβ superfamily ligands.

As demonstrated herein, a soluble BMP10pro polypeptide, which binds to various BMP proteins including BMP10, BMP9, BMP6, BMP3b, and BMP5, is effective in reducing the severity of cardiac hypertrophy, cardiac remodeling, and cardiac fibrosis as well as improving cardiac function in a transverse aortic constriction (TAC) heart failure model. Moreover, BMP10pro treatment increased survival time of heart failure patients in this study. The BMP10pro polypeptide also had similar beneficial effects in a myocardial infarction (MI) heart failure model. Furthermore, a soluble endoglin polypeptide, which binds to BMP9 and BMP10, was show to have various beneficial effect in both TAC and MI heart failure models. While not wishing to be bound to any particular mechanism, it is expected that the effects of BMP10pro polypeptides and endoglin polypeptides are caused primarily by a BMP signaling antagonist effect, particularly of one or more of BMP10, BMP9, BMP6, BMP3b, and BMP5. Regardless of the mechanism, it is apparent from the data presented herein that BMP signaling antagonists do reduce the severity of cardiac hypertrophy, decrease cardiac remodeling, decrease cardiac fibrosis, and have other positivity effects in treating heart failure. It should be noted that blood pressure, hypertrophy, cardiac remodeling, and fibrosis are dynamic, with changes depending on a balance of factors that increase blood pressure, hypertrophy, cardiac remodeling, and fibrosis and factors that decrease blood pressure, hypertrophy, cardiac remodeling, and fibrosis. Blood pressure, hypertrophy, cardiac remodeling, and fibrosis can be decreased by increasing factors that reduce blood pressure, hypertrophy, cardiac remodeling, and fibrosis; decreasing factors that promote blood pressure, hypertrophy, cardiac remodeling, and fibrosis; or both. The terms decreasing (reducing) blood pressure, hypertrophy, cardiac remodeling, and fibrosis refer to the observable physical changes in blood pressure, hypertrophy, cardiac remodeling, and fibrosis and are intended to be neutral as to the mechanism by which the changes occur.

The animal models for heart that were used in the studies described herein are considered to be predicative of efficacy in humans, and therefore, this disclosure provides methods for using BMP10pro polypeptides, endoglin polypeptides and other BMP antagonists to treat heart failure, particularly treating, preventing, or reducing the severity or duration of one or more complications of heart failure, in humans. As disclosed herein, the term BMP antagonist refers a variety of agents that may be used to antagonize BMP signaling including, for example, antagonists that inhibit one or more BMP ligands [e.g., BMP10, BMP9, BMP6, BMP3b, and BMP5]; antagonists that inhibit one or more BMP-interacting type I-, type II-, or co-receptor (e.g., ALK1, ActRIIA, ActRIIB, BMPRII, and endoglin); and antagonists that inhibit one or more downstream signaling components (e.g., Smad proteins such as Smads 2 and 3). BMP antagonists to be used in accordance with the methods and uses of the disclosure include a variety of forms, for example, ligand traps (e.g., soluble BMP10pro polypeptides, ActRIIA polypeptides, ActRIIB polypeptides, ALK1 polypeptides, and endoglin polypeptides), antibody antagonists (e.g., antibodies that inhibit one or more of BMP10, BMP9, BMP6, BMP3b, BMP5, ALK1, ActRIIA, ActRIIB, BMPRII, and endoglin), small molecule antagonists [e.g., small molecules that inhibit one or more of BMP10, BMP9, BMP6, BMP3b, BMP5, ALK1, ActRIIA, ActRIIB, BMPRII, endoglin and one or more Smad proteins (e.g., Smads 2 and 3)], and nucleotide antagonists [e.g., nucleotide sequences that inhibit one or more of BMP10, BMP9, BMP6, BMP3b, BMP5, ALK1, ActRIIA, ActRIIB, BMPRII, endoglin and one or more Smad proteins (e.g., Smads 2 and 3)].

The terms used in this specification generally have their ordinary meanings in the art, within the context of this disclosure and in the specific context where each term is used. Certain terms are discussed below or elsewhere in the specification to provide additional guidance to the practitioner in describing the compositions and methods of the disclosure and how to make and use them. The scope or meaning of any use of a term will be apparent from the specific context in which it is used.

“Homologous,” in all its grammatical forms and spelling variations, refers to the relationship between two proteins that possess a “common evolutionary origin,” including proteins from superfamilies in the same species of organism, as well as homologous proteins from different species of organism. Such proteins (and their encoding nucleic acids) have sequence homology, as reflected by their sequence similarity, whether in terms of percent identity or by the presence of specific residues or motifs and conserved positions. However, in common usage and in the instant application, the term “homologous,” when modified with an adverb such as “highly,” may refer to sequence similarity and may or may not relate to a common evolutionary origin.

The term “sequence similarity,” in all its grammatical forms, refers to the degree of identity or correspondence between nucleic acid or amino acid sequences that may or may not share a common evolutionary origin.

“Percent (%) sequence identity” with respect to a reference polypeptide (or nucleotide) sequence is defined as the percentage of amino acid residues (or nucleic acids) in a candidate sequence that are identical to the amino acid residues (or nucleic acids) in the reference polypeptide (nucleotide) sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For purposes herein, however, % amino acid (nucleic acid) sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary.

“Agonize”, in all its grammatical forms, refers to the process of activating a protein and/or gene (e.g., by activating or amplifying that protein's gene expression or by inducing an inactive protein to enter an active state) or increasing a protein's and/or gene's activity.

“Antagonize”, in all its grammatical forms, refers to the process of inhibiting a protein and/or gene (e.g., by inhibiting or decreasing that protein's gene expression or by inducing an active protein to enter an inactive state) or decreasing a protein's and/or gene's activity.

The terms “about” and “approximately” as used in connection with a numerical value throughout the specification and the claims denotes an interval of accuracy, familiar and acceptable to a person skilled in the art. In general, such interval of accuracy is ±10%. Alternatively, and particularly in biological systems, the terms “about” and “approximately” may mean values that are within an order of magnitude, preferably ≤5-fold and more preferably ≤2-fold of a given value.

Numeric ranges disclosed herein are inclusive of the numbers defining the ranges.

The terms “a” and “an” include plural referents unless the context in which the term is used clearly dictates otherwise. The terms “a” (or “an”), as well as the terms “one or more,” and “at least one” can be used interchangeably herein. Furthermore, “and/or” where used herein is to be taken as specific disclosure of each of the two or more specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

### 2. BMP10 Propeptides, ActRII Polypeptides, ALK1 Polypeptides, BMPRII Polypeptides, and Endoglin Polypeptides

In certain aspects, the disclosure relates to BMP10 propeptide (BMP10pro) polypeptides and uses thereof (e.g., treating heart failure or a complication of heart failure). As used herein, the term “BMP10 polypeptide” refers to the family of bone morphogenetic proteins of the type 10 derived from any species. The term “BMP10 polypeptide” includes any of the naturally occurring BMP10 polypeptides as well as any variants thereof (including mutants, fragments, fusions, and peptidomimetic forms) that retain a useful activity. A naturally occurring BMP10 protein is generally encoded as a larger precursor that typically contains a signal sequence at its N-terminus followed by a dibasic amino acid cleavage site and a propeptide, followed by another dibasic amino acid cleavage site and a mature domain. The human BMP10 precursor sequence (NCBI NP_055297) is shown below:

| (SEQ ID NO: 32) | |
|---|---|
| 1 | MGSLVLTLCA LFCLAAYLVS GSPIMNLEQS PLEEDMSLFG DVFSEQDGVD |
| 51 | FNTLLQSMKD EFLKTLNLSD IPTQDSAKVD PPEYMLELYN KFATDRTSMP |
| 101 | SANIIRSFKN EDLFSQPVSF NGLRKYPLLF NVSIPHHEEV IMAELRLYTL |
| 151 | VQRDRMIYDG VDRKITIFEV LESKGDNEGE RNMLVLVSGE IYGTNSEWET |
| 201 | FDVTDAIRRW QKSGSSTHQL EVHIESKHDE AEDASSGRLE IDTSAQNKHN |
| 251 | PLLIVFSDDQ SSDKERKEEL NEMISHEQLP ELDNLGLDSF SSGPGEEALL |
| 301 | QMRSNIIYDS TARIRRNAKG NYCKRTPLYI DFKEIGWDSW IIAPPGYEAY |
| 351 | ECRGVCNYPL AEHLTPTKHA IIQALVHLKN SQKASKACCV PTKLEPISIL |
| 401 | YLDKGVVTYK FKYEGMAVSE CGCR |

The signal peptide (amino acids 1-21) is underlined; the mature protein (amino acids 317-424) is double underlined; and potential N-linked glycosylation sites are boxed. FIG. 21 shows a nucleic acid sequence encoding the BMP10 precursor protein of SEQ ID NO: 32 (this nucleic acid is designated SEQ ID NO: 33).

The term “BMP10 propeptide” or “BMP10pro” is used to refer to polypeptides comprising any naturally occurring propeptide of a BMP10 family member as well as any variants thereof (including mutants, fragments and peptidomimetic forms) that retain a useful activity. Examples of useful activities of BMP10pro polypeptides include binding to the mature portion of a BMP10 protein and acting as an antagonist of an activity of a mature BMP10. As demonstrated herein, BMP10pro polypeptides may also bind to one or more of BMP9, BMP6, BMP3b, and BMP5. Thus, in some embodiments, BMP10pro polypeptides may be further used to as an antagonist of one or more of BMP9, BMP6, BMP3b, and BMP5. Functional variants of a BMP10 propeptide may be characterized by, for example, binding to mature BMP10 protein and/or the ability to competitively inhibit the binding of BMP10 to a type II receptor such as ActRIIA, ActRIIB, BMPRII; type I receptor such as ALK1; and/or a co-receptor such as endoglin.

A human BMP10 propeptide sequence is shown below:

(SEQ ID NO: 34)

GSPIMNLEQS PLEEDMSLFG
DVFSEQDGVD FNTLLQSMKD EFLKTLNLSD IPTQDSAKVD
PPEYMLELYN KFATDRTSMP SANIIRSFKN EDLFSQPVSF
NGLRKYPLLF NVSIPHHEEV IMAELRLYTL VQRDRMIYDG
VDRKITIFEV LESKGDNEGE RNMLVLVSGE IYGTNSEWET
FDVTDAIRRW QKSGSSTHQL EVHIESKHDE AEDASSGRLE
IDTSAQNKHN PLLIVFSDDQ SSDKERKEEL NEMISHEQLP
ELDNLGLDSF SSGPGEEALL QMRSNIIYDS TARIRR

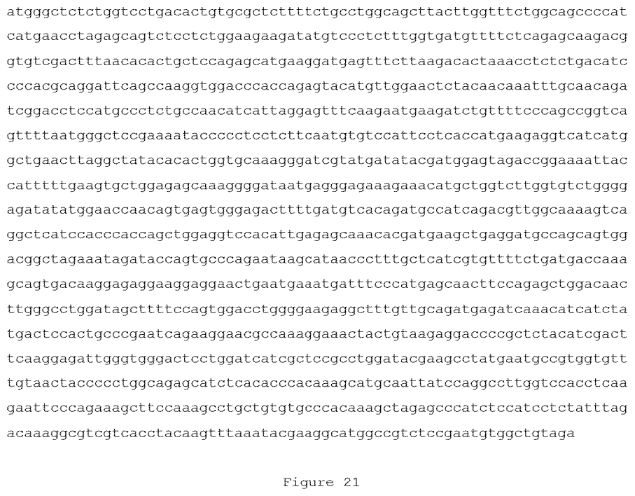

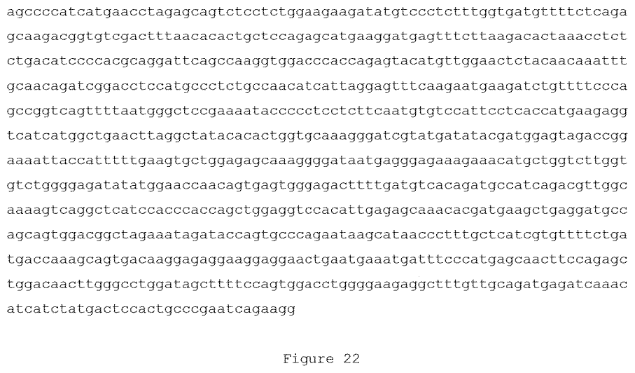

FIG. 22 shows a nucleic acid sequence encoding the BMP10 propeptide corresponding to SEQ ID NO: 34 (this nucleic acid is designated SEQ ID NO: 35).

The BMP10 propeptide is conserved among vertebrates. Therefore one could generate an alignment of BMP10 propeptide sequences from different vertebrates using techniques well known in the art and as described herein, and use these alignments to predict key amino acid positions within the propeptides domain that are important for mature BMP10-binding activities as well as to predict amino acid positions that are likely to be tolerant to substitution without significantly altering mature BMP10-binding activities. Therefore, an active, human BMP10pro variant polypeptide useful in accordance with the presently disclosed methods may include one or more amino acids at corresponding positions from the sequence of another vertebrate BMP10pro polypeptide, or may include a residue that is similar to that in the human or other vertebrate sequences.

As shown herein, a variant BMP10pro polypeptide comprising a BMP10pro domain having a deletion of the C-terminal arginine of the propeptide sequence (deletion of the amino acid at position 296 of SEQ ID NO: 34) retains high affinity for BMP10 and can be used as a BMP10 antagonist. Another variant BMP10pro polypeptide was generated comprising a BMP10pro domain having a deletion of four amino acids at the C-terminus of the propeptide sequence (deletion of amino acids at positions 293-296 of SEQ ID NO: 34). Surprisingly, the variant BMP10pro polypeptide having four amino acids deleted from the C-terminus of the propeptide sequence was a more potent antagonist of BMP10 activity than a BMP10pro polypeptide having a deletion of only the C-terminal arginine. Thus, BMP10pro polypeptide domains that stop at any one of amino acids 292, 293, 294, 295 and 296 with respect to SEQ ID NO: 34 are all expected to be active, but constructs stopping at 292 may be most active. Any of these forms may be desirable to use, depending on the clinical or experimental setting.

BMP10pro polypeptides may additionally include any of various leader sequences at the N-terminus. Such a sequence would allow the peptides to be expressed and targeted to the secretion pathway in a eukaryotic system. See, e.g., Ernst et al., U.S. Pat. No. 5,082,783 (1992). Alternatively, a native BMP10 signal sequence may be used to effect extrusion from the cell. Possible leader sequences include honeybee mellitin, TPA, and native leaders, which are disclosed herein. Examples of BMP10pro-Fc fusion proteins incorporating a TPA leader sequence include SEQ ID NOs: 82 and 85. Processing of signal peptides may vary depending on the leader sequence chosen, the cell type used and culture conditions, among other variables, and therefore actual N-terminal start sites for mature BMP10pro polypeptides may shift by 1, 2, 3, 4 or 5 amino acids at the N-terminal direction. Therefore, at the N-terminus of the BMP10pro, it is expected that a protein beginning any one of amino acids 1, 2, 3, 4, 5, or 6 with respect to SEQ ID NO: 34 are all expected to be active.

Taken together, a general formula for an active portion (e.g., mature BMP10-binding portion) of BMP10pro comprises amino acids 6-292 of SEQ ID NO: 34. Therefore, BMP10pro polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to a portion of BMP10pro beginning at a residue corresponding to any one of amino acids 1-6 (e.g., beginning at any one of amino acids 1, 2, 3, 4, 5, or 6) of SEQ ID NO: 34 and ending at a position corresponding to any one amino acids 292-296 (e.g., ending at any one of amino acids 292, 293, 294, 295, or 296) of SEQ ID NO: 34. For example, in some embodiments, a BMP10pro polypeptide of the disclosure may comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical amino acids 1-296 of SEQ ID NO: 34. In some embodiments, a BMP10pro polypeptide of the disclosure may comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical amino acids 1-295 of SEQ ID NO: 34. In some embodiments, a BMP10pro polypeptide of the disclosure may comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical amino acids 1-294 of SEQ ID NO: 34. In some embodiments, a BMP10pro polypeptide of the disclosure may comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical amino acids 1-293 of SEQ ID NO: 34. In some embodiments, a BMP10pro polypeptide of the disclosure may comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical amino acids 1-292 of SEQ ID NO: 34. In some embodiments, a BMP10pro polypeptide of the disclosure may comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical amino acids 2-295 of SEQ ID NO: 34. In some embodiments, a BMP10pro polypeptide of the disclosure may comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical amino acids 2-292 of SEQ ID NO: 34. In some embodiments, a BMP10pro polypeptide of the disclosure may comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical amino acids 3-295 of SEQ ID NO: 34. In some embodiments, a BMP10pro polypeptide of the disclosure may comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical amino acids 3-294 of SEQ ID NO: 34. In some embodiments, a BMP10pro polypeptide of the disclosure may comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical amino acids 3-292 of SEQ ID NO: 34. In some embodiments, a BMP10pro polypeptide of the disclosure may comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical amino acids 4-292 of SEQ ID NO: 34. In some embodiments, a BMP10pro polypeptide of the disclosure may comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical amino acids 5-292 of SEQ ID NO: 34. In some embodiments, a BMP10pro polypeptide of the disclosure may comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical amino acids 6-292 of SEQ ID NO: 34. Preferably, BMP10pro polypeptides are soluble. It is expected that the BMP10pro polypeptides described above will retain mature BMP10-binding and antagonizing activity. In some embodiments, such BMP10pro polypeptides may further binds to one or more of BMP9, BMP6, BMP3b and BMP5.

In certain aspects, the disclosure relates ActRII polypeptides and uses thereof (e.g., treating heart failure or a complication of heart failure). As used herein, the term “ActRII” refers to the family of type II activin receptors. This family includes activin receptor type IIA (ActRIIA) and activin receptor type IIB (ActRIIB) As used herein, the term “ActRIIB” refers to a family of activin receptor type IIB

(ActRIIB) proteins from any species and variants derived from such ActRIIB proteins by mutagenesis or other modification. Reference to ActRIIB herein is understood to be a reference to any one of the currently identified forms. Members of the ActRIIB family are generally transmembrane proteins, composed of a ligand-binding extracellular domain comprising a cysteine-rich region, a transmembrane domain, and a cytoplasmic domain with predicted serine/threonine kinase activity.

The term “ActRIIB polypeptide” includes polypeptides comprising any naturally occurring polypeptide of an ActRIIB family member as well as any variants thereof (including mutants, fragments, fusions, and peptidomimetic forms) that retain a useful activity. Examples of such variant ActRIIB polypeptides are provided throughout the present disclosure as well as in International Patent Application Publication No. WO 2006/012627, WO 2008/097541, and WO 2010/151426, which are incorporated herein by reference in its entirety. Numbering of amino acids for all ActRIIB-related polypeptides described herein is based on the numbering of the human ActRIIB precursor protein sequence provided below (SEQ ID NO: 1), unless specifically designated otherwise.

The human ActRIIB precursor protein sequence is as follows:

| (SEQ ID NO: 1) | |
|---|---|
| 1 | MTAPWVALAL LWGSLCAGSG RGEAETRECI YYNANWELER T [symbol] QSGLERCE |
| 51 | GEQDKRLHCY ASWRNSSGTI ELVKKGCWLD DFNCYDRQEC VATEENPQVY |
| 101 | FCCCEGNFCN ERFTHLPEAG GPEVTYEPPP TAPTLLTVLA YSLLPIGGLS |
| 151 | LIVLLAFWMY RHRKPPYGHV DIHEDPGPPP PSPLVGLKPL QLLEIKARGR |
| 201 | FGCVWKAQLM NDFVAVKIFP LQDKQSWQSE REIFSTPGMK HENLLQFIAA |
| 251 | EKRGSNLEVE LWLITAFHDK GSLTDYLKGN IITWNELCHV AETMSRGLSY |
| 301 | LHEDVPWCRG EGHKPSIAHR DFKSKNVLLK SDLTAVLADF GLAVRFEPGK |
| 351 | PPGDTHGQVG TRRYMAPEVL EGAINFQRDA FLRIDMYAMG LVLWELVSRC |
| 401 | KAADGPVDEY MLPFEEEIGQ HPSLEELQEV VVHKKMRPTI KDHWLKHPGL |
| 451 | AQLCVTIEEC WDHDAEARLS AGCVEERVSL IRRSVNGTTS DCLVSLVTSV |
| 501 | TNVDLPPKES SI |

The signal peptide is indicated with a single underline; the extracellular domain is indicated in bold font; and the potential, endogenous N-linked glycosylation sites are indicated with a double underline.

A processed extracellular ActRIIB polypeptide sequence is as follows:

(SEQ ID NO: 2)
GRGEAETRECIYYNANWELERTNQSGLERCEGEQDKRLHCYASWRNSSGT
IELVKKGCWLDDFNCYDRQECVATEENPQVYFCCCEGNFCNERFTHLPEA
GGPEVTYEPPPTAPT.

In some embodiments, the protein may be produced with an “SGR . . . ” sequence at the N-terminus. The C-terminal “tail” of the extracellular domain is indicated by a single underline. The sequence with the “tail” deleted (a Δ15 sequence) is as follows:

(SEQ ID NO: 3)
GRGEAETRECIYYNANWELERTNQSGLERCEGEQDKRLHCYASWRNSSG
TIELVKKGCWLDDFNCYDRQECVATEENPQVYFCCCEGNFCNERFTHLPE
A.

A form of ActRIIB with an alanine at position 64 of SEQ ID NO: 1 (A64) is also reported in the literature. See, e.g., Hilden et al. (1994) Blood, 83(8): 2163-2170. It has been ascertained that an ActRIIB-Fc fusion protein comprising an extracellular domain of ActRIIB with the A64 substitution has a relatively low affinity for activin and GDF11. By contrast, the same ActRIIB-Fc fusion protein with an arginine at position 64 (R64) has an affinity for activin and GDF11 in the low nanomolar to high picomolar range. Therefore, sequences with an R64 are used as the “wild-type” reference sequence for human ActRIIB in this disclosure.

The form of ActRIIB with an alanine at position 64 is as follows:

| (SEQ ID NO: 4) | |
|---|---|
| 1 | MTAPWVALAL LWGSLCAGSG RGEAETRECI YYNANWELER TNQSGLERCE |
| 51 | GEQDKRLHCY ASWANSSGTI ELVKKGCWLD DFNCYDRQEC VATEENPQVY |
| 101 | FCCCEGNFCN ERFTHLPEAG GPEVTYEPPP TAPTLLTVLA YSLLPIGGLS |
| 151 | LIVLLAFWMY RHRKPPYGHV DIHEDPGPPP PSPLVGLKPL QLLEIKARGR |
| 201 | FGCVWKAQLM NDFVAVKIFP LQDKQSWQSE REIFSTPGMK HENLLQFIAA |
| 251 | EKRGSNLEVE LWLITAFHDK GSLTDYLKGN IITWNELCHV AETMSRGLSY |
| 301 | LHEDVPWCRG EGHKPSIAHR DFKSKNVLLK SDLTAVLADF GLAVRFEPGK |
| 351 | PPGDTHGQVG TRRYMAPEVL EGAINFQRDA FLRIDMYAMG LVLWELVSRC |
| 401 | KAADGPVDEY MLPFEEEIGQ HPSLEELQEV VVHKKMRPTI KDHWLKHPGL |
| 451 | AQLCVTIEEC WDHDAEARLS AGCVEERVSL IRRSVNGTTS DCLVSLVTSV |
| 501 | TNVDLPPKES SI |

The signal peptide is indicated by single underline and the extracellular domain is indicated by bold font.

The processed extracellular ActRIIB polypeptide sequence of the alternative A64 form is as follows:

(SEQ ID NO: 5)
GRGEAETRECIYYNANWELERTNQSGLERCEGEQDKRLHCYASWANSSGT
IELVKKGCWLDDFNCYDRQECVATEENPQVYFCCCEGNFCNERFTHLPEA
GGPEVTYEPPPTAPT

In some embodiments, the protein may be produced with an “SGR . . . ” sequence at the N-terminus. The C-terminal “tail” of the extracellular domain is indicated by single underline. The sequence with the “tail” deleted (a Δ15 sequence) is as follows:

(SEQ ID NO: 6)
GRGEAETRECIYYNANWELERTNQSGLERCEGEQDKRLHCYASWANSSGT
IELVKKGCWLDDFNCYDRQECVATEENPQVYFCCCEGNFCNERFTHLPEA

A nucleic acid sequence encoding the human ActRIIB precursor protein is shown below (SEQ ID NO: 7), representing nucleotides 25-1560 of Genbank Reference Sequence NM_001106.3, which encode amino acids 1-513 of the ActRIIB precursor. The sequence as shown provides an arginine at position 64 and may be modified to provide an alanine instead. The signal sequence is underlined.

| (SEQ ID NO: 7) | |
|---|---|
| 1 | ATGACGGCGC CCTGGGTGGC CCTCGCCCTC CTCTGGGGAT CGCTGTGCGC |
| 51 | CGGCTCTGGG CGTGGGGAGG CTGAGACACG GGAGTGCATC TACTACAACG |
| 101 | CCAACTGGGA GCTGGAGCGC ACCAACCAGA GCGGCCTGGA GCGCTGCGAA |
| 151 | GGCGAGCAGG ACAAGCGGCT GCACTGCTAC GCCTCCTGGC GCAACAGCTC |
| 201 | TGGCACCATC GAGCTCGTGA AGAAGGGCTG CTGGCTAGAT GACTTCAACT |
| 251 | GCTACGATAG GCAGGAGTGT GTGGCCACTG AGGAGAACCC CCAGGTGTAC |
| 301 | TTCTGCTGCT GTGAAGGCAA CTTCTGCAAC GAACGCTTCA CTCATTTGCC |
| 351 | AGAGGCTGGG GGCCCGGAAG TCACGTACGA GCCACCCCCG ACAGCCCCCA |
| 401 | CCCTGCTCAC GGTGCTGGCC TACTCACTGC TGCCCATCGG GGGCCTTTCC |
| 451 | CTCATCGTCC TGCTGGCCTT TTGGATGTAC CGGCATCGCA AGCCCCCCTA |
| 501 | CGGTCATGTG GACATCCATG AGGACCCTGG GCCTCCACCA CCATCCCCTC |
| 551 | TGGTGGGCCT GAAGCCACTG CAGCTGCTGG AGATCAAGGC TCGGGGGCGC |
| 601 | TTTGGCTGTG TCTGGAAGGC CCAGCTCATG AATGACTTTG TAGCTGTCAA |
| 651 | GATCTTCCCA CTCCAGGACA AGCAGTCGTG GCAGAGTGAA CGGGAGATCT |
| 701 | TCAGCACACC TGGCATGAAG CACGAGAACC TGCTACAGTT CATTGCTGCC |
| 751 | GAGAAGCGAG GCTCCAACCT CGAAGTAGAG CTGTGGCTCA TCACGGCCTT |
| 801 | CCATGACAAG GGCTCCCTCA CGGATTACCT CAAGGGGAAC ATCATCACAT |
| 851 | GGAACGAACT GTGTCATGTA GCAGAGACGA TGTCACGAGG CCTCTCATAC |
| 901 | CTGCATGAGG ATGTGCCCTG GTGCCGTGGC GAGGGCCACA AGCCGTCTAT |
| 951 | TGCCCACAGG GACTTTAAAA GTAAGAATGT ATTGCTGAAG AGCGACCTCA |
| 1001 | CAGCCGTGCT GGCTGACTTT GGCTTGGCTG TTCGATTTGA GCCAGGGAAA |
| 1051 | CCTCCAGGGG ACACCCACGG ACAGGTAGGC ACGAGACGGT ACATGGCTCC |
| 1101 | TGAGGTGCTC GAGGGAGCCA TCAACTTCCA GAGAGATGCC TTCCTGCGCA |
| 1151 | TTGACATGTA TGCCATGGGG TTGGTGCTGT GGGAGCTTGT GTCTCGCTGC |
| 1201 | AAGGCTGCAG ACGGACCCGT GGATGAGTAC ATGCTGCCCT TTGAGGAAGA |
| 1251 | GATTGGCCAG CACCCTTCGT TGGAGGAGCT GCAGGAGGTG GTGGTGCACA |
| 1301 | AGAAGATGAG GCCCACCATT AAAGATCACT GGTTGAAACA CCCGGGCCTG |
| 1351 | GCCCAGCTTT GTGTGACCAT CGAGGAGTGC TGGGACCATG ATGCAGAGGC |
| 1401 | TCGCTTGTCC GCGGGCTGTG TGGAGGAGCG GGTGTCCCTG ATTCGGAGGT |
| 1451 | CGGTCAACGG CACTACCTCG GACTGTCTCG TTTCCCTGGT GACCTCTGTC |
| 1501 | ACCAATGTGG ACCTGCCCCC TAAAGAGTCA AGCATC |

A nucleic acid sequence encoding a processed extracellular human ActRIIB polypeptide is as follows (SEQ ID NO: 8). The sequence as shown provides an arginine at position 64, and may be modified to provide an alanine instead.

| (SEQ ID NO: 8) | |
|---|---|
| 1 | GGGCGTGGGG AGGCTGAGAC ACGGGAGTGC ATCTACTACA ACGCCAACTG |
| 51 | GGAGCTGGAG CGCACCAACC AGAGCGGCCT GGAGCGCTGC GAAGGCGAGC |
| 101 | AGGACAAGCG GCTGCACTGC TACGCCTCCT GGCGCAACAG CTCTGGCACC |
| 151 | ATCGAGCTCG TGAAGAAGGG CTGCTGGCTA GATGACTTCA ACTGCTACGA |
| 201 | TAGGCAGGAG TGTGTGGCCA CTGAGGAGAA CCCCCAGGTG TACTTCTGCT |
| 251 | GCTGTGAAGG CAACTTCTGC AACGAACGCT TCACTCATTT GCCAGAGGCT |
| 301 | GGGGGCCCGG AAGTCACGTA CGAGCCACCC CCGACAGCCC CCACC |

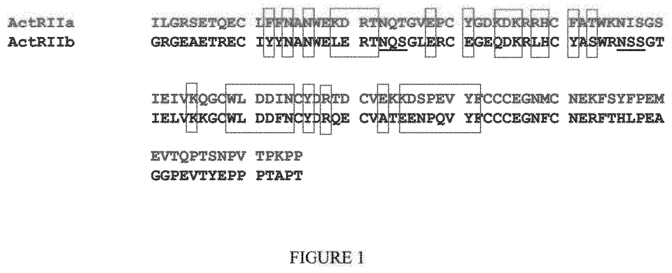

An alignment of the amino acid sequences of human ActRIIB extracellular domain and human ActRIIA extracellular domain are illustrated in FIG. 1. This alignment indicates amino acid residues within both receptors that are believed to directly contact ActRII ligands. For example, the composite ActRII structures indicated that the ActRIIB-ligand binding pocket is defined, in part, by residues Y31, N33, N35, L38 through T41, E47, E50, Q53 through K55, L57, H58, Y60, S62, K74, W78 through N83, Y85, R87, A92, and E94 through F101. At these positions, it is expected that conservative mutations will be tolerated.

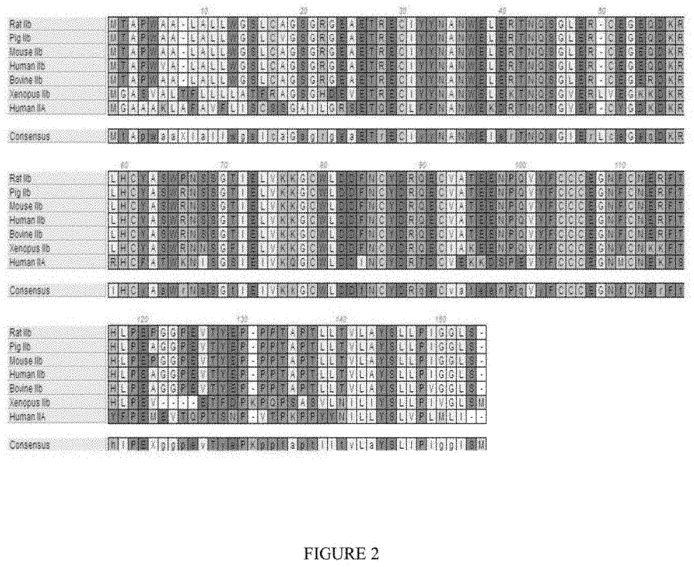

In addition, ActRIIB is well-conserved among vertebrates, with large stretches of the extracellular domain completely conserved. For example, FIG. 2 depicts a multi-sequence alignment of a human ActRIIB extracellular domain compared to various ActRIIB orthologs. Many of the ligands that bind to ActRIIB are also highly conserved. Accordingly, from these alignments, it is possible to predict key amino acid positions within the ligand-binding domain that are important for normal ActRIIB-ligand binding activities as well as to predict amino acid positions that are likely to be tolerant to substitution without significantly altering normal ActRIIB-ligand binding activities. Therefore, an active, human ActRIIB variant polypeptide useful in accordance with the presently disclosed methods may include one or more amino acids at corresponding positions from the sequence of another vertebrate ActRIIB, or may include a residue that is similar to that in the human or other vertebrate sequences. Without meaning to be limiting, the following examples illustrate this approach to defining an active ActRIIB variant. L46 in the human extracellular domain (SEQ ID NO: 103) is a valine in Xenopus ActRIIB (SEQ ID NO: 105), and so this position may be altered, and optionally may be altered to another hydrophobic residue, such as V, I or F, or a non-polar residue such as A. E52 in the human extracellular domain is a K in Xenopus, indicating that this site may be tolerant of a wide variety of changes, including polar residues, such as E, D, K, R, H, S, T, P, G, Y and probably A. T93 in the human extracellular domain is a K in Xenopus, indicating that a wide structural variation is tolerated at this position, with polar residues favored, such as S, K, R, E, D, H, G, P, G and Y. F108 in the human extracellular domain is a Y in Xenopus, and therefore Y or other hydrophobic group, such as I, V or L should be tolerated. E111 in the human extracellular domain is K in Xenopus, indicating that charged residues will be tolerated at this position, including D, R, K and H, as well as Q and N. R112 in the human extracellular domain is K in Xenopus, indicating that basic residues are tolerated at this position, including R and H. A at position 119 in the human extracellular domain is relatively poorly conserved, and appears as P in rodents and V in Xenopus, thus essentially any amino acid should be tolerated at this position.

Moreover, ActRII proteins have been characterized in the art in terms of structural and functional characteristics, particularly with respect to ligand binding [Attisano et al. (1992) Cell 68(1):97-108; Greenwald et al. (1999) Nature Structural Biology 6(1): 18-22; Allendorph et al. (2006) PNAS 103(20: 7643-7648; Thompson et al. (2003) The EMBO Journal 22(7): 1555-1566; as well as U.S. Pat. Nos. 7,709,605, 7,612,041, and 7,842,663]. In addition to the teachings herein, these references provide amply guidance for how to generate ActRIIB variants that retain one or more normal activities (e.g., ligand-binding activity).

For example, a defining structural motif known as a three-finger toxin fold is important for ligand binding by type I and type II receptors and is formed by conserved cysteine residues located at varying positions within the extracellular domain of each monomeric receptor [Greenwald et al. (1999) Nat Struct Biol 6:18-22; and Hinck (2012) FEBS Lett 586:1860-1870]. Accordingly, the core ligand-binding domains of human ActRIIB, as demarcated by the outermost of these conserved cysteines, corresponds to positions 29-109 of SEQ ID NO: 1 (ActRIIB precursor). The structurally less-ordered amino acids flanking these cysteine-demarcated core sequences can be truncated by 1, 2, 3, 4, 5, 6, 7, 8, 9, 10, 11, 12, 13, 14, 15, 16, 17, 18, 19, 20, 21, 22, 23, 24, 25, 26, 27, or 28 residues at the N-terminus and/or 1, 2, 3, 4, 5, 6, 7, 8, 9, 10, 11, 12, 13, 14, 15, 16, 17, 18, 19, 20, 21, 22, 23, 24 or 25 residues a the C-terminus without necessarily altering ligand binding. Exemplary ActRIIB extracellular domains for N-terminal and/or C-terminal truncation include SEQ ID NOs: 2, 3, 5, and 6.

Attisano et al. showed that a deletion of the proline knot at the C-terminus of the extracellular domain of ActRIIB reduced the affinity of the receptor for activin. An ActRIIB-Fc fusion protein containing amino acids 20-119 of present SEQ ID NO: 1, “ActRIIB(20-119)-Fc”, has reduced binding to GDF11 and activin relative to an ActRIIB(20-134)-Fc, which includes the proline knot region and the complete juxtamembrane domain (see, e.g., U.S. Pat. No. 7,842,663). However, an ActRIIB(20-129)-Fc protein retains similar, but somewhat reduced activity, relative to the wild-type, even though the proline knot region is disrupted.

Thus, ActRIIB extracellular domains that stop at amino acid 134, 133, 132, 131, 130 and 129 (with respect to SEQ ID NO: 1) are all expected to be active, but constructs stopping at 134 or 133 may be most active. Similarly, mutations at any of residues 129-134 (with respect to SEQ ID NO: 1) are not expected to alter ligand-binding affinity by large margins. In support of this, it is known in the art that mutations of P129 and P130 (with respect to SEQ ID NO: 1) do not substantially decrease ligand binding. Therefore, an ActRIIB polypeptide of the present disclosure may end as early as amino acid 109 (the final cysteine), however, forms ending at or between 109 and 119 (e.g., 109, 110, 111, 112, 113, 114, 115, 116, 117, 118, or 119) are expected to have reduced ligand binding. Amino acid 119 (with respect to present SEQ ID NO:1) is poorly conserved and so is readily altered or truncated. ActRIIB polypeptides and ActRIIB-based GDF traps ending at 128 (with respect to SEQ ID NO: 1) or later should retain ligand-binding activity. ActRIIB polypeptides and ActRIIB-based GDF traps ending at or between 119 and 127 (e.g., 119, 120, 121, 122, 123, 124, 125, 126, or 127), with respect to SEQ ID NO: 1, will have an intermediate binding ability. Any of these forms may be desirable to use, depending on the clinical or experimental setting.

At the N-terminus of ActRIIB, it is expected that a protein beginning at amino acid 29 or before (with respect to SEQ ID NO: 1) will retain ligand-binding activity. Amino acid 29 represents the initial cysteine. An alanine-to-asparagine mutation at position 24 (with respect to SEQ ID NO: 1) introduces an N-linked glycosylation sequence without substantially affecting ligand binding [U.S. Pat. No. 7,842,663]. This confirms that mutations in the region between the signal cleavage peptide and the cysteine cross-linked region, corresponding to amino acids 20-29, are well tolerated. In particular, ActRIIB polypeptides beginning at position 20, 21, 22, 23, and 24 (with respect to SEQ ID NO: 1) should retain general ligand-biding activity, and ActRIIB polypeptides beginning at positions 25, 26, 27, 28, and 29 (with respect to SEQ ID NO: 1) are also expected to retain ligand-biding activity. It has been demonstrated, e.g., U.S. Pat. No. 7,842,663, that, surprisingly, an ActRIIB construct beginning at 22, 23, 24, or 25 will have the most activity.

Taken together, a general formula for an active portion (e.g., ligand-binding portion) of ActRIIB comprises amino acids 29-109 of SEQ ID NO: 1. Therefore ActRIIB polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to a portion of ActRIIB beginning at a residue corresponding to any one of amino acids 20-29 (e.g., beginning at any one of amino acids 20, 21, 22, 23, 24, 25, 26, 27, 28, or 29) of SEQ ID NO: 1 and ending at a position corresponding to any one amino acids 109-134 (e.g., ending at any one of amino acids 109, 110, 111, 112, 113, 114, 115, 116, 117, 118, 119, 120, 121, 122, 123, 124, 125, 126, 127, 128, 129, 130, 131, 132, 133, or 134) of SEQ ID NO: 1. Other examples include polypeptides that begin at a position from 20-29 (e.g., any one of positions 20, 21, 22, 23, 24, 25, 26, 27, 28, or 29) or 21-29 (e.g., any one of positions 21, 22, 23, 24, 25, 26, 27, 28, or 29) of SEQ ID NO: 1 and end at a position from 119-134 (e.g., any one of positions 119, 120, 121, 122, 123, 124, 125, 126, 127, 128, 129, 130, 131, 132, 133, or 134), 119-133 (e.g., any one of positions 119, 120, 121, 122, 123, 124, 125, 126, 127, 128, 129, 130, 131, 132, or 133), 129-134 (e.g., any one of positions 129, 130, 131, 132, 133, or 134), or 129-133 (e.g., any one of positions 129, 130, 131, 132, or 133) of SEQ ID NO: 1. Other examples include constructs that begin at a position from 20-24 (e.g., any one of positions 20, 21, 22, 23, or 24), 21-24 (e.g., any one of positions 21, 22, 23, or 24), or 22-25 (e.g., any one of positions 22, 22, 23, or 25) of SEQ ID NO: 1 and end at a position from 109-134 (e.g., any one of positions 109, 110, 111, 112, 113, 114, 115, 116, 117, 118, 119, 120, 121, 122, 123, 124, 125, 126, 127, 128, 129, 130, 131, 132, 133, or 134), 119-134 (e.g., any one of positions 119, 120, 121, 122, 123, 124, 125, 126, 127, 128, 129, 130, 131, 132, 133, or 134) or 129-134 (e.g., any one of positions 129, 130, 131, 132, 133, or 134) of SEQ ID NO: 1. Variants within these ranges are also contemplated, particularly those comprising, consisting essentially of, or consisting of an amino acid sequence that has at least 70%, 75%, 80%, 85%, 86%, 87%, 88%, 89%, 90%, 91%, 92%, 93%, 94%, 95%, 96%, 97%, 98%, 99%, or 100% identity to the corresponding portion of SEQ ID NO: 1.

The variations described herein may be combined in various ways. In some embodiments, ActRIIB variants comprise no more than 1, 2, 5, 6, 7, 8, 9, 10 or 15 conservative amino acid changes in the ligand-binding pocket, optionally zero, one or more non-conservative alterations at positions 40, 53, 55, 74, 79 and/or 82 in the ligand-binding pocket. Sites outside the binding pocket, at which variability may be particularly well tolerated, include the amino and carboxy termini of the extracellular domain (as noted above), and positions 42-46 and 65-73 (with respect to SEQ ID NO: 1). An asparagine-to-alanine alteration at position 65 (N65A) does not appear to decrease ligand binding in the R64 background [U.S. Pat. No. 7,842,663]. This change probably eliminates glycosylation at N65 in the A64 background, thus demonstrating that a significant change in this region is likely to be tolerated. While an R64A change is poorly tolerated, R64K is well-tolerated, and thus another basic residue, such as H may be tolerated at position 64 [U.S. Pat. No. 7,842,663]. Additionally, the results of the mutagenesis program described in the art indicate that there are amino acid positions in ActRIIB that are often beneficial to conserve. With respect to SEQ ID NO: 1, these include position 80 (acidic or hydrophobic amino acid), position 78 (hydrophobic, and particularly tryptophan), position 37 (acidic, and particularly aspartic or glutamic acid), position 56 (basic amino acid), position 60 (hydrophobic amino acid, particularly phenylalanine or tyrosine). Thus, the disclosure provides a framework of amino acids that may be conserved in ActRIIB polypeptides. Other positions that may be desirable to conserve are as follows: position 52 (acidic amino acid), position 55 (basic amino acid), position 81 (acidic), 98 (polar or charged, particularly E, D, R or K), all with respect to SEQ ID NO: 1.

It has been previously demonstrated that the addition of a further N-linked glycosylation site (N-X-S/T) into the ActRIIB extracellular domain is well-tolerated (see, e.g., U.S. Pat. No. 7,842,663). Therefore, N-X-S/T sequences may be generally introduced at positions outside the ligand binding pocket defined, for example, in FIG. 1 in ActRIIB polypeptide of the present disclosure. Particularly suitable sites for the introduction of non-endogenous N-X-S/T sequences include amino acids 20-29, 20-24, 22-25, 109-134, 120-134 or 129-134 (with respect to SEQ ID NO: 1). N-X-S/T sequences may also be introduced into the linker between the ActRIIB sequence and an Fc domain or other fusion component as well as optionally into the fusion component itself. Such a site may be introduced with minimal effort by introducing an N in the correct position with respect to a pre-existing S or T, or by introducing an S or T at a position corresponding to a pre-existing N. Thus, desirable alterations that would create an N-linked glycosylation site are: A24N, R64N, S67N (possibly combined with an N65A alteration), E105N, R112N, G120N, E123N, P129N, A132N, R112S and R112T (with respect to SEQ ID NO: 1). Any S that is predicted to be glycosylated may be altered to a T without creating an immunogenic site, because of the protection afforded by the glycosylation. Likewise, any T that is predicted to be glycosylated may be altered to an S. Thus the alterations S67T and S44T (with respect to SEQ ID NO: 1) are contemplated. Likewise, in an A24N variant, an S26T alteration may be used. Accordingly, an ActRIIB polypeptide of the present disclosure may be a variant having one or more additional, non-endogenous N-linked glycosylation consensus sequences as described above.

In certain embodiments, the disclosure relates to ActRIIB polypeptides, which includes fragments, functional variants, and modified forms thereof as well as uses thereof (e.g., treating heart failure or a complication of heart failure). Preferably, ActRIIB polypeptides are soluble (e.g., an extracellular domain of ActRIIB) In some embodiments, ActRIIB polypeptides antagonize activity (e.g., Smad signaling) of one or more TGF-beta superfamily ligands [e.g., GDF11, GDF8, activin (activin A, activin B, activin AB, activin C, activin E) BMP6, GDF3, BMP10, and/or BMP9]. Therefore, in some embodiments, ActRIIB polypeptides bind to one or more TGF-beta superfamily ligands [e.g., GDF11, GDF8, activin (activin A, activin B, activin AB, activin C, activin E) BMP6, GDF3, BMP10, and/or BMP9]. In some embodiments, ActRIIB polypeptides of the disclosure comprise, consist essentially of, or consist of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to a portion of ActRIIB beginning at a residue corresponding to amino acids 20-29 (e.g., beginning at any one of amino acids 20, 21, 22, 23, 24, 25, 26, 27, 28, or 29) of SEQ ID NO: 1 and ending at a position corresponding to amino acids 109-134 (e.g., ending at any one of amino acids 109, 110, 111, 112, 113, 114, 115, 116, 117, 118, 119, 120, 121, 122, 123, 124, 125, 126, 127, 128, 129, 130, 131, 132, 133, or 134) of SEQ ID NO: 1. In some embodiments, ActRIIB polypeptides comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical amino acids 29-109 of SEQ ID NO: 1. In some embodiments, ActRIIB polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical amino acids 29-109 of SEQ ID NO: 1, wherein the position corresponding to L79 of SEQ ID NO: 1 is an acidic amino acid (naturally occurring acidic amino acids D and E or an artificial acidic amino acid). In certain embodiments, ActRIIB polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical amino acids 25-131 of SEQ ID NO: 1. In certain embodiments, ActRIIB polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical amino acids 25-131 of SEQ ID NO: 1, wherein the position corresponding to L79 of SEQ ID NO: 1 is an acidic amino acid. In some embodiments, ActRIIB polypeptide of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of any one of SEQ ID NOs: 1, 2, 3, 4, 5, 6, 58, 59, 60, 63, 64, 65, 66, 123, 131, 132, and 133. In some embodiments, ActRIIB polypeptide of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of any one of SEQ ID NOs: 1, 2, 3, 4, 5, 6, 58, 59, 60, 63, 64, 65, 66, 123, 131, 132, and 133, wherein the position corresponding to L79 of SEQ ID NO: 1 is an acidic amino acid. In some embodiments, ActRIIB polypeptides of the disclosure comprise, consist, or consist essentially of, at least one ActRIIB polypeptide wherein the position corresponding to L79 of SEQ ID NO: 1 is not an acidic amino acid (i.e., is not naturally occurring acid amino acids D or E or an artificial acidic amino acid residue).

In certain embodiments, the present disclosure relates to ActRIIA polypeptides. As used herein, the term “ActRIIA” refers to a family of activin receptor type IIA (ActRIIA) proteins from any species and variants derived from such ActRIIA proteins by mutagenesis or other modification. Reference to ActRIIA herein is understood to be a reference to any one of the currently identified forms. Members of the ActRIIA family are generally transmembrane proteins, composed of a ligand-binding extracellular domain comprising a cysteine-rich region, a transmembrane domain, and a cytoplasmic domain with predicted serine/threonine kinase activity.

The term “ActRIIA polypeptide” includes polypeptides comprising any naturally occurring polypeptide of an ActRIIA family member as well as any variants thereof (including mutants, fragments, fusions, and peptidomimetic forms) that retain a useful activity. Examples of such variant ActRIIA polypeptides are provided throughout the present disclosure as well as in International Patent Application Publication No. WO 2006/012627 and WO 2007/062188, which are incorporated herein by reference in their entirety. Numbering of amino acids for all ActRIIA-related polypeptides described herein is based on the numbering of the human ActRIIA precursor protein sequence provided below (SEQ ID NO: 9), unless specifically designated otherwise.

The human ActRIIA precursor protein sequence is as follows:

| (SEQ ID NO: 9) | |
|---|---|
| 1 | MGAAAKLAFA VFLISCSSGA ILGRSETQEC LFFNANWEKD RT [symbol] QTGVEPC |
| 51 | YGDKDKRRHC FATWKNISGS IEIVKQGCWL DDINCYDRTD CVEKKDSPEV |
| 101 | YFCCCEGNMC NEKFSYFPEM EVTQPTSNPV TPKPPYYNIL LYSLVPLMLI |
| 151 | AGIVICAFWV YRHHKMAYPP VLVPTQDPGP PPPSPLLGLK PLQLLEVKAR |
| 201 | GRFGCVWKAQ LLNEYVAVKI FPIQDKQSWQ NEYEVYSLPG MKHENILQFI |
| 251 | GAEKRGTSVD VDLWLITAFH EKGSLSDFLK ANVVSWNELC HIAETMARGL |
| 301 | AYLHEDIPGL KDGHKPAISH RDIKSKNVLL KNNLTACIAD FGLALKFEAG |
| 351 | KSAGDTHGQV GTRRYMAPEV LEGAINFQRD AFLRIDMYAM GLVLWELASR |
| 401 | CTAADGPVDE YMLPFEEEIG QHPSLEDMQE VVVHKKKRPV LRDYWQKHAG |
| 451 | MAMLCETIEE CWDHDAEARL SAGCVGERIT QMQRLTNIIT TEDIVTVVTM |
| 501 | VTNVDFPPKE SSL |

The signal peptide is indicated by a single underline; the extracellular domain is indicated in bold font; and the potential, endogenous N-linked glycosylation sites are indicated by a double underline.

A processed extracellular human ActRIIA polypeptide sequence is as follows:

(SEQ ID NO: 10)
ILGRSETQECLFFNANWEKDRTNQTGVEPCYGDKDKRRHCFATWKNISGS
IEIVKQGCWLDDINCYDRTDCVEKKDSPEVYFCCCEGNMCNEKFSYFPEM
EVTQPTSNPVTPKPP

The C-terminal “tail” of the extracellular domain is indicated by single underline. The sequence with the “tail” deleted (a Δ15 sequence) is as follows:

(SEQ ID NO: 11)
ILGRSETQECLFFNANWEKDRTNQTGVEPCYGDKDKRRHCFATWKNISGS
IEIVKQGCWLDDINCYDRTDCVEKKDSPEVYFCCCEGNMCNEKFSYFPEM

A nucleic acid sequence encoding human ActRIIA precursor protein is shown below (SEQ ID NO: 12), as follows nucleotides 159-1700 of Genbank Reference Sequence NM_001616.4. The signal sequence is underlined.

| (SEQ ID NO: 12) | |
|---|---|
| 1 | ATGGGAGCTG CTGCAAAGTT GGCGTTTGCC GTCTTTCTTA TCTCCTGTTC |
| 51 | TTCAGGTGCT ATACTTGGTA GATCAGAAAC TCAGGAGTGT CTTTTCTTTA |
| 101 | ATGCTAATTG GGAAAAAGAC AGAACCAATC AAACTGGTGT TGAACCGTGT |
| 151 | TATGGTGACA AAGATAAACG GCGGCATTGT TTTGCTACCT GGAAGAATAT |
| 201 | TTCTGGTTCC ATTGAAATAG TGAAACAAGG TTGTTGGCTG GATGATATCA |
| 251 | ACTGCTATGA CAGGACTGAT TGTGTAGAAA AAAAAGACAG CCCTGAAGTA |
| 301 | TATTTTTGTT GCTGTGAGGG CAATATGTGT AATGAAAAGT TTTCTTATTT |
| 351 | TCCGGAGATG GAAGTCACAC AGCCCACTTC AAATCCAGTT ACACCTAAGC |
| 401 | CACCCTATTA CAACATCCTG CTCTATTCCT TGGTGCCACT TATGTTAATT |
| 451 | GCGGGGATTG TCATTTGTGC ATTTTGGGTG TACAGGCATC ACAAGATGGC |
| 501 | CTACCCTCCT GTACTTGTTC CAACTCAAGA CCCAGGACCA CCCCCACCTT |
| 551 | CTCCATTACT AGGTTTGAAA CCACTGCAGT TATTAGAAGT GAAAGCAAGG |
| 601 | GGAAGATTTG GTTGTGTCTG GAAAGCCCAG TTGCTTAACG AATATGTGGC |
| 651 | TGTCAAAATA TTTCCAATAC AGGACAAACA GTCATGGCAA AATGAATACG |
| 701 | AAGTCTACAG TTTGCCTGGA ATGAAGCATG AGAACATATT ACAGTTCATT |
| 751 | GGTGCAGAAA AACGAGGCAC CAGTGTTGAT GTGGATCTTT GGCTGATCAC |
| 801 | AGCATTTCAT GAAAAGGGTT CACTATCAGA CTTTCTTAAG GCTAATGTGG |
| 851 | TCTCTTGGAA TGAACTGTGT CATATTGCAG AAACCATGGC TAGAGGATTG |
| 901 | GCATATTTAC ATGAGGATAT ACCTGGCCTA AAAGATGGCC ACAAACCTGC |
| 951 | CATATCTCAC AGGGACATCA AAAGTAAAAA TGTGCTGTTG AAAAACAACC |
| 1001 | TGACAGCTTG CATTGCTGAC TTTGGGTTGG CCTTAAAATT TGAGGCTGGC |
| 1051 | AAGTCTGCAG GCGATACCCA TGGACAGGTT GGTACCCGGA GGTACATGGC |
| 1101 | TCCAGAGGTA TTAGAGGGTG CTATAAACTT CCAAAGGGAT GCATTTTTGA |
| 1151 | GGATAGATAT GTATGCCATG GGATTAGTCC TATGGGAACT GGCTTCTCGC |
| 1201 | TGTACTGCTG CAGATGGACC TGTAGATGAA TACATGTTGC CATTTGAGGA |
| 1251 | GGAAATTGGC CAGCATCCAT CTCTTGAAGA CATGCAGGAA GTTGTTGTGC |
| 1301 | ATAAAAAAAA GAGGCCTGTT TTAAGAGATT ATTGGCAGAA ACATGCTGGA |
| 1351 | ATGGCAATGC TCTGTGAAAC CATTGAAGAA TGTTGGGATC ACGACGCAGA |
| 1401 | AGCCAGGTTA TCAGCTGGAT GTGTAGGTGA AAGAATTACC CAGATGCAGA |
| 1451 | GACTAACAAA TATTATTACC ACAGAGGACA TTGTAACAGT GGTCACAATG |
| 1501 | GTGACAAATG TTGACTTTCC TCCCAAAGAA TCTAGTCTA |

A nucleic acid sequence encoding processed human ActRIIA polypeptide is as follows:

| (SEQ ID NO: 13) | |
|---|---|
| 1 | ATACTTGGTA GATCAGAAAC TCAGGAGTGT CTTTTCTTTA ATGCTAATTG |
| 51 | GGAAAAAGAC AGAACCAATC AAACTGGTGT TGAACCGTGT TATGGTGACA |
| 101 | AAGATAAACG GCGGCATTGT TTTGCTACCT GGAAGAATAT TTCTGGTTCC |
| 151 | ATTGAAATAG TGAAACAAGG TTGTTGGCTG GATGATATCA ACTGCTATGA |
| 201 | CAGGACTGAT TGTGTAGAAA AAAAAGACAG CCCTGAAGTA TATTTTTGTT |
| 251 | GCTGTGAGGG CAATATGTGT AATGAAAAGT TTTCTTATTT TCCGGAGATG |
| 301 | GAAGTCACAC AGCCCACTTC AAATCCAGTT ACACCTAAGC CACCC |

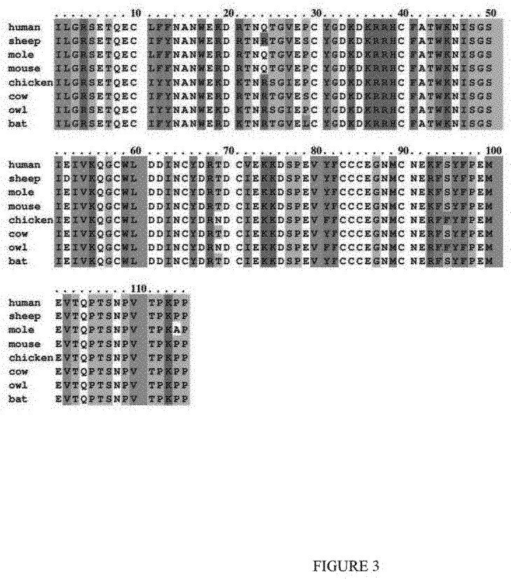

ActRIIA is well-conserved among vertebrates, with large stretches of the extracellular domain completely conserved. For example, FIG. 3 depicts a multi-sequence alignment of a human ActRIIA extracellular domain compared to various ActRIIA orthologs. Many of the ligands that bind to ActRIIA are also highly conserved. Accordingly, from these alignments, it is possible to predict key amino acid positions within the ligand-binding domain that are important for normal ActRIIA-ligand binding activities as well as to predict amino acid positions that are likely to be tolerant to substitution without significantly altering normal ActRIIA-ligand binding activities. Therefore, an active, human ActRIIA variant polypeptide useful in accordance with the presently disclosed methods may include one or more amino acids at corresponding positions from the sequence of another vertebrate ActRIIA, or may include a residue that is similar to that in the human or other vertebrate sequences.

Without meaning to be limiting, the following examples illustrate this approach to defining an active ActRIIA variant. As illustrated in FIG. 3, F13 in the human extracellular domain is Y in Ovis aries (SEQ ID NO: 108), Gallus gallus (SEQ ID NO: 111), Bos Taurus (SEQ ID NO: 112), Tyto alba (SEQ ID NO: 113), and Myotis davidii (SEQ ID NO: 114) ActRIIA, indicating that aromatic residues are tolerated at this position, including F, W, and Y. Q24 in the human extracellular domain is R in Bos Taurus ActRIIA, indicating that charged residues will be tolerated at this position, including D, R, K, H, and E. S95 in the human extracellular domain is F in Gallus gallus and Tyto alba ActRIIA, indicating that this site may be tolerant of a wide variety of changes, including polar residues, such as E, D, K, R, H, S, T, P, G, Y, and probably hydrophobic residue such as L, I, or F. E52 in the human extracellular domain is D in Ovis aries ActRIIA, indicating that acidic residues are tolerated at this position, including D and E. P29 in the human extracellular domain is relatively poorly conserved, appearing as S in Ovis aries ActRIIA and L in Myotis davidii ActRIIA, thus essentially any amino acid should be tolerated at this position.

Moreover, as discussed above, ActRII proteins have been characterized in the art in terms of structural/functional characteristics, particularly with respect to ligand binding [Attisano et al. (1992) Cell 68(1):97-108; Greenwald et al. (1999) Nature Structural Biology 6(1): 18-22; Allendorph et al. (2006) PNAS 103(20: 7643-7648; Thompson et al. (2003) The EMBO Journal 22(7): 1555-1566; as well as U.S. Pat. Nos. 7,709,605, 7,612,041, and 7,842,663]. In addition to the teachings herein, these references provide amply guidance for how to generate ActRIIA variants that retain one or more desired activities (e.g., ligand-binding activity).

For example, a defining structural motif known as a three-finger toxin fold is important for ligand binding by type I and type II receptors and is formed by conserved cysteine residues located at varying positions within the extracellular domain of each monomeric receptor [Greenwald et al. (1999) Nat Struct Biol 6:18-22; and Hinck (2012) FEBS Lett 586:1860-1870]. Accordingly, the core ligand-binding domains of human ActRIIA, as demarcated by the outermost of these conserved cysteines, corresponds to positions 30-110 of SEQ ID NO: 9 (ActRIIA precursor). Therefore, the structurally less-ordered amino acids flanking these cysteine-demarcated core sequences can be truncated by about 1, 2, 3, 4, 5, 6, 7, 8, 9, 10, 11, 12, 13, 14, 15, 16, 17, 18, 19, 20, 21, 22, 23, 24, 25, 26, 27, 28, or 29 residues at the N-terminus and by about 1, 2, 3, 4, 5, 6, 7, 8, 9, 10, 11, 12, 13, 14, 15, 16, 17, 18, 19, 20, 21, 22, 23, 24, or 25 residues at the C-terminus without necessarily altering ligand binding. Exemplary ActRIIA extracellular domains truncations include SEQ ID NOs: 10 and 11.

Accordingly, a general formula for an active portion (e.g., ligand binding) of ActRIIA is a polypeptide that comprises, consists essentially of, or consists of amino acids 30-110 of SEQ ID NO: 9. Therefore ActRIIA polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to a portion of ActRIIA beginning at a residue corresponding to any one of amino acids 21-30 (e.g., beginning at any one of amino acids 21, 22, 23, 24, 25, 26, 27, 28, 29, or 30) of SEQ ID NO: 9 and ending at a position corresponding to any one amino acids 110-135 (e.g., ending at any one of amino acids 110, 111, 112, 113, 114, 115, 116, 117, 118, 119, 120, 121, 122, 123, 124, 125, 126, 127, 128, 129, 130, 131, 132, 133, or 135) of SEQ ID NO: 9. Other examples include constructs that begin at a position selected from 21-30 (e.g., beginning at any one of amino acids 21, 22, 23, 24, 25, 26, 27, 28, 29, or 30), 22-30 (e.g., beginning at any one of amino acids 22, 23, 24, 25, 26, 27, 28, 29, or 30), 23-30 (e.g., beginning at any one of amino acids 23, 24, 25, 26, 27, 28, 29, or 30), 24-30 (e.g., beginning at any one of amino acids 24, 25, 26, 27, 28, 29, or 30) of SEQ ID NO: 9, and end at a position selected from 111-135 (e.g., ending at any one of amino acids 111, 112, 113, 114, 115, 116, 117, 118, 119, 120, 121, 122, 123, 124, 125, 126, 127, 128, 129, 130, 131, 132, 133, 134 or 135), 112-135 (e.g., ending at any one of amino acids 112, 113, 114, 115, 116, 117, 118, 119, 120, 121, 122, 123, 124, 125, 126, 127, 128, 129, 130, 131, 132, 133, 134 or 135), 113-135 (e.g., ending at any one of amino acids 113, 114, 115, 116, 117, 118, 119, 120, 121, 122, 123, 124, 125, 126, 127, 128, 129, 130, 131, 132, 133, 134 or 135), 120-135 (e.g., ending at any one of amino acids 120, 121, 122, 123, 124, 125, 126, 127, 128, 129, 130, 131, 132, 133, 134 or 135), 130-135 (e.g., ending at any one of amino acids 130, 131, 132, 133, 134 or 135), 111-134 (e.g., ending at any one of amino acids 110, 111, 112, 113, 114, 115, 116, 117, 118, 119, 120, 121, 122, 123, 124, 125, 126, 127, 128, 129, 130, 131, 132, 133, or 134), 111-133 (e.g., ending at any one of amino acids 110, 111, 112, 113, 114, 115, 116, 117, 118, 119, 120, 121, 122, 123, 124, 125, 126, 127, 128, 129, 130, 131, 132, or 133), 111-132 (e.g., ending at any one of amino acids 110, 111, 112, 113, 114, 115, 116, 117, 118, 119, 120, 121, 122, 123, 124, 125, 126, 127, 128, 129, 130, 131, or 132), or 111-131 (e.g., ending at any one of amino acids 110, 111, 112, 113, 114, 115, 116, 117, 118, 119, 120, 121, 122, 123, 124, 125, 126, 127, 128, 129, 130, or 131) of SEQ ID NO: 9. Variants within these ranges are also contemplated, particularly those comprising, consisting essentially of, or consisting of an amino acid sequence that has at least 70%, 75%, 80%, 85%, 86%, 87%, 88%, 89%, 90%, 91%, 92%, 93%, 94%, 95%, 96%, 97%, 98%, 99%, or 100% identity to the corresponding portion of SEQ ID NO: 9. Thus, in some embodiments, an ActRIIA polypeptide may comprise, consists essentially of, or consist of a polypeptide that is at least 70%, 75%, 80%, 85%, 86%, 87%, 88%, 89%, 90%, 91%, 92%, 93%, 94%, 95%, 96%, 97%, 98%, 99%, or 100% identical to amino acids 30-110 of SEQ ID NO: 9. Optionally, ActRIIA polypeptides comprise a polypeptide that is at least 70%, 75%, 80%, 85%, 86%, 87%, 88%, 89%, 90%, 91%, 92%, 93%, 94%, 95%, 96%, 97%, 98%, 99%, or 100% identical to amino acids 30-110 of SEQ ID NO: 9, and comprising no more than 1, 2, 5, 10 or 15 conservative amino acid changes in the ligand-binding pocket.

In certain embodiments, the disclosure relates to ActRIIA polypeptides, which includes fragments, functional variants, and modified forms thereof as well as uses thereof (e.g., increasing an immune response in a patient in need thereof and treating cancer). Preferably, ActRIIA polypeptides are soluble (e.g., an extracellular domain of ActRIIA). In some embodiments, ActRIIA polypeptides inhibit (e.g., Smad signaling) of one or more TGF-beta superfamily ligands [e.g., GDF11, GDF8, activin (activin A, activin B, activin AB, activin C, activin E) BMP6, GDF3, BMP10, and/or BMP9]. In some embodiments, ActRIIA polypeptides bind to one or more TGF-beta superfamily ligands [e.g., GDF11, GDF8, activin (activin A, activin B, activin AB, activin C, activin E) BMP6, GDF3, BMP10, and/or BMP9]. In some embodiments, ActRIIA polypeptide of the disclosure comprise, consist essentially of, or consist of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to a portion of ActRIIA beginning at a residue corresponding to amino acids 21-30 (e.g., beginning at any one of amino acids 21, 22, 23, 24, 25, 26, 27, 28, 29, or 30) of SEQ ID NO: 9 and ending at a position corresponding to any one amino acids 110-135 (e.g., ending at any one of amino acids 110, 111, 112, 113, 114, 115, 116, 117, 118, 119, 120, 121, 122, 123, 124, 125, 126, 127, 128, 129, 130, 131, 132, 133, or 135) of SEQ ID NO: 9. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical amino acids 30-110 of SEQ ID NO: 9. In certain embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical amino acids 21-135 of SEQ ID NO: 9. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of any one of SEQ ID NOs: 9, 10, 11, 50, 54, and 57.

In certain aspects, the present disclosure relates to GDF trap polypeptides (also referred to as “GDF traps”). In some embodiments, GDF traps of the present disclosure are variant ActRII polypeptides (e.g., ActRIIA and ActRIIB polypeptides) that comprise one or more mutations (e.g., amino acid additions, deletions, substitutions, and combinations thereof) in the extracellular domain (also referred to as the ligand-binding domain) of an ActRII polypeptide (e.g., a “wild-type” or unmodified ActRII polypeptide) such that the variant ActRII polypeptide has one or more altered ligand-binding activities than the corresponding wild-type ActRII polypeptide. In preferred embodiments, GDF trap polypeptides of the present disclosure retain at least one similar activity as a corresponding wild-type ActRII polypeptide. For example, preferable GDF traps bind to and inhibit (e.g. antagonize) the function of GDF11 and/or GDF8. In some embodiments, GDF traps of the present disclosure further bind to and inhibit one or more of ligand of the TGF-beta superfamily. Accordingly, the present disclosure provides GDF trap polypeptides that have an altered binding specificity for one or more ActRII ligands.

To illustrate, one or more mutations may be selected that increase the selectivity of the altered ligand-binding domain for GDF11 and/or GDF8 over one or more ActRII-binding ligands such as activins (activin A, activin B, activin AB, activin C, and/or activin E), particularly activin A. Optionally, the altered ligand-binding domain has a ratio of K_{d} for activin binding to K_{d} for GDF11 and/or GDF8 binding that is at least 2-, 5-, 10-, 20-, 50-, 100- or even 1000-fold greater relative to the ratio for the wild-type ligand-binding domain. Optionally, the altered ligand-binding domain has a ratio of IC₅₀ for inhibiting activin to IC₅₀ for inhibiting GDF11 and/or GDF8 that is at least 2-, 5-, 10-, 20-, 50-, 100- or even 1000-fold greater relative to the wild-type ligand-binding domain. Optionally, the altered ligand-binding domain inhibits GDF11 and/or GDF8 with an IC₅₀ at least 2-, 5-, 10-, 20-, 50-, 100- or even 1000-times less than the IC₅₀ for inhibiting activin.

In certain preferred embodiments, GDF traps of the present disclosure are designed to preferentially bind to GDF11 and/or GDF8 (also known as myostatin). Optionally, GDF11 and/or GDF8-binding traps may further bind to activin B. Optionally, GDF11 and/or GDF8-binding traps may further bind to BMP6. Optionally, GDF11 and/or GDF8-binding traps may further bind to BMP10. Optionally, GDF11 and/or GDF8-binding traps may further bind to activin B and BMP6. In certain embodiments, GDF traps of the present disclosure have diminished binding affinity for activins (e.g., activin A, activin A/B, activin B, activin C, activin E), e.g., in comparison to a wild-type ActRII polypeptide. In certain preferred embodiments, a GDF trap polypeptide of the present disclosure has diminished binding affinity for activin A.

Amino acid residues of the ActRIIB proteins (e.g., E39, K55, Y60, K74, W78, L79, D80, and F101 with respect to SEQ ID NO: 1) are in the ActRIIB ligand-binding pocket and help mediated binding to its ligands including, for example, activin A, GDF11, and GDF8. Thus the present disclosure provides GDF trap polypeptides comprising an altered-ligand binding domain (e.g., a GDF8/GDF11-binding domain) of an ActRIIB receptor which comprises one or more mutations at those amino acid residues.

As a specific example, the positively-charged amino acid residue Asp (D80) of the ligand-binding domain of ActRIIB can be mutated to a different amino acid residue to produce a GDF trap polypeptide that preferentially binds to GDF8, but not activin. Preferably, the D80 residue with respect to SEQ ID NO: 1 is changed to an amino acid residue selected from the group consisting of: an uncharged amino acid residue, a negative amino acid residue, and a hydrophobic amino acid residue. As a further specific example, the hydrophobic residue L79 of SEQ ID NO: 1 can be altered to confer altered activin-GDF11/GDF8 binding properties. For example, an L79P substitution reduces GDF11 binding to a greater extent than activin binding. In contrast, replacement of L79 with an acidic amino acid [an aspartic acid or glutamic acid; an L79D or an L79E substitution] greatly reduces activin A binding affinity while retaining GDF11 binding affinity. In exemplary embodiments, the methods described herein utilize a GDF trap polypeptide which is a variant ActRIIB polypeptide comprising an acidic amino acid (e.g., D or E) at the position corresponding to position 79 of SEQ ID NO: 1, optionally in combination with one or more additional amino acid substitutions, additions, or deletions.

The term “BMPRII polypeptide” includes polypeptides comprising any naturally occurring polypeptide of a BMPRII family member as well as any variants thereof (including mutants, fragments, fusions, and peptidomimetic forms) that retain a useful activity. Proteins described herein are the human forms unless otherwise specified. Numbering of amino acids for all BMPRII-related polypeptides described herein is based on the numbering of the human BMPRII precursor protein sequence provided below (SEQ ID NO: 14), unless specifically designated otherwise.

The amino acid sequence of the unprocessed canonical isoform of human BMPRII precursor (NCBI Reference Sequence NP 001195.2) is as follows:

| (SEQ ID NO: 14) | |
|---|---|
| 1 | MTSSLQRPWR VPWLPWTILL VSTAAASQNQ ERLCAFKDPY QQDLGIGESR |
| 51 | ISHENGTILC SKGSTCYGLW EKSKGDINLV KQGCWSHIGD PQECHYEECV |
| 101 | VTTTPPSIQN GTYRFCCCST DLCNVNFTEN FPPPDTTPLS PPHSFNRDET |
| 151 | IIIALASVSV LAVLIVALCF GYRMLTGDRK QGLHSMNMME AAASEPSLDL |
| 201 | DNLKLLELIG RGRYGAVYKG SLDERPVAVK VFSFANRQNF INEKNIYRVP |
| 251 | LMEHDNIARF IVGDERVTAD GRMEYLLVME YYPNGSLCKY LSLHTSDWVS |
| 301 | SCRLAHSVTR GLAYLHTELP RGDHYKPAIS HRDLNSRNVL VKNDGTCVIS |
| 351 | DFGLSMRLTG NRLVRPGEED NAAISEVGTI RYMAPEVLEG AVNLRDCESA |
| 401 | LKQVDMYALG LIYWEIFMRC TDLFPGESVP EYQMAFQTEV GNHPTFEDMQ |
| 451 | VLVSREKQRP KFPEAWKENS LAVRSLKETI EDCWDQDAEA RLTAQCAEER |
| 501 | MAELMMIWER NKSVSPTVNP MSTAMQNERN LSHNRRVPKI GPYPDYSSSS |
| 551 | YIEDSIHHTD SIVKNISSEH SMSSTPLTIG EKNRNSINYE RQQAQARIPS |
| 601 | PETSVTSLST NTTTTNTTGL TPSTGMTTIS EMPYPDETNL HTTNVAQSIG |
| 651 | PTPVCLQLTE EDLETNKLDP KEVDKNLKES SDENLMEHSL KQFSGPDPLS |
| 701 | STSSSLLYPL IKLAVEATGQ QDFTQTANGQ ACLIPDVLPT QIYPLPKQQN |
| 751 | LPKRPTSLPL NTKNSTKEPR LKFGSKHKSN LKQVETGVAK MNTINAAEPH |
| 801 | VVTVTMNGVA GRNHSVNSHA ATTQYANGTV LSGQTTNIVT HRAQEMLQNQ |
| 851 | FIGEDTRLNI NSSPDEHEPL LRREQQAGHD EGVLDRLVDR RERPLEGGRT |
| 901 | NSNNNNSNPC SEQDVLAQGV PSTAADPGPS KPRRAQRPNS LDLSATNVLD |
| 951 | GSSIQIGEST QDGKSGSGEK IKKRVKTPYS LKRWRPSTWV ISTESLDCEV |
| 1001 | NNNGSNRAVH SKSSTAVYLA EGGTATTMVS KDIGMNCL |

The signal peptide is underlined, and the extracellular domain is indicated in bold.

The sequence of a processed extracellular BMPRII polypeptide (SEQ ID NO: 15) is as follows:

| (SEQ ID NO: 15) | |
|---|---|
| 1 | SQNQERLCAF KDPYQQDLGI GESRISHENG TILCSKGSTC YGLWEKSKGD |
| 51 | INLVKQGCWS HIGDPQECHY EECVVTTTPP SIQNGTYRFC CCSTDLCNVN |
| 101 | FTENFPPPDT TPLSPPHSFN RDET |

Based on the positioning of cysteine residues in the sequence, a BMPRII polypeptide may comprise an amino acid sequence beginning at amino acid 1, 2, 3, 4, 5, 6, 7 or 8 of SEQ ID NO: 15 and ending at any of amino acids 97-124 of SEQ ID NO: 15. A nucleic acid sequence encoding the canonical human BMPRII precursor protein is shown below (SEQ ID NO: 16), corresponding to nucleotides 1149-4262 of NCBI Reference Sequence NM_001204.6. The signal sequence is underlined.

(SEQ ID NO: 16)
ATGACTTCCTCGCTGCAGCGGCCCTGGCGGGTGCCCTGGCTACCATGGAC
CATCCTGCTGGTCAGCACTGCGGCTGCTTCGCAGAATCAAGAACGGCTAT
GTGCGTTTAAAGATCCGTATCAGCAAGACCTTGGGATAGGTGAGAGTAGA
ATCTCTCATGAAAATGGGACAATATTATGCTCGAAAGGTAGCACCTGCTA
TGGCCTTTGGGAGAAATCAAAAGGGGACATAAATCTTGTAAAACAAGGAT
GTTGGTCTCACATTGGAGATCCCCAAGAGTGTCACTATGAAGAATGTGTA
GTAACTACCACTCCTCCCTCAATTCAGAATGGAACATACCGTTTCTGCTG
TTGTAGCACAGATTTATGTAATGTCAACTTTACTGAGAATTTTCCACCTC
CTGACACAACACCACTCAGTCCACCTCATTCATTTAACCGAGATGAGACA
ATAATCATTGCTTTGGCATCAGTCTCTGTATTAGCTGTTTTGATAGTTGC
CTTATGCTTTGGATACAGAATGTTGACAGGAGACCGTAAACAAGGTCTTC
ACAGTATGAACATGATGGAGGCAGCAGCATCCGAACCCTCTCTTGATCTA
GATAATCTGAAACTGTTGGAGCTGATTGGCCGAGGTCGATATGGAGCAGT
ATATAAAGGCTCCTTGGATGAGCGTCCAGTTGCTGTAAAAGTGTTTTCCT
TTGCAAACCGTCAGAATTTTATCAACGAAAAGAACATTTACAGAGTGCCT
TTGATGGAACATGACAACATTGCCCGCTTTATAGTTGGAGATGAGAGAGT
CACTGCAGATGGACGCATGGAATATTTGCTTGTGATGGAGTACTATCCCA
ATGGATCTTTATGCAAGTATTTAAGTCTCCACACAAGTGACTGGGTAAGC
TCTTGCCGTCTTGCTCATTCTGTTACTAGAGGACTGGCTTATCTTCACAC
AGAATTACCACGAGGAGATCATTATAAACCTGCAATTTCCCATCGAGATT
TAAACAGCAGAAATGTCCTAGTGAAAAATGATGGAACCTGTGTTATTAGT
GACTTTGGACTGTCCATGAGGCTGACTGGAAATAGACTGGTGCGCCCAGG
GGAGGAAGATAATGCAGCCATAAGCGAGGTTGGCACTATCAGATATATGG
CACCAGAAGTGCTAGAAGGAGCTGTGAACTTGAGGGACTGTGAATCAGCT
TTGAAACAAGTAGACATGTATGCTCTTGGACTAATCTATTGGGAGATATT
TATGAGATGTACAGACCTCTTCCCAGGGGAATCCGTACCAGAGTACCAGA
TGGCTTTTCAGACAGAGGTTGGAAACCATCCCACTTTTGAGGATATGCAG
GTTCTCGTGTCTAGGGAAAAACAGAGACCCAAGTTCCCAGAAGCCTGGAA
AGAAAATAGCCTGGCAGTGAGGTCACTCAAGGAGACAATCGAAGACTGTT
GGGACCAGGATGCAGAGGCTCGGCTTACTGCACAGTGTGCTGAGGAAAGG
ATGGCTGAACTTATGATGATTTGGGAAAGAAACAAATCTGTGAGCCCAAC
AGTCAATCCAATGTCTACTGCTATGCAGAATGAACGCAACCTGTCACATA
ATAGGCGTGTGCCAAAAATTGGTCCTTATCCAGATTATTCTTCCTCCTCA
TACATTGAAGACTCTATCCATCATACTGACAGCATCGTGAAGAATATTTC
CTCTGAGCATTCTATGTCCAGCACACCTTTGACTATAGGGGAAAAAAACC
GAAATTCAATTAACTATGAACGACAGCAAGCACAAGCTCGAATCCCCAGC
CCTGAAACAAGTGTCACCAGCCTCTCCACCAACACAACAACCACAAACAC
CACAGGACTCACGCCAAGTACTGGCATGACTACTATATCTGAGATGCCAT
ACCCAGATGAAACAAATCTGCATACCACAAATGTTGCACAGTCAATTGGG
CCAACCCCTGTCTGCTTACAGCTGACAGAAGAAGACTTGGAAACCAACAA
GCTAGACCCAAAAGAAGTTGATAAGAACCTCAAGGAAAGCTCTGATGAGA
ATCTCATGGAGCACTCTCTTAAACAGTTCAGTGGCCCAGACCCACTGAGC
AGTACTAGTTCTAGCTTGCTTTACCCACTCATAAAACTTGCAGTAGAAGC
AACTGGACAGCAGGACTTCACACAGACTGCAAATGGCCAAGCATGTTTGA
TTCCTGATGTTCTGCCTACTCAGATCTATCCTCTCCCCAAGCAGCAGAAC
CTTCCCAAGAGACCTACTAGTTTGCCTTTGAACACCAAAAATTCAACAAA
AGAGCCCCGGCTAAAATTTGGCAGCAAGCACAAATCAAACTTGAAACAAG
TCGAAACTGGAGTTGCCAAGATGAATACAATCAATGCAGCAGAACCTCAT
GTGGTGACAGTCACCATGAATGGTGTGGCAGGTAGAAACCACAGTGTTAA
CTCCCATGCTGCCACAACCCAATATGCCAATGGGACAGTACTATCTGGCC
AAACAACCAACATAGTGACACATAGGGCCCAAGAAATGTTGCAGAATCAG
TTTATTGGTGAGGACACCCGGCTGAATATTAATTCCAGTCCTGATGAGCA
TGAGCCTTTACTGAGACGAGAGCAACAAGCTGGCCATGATGAAGGTGTTC
TGGATCGTCTTGTGGACAGGAGGGAACGGCCACTAGAAGGTGGCCGAACT
AATTCCAATAACAACAACAGCAATCCATGTTCAGAACAAGATGTTCTTGC
ACAGGGTGTTCCAAGCACAGCAGCAGATCCTGGGCCATCAAAGCCCAGAA
GAGCACAGAGGCCTAATTCTCTGGATCTTTCAGCCACAAATGTCCTGGAT
GGCAGCAGTATACAGATAGGTGAGTCAACACAAGATGGCAAATCAGGATC
AGGTGAAAAGATCAAGAAACGTGTGAAAACTCCCTATTCTCTTAAGCGGT
GGCGCCCCTCCACCTGGGTCATCTCCACTGAATCGCTGGACTGTGAAGTC
AACAATAATGGCAGTAACAGGGCAGTTCATTCCAAATCCAGCACTGCTGT
TTACCTTGCAGAAGGAGGCACTGCTACAACCATGGTGTCTAAAGATATAG
GAATGAACTGTCTG

A nucleic acid sequence encoding processed extracellular BMPRII polypeptide (SEQ ID NO: 17) is as follows:

| (SEQ ID NO: 17) | |
|---|---|
| 1 | TCGCAGAATC AAGAACGGCT ATGTGCGTTT AAAGATCCGT ATCAGCAAGA |
| 51 | CCTTGGGATA GGTGAGAGTA GAATCTCTCA TGAAAATGGG ACAATATTAT |
| 101 | GCTCGAAAGG TAGCACCTGC TATGGCCTTT GGGAGAAATC AAAAGGGGAC |
| 151 | ATAAATCTTG TAAAACAAGG ATGTTGGTCT CACATTGGAG ATCCCCAAGA |
| 201 | GTGTCACTAT GAAGAATGTG TAGTAACTAC CACTCCTCCC TCAATTCAGA |
| 251 | ATGGAACATA CCGTTTCTGC TGTTGTAGCA CAGATTTATG TAATGTCAAC |
| 301 | TTTACTGAGA ATTTTCCACC TCCTGACACA ACACCACTCA GTCCACCTCA |
| 351 | TTCATTTAAC CGAGATGAGA CA |

A shorter isoform of human BMPRII precursor (isoform A) has been reported, which contains the same extracellular domain sequence as the canonical BMPRII precursor above. The amino acid sequence of human BMPRII precursor isoform A (NCBI Accession Number AAA86519.1) is as follows:

| (SEQ ID NO: 18) | |
|---|---|
| 1 | MTSSLQRPWR VPWLPWTILL VSTAAASQNQ ERLCAFKDPY QQDLGIGESR |
| 51 | ISHENGTILC SKGSTCYGLW EKSKGDINLV KQGCWSHIGD PQECHYEECV |
| 101 | VTTTPPSIQN GTYRFCCCST DLCNVNFTEN FPPPDTTPLS PPHSFNRDET |
| 151 | IIIALASVSV LAVLIVALCF GYRMLTGDRK QGLHSMNMME AAASEPSLDL |
| 201 | DNLKLLELIG RGRYGAVYKG SLDERPVAVK VFSFANRQNF INEKNIYRVP |
| 251 | LMEHDNIARF IVGDERVTAD GRMEYLLVME YYPNGSLCKY LSLHTSDWVS |
| 301 | SCRLAHSVTR GLAYLHTELP RGDHYKPAIS HRDLNSRNVL VKNDGTCVIS |
| 351 | DFGLSMRLTG NRLVRPGEED NAAISEVGTI RYMAPEVLEG AVNLRDCESA |
| 401 | LKQVDMYALG LIYWEIFMRC TDLFPGESVP EYQMAFQTEV GNHPTFEDMQ |
| 451 | VLVSREKQRP KFPEAWKENS LAVRSLKETI EDCWDQDAEA RLTAQCAEER |
| 501 | MAELMMIWER NKSVSPTVNP MSTAMQNERR |

The signal peptide is underlined, and the extracellular domain is indicated in bold.

A nucleic acid sequence encoding isoform A of the human BMPRII precursor protein is shown below (SEQ ID NO: 19), corresponding to nucleotides 163-1752 of NCBI accession number U25110.1. The signal sequence is underlined.

(SEQ ID NO: 19)
ATGACTTCCTCGCTGCAGCGGCCCTGGCGGGTGCCCTGGCTACCATGGAC
CATCCTGCTGGTCAGCACTGCGGCTGCTTCGCAGAATCAAGAACGGCTAT
GTGCGTTTAAAGATCCGTATCAGCAAGACCTTGGGATAGGTGAGAGTAGA
ATCTCTCATGAAAATGGGACAATATTATGCTCGAAAGGTAGCACCTGCTA
TGGCCTTTGGGAGAAATCAAAAGGGGACATAAATCTTGTAAAACAAGGAT
GTTGGTCTCACATTGGAGATCCCCAAGAGTGTCACTATGAAGAATGTGTA
GTAACTACCACTCCTCCCTCAATTCAGAATGGAACATACCGTTTCTGCTG
TTGTAGCACAGATTTATGTAATGTCAACTTTACTGAGAATTTTCCACCTC
CTGACACAACACCACTCAGTCCACCTCATTCATTTAACCGAGATGAGACA
ATAATCATTGCTTTGGCATCAGTCTCTGTATTAGCTGTTTTGATAGTTGC
CTTATGCTTTGGATACAGAATGTTGACAGGAGACCGTAAACAAGGTCTTC
ACAGTATGAACATGATGGAGGCAGCAGCATCCGAACCCTCTCTTGATCTA
GATAATCTGAAACTGTTGGAGCTGATTGGCCGAGGTCGATATGGAGCAGT
ATATAAAGGCTCCTTGGATGAGCGTCCAGTTGCTGTAAAAGTGTTTTCCT
TTGCAAACCGTCAGAATTTTATCAACGAAAAGAACATTTACAGAGTGCCT
TTGATGGAACATGACAACATTGCCCGCTTTATAGTTGGAGATGAGAGAGT
CACTGCAGATGGACGCATGGAATATTTGCTTGTGATGGAGTACTATCCCA
ATGGATCTTTATGCAAGTATTTAAGTCTCCACACAAGTGACTGGGTAAGC
TCTTGCCGTCTTGCTCATTCTGTTACTAGAGGACTGGCTTATCTTCACAC
AGAATTACCACGAGGAGATCATTATAAACCTGCAATTTCCCATCGAGATT
TAAACAGCAGAAATGTCCTAGTGAAAAATGATGGAACCTGTGTTATTAGT
GACTTTGGACTGTCCATGAGGCTGACTGGAAATAGACTGGTGCGCCCAGG
GGAGGAAGATAATGCAGCCATAAGCGAGGTTGGCACTATCAGATATATGG
CACCAGAAGTGCTAGAAGGAGCTGTGAACTTGAGGGACTGTGAATCAGCT
TTGAAACAAGTAGACATGTATGCTCTTGGACTAATCTATTGGGAGATATT
TATGAGATGTACAGACCTCTTCCCAGGGGAATCCGTACCAGAGTACCAGA
TGGCTTTTCAGACAGAGGTTGGAAACCATCCCACTTTTGAGGATATGCAG
GTTCTCGTGTCTAGGGAAAAACAGAGACCCAAGTTCCCAGAAGCCTGGAA
AGAAAATAGCCTGGCAGTGAGGTCACTCAAGGAGACAATCGAAGACTGTT
GGGACCAGGATGCAGAGGCTCGGCTTACTGCACAGTGTGCTGAGGAAAGG
ATGGCTGAACTTATGATGATTTGGGAAAGAAACAAATCTGTGAGCCCAAC
AGTCAATCCAATGTCTACTGCTATGCAGAATGAACGTAGG

A defining structural motif known as a three-finger toxin fold is important for ligand binding by TGFbeta superfamily type I and type II receptors and is formed by 10, 12, or 14 conserved cysteine residues located at varying positions within the extracellular domain of each monomeric receptor. See, e.g., Greenwald et al. (1999) Nat Struct Biol 6:18-22; Galat (2011) Cell Mol Life Sci 68:3437-3451; Hinck (2012) FEBS Lett 586:1860-1870. The core ligand-binding domain of a BMPRII receptor, as demarcated by the outermost of these conserved cysteines, comprises positions 34-123 of SEQ ID NO: 14. It is expected that a BMPRII polypeptide beginning at amino acid 34 (the initial cysteine of the ECD), or before, of SEQ ID NO: 14 and ending at amino acid 123 (the last cysteine of the ECD), or after, of SEQ ID NO: 14 will retain ligand binding activity. Examples of ligand binding BMPRII polypeptides therefore include, for example, polypeptides comprising an amino acid sequence that begins at any one of amino acids 27-34 (27, 28, 29, 30, 31, 32, 33, or 34) of SEQ ID NO: 14 and ends at any one of amino acids 123-150 (123, 124, 125, 126, 127, 128, 129, 130, 131, 132, 133, 134, 135, 136, 137, 138, 139, 140, 141, 142, 143, 144, 145, 146, 147, 148, 149, or 150) of SEQ ID NO: 14. In some embodiments, a BMPRII polypeptide of the disclosure comprises an amino acid sequence that is at least 70%, 75%, 80%, 85%, 90%, 91%, 92%, 93%, 94%, 95%, 96%, 97%, 98%, 99%, or 100% identical to amino acids 34-123 of SEQ ID NO: 14. In some embodiments, a BMPRII polypeptide of the disclosure comprises an amino acid sequence that is at least 70%, 75%, 80%, 85%, 90%, 91%, 92%, 93%, 94%, 95%, 96%, 97%, 98%, 99%, or 100% identical to amino acids 27-150 of SEQ ID NO: 14. In some embodiments, a BMPRII polypeptide of the disclosure comprises an amino acid sequence that is at least 70%, 75%, 80%, 85%, 90%, 91%, 92%, 93%, 94%, 95%, 96%, 97%, 98%, 99%, or 100% identical to 27-123 of SEQ ID NO: 14. In some embodiments, a BMPRII polypeptide of the disclosure comprises an amino acid sequence that is at least 70%, 75%, 80%, 85%, 90%, 91%, 92%, 93%, 94%, 95%, 96%, 97%, 98%, 99%, or 100% identical to 34-150 of SEQ ID NO: 14. In certain embodiments, a BMPRII polypeptide binds to BMP9, BMP10, BMP15 and/or activin B, and the BMPRII polypeptide does not show substantial binding to canonical BMP such as BMP2, BMP4, BMP6 and/or BMP7. Binding may be assessed, for example, using purified proteins in solution or in a surface plasmon resonance system, such as a Biacore™ system.

The term “ALK1 polypeptide” includes polypeptides comprising any naturally occurring polypeptide of an ALK1 family member as well as any variants thereof (including mutants, fragments, fusions, and peptidomimetic forms) that retain a useful activity.

The human ALK1 precursor protein sequence (NCBI Ref Seq NP 000011.2) is as follows:

| (SEQ ID NO: 20) | |
|---|---|
| 1 | MTLGSPRKGL LMLLMALVTQ GDPVKPSRGP LVTCTCESPH CKGPTCRGAW |
| 51 | CTVVLVREEG RHPQEHRGCG NLHRELCRGR PTEFVNHYCC DSHLCNHNVS |
| 101 | LVLEATQPPS EQPGTDGQLA LILGPVLALL ALVALGVLGL WHVRRRQEKQ |
| 151 | RGLHSELGES SLILKASEQG DSMLGDLLDS DCTTGSGSGL PFLVQRTVAR |
| 201 | QVALVECVGK GRYGEVWRGL WHGESVAVKI FSSRDEQSWF RETEIYNTVL |
| 251 | LRHDNILGFI ASDMTSRNSS TQLWLITHYH EHGSLYDFLQ RQTLEPHLAL |
| 301 | RLAVSAACGL AHLHVEIFGT QGKPAIAHRD FKSRNVLVKS NLQCCIADLG |
| 351 | LAVMHSQGSD YLDIGNNPRV GTKRYMAPEV LDEQIRTDCF ESYKWTDIWA |
| 401 | FGLVLWEIAR RTIVNGIVED YRPPFYDVVP NDPSFEDMKK VVCVDQQTPT |
| 451 | IPNRLAADPV LSGLAQMMRE CWYPNPSARL TALRIKKTLQ KISNSPEKPK |
| 501 | VIQ |

The signal peptide is indicated by a single underline and the extracellular domain is indicated in bold font.

A processed extracellular ALK1 polypeptide sequence is as follows:

(SEQ ID NO: 21)
DPVKPSRGPLVTCTCESPHCKGPTCRGAWCTVVLVREEGRHPQEHRGCGN
LHRELCRGRPTEFVNHYCCDSHLCNHNVSLVLEATQPPSEQPGTDGQ

A nucleic acid sequence encoding human ALK1 precursor protein is shown below (SEQ ID NO: 22), corresponding to nucleotides 284-1792 of Genbank Reference Sequence NM_000020.2. The signal sequence is underlined.

(SEQ ID NO: 22)
ATGACCTTGGGCTCCCCCAGGAAAGGCCTTCTGATGCTGCTGATGGCCTT
GGTGACCCAGGGAGACCCTGTGAAGCCGTCTCGGGGCCCGCTGGTGACCT
GCACGTGTGAGAGCCCACATTGCAAGGGGCCTACCTGCCGGGGGGCCTGG
TGCACAGTAGTGCTGGTGCGGGAGGAGGGGAGGCACCCCCAGGAACATCG
GGGCTGCGGGAACTTGCACAGGGAGCTCTGCAGGGGGCGCCCCACCGAGT
TCGTCAACCACTACTGCTGCGACAGCCACCTCTGCAACCACAACGTGTCC
CTGGTGCTGGAGGCCACCCAACCTCCTTCGGAGCAGCCGGGAACAGATGG
CCAGCTGGCCCTGATCCTGGGCCCCGTGCTGGCCTTGCTGGCCCTGGTGG
CCCTGGGTGTCCTGGGCCTGTGGCATGTCCGACGGAGGCAGGAGAAGCAG
CGTGGCCTGCACAGCGAGCTGGGAGAGTCCAGTCTCATCCTGAAAGCATC
TGAGCAGGGCGACAGCATGTTGGGGGACCTCCTGGACAGTGACTGCACCA
CAGGGAGTGGCTCAGGGCTCCCCTTCCTGGTGCAGAGGACAGTGGCACGG
CAGGTTGCCTTGGTGGAGTGTGTGGGAAAAGGCCGCTATGGCGAAGTGTG
GCGGGGCTTGTGGCACGGTGAGAGTGTGGCCGTCAAGATCTTCTCCTCGA
GGGATGAACAGTCCTGGTTCCGGGAGACTGAGATCTATAACACAGTGTTG
CTCAGACACGACAACATCCTAGGCTTCATCGCCTCAGACATGACCTCCCG
CAACTCGAGCACGCAGCTGTGGCTCATCACGCACTACCACGAGCACGGCT
CCCTCTACGACTTTCTGCAGAGACAGACGCTGGAGCCCCATCTGGCTCTG
AGGCTAGCTGTGTCCGCGGCATGCGGCCTGGCGCACCTGCACGTGGAGAT
CTTCGGTACACAGGGCAAACCAGCCATTGCCCACCGCGACTTCAAGAGCC
GCAATGTGCTGGTCAAGAGCAACCTGCAGTGTTGCATCGCCGACCTGGGC
CTGGCTGTGATGCACTCACAGGGCAGCGATTACCTGGACATCGGCAACAA
CCCGAGAGTGGGCACCAAGCGGTACATGGCACCCGAGGTGCTGGACGAGC
AGATCCGCACGGACTGCTTTGAGTCCTACAAGTGGACTGACATCTGGGCC
TTTGGCCTGGTGCTGTGGGAGATTGCCCGCCGGACCATCGTGAATGGCAT
CGTGGAGGACTATAGACCACCCTTCTATGATGTGGTGCCCAATGACCCCA
GCTTTGAGGACATGAAGAAGGTGGTGTGTGTGGATCAGCAGACCCCCACC
ATCCCTAACCGGCTGGCTGCAGACCCGGTCCTCTCAGGCCTAGCTCAGAT
GATGCGGGAGTGCTGGTACCCAAACCCCTCTGCCCGACTCACCGCGCTGC
GGATCAAGAAGACACTACAAAAAATTAGCAACAGTCCAGAGAAGCCTAAA
GTGATTCAA

A nucleic acid sequence encoding processed extracelluar ALK1 polypeptide is as follows:

(SEQ ID NO: 23)
GACCCTGTGAAGCCGTCTCGGGGCCCGCTGGTGACCTGCACGTGTGAGAG
CCCACATTGCAAGGGGCCTACCTGCCGGGGGGCCTGGTGCACAGTAGTGC
TGGTGCGGGAGGAGGGGAGGCACCCCCAGGAACATCGGGGCTGCGGGAAC
TTGCACAGGGAGCTCTGCAGGGGGCGCCCCACCGAGTTCGTCAACCACTA
CTGCTGCGACAGCCACCTCTGCAACCACAACGTGTCCCTGGTGCTGGAGG
CCACCCAACCTCCTTCGGAGCAGCCGGGAACAGATGGCCAG

As discussed above, a defining structural motif known as a three-finger toxin fold is important for ligand binding by TGFbeta superfamily type I and type II receptors and is formed by 10, 12, or 14 conserved cysteine residues located at varying positions within the extracellular domain of each monomeric receptor. The core ligand-binding domain of an ALK1 receptor, as demarcated by the outermost of these conserved cysteines, comprises positions 34-95 of SEQ ID NO: 20. It is expected that an ALK1 polypeptide beginning at amino acid 34 (the initial cysteine of the ECD), or before, of SEQ ID NO: 20 and ending at amino acid 95 (the last cysteine of the ECD), or after, of SEQ ID NO: 20 will retain ligand binding activity. Examples of ligand binding ALK1 polypeptides therefore include, for example, polypeptides comprising an amino acid sequence that begins at any one of amino acids 22-34 (22, 23, 24, 25, 26, 27, 28, 29, 30, 31, 32, 33, or 34) of SEQ ID NO: 20 and ends at any one of amino acids 95-118 (95, 96, 97, 98, 99, 100, 101, 102, 103, 104, 105, 106, 107, 108, 109, 110, 111, 112, 113, 114, 115, 116, 117, or 118) of SEQ ID NO: 20. In some embodiments, an ALK1 polypeptide of the disclosure comprises an amino acid sequence that is at least 70%, 75%, 80%, 85%, 90%, 91%, 92%, 93%, 94%, 95%, 96%, 97%, 98%, 99%, or 100% identical to amino acids 34-95 of SEQ ID NO: 20. In some embodiments, an ALK1 polypeptide of the disclosure comprises an amino acid sequence that is at least 70%, 75%, 80%, 85%, 90%, 91%, 92%, 93%, 94%, 95%, 96%, 97%, 98%, 99%, or 100% identical to amino acids 22-118 of SEQ ID NO: 20. In some embodiments, an ALK1 polypeptide of the disclosure comprises an amino acid sequence that is at least 70%, 75%, 80%, 85%, 90%, 91%, 92%, 93%, 94%, 95%, 96%, 97%, 98%, 99%, or 100% identical to 22-95 of SEQ ID NO: 20. In some embodiments, an ALK1 polypeptide of the disclosure comprises an amino acid sequence that is at least 70%, 75%, 80%, 85%, 90%, 91%, 92%, 93%, 94%, 95%, 96%, 97%, 98%, 99%, or 100% identical to 34-95 of SEQ ID NO: 20. In certain embodiments, an ALK1 polypeptide binds to BMP9 and BMP10. Binding may be assessed, for example, using purified proteins in solution or in a surface plasmon resonance system, such as a Biacore™ system.

The term “endoglin polypeptide” includes polypeptides comprising any naturally occurring polypeptide of an endoglin family member as well as any variants thereof (including mutants, fragments, fusions, and peptidomimetic forms) that retain a useful activity.

The human endoglin isoform 1 precursor protein sequence (GenBank NM_001114753) is as follows:

| (SEQ ID NO: 24) | |
|---|---|
| 1 | MDRGTLPLAV ALLLASCSLS PTSLAETVHC DLQPVGPERG EVTYTTSQVS KGCVAQAPNA |
| 61 | ILEVHVLFLE FPTGPSQLEL TLQASKQNGT WPREVLLVLS VNSSVFLHLQ ALGIPLHLAY |
| 121 | NSSLVTFQEP PGVNTTELPS FPKTQILEWA AERGPITSAA ELNDPQSILL RLGQAQGSLS |
| 181 | FCMLEASQDM GRTLEWRPRT PALVRGCHLE GVAGHKEAHI LRVLPGHSAG PRTVTVKVEL |
| 241 | SCAPGDLDAV LILQGPPYVS WLIDANHNMQ IWTTGEYSFK IFPEKNIRGF KLPDTPQGLL |
| 301 | GEARMLNASI VASFVELPLA SIVSLHASSC GGRLQTSPAP IQTTPPKDTC SPELLMSLIQ |
| 361 | TKCADDAMTL VLKKELVAHL KCTITGLTFW DPSCEAEDRG DKFVLRSAYS SCGMQVSASM |
| 421 | ISNEAVVNIL SSSSPQRKKV HCLNMDSLSF QLGLYLSPHF LQASNTIEPG QQSFVQVRVS |
| 481 | PSVSEFLLQL DSCHLDLGPE GGTVELIQGR AAKGNCVSLL SPSPEGDPRF SFLLHFYTVP |
| 541 | IPKTGTLSCT VALRPKTGSQ DQEVHRTVFM RLNIISPDLS GCTSKGLVLP AVLGITFGAF |
| 601 | LIGALLTAAL WYIYSHTRSP SKREPVVAVA APASSESSST NHSIGSTQST PCSTSSMA |

The leader sequence and predicted transmembrane domain are each indicated by a single underline.

A nucleic acid sequence encoding human endoglin isoform 1 precursor protein is shown below (SEQ ID NO: 25; Genbank Reference Sequence NM_001114753). The leader sequence and predicted transmembrane domain are each indicated by a single underline.

| (SEQ ID NO: 25) | |
|---|---|
| 1 | ATGGACCGCG GCACGCTCCC TCTGGCTGTT GCCCTGCTGC TGGCCAGCTG |
| 51 | CAGCCTCAGC CCCACAAGTC TTGCAGAAAC AGTCCATTGT GACCTTCAGC |
| 101 | CTGTGGGCCC CGAGAGGGGC GAGGTGACAT ATACCACTAG CCAGGTCTCG |
| 151 | AAGGGCTGCG TGGCTCAGGC CCCCAATGCC ATCCTTGAAG TCCATGTCCT |
| 201 | CTTCCTGGAG TTCCCAACGG GCCCGTCACA GCTGGAGCTG ACTCTCCAGG |
| 251 | CATCCAAGCA AAATGGCACC TGGCCCCGAG AGGTGCTTCT GGTCCTCAGT |
| 301 | GTAAACAGCA GTGTCTTCCT GCATCTCCAG GCCCTGGGAA TCCCACTGCA |
| 351 | CTTGGCCTAC AATTCCAGCC TGGTCACCTT CCAAGAGCCC CCGGGGGTCA |
| 401 | ACACCACAGA GCTGCCATCC TTCCCCAAGA CCCAGATCCT TGAGTGGGCA |
| 451 | GCTGAGAGGG GCCCCATCAC CTCTGCTGCT GAGCTGAATG ACCCCCAGAG |
| 501 | CATCCTCCTC CGACTGGGCC AAGCCCAGGG GTCACTGTCC TTCTGCATGC |
| 551 | TGGAAGCCAG CCAGGACATG GGCCGCACGC TCGAGTGGCG GCCGCGTACT |
| 601 | CCAGCCTTGG TCCGGGGCTG CCACTTGGAA GGCGTGGCCG GCCACAAGGA |
| 651 | GGCGCACATC CTGAGGGTCC TGCCGGGCCA CTCGGCCGGG CCCCGGACGG |
| 701 | TGACGGTGAA GGTGGAACTG AGCTGCGCAC CCGGGGATCT CGATGCCGTC |
| 751 | CTCATCCTGC AGGGTCCCCC CTACGTGTCC TGGCTCATCG ACGCCAACCA |
| 801 | CAACATGCAG ATCTGGACCA CTGGAGAATA CTCCTTCAAG ATCTTTCCAG |
| 851 | AGAAAAACAT TCGTGGCTTC AAGCTCCCAG ACACACCTCA AGGCCTCCTG |
| 901 | GGGGAGGCCC GGATGCTCAA TGCCAGCATT GTGGCATCCT TCGTGGAGCT |
| 951 | ACCGCTGGCC AGCATTGTCT CACTTCATGC CTCCAGCTGC GGTGGTAGGC |
| 1001 | TGCAGACCTC ACCCGCACCG ATCCAGACCA CTCCTCCCAA GGACACTTGT |
| 1051 | AGCCCGGAGC TGCTCATGTC CTTGATCCAG ACAAAGTGTG CCGACGACGC |
| 1101 | CATGACCCTG GTACTAAAGA AAGAGCTTGT TGCGCATTTG AAGTGCACCA |
| 1151 | TCACGGGCCT GACCTTCTGG GACCCCAGCT GTGAGGCAGA GGACAGGGGT |
| 1201 | GACAAGTTTG TCTTGCGCAG TGCTTACTCC AGCTGTGGCA TGCAGGTGTC |
| 1251 | AGCAAGTATG ATCAGCAATG AGGCGGTGGT CAATATCCTG TCGAGCTCAT |
| 1301 | CACCACAGCG GAAAAAGGTG CACTGCCTCA ACATGGACAG CCTCTCTTTC |
| 1351 | CAGCTGGGCC TCTACCTCAG CCCACACTTC CTCCAGGCCT CCAACACCAT |
| 1401 | CGAGCCGGGG CAGCAGAGCT TTGTGCAGGT CAGAGTGTCC CCATCCGTCT |
| 1451 | CCGAGTTCCT GCTCCAGTTA GACAGCTGCC ACCTGGACTT GGGGCCTGAG |
| 1501 | GGAGGCACCG TGGAACTCAT CCAGGGCCGG GCGGCCAAGG GCAACTGTGT |
| 1551 | GAGCCTGCTG TCCCCAAGCC CCGAGGGTGA CCCGCGCTTC AGCTTCCTCC |
| 1601 | TCCACTTCTA CACAGTACCC ATACCCAAAA CCGGCACCCT CAGCTGCACG |
| 1651 | GTAGCCCTGC GTCCCAAGAC CGGGTCTCAA GACCAGGAAG TCCATAGGAC |
| 1701 | TGTCTTCATG CGCTTGAACA TCATCAGCCC TGACCTGTCT GGTTGCACAA |
| 1751 | GCAAAGGCCT CGTCCTGCCC GCCGTGCTGG GCATCACCTT TGGTGCCTTC |
| 1801 | CTCATCGGGG CCCTGCTCAC TGCTGCACTC TGGTACATCT ACTCGCACAC |
| 1851 | GCGTTCCCCC AGCAAGCGGG AGCCCGTGGT GGCGGTGGCT GCCCCGGCCT |
| 1901 | CCTCGGAGAG CAGCAGCACC AACCACAGCA TCGGGAGCAC CCAGAGCACC |
| 1951 | CCCTGCTCCA CCAGCAGCAT GGCA |

The human endoglin isoform 2 precursor protein sequence (GenBank NM_001114753) is as follows:

| (SEQ ID NO: 26) | |
|---|---|
| 1 | MDRGTLPLAV ALLLASCSLS PTSLAETVHC DLQPVGPERG EVTYTTSQVS KGCVAQAPNA |
| 61 | ILEVHVLFLE FPTGPSQLEL TLQASKQNGT WPREVLLVLS VNSSVFLHLQ ALGIPLHLAY |
| 121 | NSSLVTFQEP PGVNTTELPS FPKTQILEWA AERGPITSAA ELNDPQSILL RLGQAQGSLS |
| 181 | FCMLEASQDM GRTLEWRPRT PALVRGCHLE GVAGHKEAHI LRVLPGHSAG PRTVTVKVEL |
| 241 | SCAPGDLDAV LILQGPPYVS WLIDANHNMQ IWTTGEYSFK IFPEKNIRGF KLPDTPQGLL |
| 301 | GEARMLNASI VASFVELPLA SIVSLHASSC GGRLQTSPAP IQTTPPKDTC SPELLMSLIQ |
| 361 | TKCADDAMTL VLKKELVAHL KCTITGLTFW DPSCEAEDRG DKFVLRSAYS SCGMQVSASM |
| 421 | ISNEAVVNIL SSSSPQRKKV HCLNMDSLSF QLGLYLSPHF LQASNTIEPG QQSFVQVRVS |
| 481 | PSVSEFLLQL DSCHLDLGPE GGTVELIQGR AAKGNCVSLL SPSPEGDPRF SFLLHFYTVP |
| 541 | IPKTGTLSCT VALRPKTGSQ DQEVHRTVFM RLNIISPDLS GCTSKGLVLP AVLGITFGAF |
| 601 | LIGALLTAAL WYIYSHTREY PRPPQ |

The leader sequence and predicted transmembrane domain are each indicated by a single underline.

A nucleic acid sequence encoding human ALK1 isoform 2 precursor protein is shown below (SEQ ID NO: 27; Genbank Reference Sequence NM_001114753). The leader sequence and predicted transmembrane domain are each indicated by a single underline.

(SEQ ID NO: 27)
ATGGACCGCGGCACGCTCCCTCTGGCTGTTGCCCTGCTGCTGGCCAGCTG
CAGCCTCAGCCCCACAAGTCTTGCAGAAACAGTCCATTGTGACCTTCAGC
CTGTGGGCCCCGAGAGGGGCGAGGTGACATATACCACTAGCCAGGTCTCG
AAGGGCTGCGTGGCTCAGGCCCCCAATGCCATCCTTGAAGTCCATGTCCT
CTTCCTGGAGTTCCCAACGGGCCCGTCACAGCTGGAGCTGACTCTCCAGG
CATCCAAGCAAAATGGCACCTGGCCCCGAGAGGTGCTTCTGGTCCTCAGT
GTAAACAGCAGTGTCTTCCTGCATCTCCAGGCCCTGGGAATCCCACTGCA
CTTGGCCTACAATTCCAGCCTGGTCACCTTCCAAGAGCCCCCGGGGGTCA
ACACCACAGAGCTGCCATCCTTCCCCAAGACCCAGATCCTTGAGTGGGCA
GCTGAGAGGGGCCCCATCACCTCTGCTGCTGAGCTGAATGACCCCCAGAG
CATCCTCCTCCGACTGGGCCAAGCCCAGGGGTCACTGTCCTTCTGCATGC
TGGAAGCCAGCCAGGACATGGGCCGCACGCTCGAGTGGCGGCCGCGTACT
CCAGCCTTGGTCCGGGGCTGCCACTTGGAAGGCGTGGCCGGCCACAAGGA
GGCGCACATCCTGAGGGTCCTGCCGGGCCACTCGGCCGGGCCCCGGACGG
TGACGGTGAAGGTGGAACTGAGCTGCGCACCCGGGGATCTCGATGCCGTC
CTCATCCTGCAGGGTCCCCCCTACGTGTCCTGGCTCATCGACGCCAACCA
CAACATGCAGATCTGGACCACTGGAGAATACTCCTTCAAGATCTTTCCAG
AGAAAAACATTCGTGGCTTCAAGCTCCCAGACACACCTCAAGGCCTCCTG
GGGGAGGCCCGGATGCTCAATGCCAGCATTGTGGCATCCTTCGTGGAGCT
ACCGCTGGCCAGCATTGTCTCACTTCATGCCTCCAGCTGCGGTGGTAGGC
TGCAGACCTCACCCGCACCGATCCAGACCACTCCTCCCAAGGACACTTGT
AGCCCGGAGCTGCTCATGTCCTTGATCCAGACAAAGTGTGCCGACGACGC
CATGACCCTGGTACTAAAGAAAGAGCTTGTTGCGCATTTGAAGTGCACCA
TCACGGGCCTGACCTTCTGGGACCCCAGCTGTGAGGCAGAGGACAGGGGT
GACAAGTTTGTCTTGCGCAGTGCTTACTCCAGCTGTGGCATGCAGGTGTC
AGCAAGTATGATCAGCAATGAGGCGGTGGTCAATATCCTGTCGAGCTCAT
CACCACAGCGGAAAAAGGTGCACTGCCTCAACATGGACAGCCTCTCTTTC
CAGCTGGGCCTCTACCTCAGCCCACACTTCCTCCAGGCCTCCAACACCAT
CGAGCCGGGGCAGCAGAGCTTTGTGCAGGTCAGAGTGTCCCCATCCGTCT
CCGAGTTCCTGCTCCAGTTAGACAGCTGCCACCTGGACTTGGGGCCTGAG
GGAGGCACCGTGGAACTCATCCAGGGCCGGGCGGCCAAGGGCAACTGTGT
GAGCCTGCTGTCCCCAAGCCCCGAGGGTGACCCGCGCTTCAGCTTCCTCC
TCCACTTCTACACAGTACCCATACCCAAAACCGGCACCCTCAGCTGCACG
GTAGCCCTGCGTCCCAAGACCGGGTCTCAAGACCAGGAAGTCCATAGGAC
TGTCTTCATGCGCTTGAACATCATCAGCCCTGACCTGTCTGGTTGCACAA
GCAAAGGCCTCGTCCTGCCCGCCGTGCTGGGCATCACCTTTGGTGCCTTC
CTCATCGGGGCCCTGCTCACTGCTGCACTCTGGTACATCTACTCGCACAC
GCGTGAGTACCCCAGGCCCCCACAG

Applicant has previously demonstrated that Fc fusion proteins comprising shorter C-terminally truncated variants of ENG polypeptides display no appreciable binding to TGF-β1 and TGF-β3 but instead display higher affinity binding to BMP9, with a markedly slower dissociation rate, compared to either ENG(26-437)-Fc or an Fc fusion protein comprising the full-length ENG ECD (see, e.g., US 2015/0307588, the teachings of which are incorporated herein by reference in its entirety). Specifically, C-terminally truncated variants ending at amino acids 378, 359, and 346 of SEQ ID NO: 24 were all found to bind BMP9 with substantially higher affinity (and to bind BMP10 with undiminished affinity) compared to ENG(26-437) or ENG(26-586). However, binding to BMP9 and BMP10 was completely disrupted by more extensive C-terminal truncations to amino acids 332, 329, or 257. Thus, ENG polypeptides that terminate between amino acid 333 and amino acid 378 are all expected to be active, but constructs ending at, or between, amino acids 346 and 359 may be most active. Forms ending at, or between, amino acids 360 and 378 are predicted to trend toward the intermediate ligand binding affinity shown by ENG(26-378). Improvements in other key parameters are expected with certain constructs ending at, or between, amino acids 333 and 378 based on improvements in protein expression and elimination half-life observed with ENG(26-346)-Fc compared to fusion proteins comprising full-length ENG ECD (see, e.g., US 2015/0307588). Any of these truncated variant forms may be desirable to use, depending on the clinical or experimental setting.

At the N-terminus, it is expected that an ENG polypeptide beginning at amino acid 26 (the initial glutamate), or before, of SEQ ID NO: 24 will retain ligand binding activity. As described herein and in US 2015/0307588, an N-terminal truncation to amino acid 61 of SEQ ID NO: 24 abolishes ligand binding, as do more extensive N-terminal truncations. However, as also disclosed herein, consensus modeling of ENG primary sequences indicates that ordered secondary structure within the region defined by amino acids 26-60 of SEQ ID NO: 24 is limited to a four-residue beta strand predicted with high confidence at positions 42-45 of SEQ ID NO: 24 and a two-residue beta strand predicted with very low confidence at positions 28-29 of SEQ ID NO: 24. Thus, an active ENG polypeptide will begin at (or before) amino acid 26, preferentially, or at any of amino acids 27-42 of SEQ ID NO: 24.

Taken together, an active portion of an ENG polypeptide may comprise an amino acid sequence beginning at any one of amino acids 27-42 (e.g., 27, 28, 29, 30, 31, 32, 33, 34, 35, 36, 37, 38, 39, 40, 41, or 42) of SEQ ID NO: 24 and ending at any one of amino acids 333-378 (333, 334, 335, 336, 337, 338, 339, 340, 341, 342, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 277, of 378) of SEQ ID NO: 24, as well as sequence having at least 70%, 75%, 80%, 85%, 90%, 91%, 92%, 93%, 94%, 95%, 96%, 97%, 98%, or 99% identity to the corresponding portion of SEQ ID NO: 24. For example, active ENG polypeptides may comprise amino acid sequences 26-333, 26-334, 26-335, 26-336, 26-337, 26-338, 26-339, 26-340, 26-341, 26-342, 26-343, 26-344, 26-345, or 26-346 of SEQ ID NO: 24, as well as variants of these sequences starting at any of amino acids 27-42 of SEQ ID NO: 24. Exemplary ENG polypeptides comprise amino acid sequences 26-346, 26-359, and 26-378 of SEQ ID NO: 24. Variants within these ranges are also contemplated, particularly those having at least 70%, 75%, 80%, 85%, 90%, 91%, 92%, 93%, 94%, 95%, 96%, 97%, 98%, or 99% identity to the corresponding portion of SEQ ID NO: 24. An ENG polypeptide may not include the sequence consisting of amino acids 379-430 of SEQ ID NO: 24. In certain embodiments, an ENG polypeptide binds to BMP-9 and BMP-10, and the ENG polypeptide does not show substantial binding to TGF-β1 or TGF-β3. Binding may be assessed using purified proteins in solution or in a surface plasmon resonance system, such as a Biacore™ system.

ENG polypeptides may additionally include any of various leader sequences at the N-terminus. Such a sequence would allow the peptides to be expressed and targeted to the secretion pathway in a eukaryotic system. See, e.g., Ernst et al., U.S. Pat. No. 5,082,783 (1992). Alternatively, a native ENG signal sequence may be used to effect extrusion from the cell. Possible leader sequences include honeybee mellitin, TPA, and native leaders. Processing of signal peptides may vary depending on the leader sequence chosen, the cell type used and culture conditions, among other variables, and therefore actual N-terminal start sites for mature ENG polypeptides may shift by 1, 2, 3, 4 or 5 amino acids in either the N-terminal or C-terminal direction. Examples of mature ENG-Fc fusion proteins include SEQ ID NOs: 28-31, as shown below with the ENG polypeptide portion underlined.

Human ENG(26-378)-hFc (truncated Fc)

```
(SEQ ID NO: 28)
        ETVHCD LQPVGPERDE VTYTTSQVSK GCVAQAPNAI
LEVHVLFLEF PTGPSQLELT LQASKQNGTW PREVLLVLSV
NSSVFLHLQA LGIPLHLAYN SSLVTFQEPP GVNTTELPSF
PKTQILEWAA ERGPITSAAE LNDPQSILLR LGQAQGSLSF
CMLEASQDMG RTLEWRPRTP ALVRGCHLEG VAGHKEAHIL
RVLPGHSAGP RTVTVKVELS CAPGDLDAVL ILQGPPYVSW
LIDANHNMQI WTTGEYSFKI FPEKNIRGFK LPDTPQGLLG
EARMLNASIV ASFVELPLAS IVSLHASSCG GRLQTSPAPI
QTTPPKDTCS PELLMSLIQT KCADDAMTLV LKKELVATGG
GTHTCPPCPA PELLGGPSVF LFPPKPKDTL MISRTPEVTC
VVVDVSHEDP EVKFNWYVDG VEVHNAKTKP REEQYNSTYR
VVSVLTVLHQ DWLNGKEYKC KVSNKALPAP IEKTISKAKG
QPREPQVYTL PPSREEMTKN QVSLTCLVKG FYPSDIAVEW
ESNGQPENNY KTTPPVLDSD GSFFLYSKLT VDKSRWQQGN
VFSCSVMHEA LHNHYTQKSL SLSPGK
Human ENG(26-359)-hFc
(SEQ ID NO: 29)
        ETVHCD LQPVGPERDE VTYTTSQVSK GCVAQAPNAI
LEVHVLFLEF PTGPSQLELT LQASKQNGTW PREVLLVLSV
NSSVFLHLQA LGIPLHLAYN SSLVTFQEPP GVNTTELPSF
PKTQILEWAA ERGPITSAAE LNDPQSILLR LGQAQGSLSF
CMLEASQDMG RTLEWRPRTP ALVRGCHLEG VAGHKEAHIL
RVLPGHSAGP RTVTVKVELS CAPGDLDAVL ILQGPPYVSW
LIDANHNMQI WTTGEYSFKI FPEKNIRGFK LPDTPQGLLG
EARMLNASIV ASFVELPLAS IVSLHASSCG GRLQTSPAPI
QTTPPKDTCS PELLMSLITG GGPKSCDKTH TCPPCPAPEL
LGGPSVFLFP PKPKDTLMIS RTPEVTCVVV DVSHEDPEVK
FNWYVDGVEV HNAKTKPREE QYNSTYRVVS VLTVLHQDWL
NGKEYKCKVS NKALPAPIEK TISKAKGQPR EPQVYTLPPS
REEMTKNQVS LTCLVKGFYP SDIAVEWESN GQPENNYKTT
PPVLDSDGSF FLYSKLTVDK SRWQQGNVFS CSVMHEALHN
HYTQKSLSLS PGK
Human ENG(26-359)-hFc (truncated Fc)
(SEQ ID NO: 30)
        ETVHCD LQPVGPERDE VTYTTSQVSK GCVAQAPNAI
LEVHVLFLEF PTGPSQLELT LQASKQNGTW PREVLLVLSV
NSSVFLHLQA LGIPLHLAYN SSLVTFQEPP GVNTTELPSF
PKTQILEWAA ERGPITSAAE LNDPQSILLR LGQAQGSLSF
CMLEASQDMG RTLEWRPRTP ALVRGCHLEG VAGHKEAHIL
RVLPGHSAGP RTVTVKVELS CAPGDLDAVL ILQGPPYVSW
LIDANHNMQI WTTGEYSFKI FPEKNIRGFK LPDTPQGLLG
EARMLNASIV ASFVELPLAS IVSLHASSCG GRLQTSPAPI
QTTPPKDTCS PELLMSLITG GGTHTCPPCP APELLGGPSV
FLFPPKPKDT LMISRTPEVT CVVVDVSHED PEVKFNWYVD
GVEVHNAKTK PREEQYNSTY RVVSVLTVLH QDWLNGKEYK
CKVSNKALPA PIEKTISKAK GQPREPQVYT LPPSREEMTK
NQVSLTCLVK GFYPSDIAVE WESNGQPENN YKTTPPVLDS
DGSFFLYSKL TVDKSRWQQG NVFSCSVMHE ALHNHYTQKS
LSLSPGK
Human ENG(26-346)-hFc (truncated Fc)
(SEQ ID NO: 31)
        ETVHCD LQPVGPERDE VTYTTSQVSK GCVAQAPNAI
LEVHVLFLEF PTGPSQLELT LQASKQNGTW PREVLLVLSV
NSSVFLHLQA LGIPLHLAYN SSLVTFQEPP GVNTTELPSF
PKTQILEWAA ERGPITSAAE LNDPQSILLR LGQAQGSLSF
CMLEASQDMG RTLEWRPRTP ALVRGCHLEG VAGHKEAHIL
RVLPGHSAGP RTVTVKVELS CAPGDLDAVL ILQGPPYVSW
LIDANHNMQI WTTGEYSFKI FPEKNIRGFK LPDTPQGLLG
EARMLNASIV ASFVELPLAS IVSLHASSCG GRLQTSPAPI
QTTPPTGGGT HTCPPCPAPE LLGGPSVFLF PPKPKDTLMI
SRTPEVTCVV VDVSHEDPEV KFNWYVDGVE VHNAKTKPRE
EQYNSTYRVV SVLTVLHQDW LNGKEYKCKV SNKALPAPIE
KTISKAKGQP REPQVYTLPP SREEMTKNQV SLTCLVKGFY
PSDIAVEWES NGQPENNYKT TPPVLDSDGS FFLYSKLTVD
KSRWQQGNVF SCSVMHEALH NHYTQKSLSL SPGK
```

In some embodiments, the present disclosure contemplates making functional variants by modifying the structure of a BMP10 propeptide polypeptide, ActRII polypeptide, BMPRII polypeptide, ALK1 polypeptide and/or endoglin polypeptide for such purposes as enhancing therapeutic efficacy or stability (e.g., shelf-life and resistance to proteolytic degradation in vivo). Variants can be produced by amino acid substitution, deletion, addition, or combinations thereof. For instance, it is reasonable to expect that an isolated replacement of a leucine with an isoleucine or valine, an aspartate with a glutamate, a threonine with a serine, or a similar replacement of an amino acid with a structurally related amino acid (e.g., conservative mutations) will not have a major effect on the biological activity of the resulting molecule. Conservative replacements are those that take place within a family of amino acids that are related in their side chains. Whether a change in the amino acid sequence of a polypeptide of the disclosure results in a functional homolog can be readily determined by assessing the ability of the variant polypeptide to produce a response in cells in a fashion similar to the wild-type polypeptide, or to bind to one or more TGF-beta ligands including, for example, BMP2, BMP2/7, BMP3, BMP4, BMP4/7, BMP5, BMP6, BMP7, BMP8a, BMP8b, BMP9, BMP10, GDF3, GDF5, GDF6/BMP13, GDF7, GDF8, GDF9b/BMP15, GDF11/BMP11, GDF15/MIC1, TGF-β1, TGF-β2, TGF-β3, activin A, activin B, activin C, activin E, activin AB, activin AC, nodal, glial cell-derived neurotrophic factor (GDNF), neurturin, artemin, persephin, MIS, and Lefty.

In certain embodiments, the present disclosure contemplates specific mutations of a BMP10 propeptide polypeptide, ActRII polypeptide, BMPRII polypeptide, ALK1 polypeptide and/or endoglin polypeptide so as to alter the glycosylation of the polypeptide. Such mutations may be selected so as to introduce or eliminate one or more glycosylation sites, such as O-linked or N-linked glycosylation sites. Asparagine-linked glycosylation recognition sites generally comprise a tripeptide sequence, asparagine-X-threonine or asparagine-X-serine (where “X” is any amino acid) which is specifically recognized by appropriate cellular glycosylation enzymes. The alteration may also be made by the addition of, or substitution by, one or more serine or threonine residues to the sequence of the polypeptide (for O-linked glycosylation sites). A variety of amino acid substitutions or deletions at one or both of the first or third amino acid positions of a glycosylation recognition site (and/or amino acid deletion at the second position) results in non-glycosylation at the modified tripeptide sequence. Another means of increasing the number of carbohydrate moieties on a polypeptide is by chemical or enzymatic coupling of glycosides to the polypeptide. Depending on the coupling mode used, the sugar(s) may be attached to (a) arginine and histidine; (b) free carboxyl groups; (c) free sulfhydryl groups such as those of cysteine; (d) free hydroxyl groups such as those of serine, threonine, or hydroxyproline; (e) aromatic residues such as those of phenylalanine, tyrosine, or tryptophan; or (f) the amide group of glutamine. Removal of one or more carbohydrate moieties present on a polypeptide may be accomplished chemically and/or enzymatically. Chemical deglycosylation may involve, for example, exposure of a polypeptide to the compound trifluoromethanesulfonic acid, or an equivalent compound. This treatment results in the cleavage of most or all sugars except the linking sugar (N-acetylglucosamine or N-acetylgalactosamine), while leaving the amino acid sequence intact. Enzymatic cleavage of carbohydrate moieties on polypeptides can be achieved by the use of a variety of endo- and exo-glycosidases as described by Thotakura et al. [Meth. Enzymol. (1987) 138:350]. The sequence of a polypeptide may be adjusted, as appropriate, depending on the type of expression system used, as mammalian, yeast, insect, and plant cells may all introduce differing glycosylation patterns that can be affected by the amino acid sequence of the peptide. In general, BMP10 propeptides polypeptides, ActRII polypeptides, BMPRII polypeptides, ALK1 polypeptides and/or endoglin polypeptides of the present disclosure for use in humans may be expressed in a mammalian cell line that provides proper glycosylation, such as HEK293 or CHO cell lines, although other mammalian expression cell lines are expected to be useful as well.

The disclosure further contemplates a method of generating mutants, particularly sets of combinatorial mutants of BMP10 propeptide polypeptides, ActRII polypeptides, BMPRII polypeptides, ALK1 polypeptides and/or endoglin polypeptides as well as truncation mutants. Pools of combinatorial mutants are especially useful for identifying functionally active (e.g., TGF-beta superfamily ligand binding) ActRII, BMPRII, ALK1, endoglin and/or BMP10 propeptide sequences. The purpose of screening such combinatorial libraries may be to generate, for example, polypeptides variants which have altered properties, such as altered pharmacokinetic or altered ligand binding. A variety of screening assays are provided below, and such assays may be used to evaluate variants. For example, BMP10 propeptide polypeptide, ActRII polypeptide, BMPRII polypeptide, ALK1 polypeptide and/or endoglin polypeptide variants may be screened for ability to bind to one or more TGF-beta superfamily ligands (e.g., BMP2, BMP2/7, BMP3, BMP4, BMP4/7, BMP5, BMP6, BMP7, BMP8a, BMP8b, BMP9, BMP10, GDF3, GDF5, GDF6/BMP13, GDF7, GDF8, GDF9b/BMP15, GDF11/BMP11, GDF15/MIC1, TGF-β1, TGF-β2, TGF-β3, activin A, activin B, activin AB, activin AC, nodal, glial cell-derived neurotrophic factor (GDNF), neurturin, artemin, persephin, MIS, and Lefty), to prevent binding of a TGF-beta superfamily ligand to a TGF-beta superfamily receptor, and/or to interfere with signaling caused by an TGF-beta superfamily ligand.

The activity of BMP10 propeptide polypeptides, ActRII polypeptides, BMPRII polypeptides, ALK1 polypeptides and/or endoglin polypeptides also may be tested in a cell-based assay or in vivo. For example, the effect of a BMP10 propeptide polypeptide, ActRII polypeptide, BMPRII polypeptide, ALK1 polypeptide and/or endoglin polypeptide on the expression of genes involved in muscle production in a muscle cell may be assessed. This may, as needed, be performed in the presence of one or more recombinant TGF-beta superfamily ligand proteins (e.g., BMP2, BMP2/7, BMP3, BMP4, BMP4/7, BMP5, BMP6, BMP7, BMP8a, BMP8b, BMP9, BMP10, GDF3, GDF5, GDF6/BMP13, GDF7, GDF8, GDF9b/BMP15, GDF11/BMP11, GDF15/MIC1, TGF-β1, TGF-β2, TGF-β3, activin A, activin B, activin C, activin E, activin AB, activin AC, nodal, glial cell-derived neurotrophic factor (GDNF), neurturin, artemin, persephin, MIS, and Lefty), and cells may be transfected so as to produce a BMP10 propeptide polypeptide, ActRII polypeptide, BMPRII polypeptide, ALK1 polypeptide and/or endoglin polypeptide, and optionally, a TGF-beta superfamily ligand. Likewise, a BMP10 propeptide polypeptide, ActRII polypeptide, BMPRII polypeptide, ALK1 polypeptide and/or endoglin polypeptide may be administered to a mouse or other animal, and one or more measurements, such as muscle formation and strength may be assessed using art-recognized methods. Similarly, the activity of a BMP10 propeptide polypeptide, ActRII polypeptide, BMPRII polypeptide, ALK1 polypeptide and/or endoglin polypeptide or variants thereof may be tested in cancer cells for any effect on growth of these cells, for example, by the assays as described herein and those of common knowledge in the art. A SMAD-responsive reporter gene may be used in such cell lines to monitor effects on downstream signaling.

Combinatorial-derived variants can be generated which have increased selectivity or generally increased potency relative to a reference BMP10 propeptide polypeptide, ActRII polypeptide, BMPRII polypeptide, ALK1 polypeptide and/or endoglin polypeptide. Such variants, when expressed from recombinant DNA constructs, can be used in gene therapy protocols. Likewise, mutagenesis can give rise to variants which have intracellular half-lives dramatically different than the corresponding unmodified BMP10 propeptide polypeptide, ActRII polypeptide, BMPRII polypeptide, ALK1 polypeptide and/or endoglin polypeptide. For example, the altered protein can be rendered either more stable or less stable to proteolytic degradation or other cellular processes which result in destruction, or otherwise inactivation, of an unmodified polypeptide. Such variants, and the genes which encode them, can be utilized to alter polypeptide complex levels by modulating the half-life of the polypeptide. For instance, a short half-life can give rise to more transient biological effects and, when part of an inducible expression system, can allow tighter control of recombinant polypeptide complex levels within the cell. In an Fc fusion protein, mutations may be made in the linker (if any) and/or the Fc portion to alter the half-life of the BMP10 propeptide polypeptide, ActRII polypeptide, BMPRII polypeptide, ALK1 polypeptide and/or endoglin polypeptide.

A combinatorial library may be produced by way of a degenerate library of genes encoding a library of polypeptides which each include at least a portion of potential ActRII BMPRII, ALK1, endoglin, and/or BMP10 propeptide sequences. For instance, a mixture of synthetic oligonucleotides can be enzymatically ligated into gene sequences such that the degenerate set of potential ActRII BMPRII, ALK1, endoglin, and/or BMP10 propeptide encoding nucleotide sequences are expressible as individual polypeptides, or alternatively, as a set of larger fusion proteins (e.g., for phage display).

There are many ways by which the library of potential homologs can be generated from a degenerate oligonucleotide sequence. Chemical synthesis of a degenerate gene sequence can be carried out in an automatic DNA synthesizer, and the synthetic genes can then be ligated into an appropriate vector for expression. The synthesis of degenerate oligonucleotides is well known in the art [Narang, S A (1983) Tetrahedron 39:3; Itakura et al. (1981) Recombinant DNA, Proc. 3rd Cleveland Sympos. Macromolecules, ed. A G Walton, Amsterdam: Elsevier pp 273-289; Itakura et al. (1984) Annu. Rev. Biochem. 53:323; Itakura et al. (1984) Science 198:1056; and Ike et al. (1983) Nucleic Acid Res. 11:477]. Such techniques have been employed in the directed evolution of other proteins [Scott et al., (1990) Science 249:386-390; Roberts et al. (1992) PNAS USA 89:2429-2433; Devlin et al. (1990) Science 249: 404-406; Cwirla et al., (1990) PNAS USA 87: 6378-6382; as well as U.S. Pat. Nos. 5,223,409, 5,198,346, and 5,096,815].

Alternatively, other forms of mutagenesis can be utilized to generate a combinatorial library. For example, BMP10 propeptide polypeptides, ActRII polypeptides, BMPRII polypeptides, ALK1 polypeptides and/or endoglin polypeptides of the disclosure can be generated and isolated from a library by screening using, for example, alanine scanning mutagenesis [Ruf et al. (1994) Biochemistry 33:1565-1572; Wang et al. (1994) J. Biol. Chem. 269:3095-3099; Balint et al. (1993) Gene 137:109-118; Grodberg et al. (1993) Eur. J. Biochem. 218:597-601; Nagashima et al. (1993) J. Biol. Chem. 268:2888-2892; Lowman et al. (1991) Biochemistry 30:10832-10838; and Cunningham et al. (1989) Science 244:1081-1085], by linker scanning mutagenesis [Gustin et al. (1993) Virology 193:653-660; and Brown et al. (1992) Mol. Cell Biol. 12:2644-2652; McKnight et al. (1982) Science 232:316], by saturation mutagenesis [Meyers et al., (1986) Science 232:613]; by PCR mutagenesis [Leung et al. (1989) Method Cell Mol Biol 1:11-19]; or by random mutagenesis, including chemical mutagenesis [Miller et al. (1992) A Short Course in Bacterial Genetics, CSHL Press, Cold Spring Harbor, N.Y.; and Greener et al. (1994) Strategies in Mol Biol 7:32-34]. Linker scanning mutagenesis, particularly in a combinatorial setting, is an attractive method for identifying truncated (bioactive) forms of BMP10 propeptide polypeptides, ActRII polypeptides, BMPRII polypeptides, ALK1 polypeptides and/or endoglin polypeptides.

A wide range of techniques are known in the art for screening gene products of combinatorial libraries made by point mutations and truncations, and, for that matter, for screening cDNA libraries for gene products having a certain property. Such techniques will be generally adaptable for rapid screening of the gene libraries generated by the combinatorial mutagenesis of BMP10 propeptide polypeptides, ActRII polypeptides, BMPRII polypeptides, ALK1 polypeptides and/or endoglin polypeptides. The most widely used techniques for screening large gene libraries typically comprise cloning the gene library into replicable expression vectors, transforming appropriate cells with the resulting library of vectors, and expressing the combinatorial genes under conditions in which detection of a desired activity facilitates relatively easy isolation of the vector encoding the gene whose product was detected. Preferred assays include TGF-beta ligand (e.g., BMP2, BMP2/7, BMP3, BMP4, BMP4/7, BMP5, BMP6, BMP7, BMP8a, BMP8b, BMP9, BMP10, GDF3, GDF5, GDF6/BMP13, GDF7, GDF8, GDF9b/BMP15, GDF11/BMP11, GDF15/MIC1, TGFβ1, TGFβ2, TGFβ3, activin A, activin B, activin C, activin E, activin AB, activin AC, nodal, glial cell-derived neurotrophic factor (GDNF), neurturin, artemin, persephin, MIS, and Lefty) binding assays and/or TGF-beta ligand-mediated cell signaling assays.

In certain embodiments, BMP10 propeptide polypeptides, ActRII polypeptides, BMPRII polypeptides, ALK1 polypeptides and/or endoglin polypeptides may further comprise post-translational modifications in addition to any that are naturally present in the ActRII, BMPRII, ALK1, endoglin, and/or BMP10 propeptide polypeptide. Such modifications include, but are not limited to, acetylation, carboxylation, glycosylation, phosphorylation, lipidation, and acylation. As a result, BMP10 propeptide polypeptides, ActRII polypeptides, BMPRII polypeptides, ALK1 polypeptides, and/or endoglin polypeptides may comprise non-amino acid elements, such as polyethylene glycols, lipids, polysaccharide or monosaccharide, and phosphates. Effects of such non-amino acid elements on the functionality of a BMP10 propeptide polypeptides, ActRII polypeptides, BMPRII polypeptides, ALK1 polypeptides and/or endoglin polypeptides may be tested as described herein for other variants. When a polypeptide of the disclosure is produced in cells by cleaving a nascent form of the polypeptide, post-translational processing may also be important for correct folding and/or function of the protein. Different cells (e.g., CHO, HeLa, MDCK, 293, WI38, NIH-3T3 or HEK293) have specific cellular machinery and characteristic mechanisms for such post-translational activities and may be chosen to ensure the correct modification and processing of the ActRII, BMPRII, ALK1, endoglin, or BMP10 propeptide polypeptide.

In certain aspects, BMP10 propeptide polypeptides, ActRII polypeptides, BMPRII polypeptides, ALK1 polypeptides and/or endoglin polypeptides of the disclosure are fusion proteins comprising at least a portion (domain) of an ActRII polypeptide (e.g., an ActRIIA or ActRIIB polypeptide), BMPRII, ALK1, endoglin, or BMP10 propeptide polypeptide and one or more heterologous portions (domains). Well-known examples of such fusion domains include, but are not limited to, polyhistidine, Glu-Glu, glutathione S-transferase (GST), thioredoxin, protein A, protein G, an immunoglobulin heavy-chain constant region (Fc), maltose binding protein (MBP), or human serum albumin. A fusion domain may be selected so as to confer a desired property. For example, some fusion domains are particularly useful for isolation of the fusion proteins by affinity chromatography. For the purpose of affinity purification, relevant matrices for affinity chromatography, such as glutathione-, amylase-, and nickel- or cobalt-conjugated resins are used. Many of such matrices are available in “kit” form, such as the Pharmacia GST purification system and the QIAexpress™ system (Qiagen) useful with (HIS₆) fusion partners. As another example, a fusion domain may be selected so as to facilitate detection of the BMP10 propeptide polypeptide, ActRII polypeptide, BMPRII polypeptide, ALK1 polypeptide and/or endoglin polypeptide. Examples of such detection domains include the various fluorescent proteins (e.g., GFP) as well as “epitope tags,” which are usually short peptide sequences for which a specific antibody is available. Well-known epitope tags for which specific monoclonal antibodies are readily available include FLAG, influenza virus haemagglutinin (HA), and c-myc tags. In some cases, the fusion domains have a protease cleavage site, such as for Factor Xa or thrombin, which allows the relevant protease to partially digest the fusion proteins and thereby liberate the recombinant proteins therefrom. The liberated proteins can then be isolated from the fusion domain by subsequent chromatographic separation. Other types of fusion domains that may be selected include multimerizing (e.g., dimerizing, tetramerizing) domains and functional domains (that confer an additional biological function) including, for example constant domains from immunoglobulins (e.g., Fc domains).

In certain aspects, BMP10 propeptide polypeptides, ActRII polypeptides, BMPRII polypeptides, ALK1 polypeptides and/or endoglin polypeptides of the present disclosure contain one or more modifications that are capable of “stabilizing” the polypeptides. By “stabilizing” is meant anything that increases the in vitro half-life, serum half-life, regardless of whether this is because of decreased destruction, decreased clearance by the kidney, or other pharmacokinetic effect of the agent. For example, such modifications enhance the shelf-life of the polypeptides, enhance circulatory half-life of the polypeptides, and/or reduce proteolytic degradation of the polypeptides. Such stabilizing modifications include, but are not limited to, fusion proteins (including, for example, fusion proteins comprising a BMP10 propeptide polypeptide, ActRII polypeptide, BMPRII polypeptide, ALK1 polypeptide and/or endoglin polypeptide domain and a stabilizer domain), modifications of a glycosylation site (including, for example, addition of a glycosylation site to a polypeptide of the disclosure), and modifications of carbohydrate moiety (including, for example, removal of carbohydrate moieties from a polypeptide of the disclosure). As used herein, the term “stabilizer domain” not only refers to a fusion domain (e.g., an immunoglobulin Fc domain) as in the case of fusion proteins, but also includes nonproteinaceous modifications such as a carbohydrate moiety, or nonproteinaceous moiety, such as polyethylene glycol. In certain preferred embodiments, a BMP10 propeptide polypeptide, ActRII polypeptide, BMPRII polypeptide, ALK1 polypeptide and/or endoglin polypeptide is fused with a heterologous domain that stabilizes the polypeptide (a “stabilizer” domain), preferably a heterologous domain that increases stability of the polypeptide in vivo. Fusions with a constant domain of an immunoglobulin (e.g., an Fc domain) are known to confer desirable pharmacokinetic properties on a wide range of proteins. Likewise, fusions to human serum albumin can confer desirable stabilizing properties.

In some embodiments, ActRII, BMPRII, ALK1, endoglin, and/or BMP10 propeptide polypeptides of the disclosure are Fc fusion proteins. An example of a native amino acid sequence that may be used for the Fc portion of human IgG1 (G1Fc) is shown below (SEQ ID NO: 36). Dotted underline indicates the hinge region, and solid underline indicates positions with naturally occurring variants. In part, the disclosure provides polypeptides comprising, consisting essential of, or consisting of amino acid sequences with 70%, 75%, 80%, 85%, 86%, 87%, 88%, 89%, 90%, 91%, 92%, 93%, 94%, 95%, 96%, 97%, 98%, 99%, or 100% identity to SEQ ID NO: 36. Naturally occurring variants in G1Fc would include E134D and M136L according to the numbering system used in SEQ ID NO: 36 (see Uniprot P01857).

| (SEQ ID NO: 36) | |
|---|---|
| 1 | 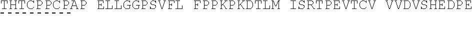 |
| 51 | VKFNWYVDGV EVHNAKTKPR EEQYNSTYRV VSVLTVLHQD WLNGKEYKCK |
| 101 | VSNKALPAPI EKTISKAKGQ PREPQVYTLP PSREEMTKNQ VSLTCLVKGF |
| 151 | YPSDIAVEWE SNGQPENNYK TTPPVLDSDG SFFLYSKLTV DKSRWQQGNV |
| 201 | FSCSVMHEAL HNHYTQKSLS LSPGK |

Optionally, the IgG1 Fc domain has one or more mutations at residues such as Asp-265, lysine 322, and Asn-434. In certain cases, the mutant IgG1 Fc domain having one or more of these mutations (e.g., Asp-265 mutation) has reduced ability of binding to the Fcγ receptor relative to a wild-type Fc domain. In other cases, the mutant Fc domain having one or more of these mutations (e.g., Asn-434 mutation) has increased ability of binding to the MHC class I-related Fc-receptor (FcRN) relative to a wild-type IgG1 Fc domain.

An example of a native amino acid sequence that may be used for the Fc portion of human IgG2 (G2Fc) is shown below (SEQ ID NO: 37). Dotted underline indicates the hinge region and double underline indicates positions where there are data base conflicts in the sequence (according to UniProt P01859). In part, the disclosure provides polypeptides comprising, consisting essential of, or consisting of amino acid sequences with 70%, 75%, 80%, 85%, 86%, 87%, 88%, 89%, 90%, 91%, 92%, 93%, 94%, 95%, 96%, 97%, 98%, 99%, or 100% identity to SEQ ID NO: 37.

| (SEQ ID NO: 37) | |
|---|---|
| 1 | 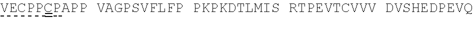 |
| 51 | FNWYVDGVEV HNAKTKPREE QFNSTFRVVS VLTVVHQDWL NGKEYKCKVS |
| 101 | NKGLPAPIEK TISKTKGQPR EPQVYTLPPS REEMTKNQVS LTCLVKGFYP |
| 151 | SDIAVEWESN GQPENNYKTT PPMLDSDGSF FLYSKLTVDK SRWQQGNVFS |
| 201 | CSVMHEALHN HYTQKSLSLS PGK |

Two examples of amino acid sequences that may be used for the Fc portion of human IgG3 (G3Fc) are shown below. The hinge region in G3Fc can be up to four times as long as in other Fc chains and contains three identical 15-residue segments preceded by a similar 17-residue segment. The first G3Fc sequence shown below (SEQ ID NO: 38) contains a short hinge region consisting of a single 15-residue segment, whereas the second G3Fc sequence (SEQ ID NO: 39) contains a full-length hinge region. In each case, dotted underline indicates the hinge region, and solid underline indicates positions with naturally occurring variants according to UniProt P01859. In part, the disclosure provides polypeptides comprising, consisting essential of, or consisting of amino acid sequences with 70%, 75%, 80%, 85%, 86%, 87%, 88%, 89%, 90%, 91%, 92%, 93%, 94%, 95%, 96%, 97%, 98%, 99%, or 100% identity to SEQ ID NOs: 38 or 39.

| (SEQ ID NO: 38) | |
|---|---|
| 1 | 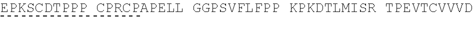 |
| 51 | VSHEDPEVQF KWYVDGVEVH NAKTKPREEQ YNSTFRVVSV LTVLHQDWLN |
| 101 | GKEYKCKVSN KALPAPIEKT ISKTKGQPRE PQVYTLPPSR EEMTKNQVSL |
| 151 | TCLVKGFYPS DIAVEWESSG QPENNYNTTP PMLDSDGSFF LYSKLTVDKS |
| 201 | RWQQGNIFSC SVMHEALHNR FTQKSLSLSP GK |
| (SEQ ID NO: 39) | |
| 1 | 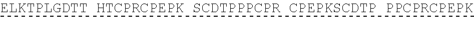 |
| 51 | 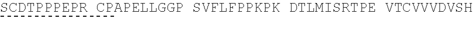 |
| 101 | EDPEVQFKWY VDGVEVHNAK TKPREEQYNS TFRVVSVLTV LHQDWLNGKE |
| 151 | YKCKVSNKAL PAPIEKTISK TKGQPREPQV YTLPPSREEM TKNQVSLTCL |
| 201 | VKGFYPSDIA VEWESSGQPE NNYNTTPPML DSDGSFFLYS KLTVDKSRWQ |
| 251 | QGNIFSCSVM HEALHNRFTQ KSLSLSPGK |

Naturally occurring variants in G3Fc (for example, see Uniprot P01860) include E68Q, P76L, E79Q, Y81F, D97N, N100D, T124A, S169N, S169del, F221Y when converted to the numbering system used in SEQ ID NO: 38, and the present disclosure provides fusion proteins comprising G3Fc domains containing one or more of these variations. In addition, the human immunoglobulin IgG3 gene (IGHG3) shows a structural polymorphism characterized by different hinge lengths [see Uniprot P01859]. Specifically, variant WIS is lacking most of the V region and all of the CH1 region. It has an extra interchain disulfide bond at position 7 in addition to the 11 normally present in the hinge region. Variant ZUC lacks most of the V region, all of the CH1 region, and part of the hinge. Variant OMM may represent an allelic form or another gamma chain subclass. The present disclosure provides additional fusion proteins comprising G3Fc domains containing one or more of these variants.

An example of a native amino acid sequence that may be used for the Fc portion of human IgG4 (G4Fc) is shown below (SEQ ID NO: 40). Dotted underline indicates the hinge region. In part, the disclosure provides polypeptides comprising, consisting essential of, or consisting of amino acid sequences with 70%, 75%, 80%, 85%, 86%, 87%, 88%, 89%, 90%, 91%, 92%, 93%, 94%, 95%, 96%, 97%, 98%, 99%, or 100% identity to SEQ ID NO: 40.

| (SEQ ID NO: 40) | |
|---|---|
| 1 | 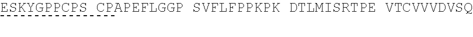 |
| 51 | EDPEVQFNWY VDGVEVHNAK TKPREEQFNS TYRVVSVLTV LHQDWLNGKE |
| 101 | YKCKVSNKGL PSSIEKTISK AKGQPREPQV YTLPPSQEEM TKNQVSLTCL |
| 151 | VKGFYPSDIA VEWESNGQPE NNYKTTPPVL DSDGSFFLYS RLTVDKSRWQ |
| 201 | EGNVFSCSVM HEALHNHYTQ KSLSLSLGK |

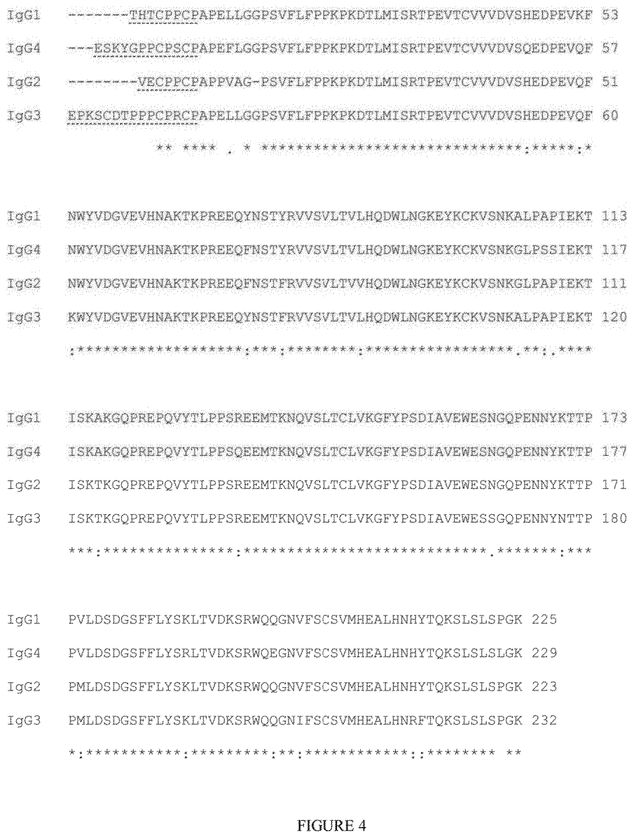

A variety of engineered mutations in the Fc domain are presented herein with respect to the G1Fc sequence (SEQ ID NO: 36), and analogous mutations in G2Fc, G3Fc, and G4Fc can be derived from their alignment with G1Fc in FIG. 4 Due to unequal hinge lengths, analogous Fc positions based on isotype alignment (FIG. 4) possess different amino acid numbers in SEQ ID NOs: 36, 37, 38, 39, and 40. It can also be appreciated that a given amino acid position in an immunoglobulin sequence consisting of hinge, C_{H}2, and C_{H}3 regions (e.g., SEQ ID NOs: 36, 37, 38, 39, and 40) will be identified by a different number than the same position when numbering encompasses the entire IgG1 heavy-chain constant domain (consisting of the C_{H}1, hinge, C_{H}2, and C_{H}3 regions) as in the Uniprot database.

The application further provides antibodies and Fc fusion proteins with Engineered or variant Fc regions. Such antibodies and Fc fusion proteins may be useful, for example, in modulating effector functions, such as, antigen-dependent cytotoxicity (ADCC) and complement-dependent cytotoxicity (CDC). Additionally, the modifications may improve the stability of the antibodies and Fc fusion proteins. Amino acid sequence variants of the antibodies and Fc fusion proteins are prepared by introducing appropriate nucleotide changes into the DNA, or by peptide synthesis. Such variants include, for example, deletions from, and/or insertions into and/or substitutions of, residues within the amino acid sequences of the antibodies and Fc fusion proteins disclosed herein. Any combination of deletion, insertion, and substitution is made to arrive at the final construct, provided that the final construct possesses the desired characteristics. The amino acid changes also may alter post-translational processes of the antibodies and Fc fusion proteins, such as changing the number or position of glycosylation sites.

Antibodies and Fc fusion proteins with reduced effector function may be produced by introducing changes in the amino acid sequence, including, but are not limited to, the Ala-Ala mutation described by Bluestone et al. (see WO 94/28027 and WO 98/47531; also see Xu et al. 2000 Cell Immunol 200; 16-26). Thus in certain embodiments, antibodies and Fc fusion proteins of the disclosure with mutations within the constant region including the Ala-Ala mutation may be used to reduce or abolish effector function. According to these embodiments, antibodies and Fc fusion proteins may comprise a mutation to an alanine at position 234 or a mutation to an alanine at position 235, or a combination thereof. In one embodiment, the antibody or Fc fusion protein comprises an IgG4 framework, wherein the Ala-Ala mutation would describe a mutation(s) from phenylalanine to alanine at position 234 and/or a mutation from leucine to alanine at position 235. In another embodiment, the antibody or Fc fusion protein comprises an IgG1 framework, wherein the Ala-Ala mutation would describe a mutation(s) from leucine to alanine at position 234 and/or a mutation from leucine to alanine at position 235. The antibody or Fc fusion protein may alternatively or additionally carry other mutations, including the point mutation K322A in the CH2 domain (Hezareh et al. 2001 J Virol. 75: 12161-8).

In some embodiments, the antibody or Fc fusion protein may be modified to either enhance or inhibit complement dependent cytotoxicity (CDC). Modulated CDC activity may be achieved by introducing one or more amino acid substitutions, insertions, or deletions in an Fc region (see, e.g., U.S. Pat. No. 6,194,551). Alternatively or additionally, cysteine residue(s) may be introduced in the Fc region, thereby allowing interchain disulfide bond formation in this region. The homodimeric antibody thus generated may have improved or reduced internalization capability and/or increased or decreased complement-mediated cell killing. See Caron et al., J. Exp Med. 176:1191-1195 (1992) and Shopes, B. J. Immunol. 148:2918-2922 (1992), WO99/51642, Duncan & Winter Nature 322: 738-40 (1988); U.S. Pat. Nos. 5,648,260; 5,624,821; and WO94/29351.

It is understood that different elements of the fusion proteins (e.g., immunoglobulin Fc fusion proteins) may be arranged in any manner that is consistent with desired functionality. For example, a BMP10 propeptide polypeptide, ActRII polypeptide, BMPRII polypeptide, ALK1 polypeptide and/or endoglin polypeptide domain may be placed C-terminal to a heterologous domain, or alternatively, a heterologous domain may be placed C-terminal to a BMP10 propeptide polypeptide, ActRII polypeptide, BMPRII polypeptide, ALK1 polypeptide and/or endoglin polypeptide domain. The BMP10 propeptide polypeptide, ActRII polypeptide, BMPRII polypeptide, ALK1 polypeptide and/or endoglin polypeptide domain and the heterologous domain need not be adjacent in a fusion protein, and additional domains or amino acid sequences may be included C- or N-terminal to either domain or between the domains.

For example, a BMP10 propeptide (ActRII, BMPRII, ALK1, or endoglin) fusion protein may comprise an amino acid sequence as set forth in the formula A-B-C. The B portion corresponds to a BMP10 propeptide (ActRII, BMPRII, ALK1, or endoglin) polypeptide domain. The A and C portions may be independently zero, one, or more than one amino acid, and both the A and C portions when present are heterologous to B. The A and/or C portions may be attached to the B portion via a linker sequence. A linker may be rich in glycine (e.g., 2-10, 2-5, 2-4, 2-3 glycine residues) or glycine and proline residues and may, for example, contain a single sequence of threonine/serine and glycines or repeating sequences of threonine/serine and/or glycines, e.g., GGG (SEQ ID NO: 41), GGGG (SEQ ID NO: 42), TGGGG (SEQ ID NO: 43), SGGGG (SEQ ID NO: 44), TGGG (SEQ ID NO: 45), SGGG (SEQ ID NO: 46), or GGGGS (SEQ ID NO: 47) singlets, or repeats. In certain embodiments, a BMP10 propeptide (ActRII, BMPRII, ALK1, or endoglin) fusion protein comprises an amino acid sequence as set forth in the formula A-B-C, wherein A is a leader (signal) sequence, B consists of a BMP10 propeptide (ActRII, BMPRII, ALK1, or endoglin) polypeptide domain, and C is a polypeptide portion that enhances one or more of in vivo stability, in vivo half-life, uptake/administration, tissue localization or distribution, formation of protein complexes, and/or purification. In certain embodiments, a BMP10 propeptide (ActRII, BMPRII, ALK1, or endoglin) fusion protein comprises an amino acid sequence as set forth in the formula A-B-C, wherein A is a TPA leader sequence, B consists of a BMP10 propeptide (ActRII, BMPRII, ALK1, or endoglin) receptor polypeptide domain, and C is an immunoglobulin Fc domain. Preferred fusion proteins comprise the amino acid sequence set forth in any one of SEQ ID NOs: 28, 29, 30, 31, 50, 54, 57, 58, 60, 63, 64, 66, 69, 71, 74, 76, 78, 80, 82, 84, 85, 87, 123, 131, and 132.

In certain preferred embodiments, a BMP10 propeptide polypeptide, ActRII polypeptide, BMPRII polypeptide, ALK1 polypeptide and/or endoglin polypeptide to be used in accordance with the methods described herein are isolated complexes. As used herein, an isolated protein (or protein complex) or polypeptide (or polypeptide complex) is one which has been separated from a component of its natural environment. In some embodiments, a polypeptide of the disclosure is purified to greater than 95%, 96%, 97%, 98%, or 99% purity as determined by, for example, electrophoretic (e.g., SDS-PAGE, isoelectric focusing (IEF), capillary electrophoresis) or chromatographic (e.g., ion exchange or reverse phase HPLC). Methods for assessment of antibody purity are well known in the art [Flatman et al., (2007) J. Chromatogr. B 848:79-87].

In certain embodiments, a BMP10 propeptide polypeptides, ActRII polypeptides, BMPRII polypeptides, ALK1 polypeptides and/or endoglin polypeptides of the disclosure can be produced by a variety of art-known techniques. For example, polypeptides of the disclosure can be synthesized using standard protein chemistry techniques such as those described in Bodansky, M. Principles of Peptide Synthesis, Springer Verlag, Berlin (1993) and Grant G. A. (ed.), Synthetic Peptides: A User's Guide, W. H. Freeman and Company, New York (1992). In addition, automated peptide synthesizers are commercially available (Advanced ChemTech Model 396; Milligen/Biosearch 9600). Alternatively, the polypeptides and complexes of the disclosure, including fragments or variants thereof, may be recombinantly produced using various expression systems [E. coli, Chinese Hamster Ovary (CHO) cells, COS cells, baculovirus] as is well known in the art. In a further embodiment, the modified or unmodified polypeptides of the disclosure may be produced by digestion of recombinantly produced full-length a BMP10 propeptide polypeptide, ActRII polypeptide, BMPRII polypeptide, ALK1 polypeptide and/or endoglin polypeptide by using, for example, a protease, e.g., trypsin, thermolysin, chymotrypsin, pepsin, or paired basic amino acid converting enzyme (PACE). Computer analysis (using commercially available software, e.g., MacVector, Omega, PCGene, Molecular Simulation, Inc.) can be used to identify proteolytic cleavage sites.

### 3. Nucleic Acids Encoding BMP10 Propeptide, ActRII, BMPRII, ALK1, and Endoglin Polypeptides

In certain embodiments, the present disclosure provides isolated and/or recombinant nucleic acids encoding ActRII, BMPRII, ALK1, endoglin and/or BMP10 propeptide polypeptides (including fragments, functional variants, and fusion proteins thereof) disclosed herein. For example, SEQ ID NO: 16 encodes a naturally occurring human BMPRII precursor polypeptide, SEQ ID NO: 17 encodes a processed extracellular domain of BMPRII. The subject nucleic acids may be single-stranded or double stranded. Such nucleic acids may be DNA or RNA molecules. These nucleic acids may be used, for example, in methods for making ActRII, BMPRII, ALK1, endoglin and/or BMP10 propeptide polypeptides as described herein.

As used herein, isolated nucleic acid(s) refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

In certain embodiments, nucleic acids encoding ActRII, BMPRII, ALK1, endoglin and/or BMP10 propeptide polypeptides of the present disclosure are understood to include any one of SEQ ID NOs: 7, 8, 12, 13, 16, 17, 19, 22, 23, 25, 27, 33, 35, 55, 61, 67, 70, 72, 79, 83, 86, 124, 125, 126, 127, 134, 135, 136, and 137 as well as variants thereof. Variant nucleotide sequences include sequences that differ by one or more nucleotide substitutions, additions, or deletions including allelic variants, and therefore, will include coding sequences that differ from the nucleotide sequence designated in any one of SEQ ID NOs: 7, 8, 12, 13, 16, 17, 19, 22, 23, 25, 27, 33, 35, 55, 61, 67, 70, 72, 79, 83, 86, 124, 125, 126, 127, 134, 135, 136, and 137.

In certain embodiments, ActRII, BMPRII, ALK1, endoglin and/or BMP10 propeptide polypeptides of the present disclosure are encoded by isolated or recombinant nucleic acid sequences that comprise, consist essentially of, or consists of a sequence that is least 70%, 75%, 80%, 85%, 86%, 87%, 88%, 89%, 90%, 91%, 92%, 93%, 94%, 95%, 96%, 97%, 98%, 99% or 100% identical to SEQ ID NOs: 7, 8, 12, 13, 16, 17, 19, 22, 23, 25, 27, 33, 35, 55, 61, 67, 70, 72, 75, 79, 83, 86, 124, 125, 126, 127, 134, 135, 136, and 137. One of ordinary skill in the art will appreciate that nucleic acid sequences that comprise, consist essentially of, or consists of a sequence complementary to a sequence that is least 70%, 75%, 80%, 85%, 86%, 87%, 88%, 89%, 90%, 91%, 92%, 93%, 94%, 95%, 96%, 97%, 98%, 99% or 100% identical to SEQ ID NOs: 7, 8, 12, 13, 16, 17, 19, 22, 23, 25, 27, 33, 35, 55, 61, 67, 70, 72, 79, 83, 86, 124, 125, 126, 127, 134, 135, 136, and 137 also within the scope of the present disclosure. In further embodiments, the nucleic acid sequences of the disclosure can be isolated, recombinant, and/or fused with a heterologous nucleotide sequence or in a DNA library.

In other embodiments, nucleic acids of the present disclosure also include nucleotide sequences that hybridize under stringent conditions to the nucleotide sequence designated in SEQ ID NOs: 7, 8, 12, 13, 16, 17, 19, 22, 23, 25, 27, 33, 35, 55, 61, 67, 70, 72, 79, 83, 86, 124, 125, 126, 127, 134, 135, 136, and 137, or fragments thereof. One of ordinary skill in the art will understand readily that appropriate stringency conditions which promote DNA hybridization can be varied. For example, one could perform the hybridization at 6.0× sodium chloride/sodium citrate (SSC) at about 45° C., followed by a wash of 2.0×SSC at 50° C. For example, the salt concentration in the wash step can be selected from a low stringency of about 2.0×SSC at 50° C. to a high stringency of about 0.2×SSC at 50° C. In addition, the temperature in the wash step can be increased from low stringency conditions at room temperature, about 22° C., to high stringency conditions at about 65° C. Both temperature and salt may be varied, or temperature or salt concentration may be held constant while the other variable is changed. In one embodiment, the disclosure provides nucleic acids which hybridize under low stringency conditions of 6×SSC at room temperature followed by a wash at 2×SSC at room temperature.

Isolated nucleic acids which differ from the nucleic acids as set forth in SEQ ID NOs: 7, 8, 12, 13, 16, 17, 19, 22, 23, 25, 27, 33, 35, 55, 61, 67, 70, 72, 79, 83, 86, 124, 125, 126, 127, 134, 135, 136, and 137 to degeneracy in the genetic code are also within the scope of the disclosure. For example, a number of amino acids are designated by more than one triplet. Codons that specify the same amino acid, or synonyms (for example, CAU and CAC are synonyms for histidine) may result in “silent” mutations which do not affect the amino acid sequence of the protein. However, it is expected that DNA sequence polymorphisms that do lead to changes in the amino acid sequences of the subject proteins will exist among mammalian cells. One skilled in the art will appreciate that these variations in one or more nucleotides (up to about 3-5% of the nucleotides) of the nucleic acids encoding a particular protein may exist among individuals of a given species due to natural allelic variation. Any and all such nucleotide variations and resulting amino acid polymorphisms are within the scope of this disclosure.

In certain embodiments, the recombinant nucleic acids of the present disclosure may be operably linked to one or more regulatory nucleotide sequences in an expression construct. Regulatory nucleotide sequences will generally be appropriate to the host cell used for expression. Numerous types of appropriate expression vectors and suitable regulatory sequences are known in the art for a variety of host cells. Typically, said one or more regulatory nucleotide sequences may include, but are not limited to, promoter sequences, leader or signal sequences, ribosomal binding sites, transcriptional start and termination sequences, translational start and termination sequences, and enhancer or activator sequences. Constitutive or inducible promoters as known in the art are contemplated by the disclosure. The promoters may be either naturally occurring promoters, or hybrid promoters that combine elements of more than one promoter. An expression construct may be present in a cell on an episome, such as a plasmid, or the expression construct may be inserted in a chromosome. In some embodiments, the expression vector contains a selectable marker gene to allow the selection of transformed host cells. Selectable marker genes are well known in the art and will vary with the host cell used.

In certain aspects of the present disclosure, the subject nucleic acid is provided in an expression vector comprising a nucleotide sequence encoding an ActRII, BMPRII, ALK1, endoglin and/or BMP10 propeptide polypeptide and operably linked to at least one regulatory sequence. Regulatory sequences are art-recognized and are selected to direct expression of ActRII, BMPRII, ALK1, endoglin and/or BMP10 propeptide polypeptides. Accordingly, the term regulatory sequence includes promoters, enhancers, and other expression control elements. Exemplary regulatory sequences are described in Goeddel; Gene Expression Technology: Methods in Enzymology, Academic Press, San Diego, Calif. (1990). For instance, any of a wide variety of expression control sequences that control the expression of a DNA sequence when operatively linked to it may be used in these vectors to express DNA sequences encoding an ActRII, BMPRII, ALK1, endoglin and/or BMP10 propeptide polypeptide. Such useful expression control sequences, include, for example, the early and late promoters of SV40, tet promoter, adenovirus or cytomegalovirus immediate early promoter, RSV promoters, the lac system, the trp system, the TAC or TRC system, T7 promoter whose expression is directed by T7 RNA polymerase, the major operator and promoter regions of phage lambda, the control regions for fd coat protein, the promoter for 3-phosphoglycerate kinase or other glycolytic enzymes, the promoters of acid phosphatase, e.g., Pho5, the promoters of the yeast a-mating factors, the polyhedron promoter of the baculovirus system and other sequences known to control the expression of genes of prokaryotic or eukaryotic cells or their viruses, and various combinations thereof. It should be understood that the design of the expression vector may depend on such factors as the choice of the host cell to be transformed and/or the type of protein desired to be expressed. Moreover, the vector's copy number, the ability to control that copy number and the expression of any other protein encoded by the vector, such as antibiotic markers, should also be considered.

A recombinant nucleic acid of the present disclosure can be produced by ligating the cloned gene, or a portion thereof, into a vector suitable for expression in either prokaryotic cells, eukaryotic cells (yeast, avian, insect or mammalian), or both. Expression vehicles for production of a recombinant ActRII, BMPRII, ALK1, endoglin and/or BMP10 propeptide polypeptides include plasmids and other vectors. For instance, suitable vectors include plasmids of the following types: pBR322-derived plasmids, pEMBL-derived plasmids, pEX-derived plasmids, pBTac-derived plasmids and pUC-derived plasmids for expression in prokaryotic cells, such as E. coli.

Some mammalian expression vectors contain both prokaryotic sequences to facilitate the propagation of the vector in bacteria, and one or more eukaryotic transcription units that are expressed in eukaryotic cells. The pcDNAI/amp, pcDNAI/neo, pRc/CMV, pSV2gpt, pSV2neo, pSV2-dhfr, pTk2, pRSVneo, pMSG, pSVT7, pko-neo and pHyg derived vectors are examples of mammalian expression vectors suitable for transfection of eukaryotic cells. Some of these vectors are modified with sequences from bacterial plasmids, such as pBR322, to facilitate replication and drug resistance selection in both prokaryotic and eukaryotic cells. Alternatively, derivatives of viruses such as the bovine papilloma virus (BPV-1), or Epstein-Barr virus (pHEBo, pREP-derived and p205) can be used for transient expression of proteins in eukaryotic cells. Examples of other viral (including retroviral) expression systems can be found below in the description of gene therapy delivery systems. The various methods employed in the preparation of the plasmids and in transformation of host organisms are well known in the art. For other suitable expression systems for both prokaryotic and eukaryotic cells, as well as general recombinant procedures [Molecular Cloning A Laboratory Manual, 3rd Ed., ed. by Sambrook, Fritsch and Maniatis Cold Spring Harbor Laboratory Press, 2001]. In some instances, it may be desirable to express the recombinant polypeptides by the use of a baculovirus expression system. Examples of such baculovirus expression systems include pVL-derived vectors (such as pVL1392, pVL1393 and pVL941), pAcUW-derived vectors (such as pAcUW1), and pBlueBac-derived vectors (such as the β-gal containing pBlueBac III).

In a preferred embodiment, a vector will be designed for production of the subject ALK4 and/or ActRII polypeptides in CHO cells, such as a Pcmv-Script vector (Stratagene, La Jolla, Calif.), pcDNA4 vectors (Invitrogen, Carlsbad, Calif.) and pCI-neo vectors (Promega, Madison, Wis.). As will be apparent, the subject gene constructs can be used to cause expression of the subject ActRII, BMPRII, ALK1, endoglin and/or BMP10 propeptide polypeptide in cells propagated in culture, e.g., to produce proteins, including fusion proteins or variant proteins, for purification.

This disclosure also pertains to a host cell transfected with a recombinant gene including a coding sequence for one or more of the subject ActRII, BMPRII, ALK1, endoglin and/or BMP10 propeptide polypeptides. The host cell may be any prokaryotic or eukaryotic cell. For example, an ActRII, BMPRII, ALK1, endoglin and/or BMP10 propeptide polypeptide may be expressed in bacterial cells such as E. coli, insect cells (e.g., using a baculovirus expression system), yeast, or mammalian cells [e.g. a Chinese hamster ovary (CHO) cell line]. Other suitable host cells are known to those skilled in the art.

Accordingly, the present disclosure further pertains to methods of producing the subject ActRII, BMPRII, ALK1, endoglin and/or BMP10 propeptide polypeptides. For example, a host cell transfected with an expression vector encoding an ActRII, BMPRII, ALK1, endoglin and/or BMP10 propeptide polypeptide can be cultured under appropriate conditions to allow expression of the ActRII, BMPRII, ALK1, endoglin and/or BMP10 propeptide polypeptide to occur. The polypeptide may be secreted and isolated from a mixture of cells and medium containing the polypeptide. Alternatively, ActRII, BMPRII, ALK1, endoglin and/or BMP10 propeptide polypeptide may be isolated from a cytoplasmic or membrane fraction obtained from harvested and lysed cells. A cell culture includes host cells, media and other byproducts. Suitable media for cell culture are well known in the art. The subject polypeptides can be isolated from cell culture medium, host cells, or both, using techniques known in the art for purifying proteins, including ion-exchange chromatography, gel filtration chromatography, ultrafiltration, electrophoresis, immunoaffinity purification with antibodies specific for particular epitopes of ActRII, BMPRII, ALK1, endoglin and/or BMP10 propeptide polypeptides and affinity purification with an agent that binds to a domain fused to ActRII, BMPRII, ALK1, endoglin and/or BMP10 propeptide polypeptide (e.g., a protein A column may be used to purify ActRII-Fc, BMPRII-Fc, ALK1-Fc, endoglin-Fc and/or BMP10 propeptide-Fc fusion proteins). In some embodiments, the ActRII, BMPRII, ALK1, endoglin and/or BMP10 propeptide polypeptide is a fusion protein containing a domain which facilitates its purification.

In some embodiments, purification is achieved by a series of column chromatography steps, including, for example, three or more of the following, in any order: protein A chromatography, Q sepharose chromatography, phenylsepharose chromatography, size exclusion chromatography, and cation exchange chromatography. The purification could be completed with viral filtration and buffer exchange. An ActRII-Fc, BMPRII-Fc, ALK1-Fc, endoglin-Fc and/or BMP10 propeptide-Fc fusion protein may be purified to a purity of >90%, >95%, >96%, >98%, or >99% as determined by size exclusion chromatography and >90%, >95%, >96%, >98%, or >99% as determined by SDS PAGE. The target level of purity should be one that is sufficient to achieve desirable results in mammalian systems, particularly non-human primates, rodents (mice), and humans.

In another embodiment, a fusion gene coding for a purification leader sequence, such as a poly-(His)/enterokinase cleavage site sequence at the N-terminus of the desired portion of the recombinant ActRII, BMPRII, ALK1, endoglin and/or BMP10 propeptide polypeptide, can allow purification of the expressed fusion protein by affinity chromatography using a Ni²⁺ metal resin. The purification leader sequence can then be subsequently removed by treatment with enterokinase to provide the purified ActRII, BMPRII, ALK1, endoglin and/or BMP10 propeptide polypeptide[Hochuli et al. (1987) J. Chromatography 411:177; and Janknecht et al. (1991) PNAS USA 88:8972].

Techniques for making fusion genes are well known. Essentially, the joining of various DNA fragments coding for different polypeptide sequences is performed in accordance with conventional techniques, employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed to generate a chimeric gene sequence. See, e.g., Current Protocols in Molecular Biology, eds. Ausubel et al., John Wiley & Sons: 1992.

### 4. Antibody Antagonists

In other aspects, the present disclosure relates to a BMP antagonist (inhibitor) that is antibody, or combination of antibodies. A BMP antagonist antibody, or combination of antibodies, may bind to, for example, BMP10, BMP9, BMP6, BMP5, and/or BMP3b or one or more BMP-interacting receptors [e.g., ActRIIA, ActRIIB, BMPRII, and endoglin]. In particular, the disclosure provides methods of using an BMP antagonist antibody, or a combination of BMP antagonist antibodies, alone or in combination with one or more additional supportive therapies and/or active agents, to achieve a desired effect in a subject in need thereof (e.g., treating heart failure or one or more complications of heart failure).

In certain aspects, a BMP antagonist antibody, or combination of antibodies, of the disclosure is an antibody that inhibits at least BMP10. Therefore, in some embodiments, a BMP antagonist antibody, or combination of antibodies, binds to at least BMP10. As used herein, a BMP10 antibody (anti-BMP10 antibody) generally refers to an antibody that binds to BMP10 with sufficient affinity such that the antibody is useful as a diagnostic and/or therapeutic agent in targeting BMP10. In certain embodiments, the extent of binding of an anti-BMP10 antibody to an unrelated, non-BMP10 protein is less than about 10%, 9%, 8%, 7%, 6%, 5%, 4%, 3%, 2%, or less than about 1% of the binding of the antibody to BMP10 as measured, for example, by a radioimmunoassay (MA), Biacore, or other protein-protein interaction or binding affinity assay. In certain embodiments, an anti-BMP10 antibody binds to an epitope of BMP10 that is conserved among BMP10 from different species. In certain preferred embodiments, an anti-BMP10 antibody binds to human BMP10. In other preferred embodiments, an anti-BMP10 antibody may inhibit BMP10 from binding to a cognate type I-, type II-, or co-receptor (e.g., ActRIIA, ActRIIB, BMPRII, ALK1, and endoglin) and thus inhibit BMP10-mediated signaling (e.g., Smad signaling) via these receptors. It should be noted that BMP10 has some sequence homology to BMP9 and therefore antibodies that bind to BMP10, in some cases, may also bind to and/or inhibit BMP9. In some embodiments, an anti-BMP10 antibody is a multispecific antibody (e.g., bi-specific antibody) that binds to one or more additional ligands (e.g., BMP9, BMP6, BMP5, and BMP3b) and/or binds to one or more type I-, type II-, and/or co-receptors (e.g., ActRIIA, ActRIIB, BMPRII, ALK1, and endoglin). In some embodiments, a BMP10 antibody further binds to BMP9. In some embodiments, the disclosure relates to combinations of antibodies, as well as uses thereof, wherein the combination of antibodies comprises an anti-BMP10 antibody and one or more additional antibodies that bind to, for example, different ligands (e.g., BMP9, BMP6, BMP5, and BMP3b) and/or binds to one or more type I-, type II-, and/or co-receptors (e.g., ActRIIA, ActRIIB, BMPRII, ALK1, and endoglin). In some embodiments, a combination antibodies comprising an anti-BMP10 antibody further comprises an anti-BMP9 antibody. Preferably, BMP10 antibodies bind to the mature BMP10 domain and bind competitively with a BMP10 propeptide.

In certain aspects, a BMP antagonist antibody, or combination of antibodies, of the disclosure is an antibody that inhibits at least BMP9. Therefore, in some embodiments, a BMP antagonist antibody, or combination of antibodies, binds to at least BMP9. As used herein, a BMP9antibody (anti-BMP9 antibody) generally refers to an antibody that binds to BMP9 with sufficient affinity such that the antibody is useful as a diagnostic and/or therapeutic agent in targeting BMP9. In certain embodiments, the extent of binding of an anti-BMP9 antibody to an unrelated, non-BMP9 protein is less than about 10%, 9%, 8%, 7%, 6%, 5%, 4%, 3%, 2%, or less than about 1% of the binding of the antibody to BMP9 as measured, for example, by a radioimmunoassay (MA), Biacore, or other protein-protein interaction or binding affinity assay. In certain embodiments, an anti-BMP9 antibody binds to an epitope of BMP9 that is conserved among BMP9 from different species. In certain preferred embodiments, an anti-BMP9 antibody binds to human BMP9. In other preferred embodiments, an anti-BMP9 antibody may inhibit BMP9 from binding to a cognate type I-, type II-, or co-receptor (e.g., ActRIIA, ActRIIB, BMPRII, ALK1, and endoglin) and thus inhibit BMP9-mediated signaling (e.g., Smad signaling) via these receptors. It should be noted that BMP9 has some sequence homology to BMP10 and therefore antibodies that bind to BMP9, in some cases, may also bind to and/or inhibit BMP10. In some embodiments, an anti-BMP9 antibody is a multispecific antibody (e.g., bi-specific antibody) that binds to one or more additional ligands [e.g., BMP10, BMP6, BMP5, and BMP3b] and/or binds to one or more type I-, type II-, and/or co-receptors (e.g., ActRIIA, ActRIIB, BMPRII, ALK1, and endoglin). In some embodiments, a BMP9 antibody further binds to BMP10. In some embodiments, the disclosure relates to combinations of antibodies, as well as uses thereof, wherein the combination of antibodies comprises an anti-BMP9 antibody and one or more additional antibodies that bind to, for example, different ligands (e.g., BMP10, BMP6, BMP5, and BMP3b) and/or binds to one or more type I-, type II-, and/or co-receptors (e.g., ActRIIA, ActRIIB, BMPRII, ALK1, and endoglin). In some embodiments, a combination antibodies comprising an anti-BMP9 antibody further comprises an anti-BMP10 antibody.

In certain aspects, a BMP antagonist antibody, or combination of antibodies, of the disclosure is an antibody that inhibits at least BMP6. Therefore, in some embodiments, a BMP antagonist antibody, or combination of antibodies, binds to at least BMP6. As used herein, a BMP6 antibody (anti-BMP6 antibody) generally refers to an antibody that binds to BMP6 with sufficient affinity such that the antibody is useful as a diagnostic and/or therapeutic agent in targeting BMP6. In certain embodiments, the extent of binding of an anti-BMP6 antibody to an unrelated, non-BMP6 protein is less than about 10%, 9%, 8%, 7%, 6%, 5%, 4%, 3%, 2%, or less than about 1% of the binding of the antibody to BMP6 as measured, for example, by a radioimmunoassay (MA), Biacore, or other protein-protein interaction or binding affinity assay. In certain embodiments, an anti-BMP6 antibody binds to an epitope of BMP6 that is conserved among BMP6 from different species. In certain preferred embodiments, an anti-BMP6 antibody binds to human BMP6. In other preferred embodiments, an anti-BMP6 antibody may inhibit BMP6 from binding to a cognate type I-, type II-, or co-receptor and thus inhibit BMP6-mediated signaling (e.g., Smad signaling) via these receptors. In some embodiments, an anti-BMP6 antibody is a multispecific antibody (e.g., bi-specific antibody) that binds to one or more additional ligands (e.g., BMP10, BMP9, BMP5, and BMP3b) and/or binds to one or more type I-, type II-, and/or co-receptors (e.g., ActRIIA, ActRIIB, BMPRII, ALK1, and endoglin). In some embodiments, a BMP6 antibody further binds to BMP9 and/or BMP10. In some embodiments, the disclosure relates to combinations of antibodies, as well as uses thereof, wherein the combination of antibodies comprises an anti-BMP6 antibody and one or more additional antibodies that bind to, for example, different ligands (e.g., BMP10, BMP9, BMP5, and BMP3b) and/or binds to one or more type I-, type II-, and/or co-receptors (e.g., ActRIIA, ActRIIB, BMPRII, ALK1, and endoglin). In some embodiments, a combination antibodies comprising an anti-BMP6 antibody further comprises an anti-BMP10 and/or BMP9 antibody.

In certain aspects, a BMP antagonist antibody, or combination of antibodies, of the disclosure is an antibody that inhibits at least BMP5. Therefore, in some embodiments, a BMP antagonist antibody, or combination of antibodies, binds to at least BMP5. As used herein, a BMP5 antibody (anti-BMP5 antibody) generally refers to an antibody that binds to BMP5 with sufficient affinity such that the antibody is useful as a diagnostic and/or therapeutic agent in targeting BMP5. In certain embodiments, the extent of binding of an anti-BMP5 antibody to an unrelated, non-BMP5 protein is less than about 10%, 9%, 8%, 7%, 6%, 5%, 4%, 3%, 2%, or less than about 1% of the binding of the antibody to BMP5 as measured, for example, by a radioimmunoassay (MA), Biacore, or other protein-protein interaction or binding affinity assay. In certain embodiments, an anti-BMP5 antibody binds to an epitope of BMP5 that is conserved among BMP5 from different species. In certain preferred embodiments, an anti-BMP5 antibody binds to human BMP5. In other preferred embodiments, an anti-BMP5 antibody may inhibit BMP5 from binding to a cognate type I-, type II-, or co-receptor and thus inhibit BMP5-mediated signaling (e.g., Smad signaling) via these receptors. In some embodiments, an anti-BMP5 antibody is a multispecific antibody (e.g., bi-specific antibody) that binds to one or more additional ligands (e.g., BMP10, BMP9, BMP6, and BMP3b) and/or binds to one or more type I-, type II-, and/or co-receptors (e.g., ActRIIA, ActRIIB, BMPRII, ALK1, and endoglin). In some embodiments, a BMP5 antibody further binds to BMP9 and/or BMP10. In some embodiments, the disclosure relates to combinations of antibodies, as well as uses thereof, wherein the combination of antibodies comprises an anti-BMP5 antibody and one or more additional antibodies that bind to, for example, different ligands (e.g., BMP10, BMP9, BMP6, and BMP3b) and/or binds to one or more type I-, type II-, and/or co-receptors (e.g., ActRIIA, ActRIIB, BMPRII, ALK1, and endoglin). In some embodiments, a combination antibodies comprising an anti-BMP5 antibody further comprises an anti-BMP10 and/or BMP9 antibody.

In certain aspects, a BMP antagonist antibody, or combination of antibodies, of the disclosure is an antibody that inhibits at least BMP3b. Therefore, in some embodiments, a BMP antagonist antibody, or combination of antibodies, binds to at least BMP3b. As used herein, a BMP3b antibody (anti-BMP3b antibody) generally refers to an antibody that binds to BMP3b with sufficient affinity such that the antibody is useful as a diagnostic and/or therapeutic agent in targeting BMP3b. In certain embodiments, the extent of binding of an anti-BMP3b antibody to an unrelated, non-BMP3b protein is less than about 10%, 9%, 8%, 7%, 6%, 5%, 4%, 3%, 2%, or less than about 1% of the binding of the antibody to BMP3b as measured, for example, by a radioimmunoassay (MA), Biacore, or other protein-protein interaction or binding affinity assay. In certain embodiments, an anti-BMP3b antibody binds to an epitope of BMP3b that is conserved among BMP3b from different species. In certain preferred embodiments, an anti-BMP3b antibody binds to human BMP5. In other preferred embodiments, an anti-BMP3b antibody may inhibit BMP3b from binding to a cognate type I-, type II-, or co-receptor and thus inhibit BMP3b-mediated signaling (e.g., Smad signaling) via these receptors. In some embodiments, an anti-BMP3b antibody is a multispecific antibody (e.g., bi-specific antibody) that binds to one or more additional ligands (e.g., BMP10, BMP9, BMP6, and BMP5) and/or binds to one or more type I-, type II-, and/or co-receptors (e.g., ActRIIA, ActRIIB, BMPRII, ALK1, and endoglin). In some embodiments, a BMP3b antibody further binds to BMP9 and/or BMP10. In some embodiments, the disclosure relates to combinations of antibodies, as well as uses thereof, wherein the combination of antibodies comprises an anti-BMP3b antibody and one or more additional antibodies that bind to, for example, different ligands (e.g., BMP10, BMP9, BMP6, and BMP5) and/or binds to one or more type I-, type II-, and/or co-receptors (e.g., ActRIIA, ActRIIB, BMPRII, ALK1, and endoglin). In some embodiments, a combination antibodies comprising an anti-BMP3b antibody further comprises an anti-BMP10 and/or BMP9 antibody.

In other aspects, a BMP antagonist antibody, or combination of antibodies, of the disclosure is an antibody that inhibits at least an ActRII receptor (e.g., ActRIIA and/or ActRIIB) Therefore, in some embodiments, a BMP antagonist antibody, or combination of antibodies, binds to at least ActRIIA, but does not bind or does not substantially bind to ActRIIB (e.g., binds to ActRIIB with a K_{D} of greater than 1×10⁻⁷ M or has relatively modest binding, e.g., about 1×10⁻⁸ M or about 1×10⁻⁹ M). In other embodiments, an ActRII antagonist antibody, or combination of antibodies, binds to at least ActRIIB, but does not bind or does not substantially bind to ActRIIA (e.g., binds to ActRIIA with a K_{D} of greater than 1×10⁻⁷ M or has relatively modest binding, e.g., about 1×10⁻⁸ M or about 1×10⁻⁹ M). In still other embodiments, an ActRII antagonist antibody, or combination of antibodies, binds to at least ActRIIA and ActRIIB As used herein, an ActRII antibody (anti-ActRII antibody) generally refers to an antibody that binds to ActRII (e.g., ActRIIA and/or ActRIIB) with sufficient affinity such that the antibody is useful as a diagnostic and/or therapeutic agent in targeting ActRII. In certain embodiments, the extent of binding of an anti-ActRII antibody to an unrelated, non-ActRII protein is less than about 10%, 9%, 8%, 7%, 6%, 5%, 4%, 3%, 2%, or less than about 1% of the binding of the antibody to ActRII as measured, for example, by a radioimmunoassay (RIA), Biacore, or other protein-protein interaction or binding affinity assay. In certain embodiments, an anti-ActRII antibody binds to an epitope of ActRII (e.g., ActRIIA and/or ActRIIB) that is conserved among ActRII from different species. In certain preferred embodiments, an anti-ActRII antibody binds to human ActRII (e.g., ActRIIA and/or ActRIIB) In other preferred embodiments, an anti-ActRII antibody may inhibit one or more ligands (e.g., BMP10, BMP9, BMP6, and BMP5) from binding to ActRII (e.g., ActRIIA and/or ActRIIB) It should be noted that ActRIIA has sequence homology to ActRIIB and therefore antibodies that bind to ActRIIA, in some cases, may also bind to and/or inhibit ActRIIB, the reverse is also true. In some embodiments, an anti-ActRII antibody is a multispecific antibody (e.g., bi-specific antibody) that binds to ActRII (e.g., ActRIIA and/or ActRIIB) and one or more ligands (e.g., BMP10, BMP9, BMP6, BMP5, and BMP3b). In some embodiments, an anti-ActRII antibody is a multispecific antibody (e.g., bi-specific antibody) that binds to ActRIIA and ActRIIB In some embodiments, the disclosure relates to combinations of antibodies, as well as uses thereof, wherein the combination of antibodies comprises at least an anti-ActRIIA antibody and at least an ActRIIB antibody. In some embodiments, the disclosure relates to combinations of antibodies, as well as uses thereof, wherein the combination of antibodies comprises an anti-ActRIIA antibody and one or more additional antibodies that bind to, for example, one or more ligands (e.g., BMP10, BMP9, BMP6, and BMP5), BMPRII, ALK1, and/or endoglin. In some embodiments, the disclosure relates to combinations of antibodies, as well as uses thereof, wherein the combination of antibodies comprises an anti-ActRIIB antibody and one or more additional antibodies that bind to, for example, one or more ligands (e.g., BMP10, BMP9, BMP6, and BMP5), BMPRII, ALK1, and/or endoglin. In some embodiments, the disclosure relates to combinations of antibodies, as well as uses thereof wherein the combination of antibodies comprises an anti-ActRIIA antibody, an anti-ActRIIB antibody, and at least one or more additional antibodies that bind to, for example, one or more ligands (e.g., BMP10, BMP9, BMP6, and BMP5), BMPRII, ALK1, and/or endoglin.

In other aspects, a BMP antagonist antibody, or combination of antibodies, of the disclosure is an antibody that inhibits at least ALK1. Therefore, in some embodiments, a BMP antagonist antibody, or combination of antibodies, binds to at least ALK1. As used herein, an ALK1 antibody (anti-ALK1 antibody) generally refers to an antibody that binds to ALK1 with sufficient affinity such that the antibody is useful as a diagnostic and/or therapeutic agent in targeting ALK1. In certain embodiments, the extent of binding of an anti-ALK1 antibody to an unrelated, non-ALK1 protein is less than about 10%, 9%, 8%, 7%, 6%, 5%, 4%, 3%, 2%, or less than about 1% of the binding of the antibody to ALK1 as measured, for example, by a radioimmunoassay (MA), Biacore, or other protein-protein interaction or binding affinity assay. In certain embodiments, an anti-ALK1 antibody binds to an epitope of ALK1 that is conserved among ALK1 from different species. In certain preferred embodiments, an anti-ALK1 antibody binds to human ALK1. In other preferred embodiments, an anti-ALK1 antibody may inhibit one or more ligands (e.g., BMP10 and BMP9) from binding to ALK1. In some embodiments, an anti-ALK1 antibody is a multispecific antibody (e.g., bi-specific antibody) that binds to ALK1 and one or more ligands (e.g. BMP9 and BMP10), BMPRII, ActRII (ActRIIA and/or ActRIIB) and/or endoglin. In some embodiments, the disclosure relates to combinations of antibodies, as well as uses thereof, wherein the combination of antibodies comprises an anti-ALK1 antibody and one or more additional antibodies that bind to, for example, one or more ligands (e.g. BMP9 and BMP10), BMPRII, ActRII (ActRIIA and/or ActRIIB) and/or endoglin.

In other aspects, a BMP antagonist antibody, or combination of antibodies, of the disclosure is an antibody that inhibits at least BMPRII. Therefore, in some embodiments, a BMP antagonist antibody, or combination of antibodies, binds to at least BMPRII. As used herein, an BMPRII antibody (anti-BMPRII antibody) generally refers to an antibody that binds to BMPRII with sufficient affinity such that the antibody is useful as a diagnostic and/or therapeutic agent in targeting BMPRII. In certain embodiments, the extent of binding of an anti-BMPRII antibody to an unrelated, non-BMPRII protein is less than about 10%, 9%, 8%, 7%, 6%, 5%, 4%, 3%, 2%, or less than about 1% of the binding of the antibody to BMPRII as measured, for example, by a radioimmunoassay (RIA), Biacore, or other protein-protein interaction or binding affinity assay. In certain embodiments, an anti-BMPRII antibody binds to an epitope of BMPRII that is conserved among BMPRII from different species. In certain preferred embodiments, an anti-BMPRII antibody binds to human BMPRII. In other preferred embodiments, an anti-BMPRII antibody may inhibit one or more ligands (e.g., BMP10 and BMP9) from binding to BMPRII. In some embodiments, an anti-BMPRII antibody is a multispecific antibody (e.g., bi-specific antibody) that binds to BMPRII and one or more ligands (e.g. BMP9 and BMP10), ALK1, ActRII (ActRIIA and/or ActRIIB) and/or endoglin. In some embodiments, the disclosure relates to combinations of antibodies, as well as uses thereof, wherein the combination of antibodies comprises an anti-BMPRII antibody and one or more additional antibodies that bind to, for example, one or more ligands (e.g. BMP9 and BMP10), ALK1, ActRII (ActRIIA and/or ActRIIB) and/or endoglin.

In other aspects, a BMP antagonist antibody, or combination of antibodies, of the disclosure is an antibody that inhibits at least endoglin. Therefore, in some embodiments, a BMP antagonist antibody, or combination of antibodies, binds to at least endoglin. As used herein, a endoglin antibody (anti-endoglin antibody) generally refers to an antibody that binds to endoglin with sufficient affinity such that the antibody is useful as a diagnostic and/or therapeutic agent in targeting endoglin. In certain embodiments, the extent of binding of an anti-endoglin antibody to an unrelated, non-endoglin protein is less than about 10%, 9%, 8%, 7%, 6%, 5%, 4%, 3%, 2%, or less than about 1% of the binding of the antibody to endoglin as measured, for example, by a radioimmunoassay (MA), Biacore, or other protein-protein interaction or binding affinity assay. In certain embodiments, an anti-endoglin antibody binds to an epitope of endoglin that is conserved among endoglin from different species. In certain preferred embodiments, an anti-endoglin antibody binds to human endoglin. In other preferred embodiments, an anti-endoglin antibody may inhibit one or more ligands (e.g., BMP10 and BMP9) from binding to endoglin. In some embodiments, an anti-endoglin antibody is a multispecific antibody (e.g., bi-specific antibody) that binds to endoglin and one or more ligands (e.g. BMP9 and BMP10), ALK1, ActRII (ActRIIA and/or ActRIIB) and/or BMPRII. In some embodiments, the disclosure relates to combinations of antibodies, as well as uses thereof, wherein the combination of antibodies comprises an anti-endoglin antibody and one or more additional antibodies that bind to, for example, one or more ligands (e.g. BMP9 and BMP10), ALK1, ActRII (ActRIIA and/or ActRIIB) and/or BMPRII.

The term antibody is used herein in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity. An antibody fragment refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; single-chain antibody molecules (e.g., scFv); and multispecific antibodies formed from antibody fragments. See, e.g., Hudson et al. (2003) Nat. Med. 9:129-134; Plückthun, in The Pharmacology of Monoclonal Antibodies, vol. 113, Rosenburg and Moore eds., (Springer-Verlag, New York), pp. 269-315 (1994); WO 93/16185; and U.S. Pat. Nos. 5,571,894, 5,587,458, and 5,869,046. Antibodies disclosed herein may be polyclonal antibodies or monoclonal antibodies. In certain embodiments, the antibodies of the present disclosure comprise a label attached thereto and able to be detected (e.g., the label can be a radioisotope, fluorescent compound, enzyme, or enzyme co-factor). In preferred embodiments, the antibodies of the present disclosure are isolated antibodies.

Diabodies are antibody fragments with two antigen-binding sites that may be bivalent or bispecific. See, e.g., EP 404,097; WO 1993/01161; Hudson et al. (2003) Nat. Med. 9:129-134 (2003); and Hollinger et al. (1993) Proc. Natl. Acad. Sci. USA 90: 6444-6448. Triabodies and tetrabodies are also described in Hudson et al. (2003) Nat. Med. 9:129-134.

Single-domain antibodies are antibody fragments comprising all or a portion of the heavy-chain variable domain or all or a portion of the light-chain variable domain of an antibody. In certain embodiments, a single-domain antibody is a human single-domain antibody. See, e.g., U.S. Pat. No. 6,248,516.

Antibody fragments can be made by various techniques, including but not limited to proteolytic digestion of an intact antibody as well as production by recombinant host cells (e.g., E. coli or phage), as described herein.

The antibodies herein may be of any class. The class of an antibody refers to the type of constant domain or constant region possessed by its heavy chain. There are five major classes of antibodies: IgA, IgD, IgE, IgG, and IgM, and several of these may be further divided into subclasses (isotypes), for example, IgG₁, IgG₂, IgG₃, IgG₄, IgA₁, and IgA₂. The heavy-chain constant domains that correspond to the different classes of immunoglobulins are called alpha, delta, epsilon, gamma, and mu.

In general, an antibody for use in the methods disclosed herein specifically binds to its target antigen, preferably with high binding affinity. Affinity may be expressed as a K_{D} value and reflects the intrinsic binding affinity (e.g., with minimized avidity effects). Typically, binding affinity is measured in vitro, whether in a cell-free or cell-associated setting. Any of a number of assays known in the art, including those disclosed herein, can be used to obtain binding affinity measurements including, for example, surface plasmon resonance (Biacore™ assay), radiolabeled antigen binding assay (MA), and ELISA. In some embodiments, antibodies of the present disclosure bind to their target antigens [e.g., BMP10, BMP9, BMP6, BMP5, BMP3b, ActRII (ActRIIA and/or ActRIIB), BMPRII, ALK1, and endoglin] with at least a K_{D} of 1×10⁻⁷ or stronger, 1×10⁻⁸ or stronger, 1×10⁻⁹ or stronger, 1×10⁻¹⁰ or stronger, 1×10⁻¹¹ or stronger, 1×10⁻¹² or stronger, 1×10⁻¹³ or stronger, or 1×10⁻¹⁴ or stronger.

In certain embodiments, K_{D} is measured by MA performed with the Fab version of an antibody of interest and its target antigen as described by the following assay. Solution binding affinity of Fabs for the antigen is measured by equilibrating Fab with a minimal concentration of radiolabeled antigen (e.g., ¹²⁵I-labeled) in the presence of a titration series of unlabeled antigen, then capturing bound antigen with an anti-Fab antibody-coated plate [see, e.g., Chen et al. (1999) J. Mol. Biol. 293:865-881]. To establish conditions for the assay, multi-well plates (e.g., MICROTITER® from Thermo Scientific) are coated (e.g., overnight) with a capturing anti-Fab antibody (e.g., from Cappel Labs) and subsequently blocked with bovine serum albumin, preferably at room temperature (e.g., approximately 23° C.). In a non-adsorbent plate, radiolabeled antigen are mixed with serial dilutions of a Fab of interest [e.g., consistent with assessment of the anti-VEGF antibody, Fab-12, in Presta et al., (1997) Cancer Res. 57:4593-4599]. The Fab of interest is then incubated, preferably overnight but the incubation may continue for a longer period (e.g., about 65 hours) to ensure that equilibrium is reached. Thereafter, the mixtures are transferred to the capture plate for incubation, preferably at room temperature for about one hour. The solution is then removed and the plate is washed times several times, preferably with polysorbate 20 and PBS mixture. When the plates have dried, scintillant (e.g., MICROSCINT® from Packard) is added, and the plates are counted on a gamma counter (e.g., TOPCOUNT® from Packard).

According to another embodiment, K_{D} is measured using surface plasmon resonance assays using, for example a BIACORE® 2000 or a BIACORE® 3000 (Biacore, Inc., Piscataway, N.J.) with immobilized antigen CMS chips at about 10 response units (RU). Briefly, carboxymethylated dextran biosensor chips (CMS, Biacore, Inc.) are activated with N-ethyl-N′-(3-dimethylaminopropyl)-carbodiimide hydrochloride (EDC) and N-hydroxysuccinimide (NETS) according to the supplier's instructions. For example, an antigen can be diluted with 10 mM sodium acetate, pH 4.8, to 5 μg/ml (about 0.2 μM) before injection at a flow rate of 5 μl/minute to achieve approximately 10 response units (RU) of coupled protein. Following the injection of antigen, 1 M ethanolamine is injected to block unreacted groups. For kinetics measurements, two-fold serial dilutions of Fab (0.78 nM to 500 nM) are injected in PBS with 0.05% polysorbate 20 (TWEEN-20) surfactant (PBST) at at a flow rate of approximately 25 μl/min. Association rates (kₒₙ) and dissociation rates (k_{off}) are calculated using, for example, a simple one-to-one Langmuir binding model (BIACORE® Evaluation Software version 3.2) by simultaneously fitting the association and dissociation sensorgrams. The equilibrium dissociation constant (K_{D}) is calculated as the ratio k_{off}/kₒₙ [see, e.g., Chen et al., (1999) J. Mol. Biol. 293:865-881]. If the on-rate exceeds, for example, 10⁶ M⁻¹ s⁻¹ by the surface plasmon resonance assay above, then the on-rate can be determined by using a fluorescent quenching technique that measures the increase or decrease in fluorescence emission intensity (e.g., excitation=295 nm; emission=340 nm, 16 nm band-pass) of a 20 nM anti-antigen antibody (Fab form) in PBS in the presence of increasing concentrations of antigen as measured in a spectrometer, such as a stop-flow equipped spectrophometer (Aviv Instruments) or a 8000-series SLM-AMINCO® spectrophotometer (ThermoSpectronic) with a stirred cuvette.

The nucleic acid and amino acid sequences of human BMP10, BMP9, BMP6, BMP5, BMP3b, ActRII (ActRIIA and/or ActRIIB), BMPRII, ALK1, and endoglin are well known in the art and thus antibody antagonists for use in accordance with this disclosure may be routinely made by the skilled artisan based on the knowledge in the art and teachings provided herein.

In certain embodiments, an antibody provided herein is a chimeric antibody. A chimeric antibody refers to an antibody in which a portion of the heavy and/or light chain is derived from a particular source or species, while the remainder of the heavy and/or light chain is derived from a different source or species. Certain chimeric antibodies are described, for example, in U.S. Pat. No. 4,816,567; and Morrison et al., (1984) Proc. Natl. Acad. Sci. USA, 81:6851-6855. In some embodiments, a chimeric antibody comprises a non-human variable region (e.g., a variable region derived from a mouse, rat, hamster, rabbit, or non-human primate, such as a monkey) and a human constant region. In some embodiments, a chimeric antibody is a “class switched” antibody in which the class or subclass has been changed from that of the parent antibody. In general, chimeric antibodies include antigen-binding fragments thereof.

In certain embodiments, a chimeric antibody provided herein is a humanized antibody. A humanized antibody refers to a chimeric antibody comprising amino acid residues from non-human hypervariable regions (HVRs) and amino acid residues from human framework regions (FRs). In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

Humanized antibodies and methods of making them are reviewed, for example, in Almagro and Fransson (2008) Front. Biosci. 13:1619-1633 and are further described, for example, in Riechmann et al., (1988) Nature 332:323-329; Queen et al. (1989) Proc. Nat'l Acad. Sci. USA 86:10029-10033; U.S. Pat. Nos. 5,821,337, 7,527,791, 6,982,321, and 7,087,409; Kashmiri et al., (2005) Methods 36:25-34 [describing SDR (a-CDR) grafting]; Padlan, Mol. Immunol. (1991) 28:489-498 (describing “resurfacing”); Dall'Acqua et al. (2005) Methods 36:43-60 (describing “FR shuffling”); Osbourn et al. (2005) Methods 36:61-68; and Klimka et al. Br. J. Cancer (2000) 83:252-260 (describing the “guided selection” approach to FR shuffling).

Human framework regions that may be used for humanization include but are not limited to: framework regions selected using the “best-fit” method [see, e.g., Sims et al. (1993) J. Immunol. 151:2296]; framework regions derived from the consensus sequence of human antibodies of a particular subgroup of light-chain or heavy-chain variable regions [see, e.g., Carter et al. (1992) Proc. Natl. Acad. Sci. USA, 89:4285; and Presta et al. (1993) J. Immunol., 151:2623]; human mature (somatically mutated) framework regions or human germline framework regions [see, e.g., Almagro and Fransson (2008) Front. Biosci. 13:1619-1633]; and framework regions derived from screening FR libraries [see, e.g., Baca et cd., (1997) J. Biol. Chem. 272:10678-10684; and Rosok et cd., (1996) J. Biol. Chem. 271:22611-22618].

In certain embodiments, an antibody provided herein is a human antibody. Human antibodies can be produced using various techniques known in the art. Human antibodies are described generally in van Dijk and van de Winkel (2001) Curr. Opin. Pharmacol. 5: 368-74 and Lonberg (2008) Curr. Opin. Immunol. 20:450-459.

Human antibodies may be prepared by administering an immunogen [e.g., BMP10, BMP9, BMP6, BMP5, BMP3b, ActRII (ActRIIA and/or ActRIIB), BMPRII, ALK1, and endoglin] to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic animals, the endogenous immunoglobulin loci have generally been inactivated. For a review of methods for obtaining human antibodies from transgenic animals, see, for example, Lonberg (2005) Nat. Biotechnol. 23:1117-1125; U.S. Pat. Nos. 6,075,181 and 6,150,584 (describing XENOMOUSE™ technology); U.S. Pat. No. 5,770,429 (describing HuMab® technology); U.S. Pat. No. 7,041,870 (describing K-M MOUSE® technology); and U.S. Patent Application Publication No. 2007/0061900 (describing VelociMouse® technology). Human variable regions from intact antibodies generated by such animals may be further modified, for example, by combining with a different human constant region.

Human antibodies provided herein can also be made by hybridoma-based methods. Human myeloma and mouse-human heteromyeloma cell lines for the production of human monoclonal antibodies have been described [see, e.g., Kozbor J. Immunol., (1984) 133: 3001; Brodeur et al. (1987) Monoclonal Antibody Production Techniques and Applications, pp. 51-63, Marcel Dekker, Inc., New York; and Boerner et al. (1991) J. Immunol., 147: 86]. Human antibodies generated via human B-cell hybridoma technology are also described in Li et al., (2006) Proc. Natl. Acad. Sci. USA, 103:3557-3562. Additional methods include those described, for example, in U.S. Pat. No. 7,189,826 (describing production of monoclonal human IgM antibodies from hybridoma cell lines) and Ni, Xiandai Mianyixue (2006) 26(4):265-268 (2006) (describing human-human hybridomas). Human hybridoma technology (Trioma technology) is also described in Vollmers and Brandlein (2005) Histol. Histopathol., 20(3):927-937 (2005) and Vollmers and Brandlein (2005) Methods Find Exp. Clin. Pharmacol., 27(3):185-91.

Human antibodies provided herein may also be generated by isolating Fv clone variable-domain sequences selected from human-derived phage display libraries. Such variable-domain sequences may then be combined with a desired human constant domain. Techniques for selecting human antibodies from antibody libraries are described herein.

For example, antibodies of the present disclosure may be isolated by screening combinatorial libraries for antibodies with the desired activity or activities. A variety of methods are known in the art for generating phage-display libraries and screening such libraries for antibodies possessing the desired binding characteristics. Such methods are reviewed, for example, in Hoogenboom et al. (2001) in Methods in Molecular Biology 178:1-37, O'Brien et al., ed., Human Press, Totowa, N.J. and further described, for example, in the McCafferty et al. (1991) Nature 348:552-554; Clackson et al., (1991) Nature 352: 624-628; Marks et al. (1992) J. Mol. Biol. 222:581-597; Marks and Bradbury (2003) in Methods in Molecular Biology 248:161-175, Lo, ed., Human Press, Totowa, N.J.; Sidhu et al. (2004) J. Mol. Biol. 338(2):299-310; Lee et al. (2004) J. Mol. Biol. 340(5):1073-1093; Fellouse (2004) Proc. Natl. Acad. Sci. USA 101(34):12467-12472; and Lee et al. (2004) J. Immunol. Methods 284(1-2): 119-132.

In certain phage display methods, repertoires of VH and VL genes are separately cloned by polymerase chain reaction (PCR) and recombined randomly in phage libraries, which can then be screened for antigen-binding phage as described in Winter et al. (1994) Ann. Rev. Immunol., 12: 433-455. Phage typically display antibody fragments, either as single-chain Fv (scFv) fragments or as Fab fragments. Libraries from immunized sources provide high-affinity antibodies to the immunogen [e.g., BMP10, BMP9, BMP6, BMP5, BMP3b, ActRII (ActRIIA and/or ActRIIB), BMPRII, ALK1, and endoglin] without the requirement of constructing hybridomas. Alternatively, the naive repertoire can be cloned (e.g., from human) to provide a single source of antibodies directed against a wide range of non-self and also self-antigens without any immunization as described by Griffiths et al. (1993) EMBO J, 12: 725-734. Finally, naive libraries can also be made synthetically by cloning un-rearranged V-gene segments from stem cells and using PCR primers containing random sequence to encode the highly variable CDR3 regions and to accomplish rearrangement in vitro, as described by Hoogenboom and Winter (1992) J. Mol. Biol., 227: 381-388. Patent publications describing human antibody phage libraries include, for example: U.S. Pat. No. 5,750,373, and U.S. Patent Publication Nos. 2005/0079574, 2005/0119455, 2005/0266000, 2007/0117126, 2007/0160598, 2007/0237764, 2007/0292936, and 2009/0002360.

In certain embodiments, an antibody provided herein is a multispecific antibody, for example, a bispecific antibody. Multispecific antibodies (typically monoclonal antibodies) have binding specificities for at least two different epitopes (e.g., two, three, four, five, or six or more) on one or more (e.g., two, three, four, five, six or more) antigens.

Engineered antibodies with three or more functional antigen binding sites, including “octopus antibodies,” are also included herein (see, e.g., US 2006/0025576A1).

In certain embodiments, the antibodies disclosed herein are monoclonal antibodies. Monoclonal antibody refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different epitopes, each monoclonal antibody of a monoclonal antibody preparation is directed against a single epitope on an antigen. Thus, the modifier “monoclonal” indicates the character of the antibody as being obtained from a substantially homogeneous population of antibodies and is not to be construed as requiring production of the antibody by any particular method. For example, the monoclonal antibodies to be used in accordance with the present methods may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

For example, by using immunogens derived from BMP10, anti-protein/anti-peptide antisera or monoclonal antibodies can be made by standard protocols [see, e.g., Antibodies: A Laboratory Manual (1988) ed. by Harlow and Lane, Cold Spring Harbor Press]. A mammal, such as a mouse, hamster, or rabbit can be immunized with an immunogenic form of the BMP10 polypeptide, an antigenic fragment which is capable of eliciting an antibody response, or a fusion protein. Techniques for conferring immunogenicity on a protein or peptide include conjugation to carriers or other techniques well known in the art. An immunogenic portion of a BMP10 polypeptide can be administered in the presence of adjuvant. The progress of immunization can be monitored by detection of antibody titers in plasma or serum. Standard ELISA or other immunoassays can be used with the immunogen as antigen to assess the levels of antibody production and/or level of binding affinity.

Following immunization of an animal with an antigenic preparation of BMP10, antisera can be obtained and, if desired, polyclonal antibodies can be isolated from the serum. To produce monoclonal antibodies, antibody-producing cells (lymphocytes) can be harvested from an immunized animal and fused by standard somatic cell fusion procedures with immortalizing cells such as myeloma cells to yield hybridoma cells. Such techniques are well known in the art, and include, for example, the hybridoma technique [see, e.g., Kohler and Milstein (1975) Nature, 256: 495-497], the human B cell hybridoma technique [see, e.g., Kozbar et al. (1983) Immunology Today, 4:72], and the EBV-hybridoma technique to produce human monoclonal antibodies [Cole et al. (1985) Monoclonal Antibodies and Cancer Therapy, Alan R. Liss, Inc. pp. 77-96]. Hybridoma cells can be screened immunochemically for production of antibodies specifically reactive with a BMP10 polypeptide, and monoclonal antibodies isolated from a culture comprising such hybridoma cells.

In certain embodiments, one or more amino acid modifications may be introduced into the Fc region of an antibody provided herein thereby generating an Fc-region variant. The Fc-region variant may comprise a human Fc-region sequence (e.g., a human IgG1, IgG2, IgG3 or IgG4 Fc region) comprising an amino acid modification (e.g., a substitution, deletion, and/or addition) at one or more amino acid positions.

For example, the present disclosure contemplates an antibody variant that possesses some but not all effector functions, which make it a desirable candidate for applications in which the half-life of the antibody in vivo is important yet for which certain effector functions [e.g., complement-dependent cytotoxicity (CDC) and antibody-dependent cellular cytotoxicity (ADCC)] are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express FcγRIII only, whereas monocytes express FcγRI, FcγRII and FcγRIII. FcR expression on hematopoietic cells is summarized in, for example, Ravetch and Kinet (1991) Annu. Rev. Immunol. 9:457-492. Non-limiting examples of in vitro assays to assess ADCC activity of a molecule of interest are described in U.S. Pat. No. 5,500,362; Hellstrom, I. et al. (1986) Proc. Nat'l Acad. Sci. USA 83:7059-7063; Hellstrom, I et al. (1985) Proc. Nat'l Acad. Sci. USA 82:1499-1502; U.S. Pat. No. 5,821,337; and Bruggemann, M. et al. (1987) J. Exp. Med. 166:1351-1361. Alternatively, non-radioactive assay methods may be employed (e.g., ACTI™, non-radioactive cytotoxicity assay for flow cytometry; CellTechnology, Inc. Mountain View, Calif.; and CytoTox 96® non-radioactive cytotoxicity assay, Promega, Madison, Wis.). Useful effector cells for such assays include peripheral blood mononuclear cells (PBMC) and natural killer (NK) cells. Alternatively, or additionally, ADCC activity of the molecule of interest may be assessed in vivo, for example, in an animal model such as that disclosed in Clynes et al. (1998) Proc. Nat'l Acad. Sci. USA 95:652-656. C1q binding assays may also be carried out to confirm that the antibody is unable to bind C1q and hence lacks CDC activity [see, e.g., C1q and C3c binding ELISA in WO 2006/029879 and WO 2005/100402]. To assess complement activation, a CDC assay may be performed [see, e.g., Gazzano-Santoro et al. (1996) J. Immunol. Methods 202:163; Cragg, M. S. et al. (2003) Blood 101:1045-1052; and Cragg, M. S, and M. J. Glennie (2004) Blood 103:2738-2743]. FcRn binding and in vivo clearance/half-life determinations can also be performed using methods known in the art [see, e.g., Petkova, S. B. et al. (2006) Int. Immunol. 18(12):1759-1769].

Antibodies of the present disclosure with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

In certain embodiments, it may be desirable to create cysteine-engineered antibodies, e.g., “thioMAbs,” in which one or more residues of an antibody are substituted with cysteine residues. In particular embodiments, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein. In certain embodiments, any one or more of the following residues may be substituted with cysteine: V205 (Kabat numbering) of the light chain; A118 (EU numbering) of the heavy chain; and S400 (EU numbering) of the heavy-chain Fc region. Cysteine engineered antibodies may be generated as described, for example, in U.S. Pat. No. 7,521,541.

In addition, the techniques used to screen antibodies in order to identify a desirable antibody may influence the properties of the antibody obtained. For example, if an antibody is to be used for binding an antigen in solution, it may be desirable to test solution binding. A variety of different techniques are available for testing interaction between antibodies and antigens to identify particularly desirable antibodies. Such techniques include ELISAs, surface plasmon resonance binding assays (e.g., the Biacore™ binding assay, Biacore AB, Uppsala, Sweden), sandwich assays (e.g., the paramagnetic bead system of IGEN International, Inc., Gaithersburg, Md.), western blots, immunoprecipitation assays, and immunohistochemistry.

In certain embodiments, amino acid sequence variants of the antibodies and/or the binding polypeptides provided herein are contemplated. For example, it may be desirable to improve the binding affinity and/or other biological properties of the antibody and/or binding polypeptide. Amino acid sequence variants of an antibody and/or binding polypeptides may be prepared by introducing appropriate modifications into the nucleotide sequence encoding the antibody and/or binding polypeptide, or by peptide synthesis. Such modifications include, for example, deletions from, and/or insertions into, and/or substitutions of residues within, the amino acid sequences of the antibody and/or binding polypeptide. Any combination of deletion, insertion, and substitution can be made to arrive at the final construct, provided that the final construct possesses the desired characteristics, e.g., target-binding (BMP10, BMP9, BMP6, BMP5, BMP3b, ActRII (ActRIIA and/or ActRIIB), BMPRII, ALK1, and endoglin binding).

Alterations (e.g., substitutions) may be made in HVRs, for example, to improve antibody affinity. Such alterations may be made in HVR “hotspots,” i.e., residues encoded by codons that undergo mutation at high frequency during the somatic maturation process (see, e.g., Chowdhury (2008) Methods Mol. Biol. 207:179-196 (2008)), and/or SDRs (a-CDRs), with the resulting variant VH or VL being tested for binding affinity. Affinity maturation by constructing and reselecting from secondary libraries has been described in the art [see, e.g., Hoogenboom et al., in Methods in Molecular Biology 178:1-37, O'Brien et al., ed., Human Press, Totowa, N.J., (2001)]. In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves HVR-directed approaches, in which several HVR residues (e.g., 4-6 residues at a time) are randomized. HVR residues involved in antigen binding may be specifically identified, e.g., using alanine scanning mutagenesis or modeling. CDR-H3 and CDR-L3 in particular are often targeted.

In certain embodiments, substitutions, insertions, or deletions may occur within one or more HVRs so long as such alterations do not substantially reduce the ability of the antibody to bind to the antigen. For example, conservative alterations (e.g., conservative substitutions as provided herein) that do not substantially reduce binding affinity may be made in HVRs. Such alterations may be outside of HVR “hotspots” or SDRs. In certain embodiments of the variant VH and VL sequences provided above, each HVR either is unaltered, or contains no more than one, two, or three amino acid substitutions.

A useful method for identification of residues or regions of the antibody and/or the binding polypeptide that may be targeted for mutagenesis is called “alanine scanning mutagenesis”, as described by Cunningham and Wells (1989) Science, 244:1081-1085. In this method, a residue or group of target residues (e.g., charged residues such as arg, asp, his, lys, and glu) are identified and replaced by a neutral or negatively charged amino acid (e.g., alanine or polyalanine) to determine whether the interaction of the antibody or binding polypeptide with antigen is affected. Further substitutions may be introduced at the amino acid locations demonstrating functional sensitivity to the initial substitutions. Alternatively, or additionally, a crystal structure of an antigen-antibody complex can be used to identify contact points between the antibody and antigen. Such contact residues and neighboring residues may be targeted or eliminated as candidates for substitution. Variants may be screened to determine whether they contain the desired properties.

Amino-acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include fusion of the N- or C-terminus of the antibody to an enzyme (e.g., for ADEPT) or a polypeptide which increases the serum half-life of the antibody.

In certain embodiments, an antibody and/or binding polypeptide provided herein may be further modified to contain additional non-proteinaceous moieties that are known in the art and readily available. The moieties suitable for derivatization of the antibody and/or binding polypeptide include but are not limited to water-soluble polymers. Non-limiting examples of water-soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody and/or binding polypeptide may vary, and if more than one polymer are attached, they can be the same or different molecules. In general, the number and/or type of polymers used for derivatization can be determined based on considerations including, but not limited to, the particular properties or functions of the antibody and/or binding polypeptide to be improved, whether the antibody derivative and/or binding polypeptide derivative will be used in a therapy under defined conditions.

### 5. Small Molecule Antagonists

In other aspects, the present disclosure relates to a BMP antagonist (inhibitor) that is small molecule, or combination of small molecules. BMP antagonist small molecules may inhibit to one or more ligands [e.g., BMP10, BMP9, BMP6, BMP5, and BMP3b] and/or one or more type I-, type II-, and/or co-receptors (e.g., ActRIIA, ActRIIB, BMPRII, ALK1, and endoglin), and/or one or more downstream signaling components (e.g., Smads 2 and/or 3). In particular, the disclosure provides methods of using an BMP antagonist small molecules, or combination of BMP antagonist small molecules, alone or in combination with one or more additional supportive therapies and/or active agents, to achieve a desired effect in a subject in need thereof (e.g., treat heart failure or one or more complications of heart failure).

In some embodiments, a BMP antagonist is a small molecule antagonist, or combination of small molecule antagonists, that inhibits at least BMP10. In some embodiments, a small molecule antagonist, or combination of small molecule antagonists, that inhibits BMP10 further inhibits one or more ligand [e.g., BMP9, BMP6, BMP5 and BMP3b], ActRIIA, ActRIIB, BMPRII, ALK1, endoglin, and/or one or more Smads (e.g., Smads 2 and 3). In some embodiments, a BMP antagonist is a small molecule antagonist, or combination of small molecule antagonists, that inhibits at least BMP9. In some embodiments, a small molecule antagonist, or combination of small molecule antagonists, that inhibits BMP9 further inhibits one or more ligand [e.g., BMP10, BMP6, BMP5 and BMP3b], ActRIIA, ActRIIB, BMPRII, ALK1, endoglin, and/or one or more Smads (e.g., Smads 2 and 3). In some embodiments, a BMP antagonist is a small molecule antagonist, or combination of small molecule antagonists, that inhibits at least BMP6. In some embodiments, a small molecule antagonist, or combination of small molecule antagonists, that inhibits BMP6 further inhibits one or more ligand [e.g., BMP10, BMP9, BMP5 and BMP3b], ActRIIA, ActRIIB, BMPRII, ALK1, endoglin, and/or one or more Smads (e.g., Smads 2 and 3). In some embodiments, a BMP antagonist is a small molecule antagonist, or combination of small molecule antagonists, that inhibits at least BMP5. In some embodiments, a small molecule antagonist, or combination of small molecule antagonists, that inhibits BMP5 further inhibits one or more ligand [e.g., BMP10, BMP9, BMP5 and BMP3b], ActRIIA, ActRIIB, BMPRII, ALK1, endoglin, and/or one or more Smads (e.g., Smads 2 and 3). In some embodiments, a BMP antagonist is a small molecule antagonist, or combination of small molecule antagonists, that inhibits at least BMP3b. In some embodiments, a small molecule antagonist, or combination of small molecule antagonists, that inhibits BMP3b further inhibits one or more ligand [e.g., BMP10, BMP9, BMP6, and BMP5], ActRIIA, ActRIIB, BMPRII, ALK1, endoglin, and/or one or more Smads (e.g., Smads 2 and 3). In some embodiments, a BMP antagonist is a small molecule antagonist, or combination of small molecule antagonists, that inhibits at least ActRIIA and/or ActRIIB In some embodiments, a small molecule antagonist, or combination of small molecule antagonists, that inhibits ActRIIA and/or ActRIIB further inhibits one or more ligand [e.g., BMP10, BMP9, BMP6, BMP5 and BMP3b], BMPRII, ALK1, endoglin, and/or one or more Smads (e.g., Smads 2 and 3). In some embodiments, a BMP antagonist is a small molecule antagonist, or combination of small molecule antagonists, that inhibits at least BMPRII. In some embodiments, a small molecule antagonist, or combination of small molecule antagonists, that inhibits BMPRII further inhibits one or more ligand [e.g., BMP10, BMP9, BMP6, BMP5 and BMP3b], ActRIIA, ActRIIB, ALK1, endoglin, and/or one or more Smads (e.g., Smads 2 and 3). In some embodiments, a BMP antagonist is a small molecule antagonist, or combination of small molecule antagonists, that inhibits at least ALK1. In some embodiments, a small molecule antagonist, or combination of small molecule antagonists, that inhibits ALK1 further inhibits one or more ligand [e.g., BMP10, BMP9, BMP6, BMP5 and BMP3b], ActRIIA, ActRIIB, BMPRII, endoglin, and/or one or more Smads (e.g., Smads 2 and 3). In some embodiments, a BMP antagonist is a small molecule antagonist, or combination of small molecule antagonists, that inhibits at least endoglin. In some embodiments, a small molecule antagonist, or combination of small molecule antagonists, that inhibits endoglin further inhibits one or more ligand [e.g., BMP10, BMP9, BMP6, BMP5 and BMP3b], ActRIIA, ActRIIB, BMPRII, ALK1, and/or one or more Smads (e.g., Smads 2 and 3). In some embodiments, a BMP antagonist is a small molecule antagonist, or combination of small molecule antagonists, that inhibits at least one or more Smads (e.g., Smads 2 and/or 3). In some embodiments, a small molecule antagonist, or combination of small molecule antagonists, that inhibits Smads further inhibits one or more ligand [e.g., BMP10, BMP9, BMP6, BMP5 and BMP3b], ActRIIA, ActRIIB, BMPRII, ALK1, and/or endoglin.

Small molecule antagonists can be direct or indirect inhibitors. For example, an indirect small molecule antagonist, or combination of small molecule antagonists, may inhibit the expression (e.g., transcription, translation, cellular secretion, or combinations thereof) of at least one or more ligands [e.g., BMP10, BMP9, BMP6, BMP5 and BMP3b], one or more type I-, type II- and/or co-receptors (e.g., ActRIIA, ActRIIB, BMPRII, and ALK), one or more co-receptors (endoglin), and/or one or more downstream signaling components (e.g., Smads 2 and/or 3). Alternatively, a direct small molecule BMP antagonist, or combination of small molecule antagonists, may directly bind to, for example, one or more of one or more ligands [e.g., BMP10, BMP9, BMP6, BMP5 and BMP3b], one or more type I-, type II- and/or co-receptors (e.g., ActRIIA, ActRIIB, BMPRII, and ALK), one or more co-receptors (endoglin), and/or one or more downstream signaling components (e.g., Smads 2 and/or 3). Combinations of one or more indirect and one or more direct small molecule antagonists may be used in accordance with the methods disclosed herein.

Binding organic small molecule antagonists of the present disclosure may be identified and chemically synthesized using known methodology (see, e.g., PCT Publication Nos. WO 00/00823 and WO 00/39585). In general, small molecule antagonists of the disclosure are usually less than about 2000 daltons in size, alternatively less than about 1500, 750, 500, 250 or 200 daltons in size, wherein such organic small molecules that are capable of binding, preferably specifically, to a polypeptide as described herein. Such small molecule antagonists may be identified without undue experimentation using well-known techniques. In this regard, it is noted that techniques for screening organic small molecule libraries for molecules that are capable of binding to a polypeptide target are well-known in the art (see, e.g., international patent publication Nos. WO00/00823 and WO00/39585).

Binding organic small molecules of the present disclosure may be, for example, aldehydes, ketones, oximes, hydrazones, semicarbazones, carbazides, primary amines, secondary amines, tertiary amines, N-substituted hydrazines, hydrazides, alcohols, ethers, thiols, thioethers, disulfides, carboxylic acids, esters, amides, ureas, carbamates, carbonates, ketals, thioketals, acetals, thioacetals, aryl halides, aryl sulfonates, alkyl halides, alkyl sulfonates, aromatic compounds, heterocyclic compounds, anilines, alkenes, alkynes, diols, amino alcohols, oxazolidines, oxazolines, thiazolidines, thiazolines, enamines, sulfonamides, epoxides, aziridines, isocyanates, sulfonyl chlorides, diazo compounds, and acid chlorides.

### 6. Nucleotide Antagonists

In other aspects, the present disclosure relates to a BMP antagonist (inhibitor) that is a polynucleotide, or combination of polynucleotides. BMP antagonist polynucleotides may inhibit to one or more ligands [e.g., BMP10, BMP9, BMP6, BMP5, and BMP3b], one or more type I-, type II- and/or co-receptors (e.g., ActRIIA, ActRIIB, BMPRII, ALK1, and endoglin), and/or one or more downstream signaling components (e.g., Smads 2 and/or 3). In particular, the disclosure provides methods of using a BMP antagonist polynucleotide, or combination of BMP antagonist polynucleotides, alone or in combination with one or more additional supportive therapies and/or active agents, to achieve a desired effect in a subject in need thereof (e.g., treat heart failure or one or more complications of heart failure.

In some embodiments, a BMP antagonist is a polynucleotide antagonist, or combination of polynucleotide antagonists, that inhibits at least BMP10. In some embodiments, a polynucleotide antagonist, or combination of polynucleotide antagonists, that inhibits BMP10 further inhibits one or more ligand [e.g., BMP9, BMP6, BMP5, and BMP3b], one or more type I-, type II- and/or co-receptors (e.g., ActRIIA, ActRIIB, BMPRII, ALK1, and endoglin), and/or one or more downstream signaling components (e.g., Smads 2 and/or 3). In some embodiments, a BMP antagonist is a polynucleotide antagonist, or combination of polynucleotide antagonists, that inhibits at least BMP9. In some embodiments, a polynucleotide antagonist, or combination of polynucleotide antagonists, that inhibits BMP9 further inhibits one or more ligand [e.g., BMP10, BMP6, BMP5, and BMP3b], one or more type I-, type II- and/or co-receptors (e.g., ActRIIA, ActRIIB, BMPRII, ALK1, and endoglin), and/or one or more downstream signaling components (e.g., Smads 2 and/or 3). In some embodiments, a BMP antagonist is a polynucleotide antagonist, or combination of polynucleotide antagonists, that inhibits at least BMP6. In some embodiments, a polynucleotide antagonist, or combination of polynucleotide antagonists, that inhibits BMP6 further inhibits one or more ligand [e.g., BMP10, BMP9, BMP5, and BMP3b], one or more type I-, type II- and/or co-receptors (e.g., ActRIIA, ActRIIB, BMPRII, ALK1, and endoglin), and/or one or more downstream signaling components (e.g., Smads 2 and/or 3). In some embodiments, a BMP antagonist is a polynucleotide antagonist, or combination of polynucleotide antagonists, that inhibits at least BMP5. In some embodiments, a polynucleotide antagonist, or combination of polynucleotide antagonists, that inhibits BMP5 further inhibits one or more ligand [e.g., BMP10, BMP9, BMP6, and BMP3b], one or more type I-, type II- and/or co-receptors (e.g., ActRIIA, ActRIIB, BMPRII, ALK1, and endoglin), and/or one or more downstream signaling components (e.g., Smads 2 and/or 3). In some embodiments, a BMP antagonist is a polynucleotide antagonist, or combination of polynucleotide antagonists, that inhibits at least BMP3b. In some embodiments, a polynucleotide antagonist, or combination of polynucleotide antagonists, that inhibits BMP3b further inhibits one or more ligand [e.g., BMP10, BMP9, BMP6, and BMP5], one or more type I-, type II- and/or co-receptors (e.g., ActRIIA, ActRIIB, BMPRII, ALK1, and endoglin), and/or one or more downstream signaling components (e.g., Smads 2 and/or 3). In some embodiments, a BMP antagonist is a polynucleotide antagonist, or combination of polynucleotide antagonists, that inhibits at least ActRIIA and/or ActRIIB In some embodiments, a polynucleotide antagonist, or combination of polynucleotide antagonists, that inhibits ActRIIA and/or ActRIIB further inhibits one or more ligand [e.g., BMP10, BMP9, BMP6, BMP5, and BMP3b], one or more type I-, type II- and/or co-receptors (e.g., BMPRII, ALK1, and endoglin), and/or one or more downstream signaling components (e.g., Smads 2 and/or 3). In some embodiments, a BMP antagonist is a polynucleotide antagonist, or combination of polynucleotide antagonists, that inhibits at least BMPRII. In some embodiments, a polynucleotide antagonist, or combination of polynucleotide antagonists, that inhibits BMPRII further inhibits one or more ligand [e.g., BMP10, BMP9, BMP6, BMP5, and BMP3b], one or more type I-, type II- and/or co-receptors (e.g., ActRIIA, ActRIIB, ALK1, and endoglin), and/or one or more downstream signaling components (e.g., Smads 2 and/or 3). In some embodiments, a BMP antagonist is a polynucleotide antagonist, or combination of polynucleotide antagonists, that inhibits at least ALK1. In some embodiments, a polynucleotide antagonist, or combination of polynucleotide antagonists, that inhibits ALK1 further inhibits one or more ligand [e.g., BMP10, BMP9, BMP6, BMP5, and BMP3b], one or more type I-, type II- and/or co-receptors (e.g., ActRIIA, ActRIIB, BMPRII, and endoglin), and/or one or more downstream signaling components (e.g., Smads 2 and/or 3). In some embodiments, a BMP antagonist is a polynucleotide antagonist, or combination of polynucleotide antagonists, that inhibits at least endoglin. In some embodiments, a polynucleotide antagonist, or combination of polynucleotide antagonists, that inhibits endoglin further inhibits one or more ligand [e.g., BMP10, BMP9, BMP6, BMP5, and BMP3b], one or more type I-, type II- and/or co-receptors (e.g., ActRIIA, ActRIIB, BMPRII, and ALK1), and/or one or more downstream signaling components (e.g., Smads 2 and/or 3). In some embodiments, a BMP antagonist is a polynucleotide antagonist, or combination of polynucleotide antagonists, that inhibits at least one or more Smads (e.g., Smads 2 and/or 3. In some embodiments, a polynucleotide antagonist, or combination of polynucleotide antagonists, that inhibits one or more Smads further inhibits one or more ligand [e.g., BMP10, BMP9, BMP6, BMP5, and BMP3b] and/or one or more type I-, type II- and/or co-receptors (e.g., ActRIIA, ActRIIB, BMPRII, ALK1, and endoglin).

The polynucleotide antagonists of the present disclosure may be an antisense nucleic acid, an RNAi molecule [e.g., small interfering RNA (siRNA), small-hairpin RNA (shRNA), microRNA (miRNA)], an aptamer and/or a ribozyme. The nucleic acid and amino acid sequences of human BMP10, BMP9, BMP6, BMP5, BMP3b, ActRIIA, ActRIIB, BMPRII, ALK1, endoglin, and Smads (e.g., Smads 2 and 3) are known in the art and thus polynucleotide antagonists for use in accordance with methods of the present disclosure may be routinely made by the skilled artisan based on the knowledge in the art and teachings provided herein.

For example, antisense technology can be used to control gene expression through antisense DNA or RNA, or through triple-helix formation. Antisense techniques are discussed, for example, in Okano (1991) J. Neurochem. 56:560; Oligodeoxynucleotides as Antisense Inhibitors of Gene Expression, CRC Press, Boca Raton, Fla. (1988). Triple helix formation is discussed in, for instance, Cooney et al. (1988) Science 241:456; and Dervan et al., (1991) Science 251:1300. The methods are based on binding of a polynucleotide to a complementary DNA or RNA. In some embodiments, the antisense nucleic acids comprise a single-stranded RNA or DNA sequence that is complementary to at least a portion of an RNA transcript of a desired gene. However, absolute complementarity, although preferred, is not required.

A sequence “complementary to at least a portion of an RNA,” referred to herein, means a sequence having sufficient complementarity to be able to hybridize with the RNA, forming a stable duplex; in the case of double-stranded antisense nucleic acids of a gene disclosed herein, a single strand of the duplex DNA may thus be tested, or triplex formation may be assayed. The ability to hybridize will depend on both the degree of complementarity and the length of the antisense nucleic acid. Generally, the larger the hybridizing nucleic acid, the more base mismatches with an RNA it may contain and still form a stable duplex (or triplex as the case may be). One skilled in the art can ascertain a tolerable degree of mismatch by use of standard procedures to determine the melting point of the hybridized complex.

Polynucleotides that are complementary to the 5′ end of the message, for example, the 5′-untranslated sequence up to and including the AUG initiation codon, should work most efficiently at inhibiting translation. However, sequences complementary to the 3′-untranslated sequences of mRNAs have been shown to be effective at inhibiting translation of mRNAs as well [see, e.g., Wagner, R., (1994) Nature 372:333-335]. Thus, oligonucleotides complementary to either the 5′- or 3′-untranslated, noncoding regions of a gene of the disclosure, could be used in an antisense approach to inhibit translation of an endogenous mRNA. Polynucleotides complementary to the 5′-untranslated region of the mRNA should include the complement of the AUG start codon. Antisense polynucleotides complementary to mRNA coding regions are less efficient inhibitors of translation but could be used in accordance with the methods of the present disclosure. Whether designed to hybridize to the 5′-untranslated, 3′-untranslated, or coding regions of an mRNA of the disclosure, antisense nucleic acids should be at least six nucleotides in length, and are preferably oligonucleotides ranging from 6 to about 50 nucleotides in length. In specific aspects, the oligonucleotide is at least 10 nucleotides, at least 17 nucleotides, at least 25 nucleotides, or at least 50 nucleotides.

In one embodiment, the antisense nucleic acid of the present disclosure is produced intracellularly by transcription from an exogenous sequence. For example, a vector or a portion thereof, is transcribed, producing an antisense nucleic acid (RNA) of a gene of the disclosure. Such a vector would contain a sequence encoding the desired antisense nucleic acid. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others known in the art, used for replication and expression in vertebrate cells. Expression of the sequence encoding desired genes of the instant disclosure, or fragments thereof, can be by any promoter known in the art to act in vertebrate, preferably human cells. Such promoters can be inducible or constitutive. Such promoters include, but are not limited to, the SV40 early promoter region [see, e.g., Benoist and Chambon (1981) Nature 29:304-310], the promoter contained in the 3′ long terminal repeat of Rous sarcoma virus [see, e.g., Yamamoto et al. (1980) Cell 22:787-797], the herpes thymidine promoter [see, e.g., Wagner et al. (1981) Proc. Natl. Acad. Sci. U.S.A. 78:1441-1445], and the regulatory sequences of the metallothionein gene [see, e.g., Brinster, et al. (1982) Nature 296:39-42].

In some embodiments, the polynucleotide antagonists are interfering RNA or RNAi molecules that target the expression of one or more genes. RNAi refers to the expression of an RNA which interferes with the expression of the targeted mRNA. Specifically, RNAi silences a targeted gene via interacting with the specific mRNA through a siRNA (small interfering RNA). The ds RNA complex is then targeted for degradation by the cell. An siRNA molecule is a double-stranded RNA duplex of 10 to 50 nucleotides in length, which interferes with the expression of a target gene which is sufficiently complementary (e.g. at least 80% identity to the gene). In some embodiments, the siRNA molecule comprises a nucleotide sequence that is at least 85, 90, 95, 96, 97, 98, 99, or 100% identical to the nucleotide sequence of the target gene.

Additional RNAi molecules include short-hairpin RNA (shRNA); also short-interfering hairpin and microRNA (miRNA). The shRNA molecule contains sense and antisense sequences from a target gene connected by a loop. The shRNA is transported from the nucleus into the cytoplasm, and it is degraded along with the mRNA. Pol III or U6 promoters can be used to express RNAs for RNAi. Paddison et al. [Genes & Dev. (2002) 16:948-958, 2002] have used small RNA molecules folded into hairpins as a means to effect RNAi. Accordingly, such short hairpin RNA (shRNA) molecules are also advantageously used in the methods described herein. The length of the stem and loop of functional shRNAs varies; stem lengths can range anywhere from about 25 to about 30 nt, and loop size can range between 4 to about 25 nt without affecting silencing activity. While not wishing to be bound by any particular theory, it is believed that these shRNAs resemble the double-stranded RNA (dsRNA) products of the DICER RNase and, in any event, have the same capacity for inhibiting expression of a specific gene. The shRNA can be expressed from a lentiviral vector. An miRNA is a single-stranded RNA of about 10 to 70 nucleotides in length that are initially transcribed as pre-miRNA characterized by a “stem-loop” structure and which are subsequently processed into mature miRNA after further processing through the RISC.

Molecules that mediate RNAi, including without limitation siRNA, can be produced in vitro by chemical synthesis (Hohjoh, FEBS Lett 521:195-199, 2002), hydrolysis of dsRNA (Yang et al., Proc Natl Acad Sci USA 99:9942-9947, 2002), by in vitro transcription with T7 RNA polymerase (Donzeet et al., Nucleic Acids Res 30:e46, 2002; Yu et al., Proc Natl Acad Sci USA 99:6047-6052, 2002), and by hydrolysis of double-stranded RNA using a nuclease such as E. coli RNase III (Yang et al., Proc Natl Acad Sci USA 99:9942-9947, 2002).

According to another aspect, the disclosure provides polynucleotide antagonists including but not limited to, a decoy DNA, a double-stranded DNA, a single-stranded DNA, a complexed DNA, an encapsulated DNA, a viral DNA, a plasmid DNA, a naked RNA, an encapsulated RNA, a viral RNA, a double-stranded RNA, a molecule capable of generating RNA interference, or combinations thereof.

In some embodiments, the polynucleotide antagonists of the disclosure are aptamers. Aptamers are nucleic acid molecules, including double-stranded DNA and single-stranded RNA molecules, which bind to and form tertiary structures that specifically bind to a target molecule, such as a BMP10, BMP9, BMP6, BMP5, BMP3b, ActRIIA, ActRIIB, BMPRII, ALK1, endoglin, and Smads (e.g., Smads 2 and 3). The generation and therapeutic use of aptamers are well established in the art. See, e.g., U.S. Pat. No. 5,475,096. Additional information on aptamers can be found in U.S. Patent Application Publication No. 20060148748. Nucleic acid aptamers are selected using methods known in the art, for example via the Systematic Evolution of Ligands by Exponential Enrichment (SELEX) process. SELEX is a method for the in vitro evolution of nucleic acid molecules with highly specific binding to target molecules as described in, e.g., U.S. Pat. Nos. 5,475,096, 5,580,737, 5,567,588, 5,707,796, 5,763,177, 6,011,577, and 6,699,843. Another screening method to identify aptamers is described in U.S. Pat. No. 5,270,163. The SELEX process is based on the capacity of nucleic acids for forming a variety of two- and three-dimensional structures, as well as the chemical versatility available within the nucleotide monomers to act as ligands (form specific binding pairs) with virtually any chemical compound, whether monomeric or polymeric, including other nucleic acid molecules and polypeptides. Molecules of any size or composition can serve as targets. The SELEX method involves selection from a mixture of candidate oligonucleotides and step-wise iterations of binding, partitioning and amplification, using the same general selection scheme, to achieve desired binding affinity and selectivity. Starting from a mixture of nucleic acids, which can comprise a segment of randomized sequence, the SELEX method includes steps of contacting the mixture with the target under conditions favorable for binding; partitioning unbound nucleic acids from those nucleic acids which have bound specifically to target molecules; dissociating the nucleic acid-target complexes; amplifying the nucleic acids dissociated from the nucleic acid-target complexes to yield a ligand enriched mixture of nucleic acids. The steps of binding, partitioning, dissociating and amplifying are repeated through as many cycles as desired to yield highly specific high affinity nucleic acid ligands to the target molecule.

Typically, such binding molecules are separately administered to the animal [see, e.g., O'Connor (1991) J. Neurochem. 56:560], but such binding molecules can also be expressed in vivo from polynucleotides taken up by a host cell and expressed in vivo [see, e.g., Oligodeoxynucleotides as Antisense Inhibitors of Gene Expression, CRC Press, Boca Raton, Fla. (1988)].

### 7. Screening Assays

In certain aspects, the present disclosure relates to the use of BMP10 propeptides to identify compounds (agents) which are BMP antagonists. Compounds identified through this screening can be tested to assess their ability to modulate cardiac tissue, to assess their ability to modulate tissue changes in vivo or in vitro. These compounds can be tested, for example, in animal models.

There are numerous approaches to screening for therapeutic agents for modulating tissue growth by targeting TGFβ superfamily ligand signaling (e.g., SMAD signaling). In certain embodiments, high-throughput screening of compounds can be carried out to identify agents that perturb TGFβ superfamily receptor-mediated effects on a selected cell line. In certain embodiments, the assay is carried out to screen and identify compounds that specifically inhibit or reduce binding of a BMP10 propeptides to a binding partner including for example, BMP10, BMP9, BMP6, BMP5, and BMP3b. Alternatively, the assay can be used to identify compounds that enhance binding of a BMP10 propeptides to a binding partner such as a ligand. In a further embodiment, the compounds can be identified by their ability to interact with a BMP10 propeptides.

A variety of assay formats will suffice and, in light of the present disclosure, those not expressly described herein will nevertheless be comprehended by one of ordinary skill in the art. As described herein, the test compounds (agents) of the invention may be created by any combinatorial chemical method. Alternatively, the subject compounds may be naturally occurring biomolecules synthesized in vivo or in vitro. Compounds (agents) to be tested for their ability to act as modulators of tissue growth can be produced, for example, by bacteria, yeast, plants or other organisms (e.g., natural products), produced chemically (e.g., small molecules, including peptidomimetics), or produced recombinantly. Test compounds contemplated by the present invention include non-peptidyl organic molecules, peptides, polypeptides, peptidomimetics, sugars, hormones, and nucleic acid molecules. In certain embodiments, the test agent is a small organic molecule having a molecular weight of less than about 2,000 Daltons.

The test compounds of the disclosure can be provided as single, discrete entities, or provided in libraries of greater complexity, such as made by combinatorial chemistry. These libraries can comprise, for example, alcohols, alkyl halides, amines, amides, esters, aldehydes, ethers and other classes of organic compounds. Presentation of test compounds to the test system can be in either an isolated form or as mixtures of compounds, especially in initial screening steps. Optionally, the compounds may be optionally derivatized with other compounds and have derivatizing groups that facilitate isolation of the compounds. Non-limiting examples of derivatizing groups include biotin, fluorescein, digoxygenin, green fluorescent protein, isotopes, polyhistidine, magnetic beads, glutathione S-transferase (GST), photoactivatible crosslinkers or any combinations thereof.

In many drug-screening programs which test libraries of compounds and natural extracts, high-throughput assays are desirable in order to maximize the number of compounds surveyed in a given period of time. Assays which are performed in cell-free systems, such as may be derived with purified or semi-purified proteins, are often preferred as “primary” screens in that they can be generated to permit rapid development and relatively easy detection of an alteration in a molecular target which is mediated by a test compound. Moreover, the effects of cellular toxicity or bioavailability of the test compound can be generally ignored in the in vitro system, the assay instead being focused primarily on the effect of the drug on the molecular target as may be manifest in an alteration of binding affinity between a BMP10 propeptides to a binding partner including for example, BMP10, BMP9, BMP6, BMP5, and BMP3b.

Merely to illustrate, in an exemplary screening assay of the present disclosure, the compound of interest is contacted with an isolated and purified BMP10 propeptide which is ordinarily capable of binding to a TGF-beta superfamily ligand, as appropriate for the intention of the assay. To the mixture of the compound and BMP10 propeptide is then added to a composition containing the appropriate ligand (e.g., BMP10, BMP9, BMP6, BMP5, and BMP3b). Detection and quantification of BMP10 propeptide-superfamily ligand complexes provides a means for determining the compound's efficacy at inhibiting (or potentiating) complex formation between the BMP10 propeptide and its binding protein. The efficacy of the compound can be assessed by generating dose-response curves from data obtained using various concentrations of the test compound. Moreover, a control assay can also be performed to provide a baseline for comparison. For example, in a control assay, isolated and purified ligand is added to a composition containing the BMP10 propeptide, and the formation of BMP10 propeptide-ligand complex is quantitated in the absence of the test compound. It will be understood that, in general, the order in which the reactants may be admixed can be varied, and can be admixed simultaneously. Moreover, in place of purified proteins, cellular extracts and lysates may be used to render a suitable cell-free assay system.

Binding of a BMP10 propeptide to another protein may be detected by a variety of techniques. For instance, modulation of the formation of complexes can be quantitated using, for example, detectably labeled proteins such as radiolabeled (e.g. ³²P, ³⁵S, ¹⁴C or ³H), fluorescently labeled (e.g., FITC), or enzymatically labeled BMP10 propeptide and/or a binding protein, by immunoassay, or by chromatographic detection.

In certain embodiments, the present disclosure contemplates the use of fluorescence polarization assays and fluorescence resonance energy transfer (FRET) assays in measuring, either directly or indirectly, the degree of interaction between a BMP10 propeptide and a binding protein. Further, other modes of detection, such as those based on optical waveguides (PCT Publication WO 96/26432 and U.S. Pat. No. 5,677,196), surface plasmon resonance (SPR), surface charge sensors, and surface force sensors, are compatible with many embodiments of the disclosure.

Moreover, the present disclosure contemplates the use of an interaction trap assay, also known as the “two-hybrid assay,” for identifying agents that disrupt or potentiate interaction between a BMP10 propeptide and a binding partner. See, e.g., U.S. Pat. No. 5,283,317; Zervos et al. (1993) Cell 72:223-232; Madura et al. (1993) J Biol Chem 268:12046-12054; Bartel et al. (1993) Biotechniques 14:920-924; and Iwabuchi et al. (1993) Oncogene 8:1693-1696). In a specific embodiment, the present disclosure contemplates the use of reverse two-hybrid systems to identify compounds (e.g., small molecules or peptides) that dissociate interactions between a BMP10 propeptide and a binding protein [Vidal and Legrain, (1999) Nucleic Acids Res 27:919-29; Vidal and Legrain, (1999) Trends Biotechnol 17:374-81; and U.S. Pat. Nos. 5,525,490; 5,955,280; and 5,965,368].

In certain embodiments, the subject compounds are identified by their ability to interact with a BMP10 propeptide. The interaction between the compound and the BMP10 propeptide may be covalent or non-covalent. For example, such interaction can be identified at the protein level using in vitro biochemical methods, including photo-crosslinking, radiolabeled ligand binding, and affinity chromatography [Jakoby W B et al. (1974) Methods in Enzymology 46:1]. In certain cases, the compounds may be screened in a mechanism-based assay, such as an assay to detect compounds which bind to a BMP10 propeptide. This may include a solid-phase or fluid-phase binding event. Alternatively, the gene encoding a BMP10 propeptide can be transfected with a reporter system (e.g., β-galactosidase, luciferase, or green fluorescent protein) into a cell and screened against the library preferably by high-throughput screening or with individual members of the library. Other mechanism-based binding assays may be used; for example, binding assays which detect changes in free energy. Binding assays can be performed with the target fixed to a well, bead or chip or captured by an immobilized antibody or resolved by capillary electrophoresis. The bound compounds may be detected usually using colorimetric endpoints or fluorescence or surface plasmon resonance.

### 8. Exemplary Therapeutic Uses

As described herein, it has discovered that a BMP antagonist a surprising effect on treating and preventing various compilations of heart failure. For example, it was shown that a soluble BMP10 propeptide (BMP10pro) polypeptide can be used to prevent or reduce the severity of cardiac hypertrophy, cardiac remodeling, and cardiac fibrosis as well as improve cardiac function in a transverse aortic constriction (TAC) heart failure model. Moreover, BMP10pro treatment increased survival time of heart failure patients. Similar benefical effects of BMP10pro treatment were observed in a myocardial infarct (MI) heart disease model. Moreover, a soluble endoglin polypeptide, which binds to BMP10 and BMP9, also displayed positive effects in both TAC and MI heart failure models. Accordingly, the disclosure provides, in part, methods of using BMP antagonists, alone or in combination with one or more additional supportive therapies and/or additional active agents, to treat, prevent, or reduce the severity of heart failure, particularly treating, preventing, or reducing the severity of one or more complications of a heart failure (e.g., cardiac hypertrophy, cardiac remodeling, and cardiac fibrosis) as well as improving cardiac function and increasing survival time of heart failure patients.

As used herein, a therapeutic that “prevents” a disorder or condition refers to a compound that, in a statistical sample, reduces the occurrence of the disorder or condition in the treated sample relative to an untreated control sample or delays the onset of the disorder or condition relative to the untreated control sample. The term “treating” as used herein includes amelioration or elimination of the condition once it has been established. In either case, prevention or treatment may be discerned in the diagnosis provided by a physician or other health care provider and the intended result of administration of the therapeutic agent.

In general, treatment or prevention of a disease or condition as described in the present disclosure is achieved by administering one or more BMP antagonists in an effective amount. An effective amount of an agent refers to an amount effective, at dosages and for periods of time necessary, to achieve the desired therapeutic or prophylactic result. A therapeutically effective amount of an agent of the present disclosure may vary according to factors such as the disease state, age, sex, and weight of the individual, and the ability of the agent to elicit a desired response in the individual. A prophylactically effective amount refers to an amount effective, at dosages and for periods of time necessary, to achieve the desired prophylactic result.

Heart failure is a clinical syndrome defined by typical symptoms and signs resulting from certain structural or functional abnormality of the heart (ESC Guidelines for the diagnosis and treatment of acute and chronic heart failure. McMurray J J et al. European Heart Journal 2012, 14(8):803-69; 2013 ACCF/AHA Guideline for the Management of Heart Failure, Yanzy C W et al. Circulation 2013, 128, e240-e327). For example, cardiac abnormalities may impair the ability to fill or eject blood, and/or lead to failure to deliver sufficient oxygen to meet the requirements of the metabolizing tissues, despite normal filling pressures, or only at the expense of increased filling pressures. As used herein, the term heart failure encompasses a variety cardiovascular conditions which include, but are not limited to, heart failure due to left ventricular dysfunction, heart failure with normal ejection fraction, heart failure due to aortic stenosis, acute heart failure, chronic heart failure, congestive heart failure, congenital heart failure, compensated heart failure, decompensated heart failure, diastolic heart failure, systolic heart failure, right-side heart (ventricle) failure, left-side heart (ventricle) failure, biventricular heart failure, forward heart failure, backward heart failure, high output heart failure, low output heart failure. Also heart failure includes heart conditions relating to fluid build-up in the heart, such as myocardial edema.

In general, clinical manifestations of heart failure include, for example, dyspnea (shortness of breath), orthopnea, paroxysmal nocturnal dyspnea, and fatigue (which may limit exercise tolerance), fluid retention (which may lead to, for example, pulmonary congestion and peripheral edema), angina, hypertension, arrhythmia, ventricular arrhythmias, cardiomyopathy, cardiac hypertrophy, cardiac asthma, nocturia, ascities, congestive hepatopathy, coagulopathy, reduced renal blood flow, renal insufficiency, myocardial infarction, and stroke.

Although the phrase “congestive heart failure” is often used to describe all types of heart failure, including the above listed types, congestive heart failure is more accurately descriptive of a symptom of heart failure relating to pulmonary congestion or fluid buildup in the lungs. This congestion is more commonly symptom of systolic and left-sided heart failure. As the efficiency of the pulmonary system declines, increased blood volume near the input side of the heart changes the pressure at the alveolar arterial interface, an interface between the lung capillaries and the alveolar space of the lungs. The change in pressure at the interface causes blood plasma to push out into the alveolar space in the lungs. Dyspnea and general fatigue are typical perceived manifestations of congestive heart failure.

There are many different ways to categorize heart failure. For example, heart failure may be characterized based on the side of the heart involved (left heart failure versus right heart failure). Right heart failure compromises pulmonary flow to the lungs. Left heart failure compromises aortic flow to the body and brain. Mixed presentations are common; left heart failure often leads to right heart failure in the longer term. Heart failure also may be classified on whether the abnormality is due to insufficient contraction (systolic dysfunction; systolic heart failure), or due to insufficient relaxation of the heart (diastolic dysfunction; diastolic heart failure), or to both. In addition, heart failure may be classified on whether the problem is primarily increased venous back pressure (preload), or failure to supply adequate arterial perfusion (afterload). Heart failure may be classified on whether the abnormality is due to low cardiac output with high systemic vascular resistance or high cardiac output with low vascular resistance (low-output heart failure vs. high-output heart failure). Also, heart failure may be classified based on the degree of coexisting illness, for example, heart failure/systemic hypertension, heart failure/pulmonary hypertension, heart failure/diabetes, and heart failure/kidney failure.

Furthermore, heart failure may be classified based on the degree of functional impairment conferred by the cardiac abnormality. Functional classification generally relies on the New York Heart Association (NYHA) functional classification. The classes (I-IV) are: class I: no limitation is experienced in any activities; there are no symptoms from ordinary activities; class II: slight, mild limitation of activity; the patient is comfortable at rest or with mild exertion; class III: marked limitation of any activity; the patient is comfortable only at rest; and class IV: any physical activity brings on discomfort and symptoms occur at rest. This score documents the severity of symptoms and can be used to assess response to treatment.

In its 2001 guidelines the American College of Cardiology/American Heart Association (ACC) working group introduced four stages of heart failure [see, e.g., Hunt, S., “ACC/AHA 2005 Guideline Update for the Diagnosis and Management of Chronic Heart Failure in the Adult: A Report of the American College of Cardiology/American Heart Association Task Force on Practice Guidelines (Writing Committee to Update the 2001 Guidelines for the Evaluation and Management of Heart Failure),” J. Am, Coll. Cardiol, 46:e1-e82 (2005]. The first stage, Stage A, is a subject at high risk for heart failure but without structural heart disease or symptoms of heart failure (for example, these are patients with hypertension, atherosclerotic disease, diabetes, obesity, metabolic syndrome or patients using cardiotoxins). The second stage, Stage B, is a subject having structural heart disease but without signs or symptoms of heart failure (for example, these are patients who have previously had a myocardial infarction, exhibit cardiac remodeling including hypertrophy and low ejection fraction, and patients with asymptomatic valvular disease). The third stage, Stage C, is a subject having structural heart disease with prior or current symptoms of heart failure (for example, these are patients who have known structural heart disease and exhibit shortness of breath and fatigue and have reduced exercise tolerance). The fourth and final stage, Stage D, is refractory heart failure requiring specialized interventions (for example, patients who have marked symptoms at rest despite maximal medical therapy (namely, those who are recurrently hospitalized or cannot be safely discharged from the hospital without specialized interventions). The ACC staging system is useful in that Stage A encompasses “pre-heart failure”—a stage where intervention with treatment can presumably prevent progression to overt symptoms. ACC Stage A does not have a corresponding NYHA class. ACC Stage B would correspond to NYHA Class I. ACC Stage C corresponds to NYHA Class II and III, while ACC Stage D overlaps with NYHA Class IV.

Cardiac remodeling, which usually precedes clinical signs of heart failure, refers to the molecular, cellular and/or interstitial changes manifested clinically as changes in size, shape and function of the heart generally resulting from cardiac load or injury (Cohn J N et al. JACC 2000. 35(3):569-82). Triggers for cardiac remodeling include, for example, myocardial infarction, hypertension, wall stress, inflammation, pressure overload, and volume overload. Alterations in myocardial structure can occur as quickly as within a few hours of injury and may progress over months and years. While initially beneficial, these changes can impair myocardial function to the point of chronic intractable heart failure over time (months to years). Hallmarks of cardiac remodeling include, for example, chamber dilation, increase in ventricular sphericity, and development of interstitial and perivascular fibrosis. Increased sphericity is positively associated with mitral regurgitation. Ventricular dilation mainly results from cardiomyocyte hypertrophy and lengthening and to a lesser extent from increases in the ventricular mass.

In some embodiments, BMP antagonists of the disclosure may be used to treat, prevent, or reduce the progression of cardiac remodeling. For example, BMP antagonists may be used to maintain myocardial structure or decrease alterations in myocardial structure of the heart in a subject. Progression of cardiac remodeling can be assessed by comparing the alterations in myocardial structure of the heart over a period of time between two groups of subjects, in which a first group (the treatment group) is treated by the methods of the present invention, and a second group (the placebo group) is treated by using a placebo in replacement or in lieu of the treatment by the methods of the present invention. If the alterations in myocardial structure of the heart in the subjects of the treatment group are less than the alterations in myocardial structure of the heart in the subjects of the placebo group, then a determination is made that there has been a reduction in the progression of cardiac remodeling. Methods for determining disease progression or development, such as cardiac remodeling, can be assessed using well known methods including, for example, physical examination, 2-dimensional echocardiogram coupled with Doppler flow studies, ultrasound, MRI, computerized tomography, cardiac catheterization, radionuclide imaging (such as radionuclide ventriculography) as well as any combinations thereof.

In general, cardiac remodeling and heart failure result from disorders and conditions that cause persistent increase in cardiac workload or injury. Disorders and conditions leading to heart failure include, for example, loss of viable myocardium after myocardial infarction, coronary artery disease, hypertension, cardiomyopathies (e.g., dilated cardiomyopathy, cardiomyopathy from infections or alcohol/drug abuse, etc.), heart valve disease and dysfunction including, for example, aortic valve diseases (e.g., aortic valve insufficiency, aortic valve regurgitation, and aortic stenosis (aortic valve stenosis)), pulmonary disorders (e.g., pulmonary hypertension), congenital heart defects, acute ischemic injury, reperfusion injury, pericardium disorders and abnormalities, myocardium disorders, great vessels disorders, endocardium disorders, atrial fibrillation, impairment of left ventricular myocardial function, impairment of right ventricular myocardial function, cardiac arrhythmias, thyroid disease, kidney disease, diabetes, weakening of the heart muscle which leave it unable to pump enough blood, thyroid disease, neurohormonal imbalances, viral infections, and anemia. As such disorders and conditions may lead to cardiac remodeling and/or heart failure, subjects having, or suspected of having, one or more of these conditions are preferred subjects for treatment with one or more BMP antagonists, optionally in combination with one or more additional active agents or supportive therapies for treating cardiac remodeling and/or heart failure, in accordance with the present invention. In some embodiments, subjects with signs of cardiac remodeling (e.g., myocardial hypertrophy and ventricular dilation) or with overt heart failure, even when the underlying etiology cannot be detected, are also suitable for treatment in accordance with the present disclosure as preventing further cardiac remodeling or treating existing cardiac remodeling or reducing cardiac remodeling would be beneficial in these subjects. In some embodiments, subjects with risk factors for cardiac remodeling and/or heart failure development (e.g. subjects with those conditions that may lead to cardiac remodeling and/or chronic heart failure described herein) are also suitable for treatment in accordance with the present disclosure.

In general, hypertension or high blood pressure refers to a resting blood pressure, as measured with, for example, a sphygmomanometer, of greater than 120 mmHg (systolic)/80 mmHg (diastolic). Blood pressure between 121-139/81-89 is considered prehypertension and above this level (140/90 mm Hg or higher) is considered high (hypertension). Unless otherwise indicated, both prehypertension and hypertension blood pressure are included in the meaning of “hypertension” as used herein. For example, resting blood pressures of 135 mmHg/87 or of 140 mmHg/90 mmHg are intended to be within the scope of the term “hypertension” even though the 135/87 is generally considered within a prehypertensive category. Blood pressures of 145 mm Hg/90 mmHg, 140 mmHg/95 mmHg, and 142 mmHg/93 mmHg are further examples of high blood pressures. It will be appreciated that blood pressure normally varies throughout the day. It can even vary slightly with each heartbeat. Normally, it increases during activity and decreases at rest. It's often higher in cold weather and can rise when under stress. More accurate blood pressure readings can be obtained by daily monitoring blood pressure, where the blood pressure reading is taken at the same time each day to minimize the effect that external factors. Several readings over time may be needed to determine whether blood pressure is high. In general chronic hypertension refers to a subject which exhibits hypertension either continuously or intermittently for an extended period of time, such as, but not limited to at least one week, at least two weeks, at least three weeks, at least four weeks, at least two months, at least six months, at least one year, at least two years, at least three years, at least four years, at least five years, at least 10 years, etc.

In general, cardiac arrhythmia refers to a condition where the muscle contraction of the heart becomes irregular. An unusually fast rhythm (e.g., more than 100 beats per minute) is called tachycardia. An unusually slow rhythm (e.g., fewer than 60 beats per minute) is called bradycardia.

In general, cardiac hypertrophy refers to cardiac enlargement, a condition characterized by an increase in the size of heart and the individual cardiac muscle cells, particularly ventricular muscle cells, and an increase in the size of the inside cavity of a chamber of the heart.

Ejection fraction is the percentage of blood pumped out of the left ventricle with each heartbeat. Ejection fraction may be measured, for example, during an echocardiogram. Ejection fraction is an important measurement of how well a heart is pumping and can be used to classify heart failure and to guide treatment. Heart failure can be classified as heart failure with preserved ejection fraction (also referred to as diastolic heart failure) or as heart failure with reduced ejection fraction (also referred to as systolic heart failure). A recent study demonstrated that the prevalence of heart failure with preserved ejection fraction increased over a 15-year period, with no marked improvement in the mortality rates. If these trends continue, heart failure with preserved ejection fraction may become the most common form of heart failure, demonstrating a growing public health problem (Owan et al., 2006, N Engl J Med; 355(3):251-9).

In some embodiments, BMP antagonists of the disclosure may be used to reduce the incidences of non-fatal or fatal cardiovascular events (e.g., myocardial infarction, stroke, angina, arrhythmias, fluid retention, and progression of heart failure). As used herein, reducing the incidences of cardiovascular events refers to maintaining or reducing the number of cardiovascular events experienced by a subject during or over the course of a period of time. A reduction in the incidence of cardiovascular events can be assessed or determined by comparing the incidences of cardiovascular events over or during the course of a period of time between two groups of subjects, in which a first group (the treatment group) is treated by the methods of the present invention, and a second group (the placebo group) is treated by using a placebo (namely, dummy pills) in replacement or in lieu of the treatment by the methods of the present invention. If the number of cardiovascular events for the treatment group is less than the number of the cardiovascular events for the placebo group, then a determination is made that there was or has been a reduction in the incidences of cardiovascular events. Alternatively, a reduction in the incidence of cardiovascular events can be assessed or determined by determining a baseline number of cardiovascular events for a subject population at a first period in time and then measuring the number of cardiovascular events for a subject population at a second, later period in time. If the number of cardiovascular events for the subject population at the second, later period in time is the same as or less then the number of cardiovascular events for the subject population at the first period in time, then a determination is made that there has been a reduction in the incidences of cardiovascular events for said subject population.

In some embodiments, BMP antagonists of the disclosure may be used to reduce incidence of hospitalizations for heart failure. As used herein, reducing the incidences of hospitalizations for heart failure refers to maintaining or reducing the number of hospitalizations for heart failure experienced by a subject during or over the course of a period of time. A reduction in the incidence of hospitalizations for heart failure can be assessed or determined by comparing the incidences of hospitalizations for heart failure over or during the course of a period of time between two groups of subjects, in which a first group (the treatment group) is treated by the methods of the present invention, and a second group (the placebo group) is treated by using a placebo (namely, dummy pills) in replacement or in lieu of the treatment by the methods of the present invention. If the number of hospitalizations for heart failure for the treatment group is less than the number of the hospitalizations for heart failure for the placebo group, then a determination is made that there was or has been a reduction in the incidences of hospitalizations for heart failure. Alternatively, a reduction in the incidence of hospitalizations for heart failure can be assessed or determined by determining a baseline number of hospitalizations for heart failure for a subject population at a first period in time and then measuring the number of hospitalizations for heart failure for a subject population at a second, later period in time. If the number of hospitalizations for heart failure for the subject population at the second, later period in time is the same as or less then the number of hospitalizations for heart failure for the subject population at the first period in time, then a determination is made that there has been a reduction in the incidences of hospitalizations for heart failure for said subject population.

In some embodiments, BMP antagonists of the disclosure may be used to improve survival of heart failure patients. As used herein, improving survival of heart failure patients refers to maintaining or reducing the number of fatal cardiovascular events experienced by a subject population during or over the course of a period of time. An improvement in survival of heart failure patients can be assessed or determined by comparing the incidences of fatal cardiovascular events over or during the course of a period of time between two groups of subjects, in which a first group (the treatment group) is treated by the methods of the present invention, and a second group (the placebo group) is treated by using a placebo (namely, dummy pills) in replacement or in lieu of the treatment by the methods of the present invention. If the number of fatal cardiovascular events for the treatment group is less than the number of the fatal cardiovascular events for the placebo group, then a determination is made that there was or has been an improvement in survival of heart failure patients. Alternatively, a reduction in the incidence of fatal cardiovascular events can be assessed or determined by determining a baseline number of fatal cardiovascular events for a subject population at a first period in time and then measuring the number of fatal cardiovascular events for a subject population at a second, later period in time. If the number of fatal cardiovascular events for the subject population at the second, later period in time is the same as or less then the number of fatal cardiovascular events for the subject population at the first period in time, then a determination is made that there has been an improvement in survival of heart failure patients for said subject population.

In some embodiments, BMP antagonists of the disclosure may be used to reduce risk of cardiovascular death in heart failure patients. As used herein, reducing risk of cardiovascular death of heart failure patients refers to maintaining or reducing the number of fatal cardiovascular events experienced by a subject population during or over the course of a period of time. A reduction in cardiovascular deaths in heart failure patients can be assessed or determined by comparing the incidences of fatal cardiovascular events over or during the course of a period of time between two groups of subjects, in which a first group (the treatment group) is treated by the methods of the present invention, and a second group (the placebo group) is treated by using a placebo (namely, dummy pills) in replacement or in lieu of the treatment by the methods of the present invention. If the number of fatal cardiovascular events for the treatment group is less than the number of the fatal cardiovascular events for the placebo group, then a determination is made that there was or has been a reduction in cardiovascular deaths in heart failure patients. Alternatively, a reduction in cardiovascular deaths in heart failure patients can be assessed or determined by determining a baseline number of fatal cardiovascular events for a subject population at a first period in time and then measuring the number of fatal cardiovascular events for a subject population at a second, later period in time. If the number of fatal cardiovascular events for the subject population at the second, later period in time is the same as or less then the number of fatal cardiovascular events for the subject population at the first period in time, then a determination is made that there has been a reduction in cardiovascular deaths in heart failure patients for said subject population.

There are a wide variety of approved drugs and supportive therapies currently in use to manage patients with heart failure as well as patients at risk for heart failure (e.g., patients with hypertension, a lipid disorder, diabetes, and vascular disorders). Such drugs include, for example, adrenergic blockers (alpha- and beta-blockers), centrally acting alpha-agonists, angiotensin-converting enzyme (ACE) inhibitors, angiotensin receptor blockers, calcium channel blockers, positive inotropes, vasodilators, benzodiazepines, renin inhibitors, antithrombotic agents, and multiple types of diuretics (e.g., loop, potassium-sparing, thiazide and thiazide-like). Surgical procedures for treating or preventing heart failure include, for example, physical manipulation in an attempt to increase the internal size of constricted arteries by balloon angioplasty or stenting. In some embodiments, the present disclosure provides methods of treating heart failure or one or more complications of heart failure comprising administration a BMP antagonist in combination with an additional active agent or supportive therapy for treating, preventing or reducing the progression of heart failure (e.g., adrenergic blockers, centrally acting alpha-agonists, ACE inhibitors, angiotensin II receptor blockers, calcium channel blockers, positive inotropes, diuretics, and various surgical procedures).

ACE inhibitors have been used for the treatment of hypertension for many years. ACE inhibitors block the formation of angiotensin II, a hormone with adverse effects on the heart and circulation, particularly in heart failure patients. Side effects of these drugs include, for example, a dry cough, low blood pressure, worsening kidney function and electrolyte imbalances, and sometimes, allergic reactions. Examples of ACE inhibitors include captopril (e.g., Capoten), enalapril (e.g., Vasotec, Renitec, and Enacard), lisinopril (Zestril and Prinivil), benazepril (Lotensin), ramipril (e.g., Altace, Prilace, Ramace, Ramiwin, Triatec, and Tritace), Zofenopril, quinapril (e.g., Accupril), perinodopril (e.g., Coversyl, Aceon, and Perindo), lisinopril (e.g., Listril, Lopril, Novatec, Prinivil, and Zestril), benazepril (e.g., Lotensin), imidapril (e.g., tanatril), trandolapril (e.g., Mavik, Odrik, and Gopten), cilazapril (e.g., Inhibace), and fosinopril (e.g., Fositen and Monopril).

In general, patients whom are intolerant of ACE inhibitors are treated with angiotensin receptor blockers. These drugs act on the same hormonal pathway as ACE inhibitors, but instead block the action of angiotensin II at its receptor site directly. Side effects of these drugs are similar to those associated with ACE inhibitors, although the dry cough is less common. Angiotensin receptor blockers that may be used in accordance with the disclosure include, for example, losartan (e.g., Cozaar), candesartan (e.g., Atacand), valsartan (e.g., Diovan), irbesartan (e.g., Avapro), telmisartan (e.g., Micardis), eprosartan (e.g., Teveten), olmesartan (e.g., Benicar and Olmetec), azilsartan (Edarbi), and Fimasartan (e.g., Kanarb).

Adrenergic blockers are drugs that block the action of certain stimulating hormones, such as epinephrine (adrenaline), norepinephrine, and other similar hormones, which act on the beta receptors of various body tissues. The natural effect of these hormones on the adrenergic receptors of the heart is a more forceful contraction of the heart muscle. The stimulating effect of these hormones, while initially useful in maintaining heart function, appears to have detrimental effects on the heart muscle over time. Beta-blockers (e.g., non-specific, β₁-selective, β₂-selective, and β₃-selective blockers) are agents that block the action of these stimulating hormones on the beta receptors. Alpha-blockers (e.g., non-specific, α₁-selective, and α₂-selective) are agents that block the action of these stimulating hormones on the alpha receptors. Generally, if chronic heart patients receive adrenergic blockers they are given at a very low dose at first which is then gradually increased. Side effects include, for example, fluid retention, low blood pressure, low pulse, and general fatigue and lightheadedness. Adrenergic blockers should also not be used in people with diseases of the airways (e.g., asthma and emphysema) or very low resting heart rates. Adrenergic blockers that may be used in accordance with the disclosure include, for example, propranolol, bucindolol, carteolol (e.g., Cartrol, Ocupress, Teoptic, Arteolol, Arteoptic, Calte, Cartéabak, Carteol, Cartéol, Cartrol, Elebloc, Endak, Glauteolol, Mikelan, Poenglaucol, and Singlauc), carvedilol (e.g., Coreg), labetalol (e.g., Normodyne and Trandate), nadolol (e.g., Corgard), oxprenolol (e.g., Trasacor, Trasicor, Coretal, Laracor, Slow-Pren, Captol, Corbeton, Slow-Trasicor, Tevacor, Trasitensin, Trasidex), penbutolol (e.g., Levatol, Levatolol, Lobeta, Paginol, Hostabloc, Betapressin), pindolol (e.g., Visken), sotalol (e.g., Betapace, Betapace AF, Sotalex, Sotacor, and Sotylize), timolol (e.g., betimol), acebutolol (e.g., Sectral and Prent), atenolol (e.g., tenormin), betaxolol (e.g., Betoptic, Betoptic S, Lokren, and Kerlone), bisoprolol (e.g., Zebeta and Concor), celiprolol (e.g., Cardem, Selectol, Celipres, Celipro, Celol, Cordiax, Dilanorm), esmolol (e.g., brevibloc), metoprolol (e.g., Lopressor and Metolar XR), nebivolol (e.g., Nebilet and Bystolic), butazamine, ICI-118,551, SR 59230A, phenoxybenzamine (e.g., Dibenzyline), phentolamine (e.g., Regitine), tolazoline, trazodone, alfuzosin (e.g., uroxatral, Xat, Xatral, Prostetrol and Alfural), doxazosin mesylate (Cardura and Carduran), prazosin (e.g., Minipress, Vasoflex, Lentopres and Hypovase), tamsulosin (Flomax), terazosin (e.g., Hytrin and Zayasel), Silodosin (e.g., Rapaflo, Silodyx, Rapilif, Silodal, Urief, Thrupas, and Urorec), atipanmezole (e.g., Antisedan), idazoxan, mirtazapine (e.g., Remeron), and yohimbine.

Diuretics are often used in the treatment of heart failure patients to prevent or alleviate the symptoms of fluid retention. These drugs help keep fluid from building up in the lungs and other tissues by promoting the flow of fluid through the kidneys. Although they are effective in relieving symptoms such as shortness of breath and leg swelling, they have not been demonstrated to positively impact long term survival. Side effects of diuretics include dehydration, electrolyte abnormalities, particularly low potassium levels, hearing disturbances, and low blood pressure. In some patients, it is important to prevent low potassium levels by providing supplements to patients as electrolyte imbalances may make patients susceptible to serious heart rhythm disturbances. Examples of various classes of diuretics include: acidifying salts (e.g., CaCl₂ and NH₄CL), arginine vasopressin receptor 2 antagonists (e.g., amphotericin B and lithium citrate), selective vasopressin V2 antagonists (e.g., tolvapatan and conivaptan), Na—H exchanger antagonists (e.g., dopamine), carbonic anhydrase inhibitors (e.g., acetazolamide and dorzolamide), loop diuretics (e.g., bumetanide, ethacrynic acid, furosemide, and torsemide), osmotic diuretics (e.g., glucose and mannitol), potassium-sparing diuretics (e.g., amiloride, spironolactone, eplerenone, triamterene, and potassium canrenoate), thiazides (e.g., bendroflumethiazide, hydrochlorothiazide, and metolazone), xanthines (e.g., theophylline and theobromine).

Calcium channel blockers disrupt the movement of calcium through calcium channels and are frequently used as antihypertensive drugs. Calcium channel blockers are particularly effective in treating large vessel stiffness, one of the common causes of elevated systolic blood pressure in elderly patients. Calcium channel blockers are also frequently used to alter heart rate, to prevent cerebral vasospasm, and reduce chest pain caused by angina pectoris. Side effects of calcium channel blockers include, for example, dizziness, headache, edema, altered heart rate, and constipation. Examples of various classes of calcium channel blockers include: dihydropyridine calcium channel blockers [e.g., amlodipine (e.g., Norvasc), aranidipine (e.g., Sapresta), azelnidipine (e.g., Calblock), barnidipine (e.g., HypoCa), benidipine (e.g., Coniel), cilnidipine (e.g., Atelec, Cinalong, and Siscard), clevidipine (e.g., Cleviprex), isradipine (e.g., DynaCirc and Prescal), efonidipine (e.g., Landel), felodipine (e.g., Plendil), lacidipine (e.g., Motens, Lacipil), lercanidipine (e.g., Zanidip), manidipine (e.g., Calslot and Madipine), nicardipine (e.g., Cardene and Carden SR), nifedipine (e.g., Procardia and Adalat), Nilvadipine (e.g., Nivadil), nimodipine (e.g., Nimotop), nisoldipine (e.g., Baymycard, Sular, and Syscor), nitrendipine (e.g., Cardif, Nitrepin, and Baylotensin), and pranidipine (e.g., Acalas)] phenylalkylamine calcium channel blockers [e.g., verapamil (e.g., Calan and Isoptin), gallopamil, and fendiline], benzothiazepine calcium channel blockers (e.g., diltiazem), mibefradil, bepridil, flunarizine, fluspirilene, fendiline, gabapentinoids (e.g., gabapentin and pregabalin), and ziconotide.

Inotropes are agents that alter the force or energy of muscular contractions. By increasing the concentration of intracellular calcium or increasing the sensitivity or receptor proteins to calcium, positive inotropic agents can increase myocardial contractility. Examples of positive inotropic agents include: digoxin, amiodarone, berberine, calcium, levosimendan, omecamtiv, catecholamines (e.g., dopamine, dobutamine, dopexamine, epinephrine, isoprenaline, and norepinephrine), angiotensin II, eicosanoids (e.g., prostaglandins), phosphodiesterase inhibitors (e.g., enoximone, milrinone, amrinone, and theophylline), glucagon, and insulin.

Vasodilators act directly on the smooth muscle of arteries to relax their walls so blood can move more easily through them. In general, vasodilators are only used in hypertensive emergencies or when other drugs have failed and are rarely given alone. The primary vasodilators used to treat heart failure include nitrates and hydralazine and derivatives thereof. Vasodilators that may be used in accordance with the disclosure include, for example, sodium nitroprusside, hydralazine (e.g., Apesoline and BiDil), isosorbide dinitrate (Dilatrate and Isordil), and isosorbide mononitrate (e.g., ISMO), nitroglycerin (e.g., Nitro-Dur, Nitrolingual, and Nitrostat).

Although controversial, benzodiazepines may play a role in lowering blood pressure. They work as an agonist of the GABA-a receptors in the brain, thus slowing down neurotransmission and dilating blood vessels. GABA is an inhibitory neurotransmitter among others (glycine, adenosine, etc.). GABA-a receptors are ion channels that are the primary target for benzodiazepines. When an agonist binds to this receptor site, the protein channel opens, allowing negative chloride ions entering the channel and penetrating the voltage-gated ion site. Thus, giving negative feedback in neurotransmission and easing stress, anxiety and tension in patients that can be associated with elevated blood pressure. In addition to GABA, benzodiazepines inhibit the re-uptake of a nucleoside chemical called adenosine, which serves as an inhibitory chemical mentioned above. It also serves as a coronary vasodilator, allowing the cardiac muscle to relax and dilating cardiac arteries. However, long-term use of benzodiazepines is associated with dependence and tolerance, which is likely the result of GABA-a receptor downregulation. Therefore, withdrawal symptoms include hypertension, even in healthy individuals.

Renin comes one level higher than angiotensin converting enzyme (ACE) in the renin-angiotensin system. Inhibitors of renin can therefore effectively reduce hyptertension. Aliskiren is a renin inhibitor which has been approved for the treatment of hypertension.

Central alpha agonists lower blood pressure by stimulating alpha-receptors in the brain which open peripheral arteries easing blood flow. These alpha 2 receptors are known as autoreceptors which provide a negative feedback in neurotransmission (in this case, the vasoconstriction effects of adrenaline). Central alpha agonists are usually prescribed when all other anti-hypertensive medications have failed. For treating hypertension, these drugs are usually administered in combination with a diuretic. Adverse effects of this class of drugs include sedation, drying of the nasal mucosa and rebound hypertension. Central alpha agonists that may be used in accordance with the present disclosure include, for example, clonidine (e.g., Catapres, Kapvay, Nexiclon, and Clophelin), guanabenz (e.g., Wytensin), guanfacine (e.g., Estulic, Tenex, and Intuniv), methyldopa (e.g., Aldomet, Aldoril, and Dopamet), and moxonidine (Physiotens), minoxidil (e.g., Loniten) guanethidine (e.g., Micromedex), mecamylamine (e.g., Inversine and Vecamyl), and reserpine (e.g., Raudixin, Serpalan, and Serpasil).

In general, an antithrombotic agent is a drug that reduces the formation of blood clots (thrombi). Antithrombotics can be used therapeutically for prevention (primary prevention, secondary prevention) or treatment of a dangerous blood clot (acute thrombus). Different antithrombotics affect different blood clotting processes. Antiplatelet drugs limit the migration or aggregation of platelets. Anticoagulants limit the ability of the blood to clot. Thrombolytic drugs act to dissolve clots after they have formed. Antithrombotic agents that may be used in accordance with present disclosure include, for example, irreversible cyclooxygenase inhibitors [e.g., aspirin and triflusal (e.g., Disgren)], adenosine diphosphate receptor inhibitors [e.g., clopidogrel (e.g., Plavix), prasugrel (e.g., Effient), ticagrelor (e.g., Brilinta), and ticlopidine (e.g., Ticlid)], phosphodiesterase inhibitors, [e.g., cilostazol (e.g., Pletal)], protease-activated receptor-1 antagonists [e.g., vorapaxar (e.g., Zontivity)], glycoprotein IIB/IIIA inhibitors [e.g., abciximab (e.g., ReoPro), eptifibatide (e.g., Integrilin), tirofiban (e.g., Aggrastat)], adenosine reuptake inhibitors [e.g., dipyridamole (e.g., Persantine)], thromboxane inhibitors [e.g., thromboxane synthase inhibitors and thromboxane receptor antagonists (e.g., Terutroban)], tissue plasminogen activators [e.g., alteplase (e.g., Activase), reteplase (e.g., Retavase), tenecteplase (e.g., TNKase)], anistreplase (e.g., Eminase), streptokinase (e.g., Kabikinase and Streptase), urokinase (e.g., Abbokinase), dabigatran, rivaroxaban, apixaban, coumarins (e.g., warfarin, brodifacoum, and difenacoum), heparin and derivatives thereof, factor Xa inhibitors (e.g., fondaparinux and idraparinux), rivaroxaban, apixaban, edoxaban, betrixaban, letaxaban, eribaxaban, hirudin, lepirudin, bivalirudin, argatroban, dabigatran, ximelagatran (e.g., Exanta), antithrombin protein (e.g., Atryn), batroxobin, hementin, and vitamin E.

In addition to pharmacological treatments for heart failure, there is a variety of supportive therapies for treating heart failure or one or more complications of heart failure including, for example, surgical procedures and medical devices.

One of the most common heart failure medical devices is a pacemaker. Pacemakers are generally small devices that are placed in the chest or abdomen of the patient to help control abnormal heart rhythms. These devices use low-energy pulses to prompt the heart to beat at a normal rate (e.g., treat arrhythmias). There are different types of pacemaker devices that provide treatment for different types of arrhythmias.

In people with severe cardiomyopathy (e.g., left ventricular ejection fraction below 35%), or in those with recurrent ventricular tachycardia or malignant arrhythmias, treatment with an automatic implantable cardioverter defibrillator is indicated to reduce the risk of severe life-threatening arrhythmias. The automatic implantable cardioverter defibrillator does not improve symptoms or reduce the incidence of malignant arrhythmias but does reduce mortality from those arrhythmias, often in conjunction with antiarrhythmic medications. In people with left ventricular ejection below 35%, the incidence of ventricular tachycardia or sudden cardiac death is high enough to warrant AICD placement.

Cardiac contractility modulation (CCM) is a treatment for people with moderate to severe left ventricular systolic heart failure (NYHA class II-IV) which enhances both the strength of ventricular contraction and the heart's pumping capacity. The CCM mechanism is based on stimulation of the cardiac muscle by non-excitatory electrical signals, which are delivered by a pacemaker-like device. CCM is particularly suitable for the treatment of heart failure with normal QRS complex duration (120 ms or less) and has been demonstrated to improve the symptoms, quality of life and exercise tolerance.

About one third of people with left ventricle ejection fraction below 35% have markedly altered conduction to the ventricles, resulting in dyssynchronous depolarization of the right and left ventricles. This is especially problematic in people with left bundle branch block (blockage of one of the two primary conducting fiber bundles that originate at the base of the heart and carries depolarizing impulses to the left ventricle). Using a special pacing algorithm, biventricular cardiac resynchronization therapy (CRT) can initiate a normal sequence of ventricular depolarization. In people with left ventricle ejection fraction below 35% and prolonged QRS duration on electrocardiogram (LBBB or QRS of 150 ms or more) there is an improvement in symptoms and mortality when CRT is added to standard medical therapy.

People with the most severe heart failure may be candidates for ventricular assist devices (VAD). VADs have commonly been used as a bridge to heart transplantation, but have been used more recently as a destination treatment for advanced heart failure. In select cases, heart transplantation can be considered. While this may resolve the problems associated with heart failure, the person must generally remain on an immunosuppressive regimen to prevent rejection, which has its own significant downsides. A major limitation of this treatment option is the scarcity of hearts available for transplantation.

### 9. Pharmaceutical Compositions

In certain aspects, BMP antagonists, or combinations of such antagonists, of the present disclosure can be administered alone or as a component of a pharmaceutical formulation (also referred to as a therapeutic composition or pharmaceutical composition). A pharmaceutical formation refers to a preparation which is in such form as to permit the biological activity of an active ingredient (e.g., an agent of the present disclosure) contained therein to be effective and which contains no additional components which are unacceptably toxic to a subject to which the formulation would be administered. The subject compounds may be formulated for administration in any convenient way for use in human or veterinary medicine. For example, one or more agents of the present disclosure may be formulated with a pharmaceutically acceptable carrier. A pharmaceutically acceptable carrier refers to an ingredient in a pharmaceutical formulation, other than an active ingredient, which is generally nontoxic to a subject. A pharmaceutically acceptable carrier includes, but is not limited to, a buffer, excipient, stabilizer, and/or preservative. In general, pharmaceutical formulations for use in the present disclosure are in a pyrogen-free, physiologically-acceptable form when administered to a subject. Therapeutically useful agents other than those described herein, which may optionally be included in the formulation as described above, may be administered in combination with the subject agents in the methods of the present disclosure.

In certain embodiments, compositions will be administered parenterally [e.g., by intravenous (I. V.) injection, intraarterial injection, intraosseous injection, intramuscular injection, intrathecal injection, subcutaneous injection, or intradermal injection]. Pharmaceutical compositions suitable for parenteral administration may comprise one or more agents of the disclosure in combination with one or more pharmaceutically acceptable sterile isotonic aqueous or nonaqueous solutions, dispersions, suspensions or emulsions, or sterile powders which may be reconstituted into sterile injectable solutions or dispersions just prior to use. Injectable solutions or dispersions may contain antioxidants, buffers, bacteriostats, suspending agents, thickening agents, or solutes which render the formulation isotonic with the blood of the intended recipient. Examples of suitable aqueous and nonaqueous carriers which may be employed in the pharmaceutical formulations of the present disclosure include water, ethanol, polyols (e.g., glycerol, propylene glycol, polyethylene glycol, etc.), vegetable oils (e.g., olive oil), injectable organic esters (e.g., ethyl oleate), and suitable mixtures thereof. Proper fluidity can be maintained, for example, by the use of coating materials (e.g., lecithin), by the maintenance of the required particle size in the case of dispersions, and by the use of surfactants.

In some embodiments, compounds will be administered to the heart including, e.g., by intra-cardial administration, intra-pericardial administration, or by implant or device. For example, access to the pericardial space may be accomplished from outside the body by making a thoracic or subxiphoid incision to access and cut or pierce the pericardial sac. Access to the pericardial space from the exterior of the body, accomplished by passing a cannula or catheter type device through the chest wall and thereafter passing the cannula or catheter or a further instrument through the pericardium into the pericardial space, is disclosed, for example, in U.S. Pat. Nos. 5,336,252, 5,827,216, 5,900,433, 5,972,013, 6,162,195, 6,206,004, and 6,592,552. In certain cases the pericardial sac may be cut by a cutting instrument as disclosed, for example, in U.S. Pat. Nos. 5,931,810, 6,156,009, and 6,231,518.

In some embodiments, a therapeutic method of the present disclosure includes administering the pharmaceutical composition systemically, or locally, from an implant or device. Further, the pharmaceutical composition may be encapsulated or injected in a form for delivery to a target tissue site (e.g., bone marrow or muscle). In certain embodiments, compositions of the present disclosure may include a matrix capable of delivering one or more of the agents of the present disclosure to a target tissue site (e.g., bone marrow or muscle), providing a structure for the developing tissue and optimally capable of being resorbed into the body. For example, the matrix may provide slow release of one or more agents of the present disclosure. Such matrices may be formed of materials presently in use for other implanted medical applications.

The choice of matrix material may be based on one or more of: biocompatibility, biodegradability, mechanical properties, cosmetic appearance, and interface properties. The particular application of the subject compositions will define the appropriate formulation. Potential matrices for the compositions may be biodegradable and chemically defined calcium sulfate, tricalciumphosphate, hydroxyapatite, polylactic acid, and polyanhydrides. Other potential materials are biodegradable and biologically well-defined including, for example, bone or dermal collagen. Further matrices are comprised of pure proteins or extracellular matrix components. Other potential matrices are non-biodegradable and chemically defined including, for example, sintered hydroxyapatite, bioglass, aluminates, or other ceramics. Matrices may be comprised of combinations of any of the above mentioned types of material including, for example, polylactic acid and hydroxyapatite or collagen and tricalciumphosphate. The bioceramics may be altered in composition (e.g., calcium-aluminate-phosphate) and processing to alter one or more of pore size, particle size, particle shape, and biodegradability.

In certain embodiments, pharmaceutical compositions of present disclosure can be administered topically. “Topical application” or “topically” means contact of the pharmaceutical composition with body surfaces including, for example, the skin, wound sites, and mucous membranes. The topical pharmaceutical compositions can have various application forms and typically comprises a drug-containing layer, which is adapted to be placed near to or in direct contact with the tissue upon topically administering the composition. Pharmaceutical compositions suitable for topical administration may comprise one or more one or more BMP antagonists of the disclosure in combination formulated as a liquid, a gel, a cream, a lotion, an ointment, a foam, a paste, a putty, a semi-solid, or a solid. Compositions in the liquid, gel, cream, lotion, ointment, foam, paste, or putty form can be applied by spreading, spraying, smearing, dabbing or rolling the composition on the target tissue. The compositions also may be impregnated into sterile dressings, transdermal patches, plasters, and bandages. Compositions of the putty, semi-solid or solid forms may be deformable. They may be elastic or non-elastic (e.g., flexible or rigid). In certain aspects, the composition forms part of a composite and can include fibers, particulates, or multiple layers with the same or different compositions.

Topical compositions in the liquid form may include pharmaceutically acceptable solutions, emulsions, microemulsions, and suspensions. In addition to the active ingredient(s), the liquid dosage form may contain an inert diluent commonly used in the art including, for example, water or other solvent, a solubilizing agent and/or emulsifier [e.g., ethyl alcohol, isopropyl alcohol, ethyl carbonate, ethyl acetate, benzyl alcohol, benzyl benzoate, propylene glycol, or 1,3-butylene glycol, an oil (e.g., cottonseed, groundnut, corn, germ, olive, castor, and sesame oil), glycerol, tetrahydrofuryl alcohol, a polyethylene glycol, a fatty acid ester of sorbitan, and mixtures thereof].

Topical gel, cream, lotion, ointment, semi-solid or solid compositions may include one or more thickening agents, such as a polysaccharide, synthetic polymer or protein-based polymer. In one embodiment of the invention, the gelling agent herein is one that is suitably nontoxic and gives the desired viscosity. The thickening agents may include polymers, copolymers, and monomers of: vinylpyrrolidones, methacrylamides, acrylamides N-vinylimidazoles, carboxy vinyls, vinyl esters, vinyl ethers, silicones, polyethyleneoxides, polyethyleneglycols, vinylalcohols, sodium acrylates, acrylates, maleic acids, NN-dimethylacrylamides, diacetone acrylamides, acrylamides, acryloyl morpholine, pluronic, collagens, polyacrylamides, polyacrylates, polyvinyl alcohols, polyvinylenes, polyvinyl silicates, polyacrylates substituted with a sugar (e.g., sucrose, glucose, glucosamines, galactose, trehalose, mannose, or lactose), acylamidopropane sulfonic acids, tetramethoxyorthosilicates, methyltrimethoxyorthosilicates, tetraalkoxyorthosilicates, trialkoxyorthosilicates, glycols, propylene glycol, glycerine, polysaccharides, alginates, dextrans, cyclodextrin, celluloses, modified celluloses, oxidized celluloses, chitosans, chitins, guars, carrageenans, hyaluronic acids, inulin, starches, modified starches, agarose, methylcelluloses, plant gums, hylaronans, hydrogels, gelatins, glycosaminoglycans, carboxymethyl celluloses, hydroxyethyl celluloses, hydroxy propyl methyl celluloses, pectins, low-methoxy pectins, cross-linked dextrans, starch-acrylonitrile graft copolymers, starch sodium polyacrylate, hydroxyethyl methacrylates, hydroxyl ethyl acrylates, polyvinylene, polyethylvinylethers, polymethyl methacrylates, polystyrenes, polyurethanes, polyalkanoates, polylactic acids, polylactates, poly(3-hydroxybutyrate), sulfonated hydrogels, AMPS (2-acrylamido-2-methyl-1-propanesulfonic acid), SEM (sulfoethylmethacrylate), SPM (sulfopropyl methacrylate), SPA (sulfopropyl acrylate), N,N-dimethyl-N-methacryloxyethyl-N-(3-sulfopropyl)ammonium betaine, methacryllic acid amidopropyl-dimethyl ammonium sulfobetaine, SPI (itaconic acid-bis(1-propyl sulfonizacid-3) ester di-potassium salt), itaconic acids, AMBC (3-acrylamido-3-methylbutanoic acid), beta-carboxyethyl acrylate (acrylic acid dimers), and maleic anhydride-methylvinyl ether polymers, derivatives thereof, salts thereof, acids thereof, and combinations thereof. In certain embodiments, pharmaceutical compositions of present disclosure can be administered orally, for example, in the form of capsules, cachets, pills, tablets, lozenges (using a flavored basis such as sucrose and acacia or tragacanth), powders, granules, a solution or a suspension in an aqueous or non-aqueous liquid, an oil-in-water or water-in-oil liquid emulsion, or an elixir or syrup, or pastille (using an inert base, such as gelatin and glycerin, or sucrose and acacia), and/or a mouth wash, each containing a predetermined amount of a compound of the present disclosure and optionally one or more other active ingredients. A compound of the present disclosure and optionally one or more other active ingredients may also be administered as a bolus, electuary, or paste.

In solid dosage forms for oral administration (e.g., capsules, tablets, pills, dragees, powders, and granules), one or more compounds of the present disclosure may be mixed with one or more pharmaceutically acceptable carriers including, for example, sodium citrate, dicalcium phosphate, a filler or extender (e.g., a starch, lactose, sucrose, glucose, mannitol, and silicic acid), a binder (e.g. carboxymethylcellulose, an alginate, gelatin, polyvinyl pyrrolidone, sucrose, and acacia), a humectant (e.g., glycerol), a disintegrating agent (e.g., agar-agar, calcium carbonate, potato or tapioca starch, alginic acid, a silicate, and sodium carbonate), a solution retarding agent (e.g. paraffin), an absorption accelerator (e.g. a quaternary ammonium compound), a wetting agent (e.g., cetyl alcohol and glycerol monostearate), an absorbent (e.g., kaolin and bentonite clay), a lubricant (e.g., a talc, calcium stearate, magnesium stearate, solid polyethylene glycols, sodium lauryl sulfate), a coloring agent, and mixtures thereof. In the case of capsules, tablets, and pills, the pharmaceutical formulation (composition) may also comprise a buffering agent. Solid compositions of a similar type may also be employed as fillers in soft and hard-filled gelatin capsules using one or more excipients including, e.g., lactose or a milk sugar as well as a high molecular-weight polyethylene glycol.

Liquid dosage forms for oral administration of the pharmaceutical composition may include pharmaceutically acceptable emulsions, microemulsions, solutions, suspensions, syrups, and elixirs. In addition to the active ingredient(s), the liquid dosage form may contain an inert diluent commonly used in the art including, for example, water or other solvent, a solubilizing agent and/or emulsifier [e.g., ethyl alcohol, isopropyl alcohol, ethyl carbonate, ethyl acetate, benzyl alcohol, benzyl benzoate, propylene glycol, or 1,3-butylene glycol, an oil (e.g., cottonseed, groundnut, corn, germ, olive, castor, and sesame oil), glycerol, tetrahydrofuryl alcohol, a polyethylene glycol, a fatty acid ester of sorbitan, and mixtures thereof]. Besides inert diluents, the oral formulation can also include an adjuvant including, for example, a wetting agent, an emulsifying and suspending agent, a sweetening agent, a flavoring agent, a coloring agent, a perfuming agent, a preservative agent, and combinations thereof.

Suspensions, in addition to the active compounds, may contain suspending agents including, for example, an ethoxylated isostearyl alcohol, polyoxyethylene sorbitol, a sorbitan ester, microcrystalline cellulose, aluminum metahydroxide, bentonite, agar-agar, tragacanth, and combinations thereof.

Prevention of the action and/or growth of microorganisms may be ensured by the inclusion of various antibacterial and antifungal agents including, for example, paraben, chlorobutanol, and phenol sorbic acid.

In certain embodiments, it may be desirable to include an isotonic agent including, for example, a sugar or sodium chloride into the compositions. In addition, prolonged absorption of an injectable pharmaceutical form may be brought about by the inclusion of an agent that delay absorption including, for example, aluminum monostearate and gelatin.

It is understood that the dosage regimen will be determined by the attending physician considering various factors which modify the action of the one or more of the agents of the present disclosure. In the case of a BMP antagonist that promotes red blood cell formation, various factors may include, but are not limited to, the patient's red blood cell count, hemoglobin level, the desired target red blood cell count, the patient's age, the patient's sex, the patient's diet, the severity of any disease that may be contributing to a depressed red blood cell level, the time of administration, and other clinical factors. The addition of other known active agents to the final composition may also affect the dosage. Progress can be monitored by periodic assessment of one or more of red blood cell levels, hemoglobin levels, reticulocyte levels, and other indicators of the hematopoietic process.

In certain embodiments, the present disclosure also provides gene therapy for the in vivo production of one or more of the agents of the present disclosure. Such therapy would achieve its therapeutic effect by introduction of the agent sequences into cells or tissues having one or more of the disorders as listed above. Delivery of the agent sequences can be achieved, for example, by using a recombinant expression vector such as a chimeric virus or a colloidal dispersion system. Preferred therapeutic delivery of one or more of agent sequences of the disclosure is the use of targeted liposomes.

Various viral vectors which can be utilized for gene therapy as taught herein include adenovirus, herpes virus, vaccinia, or an RNA virus (e.g., a retrovirus). The retroviral vector may be a derivative of a murine or avian retrovirus. Examples of retroviral vectors in which a single foreign gene can be inserted include, but are not limited to: Moloney murine leukemia virus (MoMuLV), Harvey murine sarcoma virus (HaMuSV), murine mammary tumor virus (MuMTV), and Rous Sarcoma Virus (RSV). A number of additional retroviral vectors can incorporate multiple genes. All of these vectors can transfer or incorporate a gene for a selectable marker so that transduced cells can be identified and generated. Retroviral vectors can be made target-specific by attaching, for example, a sugar, a glycolipid, or a protein. Preferred targeting is accomplished by using an antibody. Those of skill in the art will recognize that specific polynucleotide sequences can be inserted into the retroviral genome or attached to a viral envelope to allow target specific delivery of the retroviral vector containing one or more of the agents of the present disclosure.

Alternatively, tissue culture cells can be directly transfected with plasmids encoding the retroviral structural genes (gag, pol, and env), by conventional calcium phosphate transfection. These cells are then transfected with the vector plasmid containing the genes of interest. The resulting cells release the retroviral vector into the culture medium.

Another targeted delivery system for one or more of the agents of the present disclosure is a colloidal dispersion system. Colloidal dispersion systems include, for example, macromolecule complexes, nanocapsules, microspheres, beads, and lipid-based systems including oil-in-water emulsions, micelles, mixed micelles, and liposomes. In certain embodiments, the preferred colloidal system of this disclosure is a liposome. Liposomes are artificial membrane vesicles which are useful as delivery vehicles in vitro and in vivo. RNA, DNA, and intact virions can be encapsulated within the aqueous interior and be delivered to cells in a biologically active form [Fraley, et al. (1981) Trends Biochem. Sci., 6:77]. Methods for efficient gene transfer using a liposome vehicle are known in the art [Mannino, et al. (1988) Biotechniques, 6:682, 1988].

The composition of the liposome is usually a combination of phospholipids, which may include a steroid (e.g. cholesterol). The physical characteristics of liposomes depend on pH, ionic strength, and the presence of divalent cations. Other phospholipids or other lipids may also be used including, for example a phosphatidyl compound (e.g., phosphatidylglycerol, phosphatidylcholine, phosphatidylserine, phosphatidylethanolamine, a sphingolipid, a cerebroside, and a ganglioside), egg phosphatidylcholine, dipalmitoylphosphatidylcholine, and di stearoylphosphatidylcholine. The targeting of liposomes is also possible based on, for example, organ-specificity, cell-specificity, and organelle-specificity and is known in the art.

### EXEMPLIFICATION

The invention now being generally described, it will be more readily understood by reference to the following examples, which are included merely for purposes of illustration of certain embodiments and embodiments of the present invention, and are not intended to limit the invention.

### Example 1: ActRIIa-Fc Fusion Proteins

ActRIIA fusion proteins having the extracellular domain of human ActRIIA fused to a human or mouse Fc domain with a linker in between were generated. The constructs are referred to as ActRIIA-hFc and ActRIIA-mFc, respectively.

ActRIIA-hFc is shown below as purified from CHO cell lines (SEQ ID NO: 50):

ILGRSETQECLFFNANWEKDRTNQTGVEPCYGDKDKRRHCFATWKNISGS
IEIVKQGCWLDDINCYDRTDCVEKKDSPEVYFCCCEGNMCNEKFSYFPEM
EVTQPTSNPVTPKPPTGGGTHTCPPCPAPELLGGPSVFLFPPKPKDTLMI
SRTPEVTCVVVDVSHEDPEVKFNWYVDGVEVHNAKTKPREEQYNSTYRVV
SVLTVLHQDWLNGKEYKCKVSNKALPVPIEKTISKAKGQPREPQVYTLPP
SREEMTKNQVSLTCLVKGFYPSDIAVEWESNGQPENNYKTTPPVLDSDGS
FFLYSKLTVDKSRWQQGNVFSCSVMHEALHNHYTQKSLSLSPGK

The ActRIIA-hFc and ActRIIA-mFc proteins were expressed in CHO cell lines. Three different leader sequences were considered:

(i) Honey bee mellitin (HBML):

(SEQ ID NO: 51)

MKFLVNVALVFMVVYISYIYA
(ii) Tissue plasminogen activator (TPA):

(SEQ ID NO: 52)

MDAMKRGLCCVLLLCGAVFVSP
(iii) Native:

(SEQ ID NO: 53)

MGAAAKLAFAVFLISCSSGA.

The selected form employs the TPA leader and has the following unprocessed amino acid sequence:

(SEQ ID NO: 54)
MDAMKRGLCCVLLLCGAVFVSPGAAILGRSETQECLFFNANWEKDRTNQT
GVEPCYGDKDKRRHCFATWKNISGSIEIVKQGCWLDDINCYDRTDCVEKK
DSPEVYFCCCEGNMCNEKFSYFPEMEVTQPTSNPVTPKPPTGGGTHTCPP
CPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKAL
PVPIEKTISKAKGQPREPQVYTLPPSREEMTKNQVSLTCLVKGFYPSDIA
VEWESNGQPENNYKTTPPVLDSDGSFFLYSKLTVDKSRWQQGNVFSCSVM
HEALHNHYTQKSLSLSPGK

This polypeptide is encoded by the following nucleic acid sequence:

(SEQ ID NO: 55)
ATGGATGCAATGAAGAGAGGGCTCTGCTGTGTGCTGCTGCTGTGTGGAGC
AGTCTTCGTTTCGCCCGGCGCCGCTATACTTGGTAGATCAGAAACTCAGG
AGTGTCTTTTTTTAATGCTAATTGGGAAAAAGACAGAACCAATCAAACTG
GTGTTGAACCGTGTTATGGTGACAAAGATAAACGGCGGCATTGTTTTGCT
ACCTGGAAGAATATTTCTGGTTCCATTGAATAGTGAAACAAGGTTGTTGG
CTGGATGATATCAACTGCTATGACAGGACTGATTGTGTAGAAAAAAAAGA
CAGCCCTGAAGTATATTTCTGTTGCTGTGAGGGCAATATGTGTAATGAAA
AGTTTTCTTATTTTCCGGAGATGGAAGTCACACAGCCCACTTCAAATCCA
GTTACACCTAAGCCACCCACCGGTGGTGGAACTCACACATGCCCACCGTG
CCCAGCACCTGAACTCCTGGGGGGACCGTCAGTCTTCCTCTTCCCCCCAA
AACCCAAGGACACCCTCATGATCTCCCGGACCCCTGAGGTCACATGCGTG
GTGGTGGACGTGAGCCACGAAGACCCTGAGGTCAAGTTCAACTGGTACGT
GGACGGCGTGGAGGTGCATAATGCCAAGACAAAGCCGCGGGAGGAGCAGT
ACAACAGCACGTACCGTGTGGTCAGCGTCCTCACCGTCCTGCACCAGGAC
TGGCTGAATGGCAAGGAGTACAAGTGCAAGGTCTCCAACAAAGCCCTCCC
AGTCCCCATCGAGAAAACCATCTCCAAAGCCAAAGGGCAGCCCCGAGAAC
CACAGGTGTACACCCTGCCCCCATCCCGGGAGGAGATGACCAAGAACCAG
GTCAGCCTGACCTGCCTGGTCAAAGGCTTCTATCCCAGCGACATCGCCGT
GGAGTGGGAGAGCAATGGGCAGCCGGAGAACAACTACAAGACCACGCCTC
CCGTGCTGGACTCCGACGGCTCCTTCTTCCTCTATAGCAAGCTCACCGTG
GACAAGAGCAGGTGGCAGCAGGGGAACGTCTTCTCATGCTCCGTGATGCA
TGAGGCTCTGCACAACCACTACACGCAGAAGAGCCTCTCCCTGTCTCCGG
GTAAATGAGAATTC

Both ActRIIA-hFc and ActRIIA-mFc were remarkably amenable to recombinant expression. The protein was purified as a single, well-defined peak of protein. N-terminal sequencing revealed a single sequence of -ILGRSETQE (SEQ ID NO: 56). Purification could be achieved by a series of column chromatography steps, including, for example, three or more of the following, in any order: protein A chromatography, Q sepharose chromatography, phenylsepharose chromatography, size exclusion chromatography, and cation exchange chromatography. The purification could be completed with viral filtration and buffer exchange. The ActRIIA-hFc protein was purified to a purity of >98% as determined by size exclusion chromatography and >95% as determined by SDS PAGE.

ActRIIA-hFc and ActRIIA-mFc showed a high affinity for various ligands. Ligands were immobilized on a Biacore™ CMS chip using standard amine-coupling procedure. ActRIIA-hFc and ActRIIA-mFc proteins were loaded onto the system, and binding was measured. ActRIIA-hFc bound to various ligands including, for example, BMP10 with a dissociation constant (K_{D}) of 3.33×10⁻¹⁰ M, BMP9 with a K_{D} of 1.04×10⁻⁸M, BMP6 with a K_{D} of 5.56×10⁻¹⁰ M, and BMP5 with a K_{D} of 1.14×10⁻⁹M. ActRIIA-mFc behaved similarly.

A variety of ActRIIA variants that may be used according to the methods described herein are described in the International Patent Application published as WO2006/012627 and WO 2007/062188, incorporated herein by reference in their entirety. An alternative construct may have a deletion of the C-terminal tail (the final 15 amino acids of the extracellular domain of ActRIIA. The sequence for such a construct is presented below (Fc portion underlined) (SEQ ID NO: 57):

ILGRSETQECLFFNANWEKDRTNQTGVEPCYGDKDKRRHCFATWKNISGS
IEIVKQGCWLDDINCYDRTDCVEKKDSPEVYFCCCEGNMCNEKFSYFPEM
TGGGTHTCPPCPAPELLGGPSVFLFPPKPKDTLMISRTPEVTCVVVDVSH
EDPEVKFNWYVDGVEVHNAKTKPREEQYNSTYRVVSVLTVLHQDWLNGKE
YKCKVSNKALPVPIEKTISKAKGQPREPQVYTLPPSREEMTKNQVSLTCL
VKGFYPSDIAVEWESNGQPENNYKTTPPVLDSDGSFFLYSKLTVDKSRWQ
QGNVFSCSVMHEALHNHYTQKSLSLSPGK

### Example 2: Generation of ActRIIB-Fc Fusion Proteins

ActRIIB fusion proteins having the extracellular domain of human ActRIIB fused to a human or mouse Fc domain with a linker in between were constructed. The constructs are referred to as ActRIIB-hFc and ActRIIB-mFc, respectively.

ActRIIB-hFc is shown below as purified from CHO cell lines (SEQ ID NO: 58):

GRGEAETRECIYYNANWELERTNQSGLERCEGEQDKRLHCYASWRNSSGT
IELVKKGCWLDDFNCYDRQECVATEENPQVYFCCCEGNFCNERFTHLPEA
GGPEVTYEPPPTAPTGGGTHTCPPCPAPELLGGPSVFLFPPKPKDTLMIS
RTPEVTCVVVDVSHEDPEVKFNWYVDGVEVHNAKTKPREEQYNSTYRVVS
VLTVLHQDWLNGKEYKCKVSNKALPVPIEKTISKAKGQPREPQVYTLPPS
REEMTKNQVSLTCLVKGFYPSDIAVEWESNGQPENNYKTTPPVLDSDGSF
FLYSKLTVDKSRWQQGNVFSCSVMHEALHNHYTQKSLSLSPGK

The ActRIIB-hFc and ActRIIB-mFc proteins were expressed in CHO cell lines. Three different leader sequences were considered: (i) Honey bee mellitin (HBML), ii) Tissue plasminogen activator (TPA), and (iii) Native: MGAAAKLAFAVFLISCSSGA (SEQ ID NO: 59).

The selected form employs the TPA leader and has the following unprocessed amino acid sequence (SEQ ID NO: 60):

MDAMKRGLCCVLLLCGAVFVSPGASGRGEAETRECIYYNANWELERTNQS
GLERCEGEQDKRLHCYASWRNSSGTIELVKKGCWLDDFNCYDRQECVATE
ENPQVYFCCCEGNFCNERFTHLPEAGGPEVTYEPPPTAPTGGGTHTCPPC
PAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALP
VPIEKTISKAKGQPREPQVYTLPPSREEMTKNQVSLTCLVKGFYPSDIAV
EWESNGQPENNYKTTPPVLDSDGSFFLYSKLTVDKSRWQQGNVFSCSVMH
EALHNHYTQKSLSLSPGK

This polypeptide is encoded by the following nucleic acid sequence (SEQ ID NO: 61):

A TGGATGCAAT GAAGAGAGGG CTCTGCTGTG TGCTGCTGCT
GTGTGGAGCA GTCTTCGTTT CGCCCGGCGC CTCTGGGCGT
GGGGAGGCTG AGACACGGGA GTGCATCTAC TACAACGCCA
ACTGGGAGCT GGAGCGCACC AACCAGAGCG GCCTGGAGCG
CTGCGAAGGC GAGCAGGACA AGCGGCTGCA CTGCTACGCC
TCCTGGCGCA ACAGCTCTGG CACCATCGAG CTCGTGAAGA
AGGGCTGCTG GCTAGATGAC TTCAACTGCT ACGATAGGCA
GGAGTGTGTG GCCACTGAGG AGAACCCCCA GGTGTACTTC
TGCTGCTGTG AAGGCAACTT CTGCAACGAG CGCTTCACTC
ATTTGCCAGA GGCTGGGGGC CCGGAAGTCA CGTACGAGCC
ACCCCCGACA GCCCCCACCG GTGGTGGAAC TCACACATGC
CCACCGTGCC CAGCACCTGA ACTCCTGGGG GGACCGTCAG
TCTTCCTCTT CCCCCCAAAA CCCAAGGACA CCCTCATGAT
CTCCCGGACC CCTGAGGTCA CATGCGTGGT GGTGGACGTG
AGCCACGAAG ACCCTGAGGT CAAGTTCAAC TGGTACGTGG
ACGGCGTGGA GGTGCATAAT GCCAAGACAA AGCCGCGGGA
GGAGCAGTAC AACAGCACGT ACCGTGTGGT CAGCGTCCTC
ACCGTCCTGC ACCAGGACTG GCTGAATGGC AAGGAGTACA
AGTGCAAGGT CTCCAACAAA GCCCTCCCAG TCCCCATCGA
GAAAACCATC TCCAAAGCCA AAGGGCAGCC CCGAGAACCA
CAGGTGTACA CCCTGCCCCC ATCCCGGGAG GAGATGACCA
AGAACCAGGT CAGCCTGACC TGCCTGGTCA AAGGCTTCTA
TCCCAGCGAC ATCGCCGTGG AGTGGGAGAG CAATGGGCAG
CCGGAGAACA ACTACAAGAC CACGCCTCCC GTGCTGGACT
CCGACGGCTC CTTCTTCCTC TATAGCAAGC TCACCGTGGA
CAAGAGCAGG TGGCAGCAGG GGAACGTCTT CTCATGCTCC
GTGATGCATG AGGCTCTGCA CAACCACTAC ACGCAGAAGA
GCCTCTCCCT GTCTCCGGGT AAATGA

N-terminal sequencing of the CHO-cell-produced material revealed a major sequence of -GRGEAE (SEQ ID NO: 62). Notably, other constructs reported in the literature begin with an -SGR . . . sequence.

Purification could be achieved by a series of column chromatography steps, including, for example, three or more of the following, in any order: protein A chromatography, Q sepharose chromatography, phenylsepharose chromatography, size exclusion chromatography, and cation exchange chromatography. The purification could be completed with viral filtration and buffer exchange.

ActRIIB-hFc and ActRIIB-mFc showed a high affinity for various ligands. Ligands were immobilized on a Biacore™ CM5 chip using standard amine-coupling procedure. ActRIIB-hFc and ActRIIB-mFc proteins were loaded onto the system, and binding was measured. ActRIIB-hFc bound to various ligands including, for example, BMP10 with a dissociation constant (K_{D}) of 1.73×10⁻¹¹M, BMP9 with a K_{D} of 3.35×10⁻¹¹M, BMP6 with a K_{D} of 1.64×10⁻¹⁰ M, and BMP5 with a K_{D} of 2.92×10⁻⁹M. ActRIIB-mFc behaved similarly.

A series of mutations were generated in the extracellular domain of ActRIIB and these mutant proteins were produced as soluble fusion proteins between variant extracellular domain of ActRIIB and an Fc domain. The background ActRIIB-Fc fusion has the sequence of SEQ ID NO: 58. Various mutations, including N- and C-terminal truncations, were introduced into the background ActRIM-Fc protein. Based on the data presented herein, it is expected that these constructs, if expressed with a TPA leader, will lack the N-terminal serine. Mutations were generated in ActRIIB extracellular domain by PCR mutagenesis. After PCR, fragments were purified through a Qiagen column, digested with SfoI and AgeI and gel purified. These fragments were ligated into expression vector pAID4 (see WO2006/012627) such that upon ligation it created fusion chimera with human IgG1. Upon transformation into E. coli DH5 alpha, colonies were picked and DNAs were isolated. For murine constructs (mFc), a murine IgG2a was substituted for the human IgG1. Sequences of all mutants were verified. All of the mutants were produced in HEK293T cells by transient transfection. In summary, in a 500 ml spinner, HEK293T cells were set up at 6×10⁵ cells/ml in Freestyle (Invitrogen) media in 250 ml volume and grown overnight. Next day, these cells were treated with DNA:PEI (1:1) complex at 0.5 ug/ml final DNA concentration. After 4 hrs, 250 ml media was added and cells were grown for 7 days. Conditioned media was harvested by spinning down the cells and concentrated. Mutants were purified using a variety of techniques, including, for example, a protein A column, and eluted with low pH (3.0) glycine buffer. After neutralization, these were dialyzed against PBS. Mutants were also produced in CHO cells by similar methodology. Mutants were tested in binding assays and/or bioassays described in WO 2008/097541 and WO 2006/012627, incorporated by reference herein. In some instances, assays were performed with conditioned medium rather than purified proteins. Additional variations of ActRIIB are described in U.S. Pat. No. 7,842,663.

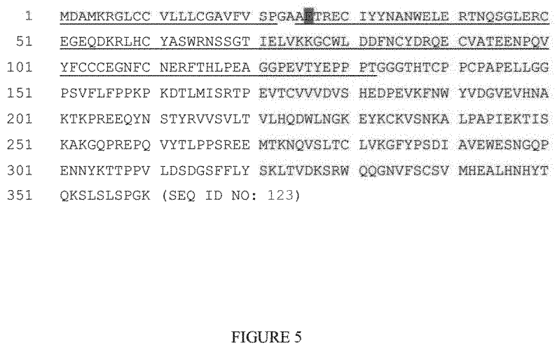

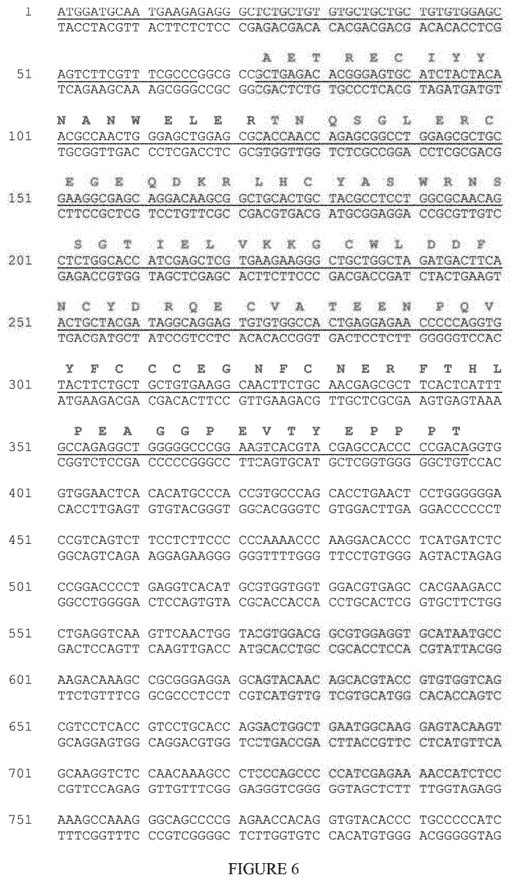

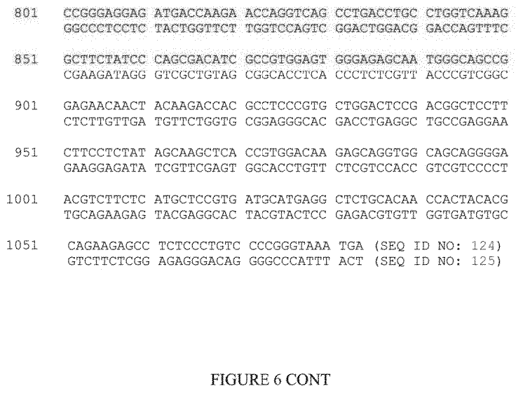

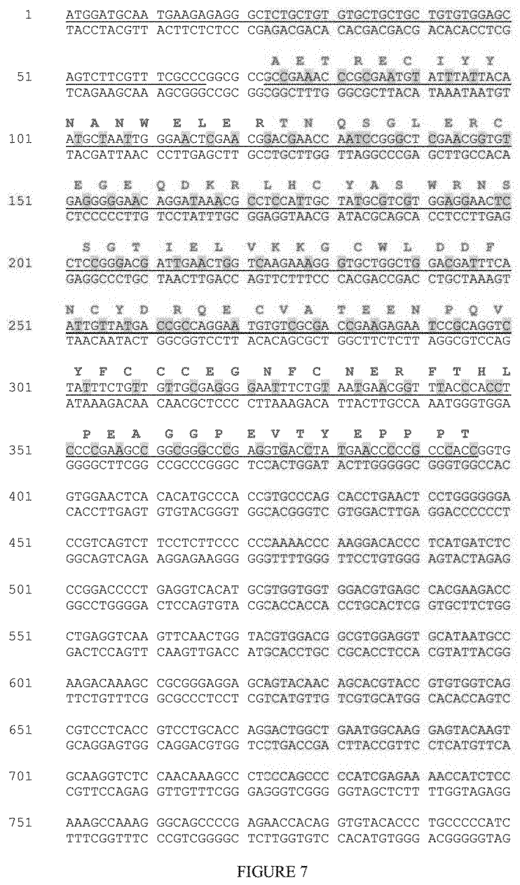

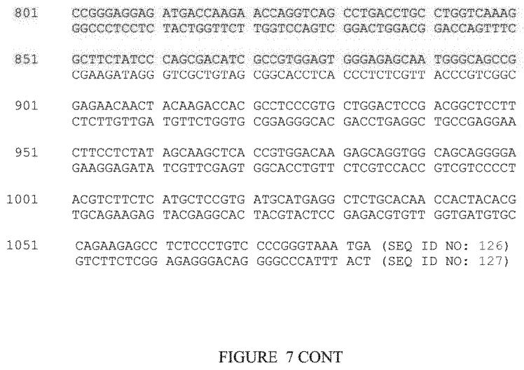

An ActRIIB(25-131)-hFc fusion protein, which comprises the human ActRIIB extracellular domain with N-terminal and C-terminal truncations (residues 25-131 of the native protein SEQ ID NO: 1) was fused N-terminally with a TPA leader sequence substituted for the native ActRIIB leader and C-terminally with a human Fc domain via a linker (FIG. 5; SEQ ID NO: 123). A nucleotide sequence encoding this fusion protein is shown in FIG. 6 (SEQ ID NO: 124 coding and SEQ ID NO: 125 complementary strand). The codons were modified and a variant nucleic acid encoding the ActRIIB(25-131)-hFc protein was found that provided substantial improvement in the expression levels of the fusion protein (FIG. 7; SEQ ID NO: 126 coding and SEQ ID NO: 127 complementary strand).

The mature protein has an amino acid sequence as follows (N-terminus confirmed by N-terminal sequencing) (SEQ ID NO: 63):

ETRECIYYNA NWELERTNQS GLERCEGEQD KRLHCYASWR
NSSGTIELVK KGCWLDDFNC YDRQECVATE ENPQVYFCCC
EGNFCNERFT HLPEAGGPEV TYEPPPTGGG THTCPPCPAP
ELLGGPSVFL FPPKPKDTLM ISRTPEVTCV VVDVSHEDPE
VKFNWYVDGV EVHNAKTKPR EEQYNSTYRV VSVLTVLHQD
WLNGKEYKCK VSNKALPAPI EKTISKAKGQ PREPQVYTLP
PSREEMTKNQ VSLTCLVKGF YPSDIAVEWE SNGQPENNYK
TTPPVLDSDG SFFLYSKLTV DKSRWQQGNV FSCSVMHEAL
HNHYTQKSLS LSPGK

Amino acids 1-107 are derived from ActRIIB.

The expressed molecule can be purified using a series of column chromatography steps, including for example, three or more of the following, in any order: Protein A chromatography, Q sepharose chromatography, phenylsepharose chromatography, size exclusion chromatography and cation exchange chromatography. The purification can be completed with viral filtration and buffer exchange.

Affinities of several ligands for ActRIIB(25-131)-hFc were evaluated in vitro with a Biacore™ instrument. Ligands were immobilized on a Biacore™ CMS chip using standard amine-coupling procedure. ActRIIB(25-131)-hFc was loaded onto the system, and binding was measured. ActRIIB(25-131)-hFc bound to various ligands including, for example, BMP10 with a dissociation constant (K_{D}) of 5.5×10⁻¹¹M, BMP9 with a K_{D} of 3.2×10¹⁰ M BMP6 with a K_{D} of 1.46×10⁻¹⁰ M, and BMP5 with a K_{D} of 2.19×10⁻⁸M.

A variant of ActRIIB(20-134) having a leucine-to-aspartate substitution at position 79 in SEQ ID NO:1 was fused to a Fc domain with a linker in between. The construct is referred to as ActRIIB(L79D 20-134)-hFc. Alternative forms with a glutamate rather than an aspartate at position 79 performed similarly (L79E) in binding assays. Alternative forms with an alanine rather than a valine at position 226 with respect to SEQ ID NO: 64, below, were also generated and performed equivalently in all respects tested. The aspartate at position 79 (relative to SEQ ID NO: 1, or position 60 relative to SEQ ID NO: 64) is indicated with double underlining below. The valine at position 226 relative to SEQ ID NO: 64 is also indicated by double underlining below.

The ActRIIB(L79D 20-134)-hFc is shown below as purified from CHO cell lines (SEQ ID NO: 64).

GRGEAETRECIYYNANWELERTNQSGLERCEGEQDKRLHCYASWRNSSGT
IELVKKGCWDDDFNCYDRQECVATEENPQVYFCCCEGNFCNERFTHLPEA
GGPEVTYEPPPTAPTGGGTHTCPPCPAPELLGGPSVFLFPPKPKDTLMIS
RTPEVTCVVVDVSHEDPEVKFNWYVDGVEVHNAKTKPREEQYNSTYRVVS
VLTVLHQDWLNGKEYKCKVSNKALPVPIEKTISKAKGQPREPQVYTLPPS
REEMTKNQVSLTCLVKGFYPSDIAVEWESNGQPENNYKTTPPVLDSDGSF
FLYSKLTVDKSRWQQGNVFSCSVMHEALHNHYTQKSLSLSPGK

The ActRIIB-derived portion of ActRIIB(L79D 20-134)-hFc has an amino acid sequence set forth below (SEQ ID NO: 65), and that portion could be used as a monomer or as a non-Fc fusion protein as a monomer, dimer, or greater-order complex.

(SEQ ID NO: 65)
GRGEAETRECIYYNANWELERTNQSGLERCEGEQDKRLHCYASWRNSSGT
IELVKKGCWDDDFNCYDRQECVATEENPQVYFCCCEGNFCNERFTHLPEA
GGPEVTYEPPPTAPT

The ActRIIB(L79D 20-134)-hFc fusion protein was expressed in CHO cell lines. Three different leader sequences were considered:

(i) Honey bee melittin (HBML), (ii) Tissue plasminogen activator (TPA), and (iii) Native.

The selected form employs the TPA leader and has the following unprocessed amino acid sequence:

(SEQ ID NO: 66)
MDAMKRGLCCVLLLCGAVFVSPGASGRGEAETRECIYYNANWELERTNQS
GLERCEGEQDKRLHCYASWRNSSGTIELVKKGCWDDDFNCYDRQECVATE
ENPQVYFCCCEGNFCNERFTHLPEAGGPEVTYEPPPTAPTGGGTHTCPPC
PAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALP
APIEKTISKAKGQPREPQVYTLPPSREEMTKNQVSLTCLVKGFYPSDIAV
EWESNGQPENNYKTTPPVLDSDGSFFLYSKLTVDKSRWQQGNVFSCSVMH
EALHNHYTQKSLSLSPGK

This polypeptide is encoded by the following nucleic acid sequence (SEQ ID NO: 67):

A TGGATGCAAT GAAGAGAGGG CTCTGCTGTG TGCTGCTGCT
GTGTGGAGCA GTCTTCGTTT CGCCCGGCGC CTCTGGGCGT
GGGGAGGCTG AGACACGGGA GTGCATCTAC TACAACGCCA
ACTGGGAGCT GGAGCGCACC AACCAGAGCG GCCTGGAGCG
CTGCGAAGGC GAGCAGGACA AGCGGCTGCA CTGCTACGCC
TCCTGGCGCA ACAGCTCTGG CACCATCGAG CTCGTGAAGA
AGGGCTGCTG GGACGATGAC TTCAACTGCT ACGATAGGCA
GGAGTGTGTG GCCACTGAGG AGAACCCCCA GGTGTACTTC
TGCTGCTGTG AAGGCAACTT CTGCAACGAG CGCTTCACTC
ATTTGCCAGA GGCTGGGGGC CCGGAAGTCA CGTACGAGCC
ACCCCCGACA GCCCCCACCG GTGGTGGAAC TCACACATGC
CCACCGTGCC CAGCACCTGA ACTCCTGGGG GGACCGTCAG
TCTTCCTCTT CCCCCCAAAA CCCAAGGACA CCCTCATGAT
CTCCCGGACC CCTGAGGTCA CATGCGTGGT GGTGGACGTG
AGCCACGAAG ACCCTGAGGT CAAGTTCAAC TGGTACGTGG
ACGGCGTGGA GGTGCATAAT GCCAAGACAA AGCCGCGGGA
GGAGCAGTAC AACAGCACGT ACCGTGTGGT CAGCGTCCTC
ACCGTCCTGC ACCAGGACTG GCTGAATGGC AAGGAGTACA
AGTGCAAGGT CTCCAACAAA GCCCTCCCAG TCCCCATCGA
GAAAACCATC TCCAAAGCCA AAGGGCAGCC CCGAGAACCA
CAGGTGTACA CCCTGCCCCC ATCCCGGGAG GAGATGACCA
AGAACCAGGT CAGCCTGACC TGCCTGGTCA AAGGCTTCTA
TCCCAGCGAC ATCGCCGTGG AGTGGGAGAG CAATGGGCAG
CCGGAGAACA ACTACAAGAC CACGCCTCCC GTGCTGGACT
CCGACGGCTC CTTCTTCCTC TATAGCAAGC TCACCGTGGA
CAAGAGCAGG TGGCAGCAGG GGAACGTCTT CTCATGCTCC
GTGATGCATG AGGCTCTGCA CAACCACTAC ACGCAGAAGA
GCCTCTCCCT GTCTCCGGGT AAATGA

Purification could be achieved by a series of column chromatography steps, including, for example, three or more of the following, in any order: protein A chromatography, Q sepharose chromatography, phenylsepharose chromatography, size exclusion chromatography, and cation exchange chromatography. The purification could be completed with viral filtration and buffer exchange. In an example of a purification scheme, the cell culture medium is passed over a protein A column, washed in 150 mM Tris/NaCl (pH 8.0), then washed in 50 mM Tris/NaCl (pH 8.0) and eluted with 0.1 M glycine, pH 3.0. The low pH eluate is kept at room temperature for 30 minutes as a viral clearance step. The eluate is then neutralized and passed over a Q-sepharose ion-exchange column and washed in 50 mM Tris pH 8.0, 50 mM NaCl, and eluted in 50 mM Tris pH 8.0, with an NaCl concentration between 150 mM and 300 mM. The eluate is then changed into 50 mM Tris pH 8.0, 1.1 M ammonium sulfate and passed over a phenyl sepharose column, washed, and eluted in 50 mM Tris pH 8.0 with ammonium sulfate between 150 and 300 mM. The eluate is dialyzed and filtered for use.

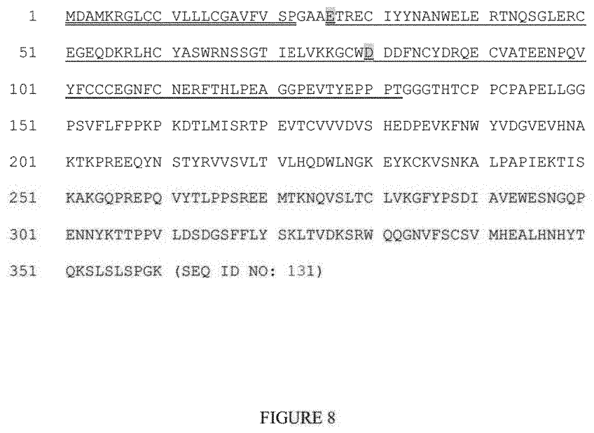

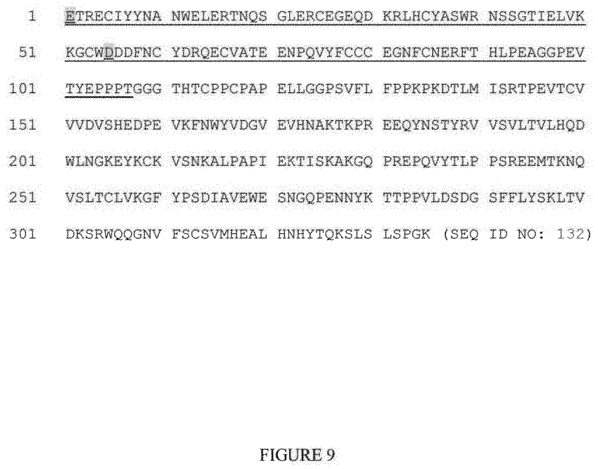

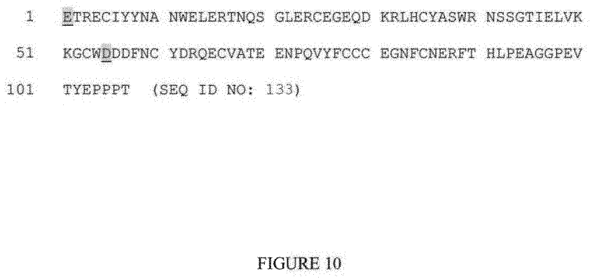

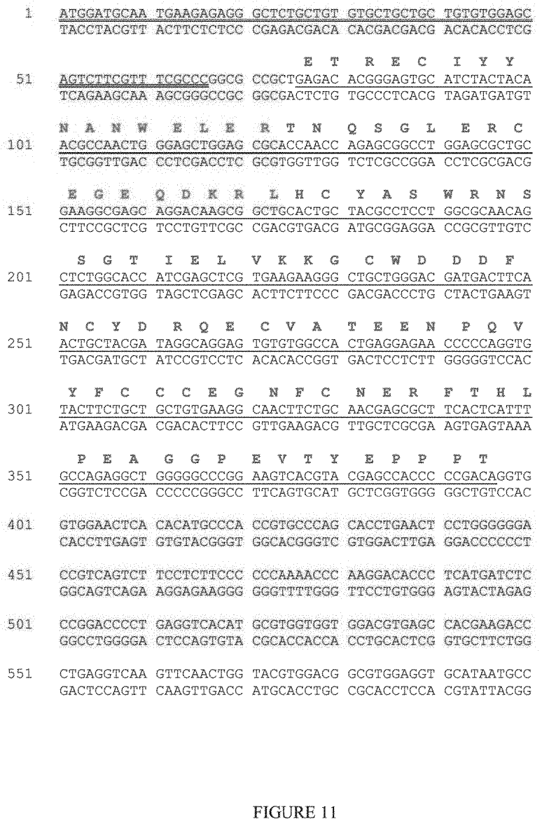

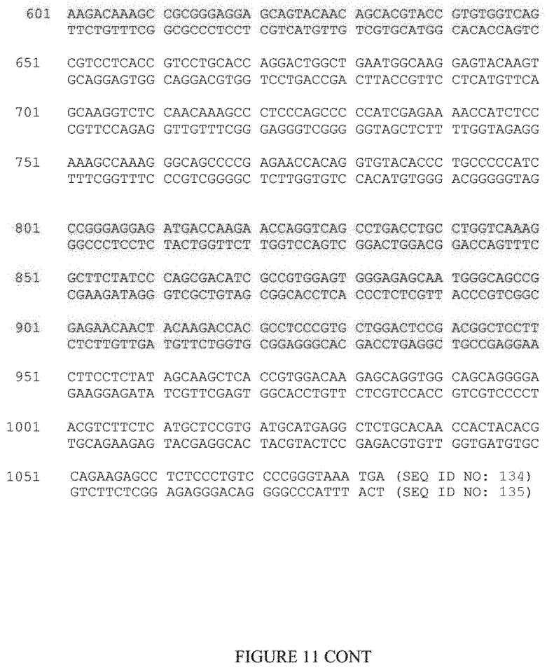

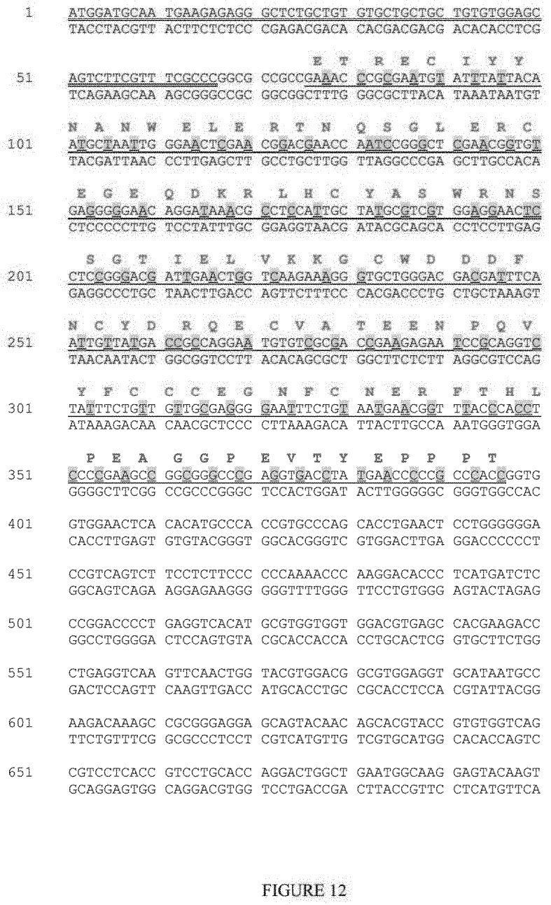

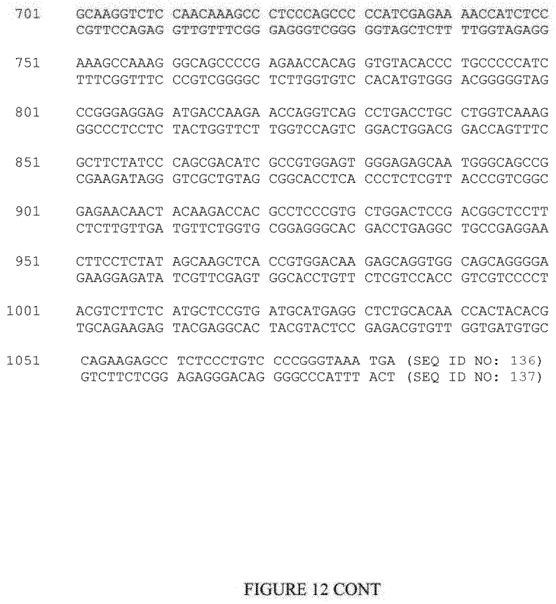

A variant of ActRIIB(25-131) having a leucine-to-aspartate substitution at position 79 in SEQ ID NO: 1 was fused to a Fc domain with a linker in between. The construct, including a TPA leader sequence, is depicted in FIG. 8 (SEQ ID NO: 131). The sequence of the cell purified form of ActRIIB(L79D 25-131)-hFc is presented in FIG. 9 (SEQ ID NO: 132) and the mature extracellular domain without the leader, linker or Fc domain is presented in FIG. 10 (SEQ ID NO: 133). One nucleotide sequence encoding this fusion protein is shown in FIG. 11 (SEQ ID NO: 134) along with its complementary sequence (SEQ ID NO: 135), and an alternative nucleotide sequence encoding exactly the same fusion protein is shown in FIG. 12 (SEQ ID NO: 136) and its complementary sequence (SEQ ID NO: 137).

Affinities of several ligands for ActRIIB(L79D 25-131)-hFc were evaluated in vitro with a Biacore™ instrument. Ligands were immobilized on a Biacore™ CMS chip using standard amine-coupling procedure. ActRIIB(L79D 25-131)-hFc was loaded onto the system, and binding was measured. ActRIIB(L79D 25-131)-hFc bound to various ligands including, for example, BMP10 with a dissociation constant (K_{D}) of 1.56×10⁻¹⁰ M and BMP6 with a K_{D} of 1.79×10⁻¹⁰ M. ActRIIB(L79D 25-131)-hFc had only transient affinity for BMP9 and no detectable binding affinity for BMP5. ActRIIB(L79D 25-131)-mFc behaved similarly.

### Example 3. Generation of a BMPRII-Fc Fusion Protein

A homodimeric BMPRII-Fc fusion protein comprising the extracellular domain of human BMPRII fused to a human immunoglobulin G1 Fc domain with a linker was generated. Leader sequences for use with BMPRII-Fc fusion polypeptide include the native human BMPRII precursor leader, MTSSLQRPWRVPWLPWTILLVSTAAA (SEQ ID NO: 68), and the tissue plasminogen activator (TPA) leader.

The human BMPRII-Fc polypeptide sequence (SEQ ID NO: 69) with a TPA leader is shown below:

| (SEQ ID NO: 69) | |
|---|---|
| 1 | MDAMKRGLCC VLLLCGAVFV SPGASQNQER LCAFKDPYQQ DLGIGESRIS |
| 51 | HENGTILCSK GSTCYGLWEK SKGDINLVKQ GCWSHIGDPQ ECHYEECVVT |
| 101 | TTPPSIQNGT YRFCCCSTDL CNVNFTENFP PPDTTPLSPP HSFNRDETGG |
| 151 | GTHTCPPCPA PELLGGPSVF LFPPKPKDTL MISRTPEVTC VVVDVSHEDP |
| 201 | EVKFNWYVDG VEVHNAKTKP REEQYNSTYR VVSVLTVLHQ DWLNGKEYKC |
| 251 | KVSNKALPAP IEKTISKAKG QPREPQVYTL PPSREEMTKN QVSLTCLVKG |
| 301 | FYPSDIAVEW ESNGQPENNY KTTPPVLDSD GSFFLYSKLT VDKSRWQQGN |
| 351 | VFSCSVMHEA LHNHYTQKSL SLSPGK |

The leader sequence and linker are underlined. The amino acid sequence of SEQ ID NO: 69 may optionally be provided with lysine removed from the C-terminus.

This BMPRII-Fc fusion protein is encoded by the following nucleic acid sequence (SEQ ID NO: 70):

| (SEQ ID NO: 70) | |
|---|---|
| 1 | ATGGATGCAA TGAAGAGAGG GCTCTGCTGT GTGCTGCTGC TGTGTGGAGC |
| 51 | AGTCTTCGTT TCGCCCGGCG CCTCGCAGAA TCAAGAACGC CTATGTGCGT |
| 101 | TTAAAGATCC GTATCAGCAA GACCTTGGGA TAGGTGAGAG TAGAATCTCT |
| 151 | CATGAAAATG GGACAATATT ATGCTCGAAA GGTAGCACCT GCTATGGCCT |
| 201 | TTGGGAGAAA TCAAAAGGGG ACATAAATCT TGTAAAACAA GGATGTTGGT |
| 251 | CTCACATTGG AGATCCCCAA GAGTGTCACT ATGAAGAATG TGTAGTAACT |
| 301 | ACCACTCCTC CCTCAATTCA GAATGGAACA TACCGTTTCT GCTGTTGTAG |
| 351 | CACAGATTTA TGTAATGTCA ACTTTACTGA GAATTTTCCA CCTCCTGACA |
| 401 | CAACACCACT CAGTCCACCT CATTCATTTA ACCGAGATGA GACCGGTGGT |
| 451 | GGAACTCACA CATGCCCACC GTGCCCAGCA CCTGAACTCC TGGGGGGACC |
| 501 | GTCAGTCTTC CTCTTCCCCC CAAAACCCAA GGACACCCTC ATGATCTCCC |
| 551 | GGACCCCTGA GGTCACATGC GTGGTGGTGG ACGTGAGCCA CGAAGACCCT |
| 601 | GAGGTCAAGT TCAACTGGTA CGTGGACGGC GTGGAGGTGC ATAATGCCAA |
| 651 | GACAAAGCCG CGGGAGGAGC AGTACAACAG CACGTACCGT GTGGTCAGCG |
| 701 | TCCTCACCGT CCTGCACCAG GACTGGCTGA ATGGCAAGGA GTACAAGTGC |
| 751 | AAGGTCTCCA ACAAAGCCCT CCCAGCCCCC ATCGAGAAAA CCATCTCCAA |
| 801 | AGCCAAAGGG CAGCCCCGAG AACCACAGGT GTACACCCTG CCCCCATCCC |
| 851 | GGGAGGAGAT GACCAAGAAC CAGGTCAGCC TGACCTGCCT GGTCAAAGGC |
| 901 | TTCTATCCCA GCGACATCGC CGTGGAGTGG GAGAGCAATG GGCAGCCGGA |
| 951 | GAACAACTAC AAGACCACGC CTCCCGTGCT GGACTCCGAC GGCTCCTTCT |
| 1001 | TCCTCTATAG CAAGCTCACC GTGGACAAGA GCAGGTGGCA GCAGGGGAAC |
| 1051 | GTCTTCTCAT GCTCCGTGAT GCATGAGGCT CTGCACAACC ACTACACGCA |
| 1101 | GAAGAGCCTC TCCCTGTCTC CGGGTAAA |

A processed BMPRII-Fc fusion polypeptide (SEQ ID NO: 71) is as follows and may optionally be provided with lysine removed from the C-terminus.

| (SEQ ID NO: 71) | |
|---|---|
| 1 | SQNQERLCAF KDPYQQDLGI GESRISHENG TILCSKGSTC YGLWEKSKGD |
| 51 | INLVKQGCWS HIGDPQECHY EECVVTTTPP SIQNGTYRFC CCSTDLCNVN |
| 101 | FTENFPPPDT TPLSPPHSFN RDETGGGTHT CPPCPAPELL GGPSVFLFPP |
| 151 | KPKDTLMISR TPEVTCVVVD VSHEDPEVKF NWYVDGVEVH NAKTKPREEQ |
| 201 | YNSTYRVVSV LTVLHQDWLN GKEYKCKVSN KALPAPIEKT ISKAKGQPRE |
| 251 | PQVYTLPPSR EEMTKNQVSL TCLVKGFYPS DIAVEWESNG QPENNYKTTP |
| 301 | PVLDSDGSFF LYSKLTVDKS RWQQGNVFSC SVMHEALHNH YTQKSLSLSP |
| 351 | GK |

This BMPRII-Fc fusion protein is encoded by the following nucleic acid sequence (SEQ ID NO: 72):

| (SEQ ID NO: 72) | |
|---|---|
| 1 | TCGCAGAATC AAGAACGCCT ATGTGCGTTT AAAGATCCGT ATCAGCAAGA |
| 51 | CCTTGGGATA GGTGAGAGTA GAATCTCTCA TGAAAATGGG ACAATATTAT |
| 101 | GCTCGAAAGG TAGCACCTGC TATGGCCTTT GGGAGAAATC AAAAGGGGAC |
| 151 | ATAAATCTTG TAAAACAAGG ATGTTGGTCT CACATTGGAG ATCCCCAAGA |
| 201 | GTGTCACTAT GAAGAATGTG TAGTAACTAC CACTCCTCCC TCAATTCAGA |
| 251 | ATGGAACATA CCGTTTCTGC TGTTGTAGCA CAGATTTATG TAATGTCAAC |
| 301 | TTTACTGAGA ATTTTCCACC TCCTGACACA ACACCACTCA GTCCACCTCA |
| 351 | TTCATTTAAC CGAGATGAGA CCGGTGGTGG AACTCACACA TGCCCACCGT |
| 401 | GCCCAGCACC TGAACTCCTG GGGGGACCGT CAGTCTTCCT CTTCCCCCCA |
| 451 | AAACCCAAGG ACACCCTCAT GATCTCCCGG ACCCCTGAGG TCACATGCGT |
| 501 | GGTGGTGGAC GTGAGCCACG AAGACCCTGA GGTCAAGTTC AACTGGTACG |
| 551 | TGGACGGCGT GGAGGTGCAT AATGCCAAGA CAAAGCCGCG GGAGGAGCAG |
| 601 | TACAACAGCA CGTACCGTGT GGTCAGCGTC CTCACCGTCC TGCACCAGGA |
| 651 | CTGGCTGAAT GGCAAGGAGT ACAAGTGCAA GGTCTCCAAC AAAGCCCTCC |
| 701 | CAGCCCCCAT CGAGAAAACC ATCTCCAAAG CCAAAGGGCA GCCCCGAGAA |
| 751 | CCACAGGTGT ACACCCTGCC CCCATCCCGG GAGGAGATGA CCAAGAACCA |
| 801 | GGTCAGCCTG ACCTGCCTGG TCAAAGGCTT CTATCCCAGC GACATCGCCG |
| 851 | TGGAGTGGGA GAGCAATGGG CAGCCGGAGA ACAACTACAA GACCACGCCT |
| 901 | CCCGTGCTGG ACTCCGACGG CTCCTTCTTC CTCTATAGCA AGCTCACCGT |
| 951 | GGACAAGAGC AGGTGGCAGC AGGGGAACGT CTTCTCATGC TCCGTGATGC |
| 1001 | ATGAGGCTCT GCACAACCAC TACACGCAGA AGAGCCTCTC CCTGTCTCCG |
| 1051 | GGTAAA |

The BMPRII-Fc fusion polypeptide of SEQ ID NO: 71 may be expressed and purified from a CHO cell line to give rise to a homodimeric BMPRII-Fc fusion protein complex.

Purification of various BMPRII-Fc complexes could be achieved by a series of column chromatography steps, including, for example, three or more of the following, in any order: protein A chromatography, Q sepharose chromatography, phenylsepharose chromatography, size exclusion chromatography, and cation exchange chromatography. The purification could be completed with viral filtration and buffer exchange.

A Biacore™-based binding assay was used to determine the ligand binding selectivity of the BMPRII-Fc protein complex described above. The BMPRII-Fc homodimer was captured onto the system using an anti-Fc antibody, and ligands were injected and allowed to flow over the captured receptor protein. Results are summarized in the table below.

Ligand binding profile of BMPRII-Fc homodimer

| | kₐ | k_{d} | K_{D} |
|---|---|---|---|
| Ligand | (1/Ms) | (1/s) | (pM) |
| BMP10 | 2.6 × 10⁷ | 2.5 × 10⁻³ | 100 |
| BMP9 | 1.2 × 10⁷ | 2.6 × 10⁻² | 2100 |

| | | |
|---|---|---|
| BMP6 | Transient* | 8900 |

*Indeterminate due to transient nature of interaction

These ligand binding data demonstrate that homodimeric BMPRII-Fc fusion protein binds with high picomolar affinity to BMP10 and with approximately ten-fold lower affinity to BMP9. As ligand traps, BMPRII-Fc polypeptides should preferably exhibit a slow rate of ligand dissociation, so the off-rates observed for BMP10 in particular is desirable. Surprisingly, despite literature suggesting that BMPRII acts as the major type II receptor for canonical BMP proteins such as BMP2, BMP4, BMP6 or BMP7, BMPRII-Fc fusion protein shows no substantial binding to any of BMP2, BMP4, BMP6 or BMP7. Accordingly, homodimeric BMPRII-Fc will be useful in certain therapeutic applications where antagonism of BMP10 and BMP9 is advantageous.

### Example 4: ALK1-Fc Fusion Proteins

An ALK1 fusion protein was generated that has the extracellular domain of human ALK1 fused to a human Fc or mouse ALK1 fused to a murine Fc domain with a linker in between. The constructs are referred to as ALK1-hFc and mALK1-mFc, respectively.

Notably, while the conventional C-terminus of the extracellular domain of human ALK1 protein is amino acid 118 of SEQ ID NO: 20, we have determined that it is desirable to avoid having a domain that ends at a glutamine residue. Accordingly, the portion of SEQ ID NO: 76 that derives from human ALK1 incorporates two residues C-terminal to Q118, a leucine and an alanine. The disclosure therefore provides ALK1 ECD polypeptides (including Fc fusion proteins) having a C-terminus of the ALK1 derived sequence that is anywhere from 1 to 5 amino acids upstream (113-117 relative to SEQ ID NO: 20) or downstream (119-123) of Q118.

The ALK1-hFc and ALK1-mFc proteins were expressed in CHO cell lines. Three different leader sequences were considered: (i) Honey bee mellitin (HBML), (ii) Tissue Plasminogen Activator (TPA), and Native: MTLGSPRKGLLMLLMALVTQG (SEQ ID NO: 73).

The selected ALK1-hFc form employs the TPA leader and has the unprocessed amino acid sequence shown in below:

(SEQ ID NO: 74)

MDAMKRGLCCVLLLCGAVFVSPGADPVKPSRGPLVTCTCESPHCKGPTCR
GAWCTVVLVREEGRHPQEHRGCGNLHRELCRGRPTEFVNHYCCDSHLCNH
NVSLVLEATQPPSEQPGTDGQLATGGGTHTCPPCPAPEALGAPSVFLFPP
KPKDTLMISRTPEVTCVVVDVSHEDPEVKFNWYVDGVEVHNAKTKPREEQ
YNSTYRVVSVLTVLHQDWLNGKEYKCKVSNKALPVPIEKTISKAKGQPRE
PQVYTLPPSREEMTKNQVSLTCLVKGFYPSDIAVEWESNGQPENNYKTTP
PVLDSDGPFFLYSKLTVDKSRWQQGNVFSCSVMHEALHNHYTQKSLSLSP
GK

The ALK1 extracellular domain is underlined.

The ALK1-hFc as purified from CHO cell lines is shown below:

(SEQ ID NO: 76)

DPVKPSRGPLVTCTCESPHCKGPTCRGAWCTVVLVREEGRHPQEHRGCGN
LHRELCRGRPTEFVNHYCCDSHLCNHNVSLVLEATQPPSEQPGTDGQLAT
GGGTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKEY
KCKVSNKALPVPIEKTISKAKGQPREPQVYTLPPSREEMTKNQVSLTCLV
KGFYPSDIAVEWESNGQPENNYKTTPPVLDSDGSFFLYSKLTVDKSRWQQ
GNVFSCSVMHEALHNHYTQKSLSLSPGK

Purification can be achieved by a series of column chromatography steps, including, for example, three or more of the following, in any order: protein A chromatography, Q sepharose chromatography, phenylsepharose chromatography, size exclusion chromatography, and cation exchange chromatography. The purification can be completed with viral filtration and buffer exchange. The ALK1-hFc protein was purified to a purity of >98% as determined by size exclusion chromatography and >95% as determined by SDS PAGE.

Affinities of several ligands for ALK1-hFc were evaluated in vitro with a Biacore™ instrument. Various ligands were immobilized on a Biacore™ CM5 chip using standard amine-coupling procedure. ALK1-hFc was loaded onto the system, and binding was measured. ALK1-hFc bound to BMP10 with a dissociation constant (K_{D}) of 1.49×10⁻¹¹M and BMP9 with a K_{D} of 3.14×10⁻¹¹M. ALK1-hFc had no detectable binding affinity for BMP5 or BMP6. ALK1-mFc behaved similarly.

### Example 5: Generation of ENG-Fc Fusion Proteins

Endoglin (ENG) fusion protein [hENG(26-586)-hFc] in which the full-length extracellular domain (ECD) of human ENG (amino acids 26-586 of SEQ ID NO: 24) was attached to a human IgG1 Fc domain with a linker between these domains.

Three different leader sequences were considered: (i) Honey bee mellitin (HBML), (ii) Tissue plasminogen activator (TPA), and (iii) native human ENG: MDRGTLPLAVALLLASCSLSPTSLA (SEQ ID NO: 77)

The selected form of hENG(26-586)-hFc uses the TPA leader, has the unprocessed amino acid sequence shown in below:

| (SEQ ID NO: 78) | |
|---|---|
| 1 | MDAMKRGLCC VLLLCGAVFV SPGAETVHCD LQPVGPERDE VTYTTSQVSK |
| 51 | GCVAQAPNAI LEVHVLFLEF PTGPSQLELT LQASKQNGTW PREVLLVLSV |
| 101 | NSSVFLHLQA LGIPLHLAYN SSLVTFQEPP GVNTTELPSF PKTQILEWAA |
| 151 | ERGPITSAAE LNDPQSILLR LGQAQGSLSF CMLEASQDMG RTLEWRPRTP |
| 201 | ALVRGCHLEG VAGHKEAHIL RVLPGHSAGP RTVTVKVELS CAPGDLDAVL |
| 251 | ILQGPPYVSW LIDANHNMQI WTTGEYSFKI FPEKNIRGFK LPDTPQGLLG |
| 301 | EARMLNASIV ASFVELPLAS IVSLHASSCG GRLQTSPAPI QTTPPKDTCS |
| 351 | PELLMSLIQT KCADDAMTLV LKKELVAHLK CTITGLTFWD PSCEAEDRGD |
| 401 | KFVLRSAYSS CGMQVSASMI SNEAVVNILS SSSPQRKKVH CLNMDSLSFQ |
| 451 | LGLYLSPHFL QASNTIEPGQ QSFVQVRVSP SVSEFLLQLD SCHLDLGPEG |
| 501 | GTVELIQGRA AKGNCVSLLS PSPEGDPRFS FLLHFYTVPI PKTGTLSCTV |
| 551 | ALRPKTGSQD QEVHRTVFMR LNIISPDLSG CTSKGTGGGP KSCDKTHTCP |
| 601 | PCPAPELLGG PSVFLFPPKP KDTLMISRTP EVTCVVVDVS HEDPEVKFNW |
| 651 | YVDGVEVHNA KTKPREEQYN STYRVVSVLT VLHQDWLNGK EYKCKVSNKA |
| 701 | LPAPIEKTIS KAKGQPREPQ VYTLPPSREE MTKNQVSLTC LVKGFYPSDI |
| 751 | AVEWESNGQP ENNYKTTPPV LDSDGSFFLY SKLTVDKSRW QQGNVFSCSV |
| 801 | MHEALHNHYT QKSLSLSPGK |

The ENG extracellular domain is denoted with a single underline; the TPA leader is denoted with a double underline.

The hENG(26-586)-hFc described above is encoded by the nucleotide sequence shown below:

| (SEQ ID NO: 79) | |
|---|---|
| 1 | ATGGATGCAA TGAAGAGAGG GCTCTGCTGT GTGCTGCTGC TGTGTGGAGC |
| | AGTCTTCGTT TCGCCCGGCG CCGAAACAGT CCATTGTGAC CTTCAGCCTG |
| 101 | TGGGCCCCGA GAGGGACGAG GTGACATATA CCACTAGCCA GGTCTCGAAG |
| | GGCTGCGTGG CTCAGGCCCC CAATGCCATC CTTGAAGTCC ATGTCCTCTT |
| 201 | CCTGGAGTTC CCAACGGGCC CGTCACAGCT GGAGCTGACT CTCCAGGCAT |
| | CCAAGCAAAA TGGCACCTGG CCCCGAGAGG TGCTTCTGGT CCTCAGTGTA |
| 301 | AACAGCAGTG TCTTCCTGCA TCTCCAGGCC CTGGGAATCC CACTGCACTT |
| | GGCCTACAAT TCCAGCCTGG TCACCTTCCA AGAGCCCCCG GGGGTCAACA |
| 401 | CCACAGAGCT GCCATCCTTC CCCAAGACCC AGATCCTTGA GTGGGCAGCT |
| | GAGAGGGGCC CCATCACCTC TGCTGCTGAG CTGAATGACC CCCAGAGCAT |
| 501 | CCTCCTCCGA CTGGGCCAAG CCCAGGGGTC ACTGTCCTTC TGCATGCTGG |
| | AAGCCAGCCA GGACATGGGC CGCACGCTCG AGTGGCGGCC GCGTACTCCA |
| 601 | GCCTTGGTCC GGGGCTGCCA CTTGGAAGGC GTGGCCGGCC ACAAGGAGGC |
| | GCACATCCTG AGGGTCCTGC CGGGCCACTC GGCCGGGCCC CGGACGGTGA |
| 701 | CGGTGAAGGT GGAACTGAGC TGCGCACCCG GGGATCTCGA TGCCGTCCTC |
| | ATCCTGCAGG GTCCCCCCTA CGTGTCCTGG CTCATCGACG CCAACCACAA |
| 801 | CATGCAGATC TGGACCACTG GAGAATACTC CTTCAAGATC TTTCCAGAGA |
| | AAAACATTCG TGGCTTCAAG CTCCCAGACA CACCTCAAGG CCTCCTGGGG |
| 901 | GAGGCCCGGA TGCTCAATGC CAGCATTGTG GCATCCTTCG TGGAGCTACC |
| | GCTGGCCAGC ATTGTCTCAC TTCATGCCTC CAGCTGCGGT GGTAGGCTGC |
| 1001 | AGACCTCACC CGCACCGATC CAGACCACTC CTCCCAAGGA CACTTGTAGC |
| | CCGGAGCTGC TCATGTCCTT GATCCAGACA AAGTGTGCCG ACGACGCCAT |
| 1101 | GACCCTGGTA CTAAAGAAAG AGCTTGTTGC GCATTTGAAG TGCACCATCA |
| | CGGGCCTGAC CTTCTGGGAC CCCAGCTGTG AGGCAGAGGA CAGGGGTGAC |
| 1201 | AAGTTTGTCT TGCGCAGTGC TTACTCCAGC TGTGGCATGC AGGTGTCAGC |
| | AAGTATGATC AGCAATGAGG CGGTGGTCAA TATCCTGTCG AGCTCATCAC |
| 1301 | CACAGCGGAA AAAGGTGCAC TGCCTCAACA TGGACAGCCT CTCTTTCCAG |
| | CTGGGCCTCT ACCTCAGCCC ACACTTCCTC CAGGCCTCCA ACACCATCGA |
| 1401 | GCCGGGGCAG CAGAGCTTTG TGCAGGTCAG AGTGTCCCCA TCCGTCTCCG |
| | AGTTCCTGCT CCAGTTAGAC AGCTGCCACC TGGACTTGGG GCCTGAGGGA |
| 1501 | GGCACCGTGG AACTCATCCA GGGCCGGGCG GCCAAGGGCA ACTGTGTGAG |
| | CCTGCTGTCC CCAAGCCCCG AGGGTGACCC GCGCTTCAGC TTCCTCCTCC |
| 1601 | ACTTCTACAC AGTACCCATA CCCAAAACCG GCACCCTCAG CTGCACGGTA |
| | GCCCTGCGTC CCAAGACCGG GTCTCAAGAC CAGGAAGTCC ATAGGACTGT |
| 1701 | CTTCATGCGC TTGAACATCA TCAGCCCTGA CCTGTCTGGT TGCACAAGCA |
| | AAGGCACCGG TGGTGGACCC AAATCTTGTG ACAAAACTCA CACATGCCCA |
| 1801 | CCGTGCCCAG CACCTGAACT CCTGGGGGGA CCGTCAGTCT TCCTCTTCCC |
| | CCCAAAACCC AAGGACACCC TCATGATCTC CCGGACCCCT GAGGTCACAT |
| 1901 | GCGTGGTGGT GGACGTGAGC CACGAAGACC CTGAGGTCAA GTTCAACTGG |
| | TACGTGGACG GCGTGGAGGT GCATAATGCC AAGACAAAGC CGCGGGAGGA |
| 2001 | GCAGTACAAC AGCACGTACC GTGTGGTCAG CGTCCTCACC GTCCTGCACC |
| | AGGACTGGCT GAATGGCAAG GAGTACAAGT GCAAGGTCTC CAACAAAGCC |
| 2101 | CTCCCAGCCC CCATCGAGAA AACCATCTCC AAAGCCAAAG GGCAGCCCCG |
| | AGAACCACAG GTGTACACCC TGCCCCCATC CCGGGAGGAG ATGACCAAGA |
| 2201 | ACCAGGTCAG CCTGACCTGC CTGGTCAAAG GCTTCTATCC CAGCGACATC |
| | GCCGTGGAGT GGGAGAGCAA TGGGCAGCCG GAGAACAACT ACAAGACCAC |
| 2301 | GCCTCCCGTG CTGGACTCCG ACGGCTCCTT CTTCCTCTAT AGCAAGCTCA |
| | CCGTGGACAA GAGCAGGTGG CAGCAGGGGA ACGTCTTCTC ATGCTCCGTG |
| 2401 | ATGCATGAGG CTCTGCACAA CCACTACACG CAGAAGAGCC TCTCCCTGTC |
| | CCCGGGTAAA TGA |

The ENG extracellular domain is denoted with a single underline; the TPA leader is denoted with a double underline.

An alternative hENG(26-586)-hFc sequence with TPA leader comprising an N-terminally truncated hFc domain attached to hENG(26-586) by a T linker was also envisioned:

| (SEQ ID NO: 80) | |
|---|---|
| 1 | MDAMKRGLCC VLLLCGAVFV SPGAETVHCD LQPVGPERDE VTYTTSQVSK |
| 51 | GCVAQAPNAI LEVHVLFLEF PTGPSQLELT LQASKQNGTW PREVLLVLSV |
| 101 | NSSVFLHLQA LGIPLHLAYN SSLVTFQEPP GVNTTELPSF PKTQILEWAA |
| 151 | ERGPITSAAE LNDPQSILLR LGQAQGSLSF CMLEASQDMG RTLEWRPRTP |
| 201 | ALVRGCHLEG VAGHKEAHIL RVLPGHSAGP RTVTVKVELS CAPGDLDAVL |
| 251 | ILQGPPYVSW LIDANHNMQI WTTGEYSFKI FPEKNIRGFK LPDTPQGLLG |
| 301 | EARMLNASIV ASFVELPLAS IVSLHASSCG GRLQTSPAPI QTTPPKDTCS |
| 351 | PELLMSLIQT KCADDAMTLV LKKELVAHLK CTITGLTFWD PSCEAEDRGD |
| 401 | KFVLRSAYSS CGMQVSASMI SNEAVVNILS SSSPQRKKVH CLNMDSLSFQ |
| 451 | LGLYLSPHFL QASNTIEPGQ QSFVQVRVSP SVSEFLLQLD SCHLDLGPEG |
| 501 | GTVELIQGRA AKGNCVSLLS PSPEGDPRFS FLLHFYTVPI PKTGTLSCTV |
| 551 | ALRPKTGSQD QEVHRTVFMR LNIISPDLSG CTSKGTGGGT HTCPPCPAPE |
| 601 | LLGGPSVFLF PPKPKDTLMI SRTPEVTCVV VDVSHEDPEV KFNWYVDGVE |
| 651 | VHNAKTKPRE EQYNSTYRVV SVLTVLHQDW LNGKEYKCKV SNKALPAPIE |
| 701 | KTISKAKGQP REPQVYTLPP SREEMTKNQV SLTCLVKGFY PSDIAVEWES |
| 751 | NGQPENNYKT TPPVLDSDGS FFLYSKLTVD KSRWQQGNVF SCSVMHEALH |
| 801 | NHYTQKSLSL SPGK |

Purification was achieved using a variety of techniques, including, for example, filtration of conditioned media, followed by protein A chromatography, elution with low-pH (3.0) glycine buffer, sample neutralization, and dialysis against PBS. Purity of samples was evaluated by analytical size-exclusion chromatography, SDS-PAGE, silver staining, and Western blot. Analysis of mature protein confirmed the expected N-terminal sequence.

Considered a co-receptor, ENG is widely thought to function by facilitating the binding of TGF-β1 and -3 to multi-protein complexes of type I and type II receptors. To investigate the possibility of direct ligand binding by isolated ENG, surface plasmon resonance (SPR) methodology (Biacore™ instrument) was used to screen for binding of captured proteins comprising the full-length extracellular domain of ENG to a variety of soluble human TGF-β family ligands.

Construct Binding

| Ligand | hENG(26-586)-hFc* | hENG(26-586)** |
|---|---|---|
| hBMP-2 | − | − |
| hBMP-2/7 | − | − |
| hBMP-7 | − | − |
| hBMP-9 | ++++ | ++++ |
| hBMP-10 | ++++ | ++++ |
| hTGF-β1 | − | − |
| hTGF-β2 | − | − |
| hTGF-β3 | − | − |
| hActivin A | − | − |

*[hBMP-9], [hBMP-10] = 2.5 nM; all other ligands tested at 100 nM
**[hBMP-9], [hBMP-10] = 2.5 nM; all other ligands tested at 25 nM

As shown in this table, binding affinity to hENG(26-586)-hFc was high (++++, K_{D}<1 nM) for hBMP9 and hBMP10 as evaluated at low ligand concentrations. Even at concentrations 40-fold higher, binding of TGF-β1, TGF-β2, TGF-β3, activin A, BMP2, and BMP7 to hENG(26-586)-hFc was undetectable (−). For this latter group of ligands, lack of direct binding to isolated ENG fusion protein is noteworthy because multiprotein complexes of type I and type II receptors have been shown to bind most of them better in the presence of ENG than in its absence. As also shown in the table above, similar results were obtained when ligands were screened for their ability to bind immobilized hENG(26-586) (R&D Systems, catalog #1097-EN), a human variant with no Fc domain. Characterization by SPR determined that captured hENG(26-586)-hFc binds soluble BMP9 with a K_{D} of 29 pM and soluble BMP10 with a K_{D} of 400 pM. Thus, selective high-affinity binding of BMP9 and BMP10 is a previously unrecognized property of the ENG extracellular domain.

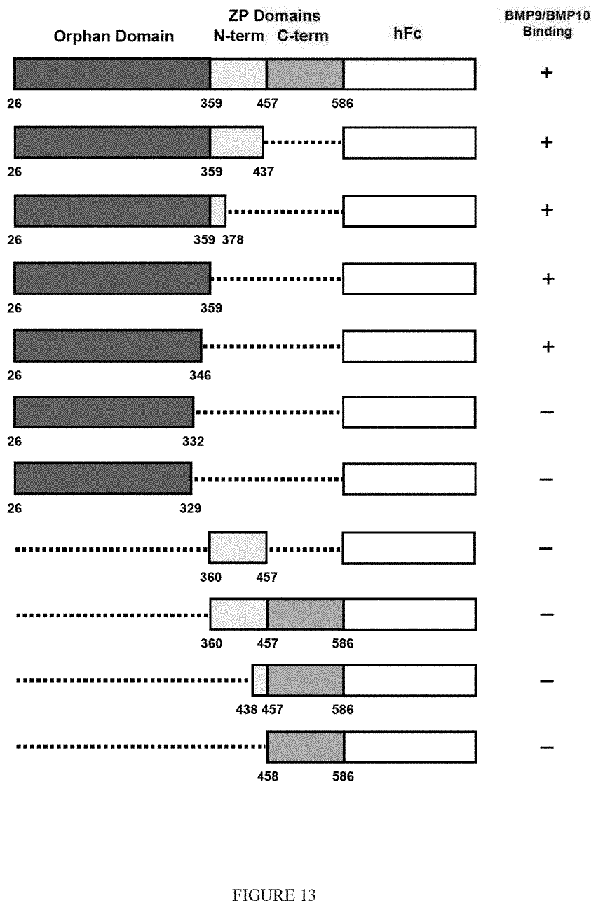

ENG fusion proteins in which truncated variants of the human ENG ECD were fused to a human IgG₁ Fc domain with a linker between where also generated. These variants are listed below, and the structures of selected variants are shown schematically in FIG. 13.

| | | | | Stable |
|---|---|---|---|---|
| | | Transient | | Expression |
| | Human Construct | Expression | Purified | (CHO Cells) |
| Full Length | hENG(26-586)-hFc | HEK 293 | Yes | Yes |
| Carboxy- | hENG(26-581)-hFc | HEK 293 | Yes | No |
| Terminal | hENG(26-437)-hFc | HEK 293 | Yes | No |
| Truncations | hENG(26-378)-hFc | HEK 293 | Yes | No |
| | hENG(26-359)-hFc | HEK 293 | Yes | Yes |
| | hENG(26-346)-hFc | HEK 293 | Yes | Yes |
| | hENG(26-332)-hFc | HEK 293 | Yes | No |
| | hENG(26-329)-hFc | HEK 293 | Yes | No |
| | hENG(26-257)-hFc | HEK 293 | Yes | No |
| Amino- | hENG(360-586)-hFc | HEK 293 | Yes | No |
| Terminal | hENG(438-586)-hFc | HEK 293 | Yes | No |
| Truncations | hENG(458-586)-hFc | COS | No | No |
| Double | hENG(61-346)-hFc | HEK 293 | Yes | No |
| Truncations | hENG(129-346)-hFc | HEK 293 | Yes | No |
| | hENG(133-346)-hFc | HEK 293 | Yes | No |
| | hENG(166-346)-hFc | HEK 293 | Yes | No |
| | hENG(258-346)-hFc | HEK 293 | Yes | No |
| | hENG(360-581)-hFc | HEK 293 | Yes | No |
| | hENG(360-457)-hFc | COS | No | No |
| | hENG(360-437)-hFc | COS | No | No |
| | hENG(458-581)-hFc | COS | No | No |

These variants were expressed by transient transfection in HEK 293 cells or COS cells, as indicated.

SPR methodology was used to screen these hENG-hFc protein variants for high-affinity binding to human BMP9 and BMP10. In these experiments, captured hENG-hFc proteins were exposed to soluble BMP9 or BMP10 at 100 nM each.

| | | Binding to hBMP9 |
|---|---|---|
| | Human Construct | and hBMP10 |
| Full Length | hENG(26-586)-hFc | ++++ |
| Carboxy-Terminal | hENG(26-581)-hFc | ++++ |
| Truncations | hENG(26-437)-hFc | ++++ |
| | hENG(26-378)-hFc | ++++ |
| | hENG(26-359)-hFc | ++++ |
| | hENG(26-346)-hFc | ++++ |
| | hENG(26-332)-hFc | − |
| | hENG(26-329)-hFc | − |
| | hENG(26-257)-hFc | − |
| Amino-Terminal | hENG(360-586)-hFc | − |
| Truncations | hENG(438-586)-hFc | − |
| | hENG(458-586)-hFc | − |
| Double Truncations | hENG(61-346)-hFc | − |
| | hENG(129-346)-hFc | − |
| | hENG(133-346)-hFc | − |
| | hENG(166-346)-hFc | − |
| | hENG(258-346)-hFc | − |
| | hENG(360-581)-hFc | − |
| | hENG(360-457)-hFc | − |
| | hENG(360-437)-hFc | − |
| | hENG(458-581)-hFc | − |

++++ KD <1 nM
− Binding undetectable

As indicated in the table above, high-affinity binding to BMP9 and BMP10 was observed only for the full-length construct and for C-terminally truncated variants as short as hENG(26-346)-hFc. High-affinity binding to BMP9 and BMP10 was lost for all N-terminal truncations of greater than 61 amino acids that were tested.

A panel of ligands were screened for potential binding to the C-terminal truncated variants hENG(26-346)-hFc, hENG(26-359)-hFc, and hENG(26-437)-hFc. High-affinity binding of these three proteins was selective for BMP9 and BMP10. Neither hENG(26-346)-hFc, hENG(26-359)-hFc, nor hENG(26-437)-hFc displayed detectable binding to BMP2, BMP7, TGFβ1, TGFβ2, TGFβ3, or activin A, even at high ligand concentrations.

| | Construct Binding | | |
|---|---|---|---|
| | hENG(26-346)- | hENG(26-359)- | hENG(26-437)- |
| Ligand | hFc* | hFc** | hFc** |
| hBMP-2 | − | − | − |
| hBMP-2/7 | − | − | − |
| hBMP-7 | − | − | − |
| hBMP-9 | ++++ | ++++ | ++++ |
| hBMP-10 | ++++ | ++++ | ++++ |
| hTGF-β1 | − | − | − |
| hTGF-β2 | − | − | − |
| hTGF-β3 | − | − | − |
| hActivin A | − | − | − |

*[hBMP-9], [hBMP-10] = 5 nM; [hTGF-β3] = 50 nM; all other ligands tested at 100 nM
**[hBMP-9], [hBMP-10] = 5 nM; [hTGF-β3] = 50 nM; all other ligands tested at 100 nM
++++ KD <1 nM
− Binding undetectable

SPR methodology was to compare the kinetics of BMP9 binding by five constructs: hENG(26-586)-hFc, hENG(26-437)-hFc, hENG(26-378)-hFc, hENG(26-359)-hFc, and hENG(26-346)-hFc. The affinity of human BMP-9 for hENG(26-359)-hFc or hENG(26-346)-hFc (with K_{D}s in the low picomolar range) was nearly an order of magnitude stronger than for the full-length construct. It is highly desirable for ligand traps such as ENG-Fc to exhibit a relatively slow rate of ligand dissociation, so the ten-fold improvement (decrease) in the BMP9 dissociation rate for hENG(26-346)-hFc compared to the full-length construct is particularly noteworthy.

| Ligand | Construct | K_{D} (×10⁻¹²M) | k_{d} (×10⁻⁴ s⁻¹) |
|---|---|---|---|
| hBMP-9 | hENG(26-586)-hFc* | 33 | 25 |
| | hENG(26-437)-hFc** | 19 | 14 |
| | hENG(26-378)-hFc** | 6.7 | 3.4 |
| | hENG(26-359)-hFc* | 4.2 | 3.5 |
| | hENG(26-346)-hFc* | 4.3 | 2.4 |

*CHO-cell-derived protein
**HEK293-cell-derived protein

As shown below, each of the truncated variants also bound BMP10 with higher affinity, and with better kinetics, compared to the full-length construct. Even so, the truncated variants differed in their degree of preference for BMP9 over BMP10 (based on K_{D} ratio), with hENG(26-346)-hFc displaying the largest differential and hENG(26-437)-hFC the smallest. This difference in degree of ligand preference among the truncated variants could potentially translate into meaningful differences in their activity in vivo.

| Ligand | Construct | K_{D} (×10⁻¹²M) | k_{d} (×10⁻⁴ s⁻¹) |
|---|---|---|---|
| hBMP-10 | hENG(26-586)-hFc* | 490 | 110 |
| | hENG(26-437)-hFc** | 130 | 28 |
| | hENG(26-378)-hFc** | 95 | 19 |
| | hENG(26-359)-hFc* | 86 | 23 |
| | hENG(26-346)-hFc* | 140 | 28 |

*CHO-cell-derived protein
**HEK293-cell-derived protein

The foregoing results indicate that fusion proteins comprising certain C-terminally truncated variants of the hENG ECD display high-affinity binding to BMP9 and BMP10 but not to a variety of other TGF-β family ligands, including TGFβ1 and TGFβ3. In particular, the truncated variants hENG(26-359)-hFc, hENG(26-346)-hFc, and hENG(26-378)-hFc display higher binding affinity at equilibrium and improved kinetic properties for BMP-9 compared to both the full-length construct hENG(26-586)-hFc and the truncated variant hENG(26-437)-hFc.

As disclosed above, N-terminal truncations as short as 36 amino acids (hENG(61-346)-hFc) were found to abolish ligand binding to ENG polypeptides. To anticipate the effect of even shorter N-terminal truncations on ligand binding, the secondary structure for the human endoglin orphan domain was predicted computationally with a modified Psipred version 3 (Jones, 1999, J Mol Biol 292:195-202). The analysis indicates that ordered secondary structure within the ENG polypeptide region defined by amino acids 26-60 of SEQ ID NO: 24 is limited to a four-residue beta strand predicted with high confidence at positions 42-45 of SEQ ID NO: 24 and a two-residue beta strand predicted with very low confidence at positions 28-29 of SEQ ID NO: 24. Accordingly, ENG polypeptide variants beginning at amino acids 27 or 28 and optionally those beginning at any of amino acids 29-42 of SEQ ID NO: 24 are likely to retain important structural elements and ligand binding.

For the mouse studies described below, an ENG-Fc fusion protein was constructed by fusing a truncated portion of the extracellular domain of human endoglin (i.e., amino acids 27-581) to an Fc domain of mouse IgG1 with a minimal linker (TGGG) positioned between the two domains. This construct is designated as hENG(27-581)-mFc and desmonstrated similar binding affinities as described above for hENG(26-581)-hFc.

### Example 6: BMP10 Propeptide Fusion Proteins

A BMP10 propeptide-Fc fusion protein comprising a C-terminal truncated, human BMP10 propeptide domain (amino acids 22-315 of SEQ ID NO: 32 fused to a human immunoglobulin G1 Fc domain with an optional linker was generated. This fusion protein was designated as BMP10pro(22-315)-hFc. A similar fusion protein was generated using a mouse immunoglobulin G1 Fc domain, which is designated as BMP10pro(22-315)-mFc. Signal sequences for use with BMP10 propeptide-Fc fusion protein that were considered include, for example, the native human BMP10 precursor leader, MGSLVLTLCALFCLAAYLVSG (SEQ ID NO: 81), honeybee mellitin, and the tissue TPA leader.

The human BMP10pro(22-315)-hFc polypeptide sequence (SEQ ID NO: 82) with a TPA leader is shown below:

| (SEQ ID NO: 82) | |
|---|---|
| 1 | MDAMKRGLCC VLLLCGAVFV SPGASPIMNL EQSPLEEDMS LFGDVFSEQD |
| 51 | GVDFNTLLQS MKDEFLKTLN LSDIPTQDSA KVDPPEYMLE LYNKFATDRT |
| 101 | SMPSANIIRS FKNEDLFSQP VSFNGLRKYP LLFNVSIPHH EEVIMAELRL |
| 151 | YTLVQRDRMI YDGVDRKITI FEVLESKGDN EGERNMLVLV SGEIYGTNSE |
| 201 | WETFDVTDAI RRWQKSGSST HQLEVHIESK HDEAEDASSG RLEIDTSAQN |
| 251 | KHNPLLIVFS DDQSSDKERK EELNEMISHE QLPELDNLGL DSFSSGPGEE |
| 301 | ALLQMRSNII YDSTARIRTG GGTHTCPPCP APELLGGPSV FLFPPKPKDT |
| 351 | LMISRTPEVT CVVVDVSHED PEVKFNWYVD GVEVHNAKTK PREEQYNSTY |
| 401 | RVVSVLTVLH QDWLNGKEYK CKVSNKALPA PIEKTISKAK GQPREPQVYT |
| 451 | LPPSREEMTK NQVSLTCLVK GFYPSDIAVE WESNGQPENN YKTTPPVLDS |
| 501 | DGSFFLYSKL TVDKSRWQQG NVFSCSVMHE ALHNHYTQKS LSLSPGK |

The BMP propeptide domain is underlined. The amino acid sequence of SEQ ID NO: 82 may optionally be provided with lysine removed from the C-terminus.

This BMP10pro(22-315)-hFc fusion protein is encoded by the following nucleic acid sequence (SEQ ID NO: 83):

(SEQ ID NO: 83)

| | |
|---|---|
| 1 | ATGGATGCAA TGAAGAGAGG GCTCTGCTGT GTGCTGCTGC |
| | TGTGTGGAGC |
| 51 | AGTCTTCGTT TCGCCCGGCG CCAGCCCCAT CATGAACCTA |
| | GAGCAGTCTC |
| 101 | CTCTGGAAGA AGATATGTCC CTCTTTGGTG ATGTTTTCTC |
| | AGAGCAAGAC |
| 151 | GGTGTCGACT TTAACACACT GCTCCAGAGC ATGAAGGATG |
| | AGTTTCTTAA |
| 201 | GACACTAAAC CTCTCTGACA TCCCCACGCA GGATTCAGCC |
| | AAGGTGGACC |
| 251 | CACCAGAGTA CATGTTGGAA CTCTACAACA AATTTGCAAC |
| | AGATCGGACC |
| 301 | TCCATGCCCT CTGCCAACAT CATTAGGAGT TTCAAGAATG |
| | AAGATCTGTT |
| 351 | TTCCCAGCCG GTCAGTTTTA ATGGGCTCCG AAAATACCCC |
| | CTCCTCTTCA |
| 401 | ATGTGTCCAT TCCTCACCAT GAAGAGGTCA TCATGGCTGA |
| | ACTTAGGCTA |
| 451 | TACACACTGG TGCAAAGGGA TCGTATGATA TACGATGGAG |
| | TAGACCGGAA |
| 501 | AATTACCATT TTTGAAGTGC TGGAGAGCAA AGGGGATAAT |
| | GAGGGAGAAA |
| 551 | GAAACATGCT GGTCTTGGTG TCTGGGGAGA TATATGGAAC |
| | CAACAGTGAG |
| 601 | TGGGAGACTT TTGATGTCAC AGATGCCATC AGACGTTGGC |
| | AAAAGTCAGG |
| 651 | CTCATCCACC CACCAGCTGG AGGTCCACAT TGAGAGCAAA |
| | CACGATGAAG |
| 701 | CTGAGGATGC CAGCAGTGGA CGGCTAGAAA TAGATACCAG |
| | TGCCCAGAAT |
| 751 | AAGCATAACC CTTTGCTCAT CGTGTTTTCT GATGACCAAA |
| | GCAGTGACAA |
| 801 | GGAGAGGAAG GAGGAACTGA ATGAAATGAT TTCCCATGAG |
| | CAACTTCCAG |
| 851 | AGCTGGACAA CTTGGGCCTG GATAGCTTTT CCAGTGGACC |
| | TGGGGAAGAG |
| 901 | GCTTTGTTGC AGATGAGATC AAACATCATC TATGACTCCA |
| | CTGCCCGAAT |
| 951 | CAGAACCGGT GGTGGAACTC ACACATGCCC ACCGTGCCCA |
| | GCACCTGAAC |
| 1001 | TCCTGGGGGG ACCGTCAGTC TTCCTCTTCC CCCCAAAACC |
| | CAAGGACACC |
| 1051 | CTCATGATCT CCCGGACCCC TGAGGTCACA TGCGTGGTGG |
| | TGGACGTGAG |
| 1101 | CCACGAAGAC CCTGAGGTCA AGTTCAACTG GTACGTGGAC |
| | GGCGTGGAGG |
| 1151 | TGCATAATGC CAAGACAAAG CCGCGGGAGG AGCAGTACAA |
| | CAGCACGTAC |
| 1201 | CGTGTGGTCA GCGTCCTCAC CGTCCTGCAC CAGGACTGGC |
| | TGAATGGCAA |
| 1251 | GGAGTACAAG TGCAAGGTCT CCAACAAAGC CCTCCCAGCC |
| | CCCATCGAGA |
| 1301 | AAACCATCTC CAAAGCCAAA GGGCAGCCCC GAGAACCACA |
| | GGTGTACACC |
| 1351 | CTGCCCCCAT CCCGGGAGGA GATGACCAAG AACCAGGTCA |
| | GCCTGACCTG |
| 1401 | CCTGGTCAAA GGCTTCTATC CCAGCGACAT CGCCGTGGAG |
| | TGGGAGAGCA |
| 1451 | ATGGGCAGCC GGAGAACAAC TACAAGACCA CGCCTCCCGT |
| | GCTGGACTCC |
| 1501 | GACGGCTCCT TCTTCCTCTA TAGCAAGCTC ACCGTGGACA |
| | AGAGCAGGTG |
| 1551 | GCAGCAGGGG AACGTCTTCT CATGCTCCGT GATGCATGAG |
| | GCTCTGCACA |
| 1601 | ACCACTACAC GCAGAAGAGC CTCTCCCTGT CTCCGGGTAA |
| | ATGA |

A processed BMP10pro(22-315)-hFc fusion protein (SEQ ID NO: 84) is as follows and may optionally be provided with lysine removed from the C-terminus.

| (SEQ ID NO: 84) | |
|---|---|
| 1 | SPIMNL EQSPLEEDMS LFGDVFSEQD |
| 51 | GVDFNTLLQS MKDEFLKTLN LSDIPTQDSA KVDPPEYMLE LYNKFATDRT |
| 101 | SMPSANIIRS FKNEDLFSQP VSFNGLRKYP LLFNVSIPHH EEVIMAELRL |
| 151 | YTLVQRDRMI YDGVDRKITI FEVLESKGDN EGERNMLVLV SGEIYGTNSE |
| 201 | WETFDVTDAI RRWQKSGSST HQLEVHIESK HDEAEDASSG RLEIDTSAQN |
| 251 | KHNPLLIVFS DDQSSDKERK EELNEMISHE QLPELDNLGL DSFSSGPGEE |
| 301 | ALLQMRSNII YDSTARIRTG GGTHTCPPCP APELLGGPSV FLFPPKPKDT |
| 351 | LMISRTPEVT CVVVDVSHED PEVKFNWYVD GVEVHNAKTK PREEQYNSTY |
| 401 | RVVSVLTVLH QDWLNGKEYK CKVSNKALPA PIEKTISKAK GQPREPQVYT |
| 451 | LPPSREEMTK NQVSLTCLVK GFYPSDIAVE WESNGQPENN YKTTPPVLDS |
| 501 | DGSFFLYSKL TVDKSRWQQG NVFSCSVMHE ALHNHYTQKS LSLSPGK |

The BMP propeptide domain is underlined.

Another BMP10 propeptide-Fc fusion protein was generated comprising a greater C-terminal truncation of the human BMP10 propeptide domain (amino acids 22-312 of SEQ ID NO: 32) fused to a human immunoglobulin G1 Fc domain with an optional linker. This fusion protein was designated as BMP10pro(22-312)-hFc. A similar fusion protein was generated using a mouse immunoglobulin G1 Fc domain, which is designated as BMP10pro(22-312)-mFc. Signal sequences for use with the BMP10pro(22-312)-Fc fusion proteins that were considered include, for example, the native human BMP10 precursor leader, honeybee mellitin, and TPA leader.

The human BMP10pro(22-312)-hFc polypeptide sequence (SEQ ID NO: 85) with a TPA leader is shown below:

| (SEQ ID NO: 85) | |
|---|---|
| 1 | MDAMKRGLCC VLLLCGAVFV SPGASPIMNL EQSPLEEDMS LFGDVFSEQD |
| 51 | GVDFNTLLQS MKDEFLKTLN LSDIPTQDSA KVDPPEYMLE LYNKFATDRT |
| 101 | SMPSANIIRS FKNEDLFSQP VSFNGLRKYP LLFNVSIPHH EEVIMAELRL |
| 151 | YTLVQRDRMI YDGVDRKITI FEVLESKGDN EGERNMLVLV SGEIYGTNSE |
| 201 | WETFDVTDAI RRWQKSGSST HQLEVHIESK HDEAEDASSG RLEIDTSAQN |
| 251 | KHNPLLIVFS DDQSSDKERK EELNEMISHE QLPELDNLGL DSFSSGPGEE |
| 301 | ALLQMRSNII YDSTATGGGT HTCPPCPAPE LLGGPSVFLF PPKPKDTLMI |
| 351 | SRTPEVTCVV VDVSHEDPEV KFNWYVDGVE VHNAKTKPRE EQYNSTYRVV |
| 401 | SVLTVLHQDW LNGKEYKCKV SNKALPAPIE KTISKAKGQP REPQVYTLPP |
| 451 | SREEMTKNQV SLTCLVKGFY PSDIAVEWES NGQPENNYKT TPPVLDSDGS |
| 501 | FFLYSKLTVD KSRWQQGNVF SCSVMHEALH NHYTQKSLSL SPGK |

The BMP propeptide domain is underlined. The amino acid sequence of SEQ ID NO: 85 may optionally be provided with lysine removed from the C-terminus.

This BMP10pro(22-312)-hFc fusion protein is encoded by the following nucleic acid sequence (SEQ ID NO: 86):

| (SEQ ID NO: 86) | |
|---|---|
| 1 | ATGGATGCAA TGAAGAGAGG GCTCTGCTGT GTGCTGCTGC TGTGTGGAGC |
| 51 | AGTCTTCGTT TCGCCCGGCG CCAGCCCCAT CATGAACCTA GAGCAGTCTC |
| 101 | CTCTGGAAGA AGATATGTCC CTCTTTGGTG ATGTTTTCTC AGAGCAAGAC |
| 151 | GGTGTCGACT TTAACACACT GCTCCAGAGC ATGAAGGATG AGTTTCTTAA |
| 201 | GACACTAAAC CTCTCTGACA TCCCCACGCA GGATTCAGCC AAGGTGGACC |
| 251 | CACCAGAGTA CATGTTGGAA CTCTACAACA AATTTGCAAC AGATCGGACC |
| 301 | TCCATGCCCT CTGCCAACAT CATTAGGAGT TTCAAGAATG AAGATCTGTT |
| 351 | TTCCCAGCCG GTCAGTTTTA ATGGGCTCCG AAAATACCCC CTCCTCTTCA |
| 401 | ATGTGTCCAT TCCTCACCAT GAAGAGGTCA TCATGGCTGA ACTTAGGCTA |
| 451 | TACACACTGG TGCAAAGGGA TCGTATGATA TACGATGGAG TAGACCGGAA |
| 501 | AATTACCATT TTTGAAGTGC TGGAGAGCAA AGGGGATAAT GAGGGAGAAA |
| 551 | GAAACATGCT GGTCTTGGTG TCTGGGGAGA TATATGGAAC CAACAGTGAG |
| 601 | TGGGAGACTT TTGATGTCAC AGATGCCATC AGACGTTGGC AAAAGTCAGG |
| 651 | CTCATCCACC CACCAGCTGG AGGTCCACAT TGAGAGCAAA CACGATGAAG |
| 701 | CTGAGGATGC CAGCAGTGGA CGGCTAGAAA TAGATACCAG TGCCCAGAAT |
| 751 | AAGCATAACC CTTTGCTCAT CGTGTTTTCT GATGACCAAA GCAGTGACAA |
| 801 | GGAGAGGAAG GAGGAACTGA ATGAAATGAT TTCCCATGAG CAACTTCCAG |
| 851 | AGCTGGACAA CTTGGGCCTG GATAGCTTTT CCAGTGGACC TGGGGAAGAG |
| 901 | GCTTTGTTGC AGATGAGATC AAACATCATC TATGACTCCA CTGCCACCGG |
| 951 | TGGTGGAACT CACACATGCC CACCGTGCCC AGCACCTGAA CTCCTGGGGG |
| 1001 | GACCGTCAGT CTTCCTCTTC CCCCCAAAAC CCAAGGACAC CCTCATGATC |
| 1051 | TCCCGGACCC CTGAGGTCAC ATGCGTGGTG GTGGACGTGA GCCACGAAGA |
| 1101 | CCCTGAGGTC AAGTTCAACT GGTACGTGGA CGGCGTGGAG GTGCATAATG |
| 1151 | CCAAGACAAA GCCGCGGGAG GAGCAGTACA ACAGCACGTA CCGTGTGGTC |
| 1201 | AGCGTCCTCA CCGTCCTGCA CCAGGACTGG CTGAATGGCA AGGAGTACAA |
| 1251 | GTGCAAGGTC TCCAACAAAG CCCTCCCAGC CCCCATCGAG AAAACCATCT |
| 1301 | CCAAAGCCAA AGGGCAGCCC CGAGAACCAC AGGTGTACAC CCTGCCCCCA |
| 1351 | TCCCGGGAGG AGATGACCAA GAACCAGGTC AGCCTGACCT GCCTGGTCAA |
| 1401 | AGGCTTCTAT CCCAGCGACA TCGCCGTGGA GTGGGAGAGC AATGGGCAGC |
| 1451 | CGGAGAACAA CTACAAGACC ACGCCTCCCG TGCTGGACTC CGACGGCTCC |
| 1501 | TTCTTCCTCT ATAGCAAGCT CACCGTGGAC AAGAGCAGGT GGCAGCAGGG |
| 1551 | GAACGTCTTC TCATGCTCCG TGATGCATGA GGCTCTGCAC AACCACTACA |
| 1601 | CGCAGAAGAG CCTCTCCCTG TCTCCGGGTA AATGA |

A processed BMP10pro(22-312)-hFc fusion protein (SEQ ID NO: 87) is as follows and may optionally be provided with lysine removed from the C-terminus.

| (SEQ ID NO: 87) | |
|---|---|
| 1 | SPIMNL EQSPLEEDMS LFGDVFSEQD |
| 51 | GVDFNTLLQS MKDEFLKTLN LSDIPTQDSA KVDPPEYMLE LYNKFATDRT |
| 101 | SMPSANIIRS FKNEDLFSQP VSFNGLRKYP LLFNVSIPHH EEVIMAELRL |
| 151 | YTLVQRDRMI YDGVDRKITI FEVLESKGDN EGERNMLVLV SGEIYGTNSE |
| 201 | WETFDVTDAI RRWQKSGSST HQLEVHIESK HDEAEDASSG RLEIDTSAQN |
| 251 | KHNPLLIVFS DDQSSDKERK EELNEMISHE QLPELDNLGL DSFSSGPGEE |
| 301 | ALLQMRSNII YDSTATGGGT HTCPPCPAPE LLGGPSVFLF PPKPKDTLMI |
| 351 | SRTPEVTCVV VDVSHEDPEV KFNWYVDGVE VHNAKTKPRE EQYNSTYRVV |
| 401 | SVLTVLHQDW LNGKEYKCKV SNKALPAPIE KTISKAKGQP REPQVYTLPP |
| 451 | SREEMTKNQV SLTCLVKGFY PSDIAVEWES NGQPENNYKT TPPVLDSDGS |
| 501 | FFLYSKLTVD KSRWQQGNVF SCSVMHEALH NHYTQKSLSL SPGK |

The BMP propeptide domain is underlined.

The BMP10pro-Fc fusion proteins described above may be expressed and purified from a COS or CHO cell line to give rise to a homodimeric BMP10pro-Fc fusion protein complex.

Purification of various BMP10pro-Fc fusion proteins complexes can be achieved by a series of column chromatography steps, including, for example, three or more of the following, in any order: protein A chromatography, Q sepharose chromatography, phenylsepharose chromatography, size exclusion chromatography, and cation exchange chromatography. The purification was completed with viral filtration and buffer exchange.

A panel of ligands were screened for potential binding to the C-terminal truncated variants BMP10pro(22-315)-hFc and BMP10pro(22-312)-hFc, as well as the corresponding mouse fusion proteins, using a Biacore™-based binding assay. The BMP10pro-Fc proteins were separately was captured onto the system using an anti-Fc antibody, and ligands were injected and allowed to flow over the captured receptor protein. BMP10pro(22-315)-hFc and BMP10pro(22-312)-hFc both showed high affinity for BMP10 and BMP9. Both constructs also displayed high to moderate affinity for BMP6 and BMP5. The mouse Fc equivalent constructs behaved similarly. BMP10pro(22-312)-hFc also displayed high affinity for BMP3b. The affinity of BMP10pro(22-315)-hFc for BMP3b was not assessed.

Using a luciferase reporter construct under the control of four sequential consensus SBE sites (SBE4-luc), which are responsive to Smad 1/4/8-mediated signaling, the mature BMP10-mediated activity in the presence and absence of BMP10pro(22-315)-hFc and BMP10pro(22-312)-hFc, separately, was measured in HMVEC cells. Results are show in the table below

IC₅₀ (BMP10)

| Fc fusion protein | (ng/ml) | (pM) |
|---|---|---|
| BMP10pro(22-315)-hFc | 61.20 | 516.8 |
| BMP10pro(22-312)-hFc | 17.74 | 149.81 |

The data indicate that BMP10 propeptides can tolerate C-terminal truncations of 1, 2, 3, or 4 amino acids without losing BMP10 antagonizing activity. Moreover, while both fusion proteins were shown to be potent inhibitors of BMP10 activity, the BMP10pro(22-312)-hFc fusion protein was determined to antagonize BMP10 activity threefold or greater than the BMP10pro(22-315)-hFc. The increased activity of BMP10pro(22-312)-hFc is surprising given that it has a greater C-terminal truncation than BMP10pro(22-315)-hFc. Therefore, in certain uses where it is desirable to maximize BMP10 inhibition, a polypeptide comprising a BMP10pro domain ending at residue 312 with respect to SEQ ID NO: 32 may be preferable to a BMP10pro domain ending at any one or residues 313-315 with respect to SEQ ID NO: 32. In addition, to having greater activity, the shorter BMP10pro domain may be preferable in certain therapeutic applications where it is desirable to reduce risk of immune reaction against the BMP10pro polypeptide, i.e., less amino acids reduces the number of potential epitopes that may be recognized by a patient's immune system.

### Example 7. Effects of BMP10 Propeptide Treatment in Heart Failure Model

The effects of BMP10pro(22-312)-Fc on the progression of heart failure were investigated using a mouse Transverse Aortic Constriction (TAC) model, which mimics aortic stenosis. See, e.g., Nakamura et al. (2001) Am J Physiol Heart Circ Physiol. 281: H1104-H1112.

Thirty 10 week-old C57/B6 male mice underwent TAC surgery and ten age-matched animals underwent the same surgical procedure except for TAC (Sham mice) at day 0. After waking from the surgery, TAC mice were randomized into two groups. One group of 15 mice were injected subcutaneously with BMP10pro (22-312)-mFc at a dose of 10 mg/kg (TAC-BMP10pro mice) and a second group of 15 mice were injected subcutaneously with phosphate buffered saline (vehicle control mice; TAC-PBS mice), every 3 days for 21 days. At the end of the study, echocardiography was performed to measure left ventricular function and remodeling before animals were euthanized for heart collection. Each heart was photographed, fixed in 10% formalin, and sectioned for Masson's trichrome stain to assess fibrosis.

In vivo cardiac function was assessed by transthoracic echocardiography (Acuson P300, 18 MHz transducer; Siemens) in conscious mice. From left ventricle short axis view, M-mode echocardiogram was acquired to measure interventricular septal thickness at end diastole, left ventricular posterior wall thickness at end diastole, left ventricle end diastolic diameter, and left ventricle end systolic diameter. Fractional shortening (FS) was calculated from the end diastolic diameter (EDD) and end systolic diameter (ESD) using the following equation: FS=100%×[(EDD−ESD)/EDD]. Early diastolic filling peak velocity (E), late filling peak velocity (A), and isovolumetric relaxation time (IVRT) were measured from the medial or septal wall at the mitral valve level from tissue Doppler image. Left ventricle diastolic function was assessed by measuring the E/A ratio and IVRT. Three to five beats were averaged for each mouse measurement. The overall 21-day mortality rates were also calculated.

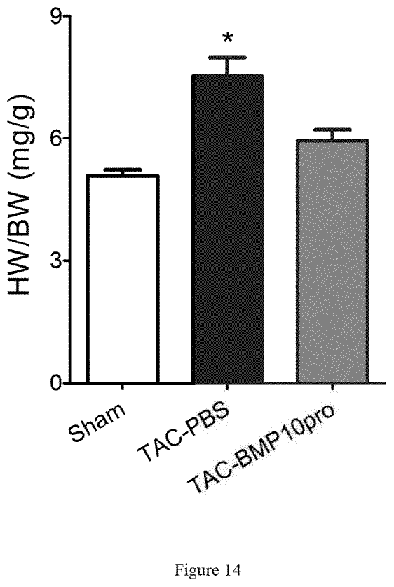

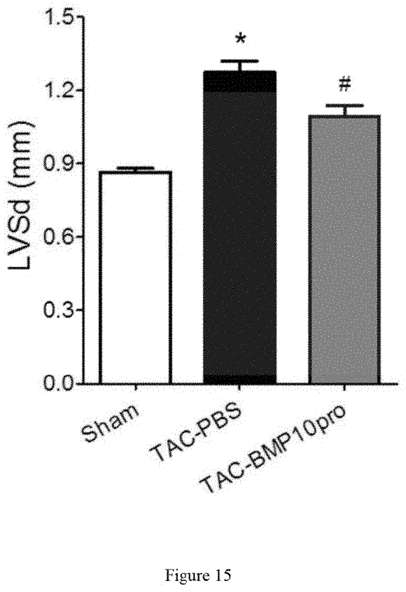

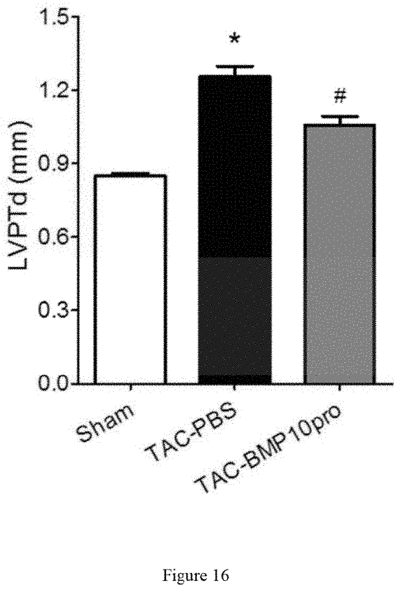

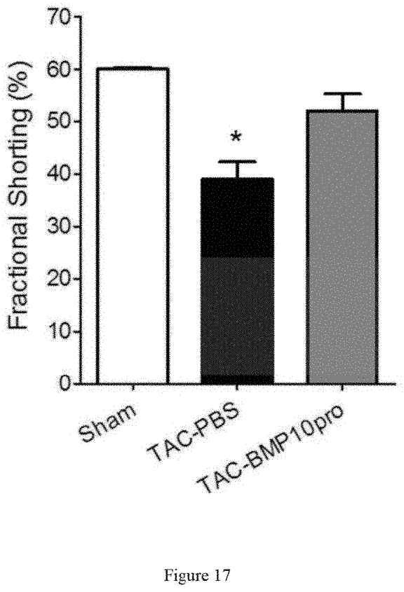

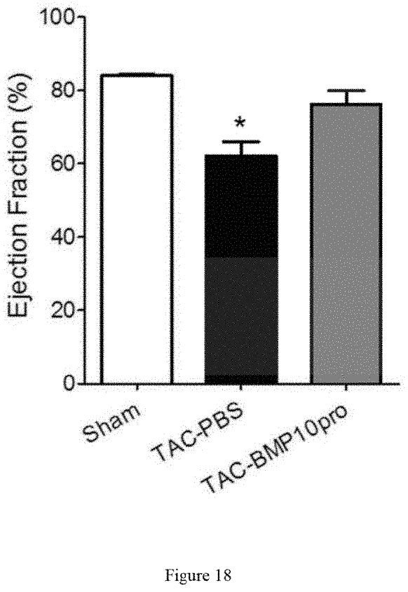

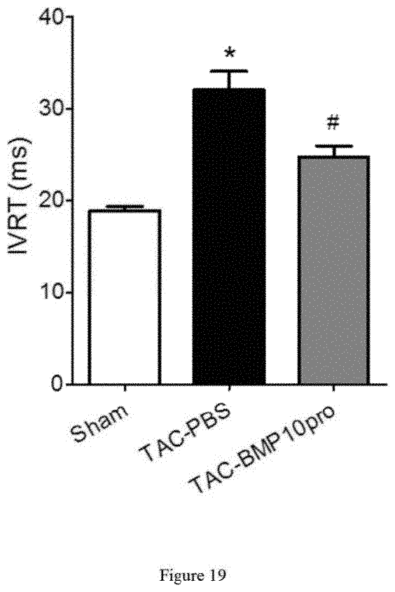

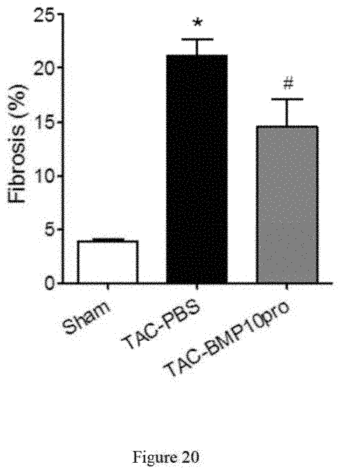

In this study, treatment of mice with BMP10pro(22-312)-Fc significantly inhibited cardiac hypertrophy (see FIG. 14), suppressed cardiac remodeling (see FIGS. 15 and 16), improved cardiac function (see FIGS. 17-19), and histology data confirmed inhibition of cardiac fibrosis (see FIG. 20). Furthermore, BMP10pro(22-312)-Fc treatment increased survival time of animals in this heart failure model. Together, these data demonstrate that BMP10 propeptide treatment is effective at treating heart failure, surprisingly effective in treating many distinct complications of heart failure. This indicates that BMP10 propeptides may have an advantage in treating heart failure over current standard of care treatments that only treat one or a few complications of heart failure. Furthermore, as BMP10 propeptides have a selective BMP-binding profile (particular binding with high affinity to BMP10, BMP9, BMP6, and BMP3b and to a lesser extent BMP5), the data presented herein suggests that other BMP antagonists with similar binding-properties may have similar beneficial effects in treating heart failure, particularly preventing or reducing the severity of various complications of heart failure.

### Example 8. Effects of Endo-Fc Treatment in Heart Failure Model

The effects of hENG(27-581)-mFc on the progression of heart failure were investigated using a mouse Transverse Aortic Constriction (TAC) model, which mimics aortic stenosis. See, e.g., Nakamura et al. (2001) Am J Physiol Heart Circ Physiol. 281: H1104-H1112.

Thirty 10 week-old C57/B6 male mice underwent TAC surgery and ten age-matched animals underwent the same surgical procedure except for TAC (SHAM mice) at day 0. After waking from the surgery, TAC mice were randomized into two groups. One group of 15 mice were injected subcutaneously with hENG(27-581)-mFc at a dose of 10 mg/kg (TAC-Endo) and a second group of 15 mice were injected subcutaneously with phosphate buffered saline (vehicle control mice; TAC-PBS mice), every 3 days for 21 days. At the end of the study, echocardiography was performed to measure left ventricular function and remodeling before animals were euthanized for heart collection. Each heart was photographed, fixed in 10% formalin, and sectioned for Masson's trichrome stain to assess fibrosis.

In vivo cardiac function was assessed by transthoracic echocardiography (Acuson P300, 18 MHz transducer; Siemens) in conscious mice. From left ventricle short axis view, M-mode echocardiogram was acquired to measure interventricular septal thickness at end diastole, left ventricular posterior wall thickness at end diastole, left ventricle end diastolic diameter, and left ventricle end systolic diameter. Fractional shortening (FS) was calculated from the end diastolic diameter (EDD) and end systolic diameter (ESD) using the following equation: FS=100%×[(EDD−ESD)/EDD]. Early diastolic filling peak velocity (E), late filling peak velocity (A), and isovolumetric relaxation time (IVRT) were measured from the medial or septal wall at the mitral valve level from tissue Doppler image. Left ventricle diastolic function was assessed by measuring the E/A ratio and IVRT. Three to five beats were averaged for each mouse measurement. The overall 21-day mortality rates were also calculated.

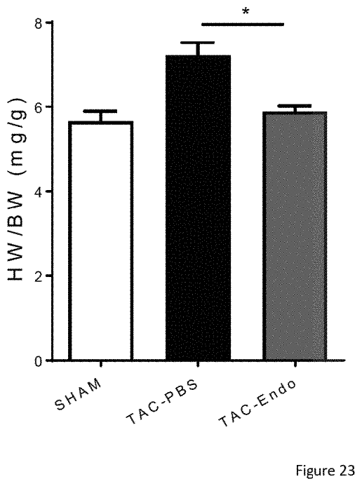

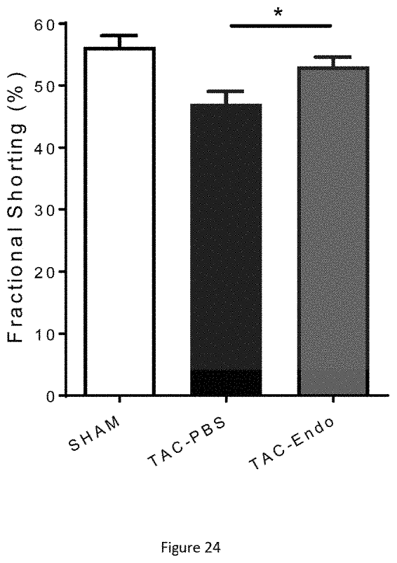

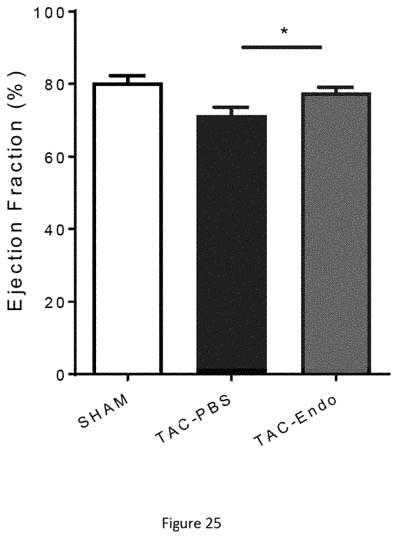

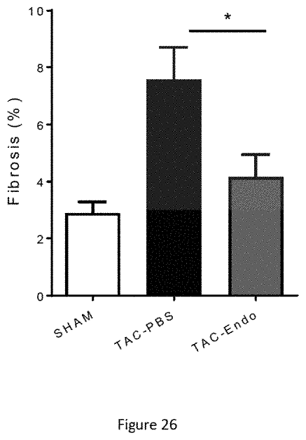

In this study, treatment of mice with hENG(27-581)-mFc significantly inhibited cardiac hypertrophy (see FIG. 23), improved cardiac function (see FIGS. 24 and 25), and histology data confirmed inhibition of cardiac fibrosis (see FIG. 26). Furthermore, hENG(27-581)-mFc treatment increased survival time of animals in this heart failure model. Together, these data demonstrate that endoglin polypeptide treatment is effective at treating heart failure, surprisingly effective in treating many distinct complications of heart failure. This indicates that endoglin polypeptides may have an advantage in treating heart failure over current standard of care treatments that only treat one or a few complications of heart failure.

### Example 9. Effects of BMP10 Propeptide and Endo-Fc Treatment in Myocardial Infarction Model

The effects of BMP10pro(22-312)-Fc and hENG(27-581)-mFc on the progression of heart failure were investigated using a mouse myocardial infarction (MI) model. See, e.g., Patten R. D. et al. (1998) Am J Physiol. 274: H1812-1820.

Thirty 10 week-old C57/B6 male mice underwent LAD surgery (MI mice) to induce myocardial infarction and ten age-matched animals underwent the same surgical procedure except for LAD (SHAM mice) at day 0. After waking from the surgery, MI mice were randomized into three groups. One group of 15 mice were injected subcutaneously with BMP10pro(22-312)-Fc at a dose of 10 mg/kg (MI-BMP10pro), a second group of 15 mice were subcutaneously injected with hENG(27-581)-mFc at a dose of 10 mg/kg (MI-Endo) and a third group of 15 mice were injected subcutaneously with phosphate buffered saline (vehicle control mice; MI-PBS mice), every 3 days for 28 days. At the end of the study, echocardiography was performed to measure left ventricular function and remodeling before animals were euthanized for heart collection. Each heart was photographed, fixed in 10% formalin, and sectioned for Masson's trichrome stain to assess fibrosis.

In vivo cardiac function was assessed by transthoracic echocardiography (Acuson P300, 18 MHz transducer; Siemens) in conscious mice. From left ventricle short axis view, M-mode echocardiogram was acquired to measure interventricular septal thickness at end diastole, left ventricular posterior wall thickness at end diastole, left ventricle end diastolic diameter, and left ventricle end systolic diameter. Fractional shortening (FS) was calculated from the end diastolic diameter (EDD) and end systolic diameter (ESD) using the following equation: FS=100%×[(EDD−ESD)/EDD]. Early diastolic filling peak velocity (E), late filling peak velocity (A), and isovolumetric relaxation time (IVRT) were measured from the medial or septal wall at the mitral valve level from tissue Doppler image. Left ventricle diastolic function was assessed by measuring the E/A ratio and IVRT. Three to five beats were averaged for each mouse measurement. The overall 28-day mortality rates were also calculated.

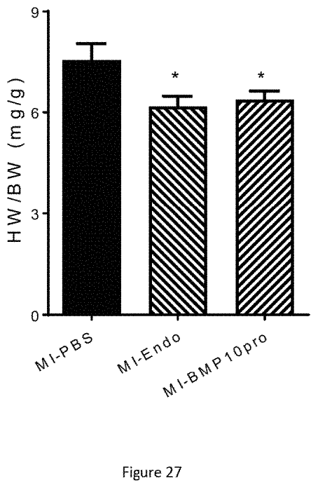

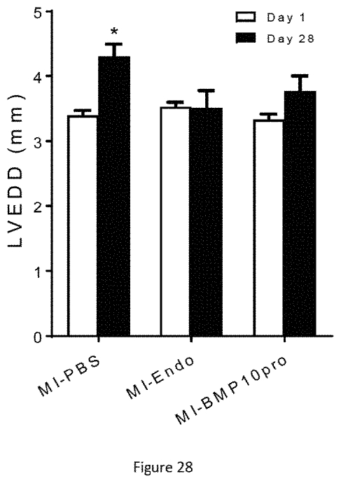

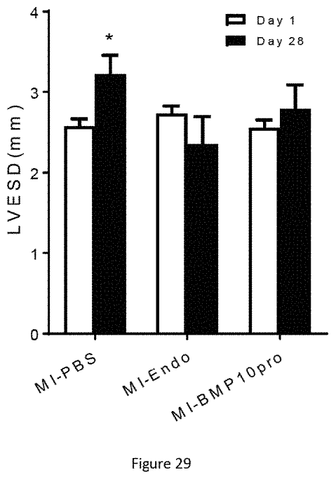

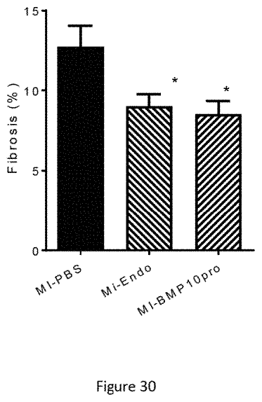

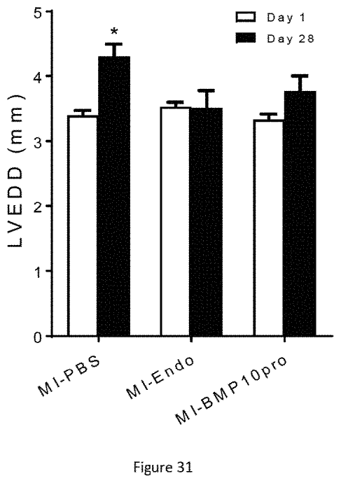

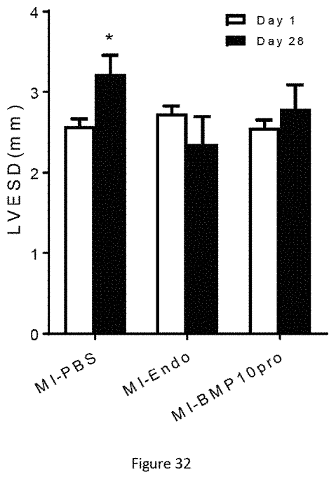

In this study, treatment of mice with BMP10pro(22-312)-Fc or hENG(27-581)-mFc significantly inhibited cardiac hypertrophy (see FIG. 27), suppressed cardiac remodeling (see FIGS. 28 and 29) and inhibited of cardiac fibrosis (see FIG. 30). Treatment with hENG(27-581)-mFc further improved cardiac function (see FIGS. 31 and 32). Furthermore, hENG(27-581)-mFc or BMP10pro(22-312)-Fc treatment increased survival time of animals in this heart failure model. Together, these data demonstrate that endoglin polypeptide BMP10pro polypeptides treatment is effective at treating heart failure following myocardial infarction, surprisingly effective in treating many distinct complications of heart failure. This indicates that endoglin polypeptides and BMP10pro polypeptides may have an advantage in treating heart failure over current standard of care treatments that only treat one or a few complications of heart failure.

### INCORPORATION BY REFERENCE

All publications and patents mentioned herein are hereby incorporated by reference in their entirety as if each individual publication or patent was specifically and individually indicated to be incorporated by reference.

While specific embodiments of the subject matter have been discussed, the above specification is illustrative and not restrictive. Many variations will become apparent to those skilled in the art upon review of this specification and the claims below. The full scope of the invention should be determined by reference to the claims, along with their full scope of equivalents, and the specification, along with such variations.

We claim:

1. A method of reducing the risk of death and/or hospitalization of a patient having heart failure, comprising administering to a patient in need thereof an effective amount of a BMP antagonist.

2. The method of claim 1, wherein the death of the patient is from any cause.

3. The method of claim 1, wherein the death of the patient is from a cardiovascular event.

4. The method of claim 3, wherein the cardiovascular event is selected from the group consisting of: myocardial infarction, stroke, angina, arrhythmia, fluid retention, and progression of heart failure.

5. The method of claim 1, wherein the hospitalization of the patient is from any cause.

6. The method of claim 1, wherein the hospitalization of the patient is from a cardiovascular event.

7. The method of claim 6, wherein the cardiovascular event is selected from the group consisting of: myocardial infarction, stroke, angina, arrhythmias, fluid retention, progression of heart failure.

8. A method of reducing progression of heart failure in a patient, comprising administering to a patient in need thereof an effective amount of a BMP antagonist.

9. A method of reducing incidence of cardiovascular events in a patient, comprising administering to a patient in need thereof an effective amount of a BMP antagonist.

10. The method of claim 1, wherein the cardiovascular event is selected from the group consisting of: myocardial infarction, stroke, angina, arrhythmia, fluid retention, progression of heart failure.

11. The method of claim 9 or 10, wherein the cardiovascular event would result in patient hospitalization.

12. A method of treating, preventing, or reducing the severity of cardiac fibrosis in a patient, comprising administering to a patient in need thereof an effective amount of a BMP antagonist.

13. A method of treating, preventing, or reducing the severity of cardiac hypertrophy in a patient, comprising administering to a patient in need thereof an effective amount of a BMP antagonist.

14. The method of claim 13, wherein the myocardial hypertrophy is concentric and/or eccentric hypertrophy.

15. A method of treating, preventing, or reducing the severity of cardiac remodeling in a patient, comprising administering to a patient in need thereof an effective amount of a BMP antagonist.

16. The method of claim 15, wherein the cardiac remodeling is ventricle remodeling.

17. The method of claim 15, wherein the cardiac remodeling is ventricular dilation.

18. The method of claim 15, wherein the method decreases interventricular septal end diastole.

19. The method of claim 15, wherein the method decreases posterior wall end diastole.

20. A method of treating, preventing, or reducing the severity of cardiac dysfunction in a patient, comprising administering to a patient in need thereof an effective amount of a BMP antagonist.

21. The method of claim 20, wherein the method increases cardiac ejection fraction.

22. The method of claim 21, wherein the method increases cardiac ejection fraction by at least 5% (e.g., at least 5%, 6%, 7%, 8%, 9%, 10%, 15%, 20%, 25%, 30%, 35%, 40%, 50%, 60%, 65% or more).

23. The method of any one of claims 20-22, wherein the method decreases isovolumic relaxation time.

24. The method of claim 23, wherein the method decreases isovolumic relaxation time by at least 2 ms (e.g., at least 2, 3, 4, 5, 6, 7, 8, 9, 10, 11, 12, 13, 14, 15, 16, 17, 18, 19, 20 or more ms).

25. The method of any one of claims 20-24, wherein the method increases fractional shorting.

26. The method of claim 25, wherein the method increase fractional shorting by at least 5% (e.g., at least 5%, 6%, 7%, 8%, 9%, 10%, 15%, 20%, 25%, 30%, 35%, 40%, 50%, or more).

27. A method of treating, preventing, or reducing the severity of hypertension in a patient, comprising administering to a patient in need thereof an effective amount of a BMP antagonist.

28. The method of claim 27, wherein the method reduces the patient's blood pressure.

29. The method of claim 28, wherein the method reduces systolic blood pressure.

30. The method of claim 29, wherein the method reduces systolic blood pressure by at least 4 mm Hg (e.g., at least 4, 5, 6, 7, 8, 9, 10, 11, 12, 13, 14, 15, 16, 17, 18, 19, 20, or more mm Hg).

31. The method of claim 27, wherein the method reduces diastolic blood pressure.

32. The method of claim 31, wherein the method reduces diastolic blood pressure by at least 2 mm Hg (e.g., at least 2, 3, 4, 5, 6, 7, 8, 9, 10, 11, 12, 13, 14, 15, 16, 17, 18, 19, 20, or more mm Hg).

33. The method of any one of claims 28-32, wherein the blood pressure is measured as resting blood pressure.

34. The method of any one of claims 28-32, wherein the blood pressure is measured as ambulatory blood pressure.

35. A method of treating, preventing, or reducing the severity of heart disease or one or more complications of heart disease, comprising administering to a patient in need thereof an effective amount of a BMP antagonist.

36. The method of claim 35, wherein the one or more complication of heart disease is selected from the group consisting of: dyspnea (shortness of breath), orthopnea, paroxysmal nocturnal dyspnea, and fatigue (which may limit exercise tolerance), fluid retention (which may lead to, for example, pulmonary congestion and peripheral edema), angina, hypertension, arrhythmia, ventricular arrhythmias, cardiomyopathy, cardiac hypertrophy, cardiac asthma, nocturia, ascities, congestive hepatopathy, coagulopathy, reduced renal blood flow, renal insufficiency, myocardial infarction, and stroke.

37. The method of any preceding claim, wherein the BMP antagonist is administered to the patient after myocardial infarction.

38. The method of any preceding claim, wherein the patient has left ventricular systolic dysfunction.

39. The method of any preceding claim, wherein the patient has ≤40% ejection fraction.

40. The method of any one of claims 1-38, wherein the patient has ≤35% ejection fraction.

41. The method of claim 39 or 40, wherein the ejection fraction is measured by one or more of radionuclide ventriculography, radionuclide angiography, echocardiography, or ventricular contrast angiography.

42. The method of any preceding claim, wherein the patient has one or more types of heart failure selected from the group consisting of: heart failure due to left ventricular dysfunction, heart failure with normal ejection fraction, aortic stenosis heart failure, acute heart failure, chronic heart failure, congestive heart failure, congenital heart failure, compensated heart failure, decompensated heart failure, diastolic heart failure, systolic heart failure, right-side heart (ventricle) failure, left-side heart (ventricle) failure, forward heart failure, backward heart failure, high output heart failure, low output heart failure, and myocardial edema.

43. The method of any preceding claim, wherein the patient has one or more complications selected from the group consisting of: dyspnea, orthopnea, aortic stenosis, paroxysmal nocturnal dyspnea, fatigue, fluid retention, pulmonary congestion, edema, peripheral edema, angina, hypertension, arrhythmia, ventricular arrhythmia, cardiomyopathy, cardiac hypertrophy, reduced renal blood flow, renal insufficiency, myocardial infarct, cardiac remodeling, cardiac fibrosis, cardiac hypertension, cardiac wall stress, cardiac inflammation, cardiac pressure overload, cardiac volume overload, stroke, cardiac chamber dilation, increase in ventricular sphericity, interstitial fibrosis, perivascular fibrosis, cardiomyocyte hypertrophy, cardiac asthma, nocturia, ascities, congestive hepatopathy, coagulopathy, acute ischemic injury, reperfusion injury, impairment of left ventricle function, and impairment of right ventricle function.

44. The method of any preceding claim, wherein the method treats, prevents, or reduces the severity of one or more complications selected from the group consisting of: dyspnea, orthopnea, paroxysmal nocturnal dyspnea, fatigue, fluid retention, pulmonary congestion, edema, peripheral edema, angina, hypertension, arrhythmia, ventricular arrhythmia, cardiomyopathy, cardiac hypertrophy, reduced renal blood flow, aortic stenosis, renal insufficiency, myocardial infarct, cardiac remodeling, cardiac fibrosis, cardiac hypertension, cardiac wall stress, cardiac inflammation, cardiac pressure overload, cardiac volume overload, stroke, cardiac chamber dilation, increase in ventricular sphericity, interstitial fibrosis, perivascular fibrosis, cardiomyocyte hypertrophy, cardiac asthma, nocturia, ascities, congestive hepatopathy, coagulopathy, acute ischemic injury, reperfusion injury, impairment of left ventricle function, and impairment of right ventricle function.

45. The method of any preceding claim, wherein the patient has one or more conditions selected from the group consisting of: systemic hypertension, pulmonary hypertension, diabetes, kidney (renal) failure (e.g., acute or chronic renal failure), coronary artery disease, hypertension, left ventricular dysfunction, heart valve disease, congenital heart defects, acute ischemic injury, reperfusion injury, cardiac remodeling pericardium disorders, myocardium disorders, great vessel disorders, and endocardium disorders,

46. The method of any preceding claim, wherein the patient has at least class I heart failure in accordance with the New York Heart Association (NYHA) functional classification.

47. The method of claim 46, wherein the patient has class II, class III, or class IV heart failure in accordance with the NYHA functional classification.

48. The method of claim 46, wherein the patient has class II or class III heart failure in accordance with the NYHA functional classification.

49. The method of claim 46, wherein the patient has class III or class IV heart failure in accordance with the NYHA functional classification.

50. The method of claim 46, wherein the patient has class IV heart failure in accordance with the NYHA functional classification.

51. The method of any preceding claim, wherein the method improves the patient's heart failure score in accordance with the NYHA functional classification system by at least one class (e.g., improvement from class IV to class III heart failure, from class IV to class II heart failure, from class IV to class I heart failure, from stage III to stage II heart failure, from stage III to stage I heart failure, or from class II to class I heart failure).

52. The method of any preceding claim, wherein the method prevents or delay progression of the patient's heart failure score in accordance with the NYHA functional classification system by at least one class (e.g., delays progression from class I to class II heart failure, delays progression from class I to class III heart failure, delays progression from class I to class IV heart failure, delays progression from class II to class III heart failure, delays progression from class II to class IV heart failure, or delays progression from class III to class IV heart failure.

53. The method of any preceding claim, wherein the patient has at least stage A heart failure in accordance with the American College of Cardiology/American Heart Association working group (AAC) functional classification.

54. The method of claim 53, wherein the patient has stage B, stage C, or stage D heart failure in accordance with the ACC functional classification.

55. The method of any preceding claim, wherein the method improves the patient's heart failure score in accordance with the ACC functional classification system by at least one stage (e.g., improvement from stage D to stage C heart failure, from stage D to stage B heart failure, from stage D to stage A heart failure, from stage C to stage B heart failure, from stage C to stage A heart failure, or from stage B to stage A heart failure).

56. The method of any preceding claim, wherein the method prevents or delay progression of the patient's heart failure score in accordance with the ACC functional classification system by at least one stage (e.g., prevents or delays progression from stage A to stage B heart failure, delays progression from stage A to stage C heart failure, delays progression from stage A to stage D heart failure, delays progression from stage B to stage C heart failure, delays progression from stage B to stage D heart failure, or delays progression from stage C to stage D heart failure.

57. The method of any preceding claim, wherein the method treats, prevents, or reduces the severity of cardiac fibrosis.

58. The method of any preceding claim, wherein the method treats, prevents, or reduces the severity of cardiac hypertrophy.

59. The method of claim 58, wherein the myocardial hypertrophy is concentric and/or eccentric hypertrophy.

60. The method of any preceding claim, wherein the method treats, prevents, or reduces the severity of cardiac remodeling in a patient, comprising administering to a patient in need thereof an effective amount of a BMP antagonist.

61. The method of claim 60, wherein the cardiac remodeling is ventricle remodeling and/or ventricular dilation.

62. The method of claim 60, wherein the method decreases interventricular septal end diastole and/or posterior wall end diastole.

63. The method of any preceding claim, wherein the method treats, prevents, or reduces the severity of cardiac dysfunction in a patient.

64. The method of any preceding claim, wherein the method increases cardiac ejection fraction.

65. The method of claim 64, wherein the method increases cardiac ejection fraction by at least 5% (e.g., at least 5%, 6%, 7%, 8%, 9%, 10%, 15%, 20%, 25%, 30%, 35%, 40%, 50%, 60%, 65% or more).

66. The method of any preceding claim, wherein the method decreases isovolumic relaxation time.

67. The method of claim 66, wherein the method decreases isovolumic relaxation time by at least 2 ms (e.g., at least 2, 3, 4, 5, 6, 7, 8, 9, 10, 11, 12, 13, 14, 15, 16, 17, 18, 19, 20 or more ms).

68. The method of any preceding claim, wherein the method increases fractional shorting.

69. The method of claim 68, wherein the method increase fractional shorting by at least 5% (e.g., at least 5%, 6%, 7%, 8%, 9%, 10%, 15%, 20%, 25%, 30%, 35%, 40%, 50%, or more).

70. The method of any preceding claim, method treats, prevents, or reduces the severity of hypertension in a patient.

71. The method of claim 70, wherein the method reduces the patient's blood pressure.

72. The method of claim 71, wherein the method reduces systolic blood pressure.

73. The method of claim 72, wherein the method reduces systolic blood pressure by at least 4 mm Hg (e.g., at least 4, 5, 6, 7, 8, 9, 10, 11, 12, 13, 14, 15, 16, 17, 18, 19, 20, or more mm Hg).

74. The method of claim 70, wherein the method reduces diastolic blood pressure.

75. The method of claim 74, wherein the method reduces diastolic blood pressure by at least 2 mm Hg (e.g., at least 2, 3, 4, 5, 6, 7, 8, 9, 10, 11, 12, 13, 14, 15, 16, 17, 18, 19, 20, or more mm Hg).

76. The method of any preceding claim, wherein the BMP antagonists is an ActRIIA polypeptide.

77. The method of claim 76, wherein the ActRIIA polypeptide is selected from the group consisting of:
a. a polypeptide comprising an amino acid sequence that is at least 70%, 75% 80%, 85%, 90%, 91%, 92%, 93%, 94%, 95%, 96%, 97%, 98%, 99%, or 100% identical to the amino acid sequence of SEQ ID NO: 10;
b. a polypeptide comprising an amino acid sequence that is at least 70%, 75% 80%, 85%, 90%, 91%, 92%, 93%, 94%, 95%, 96%, 97%, 98%, 99%, or 100% identical to the amino acid sequence of SEQ ID NO: 11; and
c. a polypeptide comprising an amino acid sequence that is at least 70%, 75% 80%, 85%, 90%, 91%, 92%, 93%, 94%, 95%, 96%, 97%, 98%, 99%, or 100% identical to the sequence of amino acid 30-110 of SEQ ID NO: 9.

78. The method of any one of claims 1-75, wherein the BMP antagonist is an ActRIIB polypeptide.

79. The method of claim 78, wherein the ActRIIB polypeptide is selected from the group consisting of:
a. a polypeptide comprising an amino acid sequence that is at least 70%, 75% 80%, 85%, 90%, 91%, 92%, 93%, 94%, 95%, 96%, 97%, 98%, 99%, or 100% identical to amino acids 29-109 of SEQ ID NO: 1;
b. a polypeptide comprising an amino acid sequence that is at least 70%, 75% 80%, 85%, 90%, 91%, 92%, 93%, 94%, 95%, 96%, 97%, 98%, 99%, or 100% identical to amino acids 25-131 of SEQ ID NO: 1;
c. a polypeptide comprising an amino acid sequence that is at least 70%, 75% 80%, 85%, 90%, 91%, 92%, 93%, 94%, 95%, 96%, 97%, 98%, 99%, or 100% identical to the amino acid sequence of SEQ ID NO: 2;
d. a polypeptide comprising an amino acid sequence that is at least 70%, 75% 80%, 85%, 90%, 91%, 92%, 93%, 94%, 95%, 96%, 97%, 98%, 99%, or 100% identical to the amino acid sequence of SEQ ID NO: 3;
e. a polypeptide comprising an amino acid sequence that is at least 70%, 75% 80%, 85%, 90%, 91%, 92%, 93%, 94%, 95%, 96%, 97%, 98%, 99%, or 100% identical to the amino acid sequence of SEQ ID NO: 5;
f. a polypeptide comprising an amino acid sequence that is at least 70%, 75% 80%, 85%, 90%, 91%, 92%, 93%, 94%, 95%, 96%, 97%, 98%, 99%, or 100% identical to the amino acid sequence of SEQ ID NO: 6;
g. a polypeptide comprising an amino acid sequence that is at least 70%, 75% 80%, 85%, 90%, 91%, 92%, 93%, 94%, 95%, 96%, 97%, 98%, 99%, or 100% identical to the amino acid sequence of SEQ ID NO: 65; and
h. a polypeptide comprising an amino acid sequence that is at least 70%, 75% 80%, 85%, 90%, 91%, 92%, 93%, 94%, 95%, 96%, 97%, 98%, 99%, or 100% identical to the amino acid sequence of SEQ ID NO: 133.

80. The method of claim 79, wherein the polypeptide does not comprise an acidic amino acid at position 79 with respect to SEQ ID NO: 1.

81. The method of claim 80, wherein the polypeptide does not comprise a D or E at position 79 with respect to SEQ ID NO: 1.

82. The method of any one of claims 1-75, wherein the BMP antagonist is a BMPRII polypeptide.

83. The method of claim 82, wherein the BMPRII polypeptide is selected from the group consisting of:
a. a polypeptide comprising an amino acid sequence that is at least 70%, 75% 80%, 85%, 90%, 91%, 92%, 93%, 94%, 95%, 96%, 97%, 98%, 99%, or 100% identical to amino acids 27-150 of SEQ ID NO: 14;
b. a polypeptide comprising an amino acid sequence that is at least 70%, 75% 80%, 85%, 90%, 91%, 92%, 93%, 94%, 95%, 96%, 97%, 98%, 99%, or 100% identical to amino acids 34-123 of SEQ ID NO: 14; and
c. a polypeptide comprising an amino acid sequence that is at least 70%, 75% 80%, 85%, 90%, 91%, 92%, 93%, 94%, 95%, 96%, 97%, 98%, 99%, or 100% identical to the amino acid sequence of SEQ ID NO: 15.

84. The method of any one of claims 1-75, wherein the BMP antagonist is an ALK1 polypeptide.

85. The method of claim 84, wherein the ALK1 polypeptide is selected from the group consisting of:
a. a polypeptide comprising an amino acid sequence that is at least 70%, 75% 80%, 85%, 90%, 91%, 92%, 93%, 94%, 95%, 96%, 97%, 98%, 99%, or 100% identical to amino acids 22-118 of SEQ ID NO: 20;
b. a polypeptide comprising an amino acid sequence that is at least 70%, 75% 80%, 85%, 90%, 91%, 92%, 93%, 94%, 95%, 96%, 97%, 98%, 99%, or 100% identical to amino acids 34-95 of SEQ ID NO: 20; and
c. a polypeptide comprising an amino acid sequence that is at least 70%, 75% 80%, 85%, 90%, 91%, 92%, 93%, 94%, 95%, 96%, 97%, 98%, 99%, or 100% identical to the amino acid sequence of SEQ ID NO: 21.

86. The method of any one of claims 1-75, wherein the BMP antagonist is an endoglin polypeptide.

87. The method of claim 86, wherein the endoglin polypeptide is selected from the group consisting of:
a. a polypeptide comprising an amino acid sequence that is at least 70%, 75% 80%, 85%, 90%, 91%, 92%, 93%, 94%, 95%, 96%, 97%, 98%, 99%, or 100% identical to amino acids 26-378 of SEQ ID NO: 24;
b. a polypeptide comprising an amino acid sequence that is at least 70%, 75% 80%, 85%, 90%, 91%, 92%, 93%, 94%, 95%, 96%, 97%, 98%, 99%, or 100% identical to amino acids 42-333 of SEQ ID NO: 24;
c. a polypeptide comprising an amino acid sequence that is at least 70%, 75% 80%, 85%, 90%, 91%, 92%, 93%, 94%, 95%, 96%, 97%, 98%, 99%, or 100% identical to amino acids 26-346 of SEQ ID NO: 24;
d. a polypeptide comprising an amino acid sequence that is at least 70%, 75% 80%, 85%, 90%, 91%, 92%, 93%, 94%, 95%, 96%, 97%, 98%, 99%, or 100% identical to amino acids 27-3581 of SEQ ID NO: 24; and
e. a polypeptide comprising an amino acid sequence that is at least 70%, 75% 80%, 85%, 90%, 91%, 92%, 93%, 94%, 95%, 96%, 97%, 98%, 99%, or 100% identical to amino acids 26-359 of SEQ ID NO: 24.

88. The method of claim 87, wherein the endoglin polypeptide does not comprise a sequence consisting of amino acids 379-430 of SEQ ID NO: 24.

89. The method of claim 87, wherein the endoglin polypeptide does not comprise more than 50 consecutive amino acids from a sequence consisting of amino acids 379-586 of SEQ ID NO: 24.

90. The method of any one of claims 1-75, wherein the BMP antagonist is a BMP10 propeptide polypeptide.

91. The method of claim 90, wherein the BMP10 propeptide polypeptide is selected from the group consisting of:
a. a polypeptide comprising an amino acid sequence that is at least 70%, 75%, 80%, 85%, 90%, 91%, 92%, 93%, 94%, 95%, 96%, 97%, 98%, 99%, or 100% identical to a sequence that begins at a position corresponding to any one of amino acids 1-6 of SEQ ID NO: 34 and ends at a position corresponding any one of amino acids 292-296 of SEQ ID NO: 34;
b. a polypeptide comprising an amino acid sequence that is at least 70%, 75%, 80%, 85%, 90%, 91%, 92%, 93%, 94%, 95%, 96%, 97%, 98%, 99%, or 100% identical to a sequence that begins at a position corresponding to any one of amino acids 1-6 of SEQ ID NO: 34 and ends at a position corresponding any one of amino acids 292-295 of SEQ ID NO: 34, wherein the polypeptide does not comprise the sequence of amino acids RIRR;
c. a polypeptide comprises an amino acid sequence that is at least 70%, 75%, 80%, 85%, 90%, 91%, 92%, 93%, 94%, 95%, 96%, 97%, 98%, 99%, or 100% identical to amino acids 1-292 of SEQ ID NO: 34;
d. a polypeptide comprises an amino acid sequence that is at least 70%, 75%, 80%, 85%, 90%, 91%, 92%, 93%, 94%, 95%, 96%, 97%, 98%, 99%, or 100% identical to amino acids 1-292 of SEQ ID NO: 34, wherein the polypeptide does not comprise the sequence of amino acids RIRR;
e. a polypeptide comprises an amino acid sequence that is at least 70%, 75%, 80%, 85%, 90%, 91%, 92%, 93%, 94%, 95%, 96%, 97%, 98%, 99%, or 100% identical to amino acids 1-295 of SEQ ID NO: 34;
f. a polypeptide comprises an amino acid sequence that is at least 70%, 75%, 80%, 85%, 90%, 91%, 92%, 93%, 94%, 95%, 96%, 97%, 98%, 99%, or 100% identical to amino acids 1-295 of SEQ ID NO: 34, wherein the polypeptide does not comprise the sequence of amino acids RIRR;
g. a polypeptide comprising an amino acid sequence that is at least 70%, 75%, 80%, 85%, 90%, 91%, 92%, 93%, 94%, 95%, 96%, 97%, 98%, 99%, or 100% identical to a sequence that begins at a position corresponding to any one of amino acids 1-6 of SEQ ID NO: 34 and ends at a position corresponding any one of amino acids 292-295 of SEQ ID NO: 34, wherein the C-terminus of the polypeptide is not R296 of SEQ ID NO: 34;
h. a polypeptide comprising an amino acid sequence that is at least 70%, 75%, 80%, 85%, 90%, 91%, 92%, 93%, 94%, 95%, 96%, 97%, 98%, 99%, or 100% identical to a sequence that begins at a position corresponding to any one of amino acids 1-6 of SEQ ID NO: 34 and ends at a position corresponding any one of amino acids 292-295 of SEQ ID NO: 34, wherein the C-terminus of the polypeptide is not R296 of SEQ ID NO: 34;
i. a polypeptide comprises an amino acid sequence that is at least 70%, 75%, 80%, 85%, 90%, 91%, 92%, 93%, 94%, 95%, 96%, 97%, 98%, 99%, or 100% identical to amino acids 1-292 of SEQ ID NO: 34, wherein the C-terminus of the polypeptide is not R296 of SEQ ID NO: 34; and
j. a polypeptide comprises an amino acid sequence that is at least 70%, 75%, 80%, 85%, 90%, 91%, 92%, 93%, 94%, 95%, 96%, 97%, 98%, 99%, or 100% identical to amino acids 1-295 of SEQ ID NO: 34, wherein the C-terminus of the polypeptide is not R296 of SEQ ID NO: 34.

92. The method of any one of claims 76-91 wherein the ActRIIA, ActRIIB, ALK1, endoglin, or BMP10pro polypeptide is a fusion protein comprising an immunoglobulin Fc domain.

93. The method of claim 92, wherein the immunoglobulin Fc domain is an IgG1 Fc immunoglobulin domain.

94. The method of claim 92 or 93, wherein the fusion protein comprises a linker domain positioned between the ActRIIA, ActRIIB, ALK1, endoglin, or BMP10pro polypeptide domain and the Fc immunoglobulin domain.

95. The method of claim 94, wherein the linker is selected from the group consisting of: GGG (SEQ ID NO: 41), GGGG (SEQ ID NO: 42), TGGGG (SEQ ID NO: 43), SGGGG (SEQ ID NO: 44), TGGG (SEQ ID NO: 45), SGGG (SEQ ID NO: 46), or GGGGS (SEQ ID NO: 47).

96. The method of preceding claim, wherein the ActRIIA-Fc fusion protein is selected from the group consisting of:
a. a polypeptide comprising an amino acid sequence that is at least 70%, 75% 80%, 85%, 90%, 91%, 92%, 93%, 94%, 95%, 96%, 97%, 98%, 99%, or 100% identical to the amino acid sequence of SEQ ID NO: 50;
b. a polypeptide comprising an amino acid sequence that is at least 70%, 75% 80%, 85%, 90%, 91%, 92%, 93%, 94%, 95%, 96%, 97%, 98%, 99%, or 100% identical to the amino acid sequence of SEQ ID NO: 54; and
c. c) a polypeptide comprising an amino acid sequence that is at least 70%, 75% 80%, 85%, 90%, 91%, 92%, 93%, 94%, 95%, 96%, 97%, 98%, 99%, or 100% identical to the amino acid sequence of SEQ ID NO: 57.

97. The method of any preceding claim, wherein the fusion protein is an ActRIIB-Fc fusion protein selected from the group consisting of:
a. a polypeptide comprising an amino acid sequence that is at least 70%, 75% 80%, 85%, 90%, 91%, 92%, 93%, 94%, 95%, 96%, 97%, 98%, 99%, or 100% identical to the amino acid sequence of SEQ ID NO: 58;
b. a polypeptide comprising an amino acid sequence that is at least 70%, 75% 80%, 85%, 90%, 91%, 92%, 93%, 94%, 95%, 96%, 97%, 98%, 99%, or 100% identical to the amino acid sequence of SEQ ID NO: 60;
c. a polypeptide comprising an amino acid sequence that is at least 70%, 75% 80%, 85%, 90%, 91%, 92%, 93%, 94%, 95%, 96%, 97%, 98%, 99%, or 100% identical to the amino acid sequence of SEQ ID NO: 63;
d. a polypeptide comprising an amino acid sequence that is at least 70%, 75% 80%, 85%, 90%, 91%, 92%, 93%, 94%, 95%, 96%, 97%, 98%, 99%, or 100% identical to the amino acid sequence of SEQ ID NO: 64;
e. a polypeptide comprising an amino acid sequence that is at least 70%, 75% 80%, 85%, 90%, 91%, 92%, 93%, 94%, 95%, 96%, 97%, 98%, 99%, or 100% identical to the amino acid sequence of SEQ ID NO: 66;
f. a polypeptide comprising an amino acid sequence that is at least 70%, 75% 80%, 85%, 90%, 91%, 92%, 93%, 94%, 95%, 96%, 97%, 98%, 99%, or 100% identical to the amino acid sequence of SEQ ID NO: 123;
g. a polypeptide comprising amino acid sequence that is at least 70%, 75% 80%, 85%, 90%, 91%, 92%, 93%, 94%, 95%, 96%, 97%, 98%, 99%, or 100% identical to the amino acid sequence of SEQ ID NO: 131; and
h. a polypeptide comprising an amino acid sequence that is at least 70%, 75% 80%, 85%, 90%, 91%, 92%, 93%, 94%, 95%, 96%, 97%, 98%, 99%, or 100% identical to the amino acid sequence of SEQ ID NO: 132.

98. The method of claim 97, wherein the ActRIIB-Fc fusion protein does not comprise an ActRIIB polypeptide domain comprising an acidic amino acid at position 79 with respect to SEQ ID NO: 1.

99. The method of claim 98, wherein the ActRIIB polypeptide domain does not comprise a D or E at position 79 with respect to SEQ ID NO: 1.

100. The method of any preceding claim, wherein the BMPRII-Fc fusion protein is selected from the group consisting of:
a. a polypeptide comprising an amino acid sequence that is at least 70%, 75% 80%, 85%, 90%, 91%, 92%, 93%, 94%, 95%, 96%, 97%, 98%, 99%, or 100% identical to the amino acid sequence of SEQ ID NO: 69; and
b. a polypeptide comprising an amino acid sequence that is at least 70%, 75% 80%, 85%, 90%, 91%, 92%, 93%, 94%, 95%, 96%, 97%, 98%, 99%, or 100% identical to the amino acid sequence of SEQ ID NO: 71.

101. The method of any preceding claim, wherein the ALK1-Fc fusion protein is selected from the group consisting of:
a. a polypeptide comprising an amino acid sequence that is at least 70%, 75% 80%, 85%, 90%, 91%, 92%, 93%, 94%, 95%, 96%, 97%, 98%, 99%, or 100% identical to the amino acid sequence of SEQ ID NO: 74; and
b. a polypeptide comprising an amino acid sequence that is at least 70%, 75% 80%, 85%, 90%, 91%, 92%, 93%, 94%, 95%, 96%, 97%, 98%, 99%, or 100% identical to the amino acid sequence of SEQ ID NO: 76.

102. The method of any preceding claim, wherein the endoglin-Fc fusion protein is selected from the group consisting of:
a. a polypeptide comprising an amino acid sequence that is at least 70%, 75% 80%, 85%, 90%, 91%, 92%, 93%, 94%, 95%, 96%, 97%, 98%, 99%, or 100% identical to the amino acid sequence of SEQ ID NO: 78;
b. a polypeptide comprising an amino acid sequence that is at least 70%, 75% 80%, 85%, 90%, 91%, 92%, 93%, 94%, 95%, 96%, 97%, 98%, 99%, or 100% identical to the amino acid sequence of SEQ ID NO: 80;
c. a polypeptide comprising an amino acid sequence that is at least 70%, 75% 80%, 85%, 90%, 91%, 92%, 93%, 94%, 95%, 96%, 97%, 98%, 99%, or 100% identical to the amino acid sequence of SEQ ID NO: 28;
d. a polypeptide comprising an amino acid sequence that is at least 70%, 75% 80%, 85%, 90%, 91%, 92%, 93%, 94%, 95%, 96%, 97%, 98%, 99%, or 100% identical to the amino acid sequence of SEQ ID NO: 29;
e. a polypeptide comprising an amino acid sequence that is at least 70%, 75% 80%, 85%, 90%, 91%, 92%, 93%, 94%, 95%, 96%, 97%, 98%, 99%, or 100% identical to the amino acid sequence of SEQ ID NO: 30; and
f. a polypeptide comprising an amino acid sequence that is at least 70%, 75% 80%, 85%, 90%, 91%, 92%, 93%, 94%, 95%, 96%, 97%, 98%, 99%, or 100% identical to the amino acid sequence of SEQ ID NO: 31.

103. The method of any preceding claim, wherein the BMP10 propeptide-Fc fusion protein is selected from the group consisting of:
a. a polypeptide comprising an amino acid sequence that is at least 70%, 75% 80%, 85%, 90%, 91%, 92%, 93%, 94%, 95%, 96%, 97%, 98%, 99%, or 100% identical to the amino acid sequence of SEQ ID NO: 82;
b. a polypeptide comprising an amino acid sequence that is at least 70%, 75% 80%, 85%, 90%, 91%, 92%, 93%, 94%, 95%, 96%, 97%, 98%, 99%, or 100% identical to the amino acid sequence of SEQ ID NO: 84;
c. a polypeptide comprising an amino acid sequence that is at least 70%, 75% 80%, 85%, 90%, 91%, 92%, 93%, 94%, 95%, 96%, 97%, 98%, 99%, or 100% identical to the amino acid sequence of SEQ ID NO: 85; and
d. a polypeptide comprising an amino acid sequence that is at least 70%, 75% 80%, 85%, 90%, 91%, 92%, 93%, 94%, 95%, 96%, 97%, 98%, 99%, or 100% identical to the amino acid sequence of SEQ ID NO: 87.

104. The method of any one of claims 76-103, wherein the polypeptide or fusion protein comprises one or more amino acid modifications selected from the group consisting of: a glycosylated amino acid, a PEGylated amino acid, a farnesylated amino acid, an acetylated amino acid, a biotinylated amino acid, and an amino acid conjugated to a lipid moiety.

105. The method of claim 104, wherein the polypeptide or fusion protein has a glycosylation pattern obtainable from a Chinese hamster ovary cell line.

106. The method of any one of claims 76-104, wherein the polypeptide or fusion protein binds to BMP10 and/or BMP9.

107. The method of claim 106, wherein the polypeptide or fusion protein further binds to one or more ligands selected from the group consisting of: BMP6, BMP3b, and BMP5.

108. The method of any one of claims 76-107, wherein the polypeptide or fusion protein inhibits BMP10 and/or BMP9.

109. The method of 108, wherein the polypeptide or fusion protein inhibits BMP10 and/or BMP9 activity in a cell-based assay.

110. The method of claim 108 or 109, wherein the polypeptide or fusion protein further inhibits one or more ligands selected from the group consisting of BMP6, BMP3b, and BMP5.

111. The method of claim 110 wherein the polypeptide or fusion protein inhibits activity of one or more of BMP6, BMP3b, and BMP5 in a cell-based assay.

112. The method of any one of claims 92-110, wherein the fusion protein is a homodimer.

113. The method of any one of claims 1-75, wherein the BMP antagonist is an antibody or combination of antibodies.

114. The method of claim 113, wherein the antibody or combination of antibodies binds to BMP10 and/or BMP9.

115. The method of claim 114, wherein the antibody or combination of antibodies further binds to one or more ligands selected from the group consisting of BMP6, BMP3b, and BMP5.

116. The method of 113, wherein the antibody or combination of antibodies inhibits BMP10 and/or BMP9 activity.

117. The method of claim 115, wherein the antibody or combination of antibodies further inhibits activity of one or more ligands selected from the group consisting: of BMP6, BMP3b, and BMP5.

118. The method of claim 113, wherein the antibody or combination of antibodies binds to one or more polypeptides selected from the group consisting of: BMP10, BMP9, BMP6, BMP3b, BMP5, ActRIIA, ActRIIB, BMPRII, ALK1, and endoglin.

119. The method of any one of claims 113-118, wherein the antibody or combination of antibodies binds to at least mature BMP10 and binds competitively with a BMP10 propeptide.

120. The method of any one of claims 113-119, wherein the antibody or combination of antibodies binds to one or more of BMP9, BMP6, BMP3b, and BMP5 and binds competitively with a BMP10 propeptide.

121. The method of any one of claims 1-75, wherein the BMP antagonist is a small molecule.

122. The method of claim 121, wherein the small molecule inhibits activity of BMP9 and/or BMP10.

123. The method of claim 122, wherein the small molecule further inhibits activity of one or more ligands selected from the group consisting of: BMP6, BMP3b, and BMP5.

124. The method of claim 121, wherein the small molecule inhibits activity one or more agents selected from the group consisting of: BMP10, BMP9, BMP6, BMP3b, BMP5, ActRIIA, ActRIIB, BMPRII, ALK1, endoglin, and one or more Smads (e.g., Smads 2 and/or 3).

125. The method of any one of claims 1-75, wherein the BMP antagonist is a nucleotide.

126. The method of claim 125, wherein the nucleotide inhibits activity of BMP9 and/or BMP10.

127. The method of claim 126, wherein the nucleotide further inhibits activity of one or more ligands selected from the group consisting of: BMP6, BMP3b, and BMP5.

128. The method of claim 125, wherein the nucleotide inhibits activity one or more agents selected from the group consisting of: BMP10, BMP9, BMP6, BMP3b, BMP5, ActRIIA, ActRIIB, BMPRII, ALK1, endoglin, and one or more Smads (e.g., Smads 2 and/or 3).

129. The method of preceding claim, wherein the patient is administered an additional active agent or other supportive therapy for treating, preventing, or reducing the severity of heart failure or one or more complications of heart failure.

130. The method of claim 129, wherein the additional active agent or other supportive therapy for treating, preventing, or reducing the severity of heart failure or one or more complications of heart failure is selected from the group consisting of: pacemaker, implantable cardiac defibrillator, cardiac contractility modulation, cardiac resynchronization therapy, ventricular assist device, biventricular cardiac resynchronization therapy, heart transplant, adrenergic blockers (alpha- and beta-blockers), centrally acting alpha-agonists, angiotensin-converting enzyme (ACE) inhibitors, angiotensin receptor blockers, calcium channel blockers, positive inotropes, vasodilators, benzodiazepines, renin inhibitors, antithrombotic agents, multiple types of diuretics, captopril, enalapril, lisinopril, benazepril, ramipril, Zofenopril, quinapril, perinodopril, lisinopril, benazepril, imidapril, trandolapril, cilazapril, and fosinopril, losartan, candesartan, valsartan, irbesartan, telmisartan, eprosartan, olmesartan, azilsartan, Fimasartan, propranolol, bucindolol, carteolol, carvedilol, labetalol, nadolol, oxprenolol, penbutolol, pindolol, sotalol, timolol, acebutolol, atenolol, betaxolol, bisoprolol, celiprolol, esmolol, metoprolol, nebivolol, butazamine, ICI-118,551, SR 59230A, phenoxybenzamine, phentolamine, tolazoline, trazodone, alfuzosin, doxazosin mesylate (Cardura and Carduran), prazosin, tamsulosin, terazosin, Silodosin, atipanmezole (e.g., Antisedan), idazoxan, mirtazapine, yohimbine, acidifying salts (e.g., CaCl₂ and NH₄CL), arginine vasopressin receptor 2 antagonists, selective vasopressin V2 antagonists, Na—H exchanger antagonists, carbonic anhydrase inhibitors, loop diuretics, osmotic diuretics, potassium-sparing diuretics, thiazides, xanthines, dihydropyridine, amlodipine, aranidipine, azelnidipine, barnidipine, benidipine, cilnidipine, clevidipine, isradipine, efonidipine, felodipine, lacidipine, lercanidipine, manidipine, nicardipine, nifedipine, Nilvadipine, nimodipine, nisoldipine, nitrendipine, pranidipine, phenylalkylamine calcium channel blockers, verapamil, gallopamil, fendiline, benzothiazepine calcium channel blockers, diltiazem, mibefradil, bepridil, flunarizine, fluspirilene, fendiline, gabapentinoids, ziconotide, digoxin, amiodarone, berberine, levosimendan, omecamtiv, catecholamines, eicosanoids, phosphodiesterase inhibitors, enoximone, milrinone, amrinone, theophylline, glucagon, insulin, sodium nitroprusside, hydralazine, isosorbide dinitrate, and isosorbide mononitrate, nitroglycerin, benzodiazepines, renin inhibitors, clonidine, guanabenz, guanfacine, methyldopa, and moxonidine, minoxidil, guanethidine, mecamylamine, reserpine, irreversible cyclooxygenase inhibitors, adenosine diphosphate receptor inhibitors, clopidogrel, prasugrel, ticagrelor, and ticlopidine, phosphodiesterase inhibitors, cilostazol, protease-activated receptor-1 antagonists, vorapaxar, glycoprotein inhibitors, abciximab, eptifibatide, tirofiban, adenosine reuptake inhibitors, dipyridamole, thromboxane inhibitors, thromboxane synthase inhibitors, and thromboxane receptor antagonists, tissue plasminogen activators, alteplase, reteplase, tenecteplase, anistreplase, streptokinase, urokinase, dabigatran, rivaroxaban, apixaban, coumarins, heparin and derivatives thereof, factor Xa inhibitors, rivaroxaban, apixaban, edoxaban, betrixaban, letaxaban, eribaxaban, hirudin, lepirudin, bivalirudin, argatroban, dabigatran, ximelagatran, antithrombin protein, batroxobin, hementin, and vitamin E.

131. A BMP10 propeptide (BMP10pro) polypeptide comprising an amino acid sequence that is at least 70%, 75%, 80%, 85%, 90%, 91%, 92%, 93%, 94%, 95%, 96%, 97%, 98%, 99%, or 100% identical to a sequence that begins at a position corresponding to any one of amino acids 1-6 of SEQ ID NO: 34 and ends at a position corresponding any one of amino acids 292-295 of SEQ ID NO: 34, wherein the polypeptide does not comprise the sequence of amino acids RIRR.

132. The BMP10pro polypeptide of claim 131, wherein the polypeptide comprises an amino acid sequence that is at least 70%, 75%, 80%, 85%, 90%, 91%, 92%, 93%, 94%, 95%, 96%, 97%, 98%, 99%, or 100% identical to amino acids 1-292 of SEQ ID NO: 34.

133. The BMP10pro polypeptide of claim 131, wherein the polypeptide comprises an amino acid sequence that is at least 70%, 75%, 80%, 85%, 90%, 91%, 92%, 93%, 94%, 95%, 96%, 97%, 98%, 99%, or 100% identical to amino acids 1-295 of SEQ ID NO: 34.

134. A BMP propeptide (BMP10pro) polypeptide comprising an amino acid sequence that is at least 70%, 75%, 80%, 85%, 90%, 91%, 92%, 93%, 94%, 95%, 96%, 97%, 98%, 99%, or 100% identical to a sequence that begins at a position corresponding to any one of amino acids 1-6 of SEQ ID NO: 34 and ends at a position corresponding any one of amino acids 292-295 of SEQ ID NO: 34, wherein the C-terminus of the polypeptide is not R296 of SEQ ID NO: 34.

135. The BMP10pro polypeptide of claim 134, wherein the polypeptide comprises an amino acid sequence that is at least 70%, 75%, 80%, 85%, 90%, 91%, 92%, 93%, 94%, 95%, 96%, 97%, 98%, 99%, or 100% identical to amino acids 1-292 of SEQ ID NO: 34.

136. The BMP10pro polypeptide of claim 134, wherein the polypeptide comprises an amino acid sequence that is at least 70%, 75%, 80%, 85%, 90%, 91%, 92%, 93%, 94%, 95%, 96%, 97%, 98%, 99%, or 100% identical to amino acids 1-295 of SEQ ID NO: 34.

137. The BMP10pro polypeptide of any one of claims 131-136, wherein the BMP10pro polypeptide is a fusion protein comprising an immunoglobulin Fc domain.

138. The BMP10pro polypeptide of claim 137, wherein the immunoglobulin Fc domain is an IgG1 Fc immunoglobulin domain.

139. A BMP10pro-Fc fusion protein comprising a BMP10pro domain and a Fc immunoglobulin domain, wherein the BMP10pro domain consists of an amino acid sequence that is at least 70%, 75% 80%, 85%, 90%, 91%, 92%, 93%, 94%, 95%, 96%, 97%, 98%, 99%, or 100% to a sequence that begins at a position corresponding to any one of amino acids 1-6 of SEQ ID NO: 34 and ends at a position corresponding any one of amino acids 292-295 of SEQ ID NO: 34.

140. The BMP10pro-Fc fusion protein of claim 139, wherein the BMP10pro domain consists of an amino acid sequence that is at least 70%, 75%, 80%, 85%, 90%, 91%, 92%, 93%, 94%, 95%, 96%, 97%, 98%, 99%, or 100% identical to amino acids 1-292 of SEQ ID NO: 34.

141. The BMP10pro-Fc fusion protein of claim 139, wherein the BMP10pro domain consists of an amino acid sequence that is at least 70%, 75%, 80%, 85%, 90%, 91%, 92%, 93%, 94%, 95%, 96%, 97%, 98%, 99%, or 100% identical to amino acids 1-295 of SEQ ID NO: 34.

142. The BMP10pro-Fc fusion protein of any one of claim 139-141, wherein the BMP10pro domain does not comprise the sequence of amino acids RIRR.

143. The BMP10pro-Fc fusion protein of any one of claim 139-141, wherein the C-terminus of the polypeptide is not R296 of SEQ ID NO: 34.

144. The BMP10pro-Fc fusion protein of any one of claims 137-143, wherein the fusion protein comprises a linker domain positioned between the BMP10pro polypeptide domain and the Fc immunoglobulin domain.

145. The BMP10pro-Fc fusion protein of claim 144, wherein the linker is selected from the group consisting of: GGG (SEQ ID NO: 41), GGGG (SEQ ID NO: 42), TGGGG (SEQ ID NO: 43), SGGGG (SEQ ID NO: 44), TGGG (SEQ ID NO: 45), SGGG (SEQ ID NO: 46), or GGGGS (SEQ ID NO: 47).

146. The BMP10pro-Fc fusion protein of claim 131, wherein the fusion protein is selected from the group consisting of:
a. a polypeptide comprising an amino acid sequence that is at least 70%, 75% 80%, 85%, 90%, 91%, 92%, 93%, 94%, 95%, 96%, 97%, 98%, 99%, or 100% identical to the amino acid sequence of SEQ ID NO: 82;
b. a polypeptide comprising an amino acid sequence that is at least 70%, 75% 80%, 85%, 90%, 91%, 92%, 93%, 94%, 95%, 96%, 97%, 98%, 99%, or 100% identical to the amino acid sequence of SEQ ID NO: 84;
c. a polypeptide comprising an amino acid sequence that is at least 70%, 75% 80%, 85%, 90%, 91%, 92%, 93%, 94%, 95%, 96%, 97%, 98%, 99%, or 100% identical to the amino acid sequence of SEQ ID NO: 85; and
d. a polypeptide comprising an amino acid sequence that is at least 70%, 75% 80%, 85%, 90%, 91%, 92%, 93%, 94%, 95%, 96%, 97%, 98%, 99%, or 100% identical to the amino acid sequence of SEQ ID NO: 87.

147. The BMP10pro polypeptide of any one of claims 131-146 wherein the polypeptide or fusion protein comprises one or more amino acid modifications selected from the group consisting of: a glycosylated amino acid, a PEGylated amino acid, a farnesylated amino acid, an acetylated amino acid, a biotinylated amino acid, and an amino acid conjugated to a lipid moiety.

148. The method of claim 147, wherein the polypeptide or fusion protein has a glycosylation pattern obtainable from a Chinese hamster ovary cell line.

149. The BMP10pro polypeptide or BMP10pro-Fc fusion protein of any one of claims 131-148, wherein the polypeptide or fusion protein binds to BMP10 and/or BMP9.

150. The BMP10pro polypeptide or BMP10pro-Fc fusion protein of claim 149, wherein the polypeptide or fusion protein further binds to one or more ligands selected from the group consisting of: BMP6, BMP3b, and BMP5.

151. The BMP10pro polypeptide or BMP10pro-Fc fusion protein of any one of claims 131-150, wherein the polypeptide or inhibits activity of BMP10 and/or BMP9.

152. The BMP10pro polypeptide or BMP10pro-Fc fusion protein of claim 151, wherein the polypeptide or fusion protein further inhibits activity of one or more ligands selected from the group consisting of: BMP6, BMP3b, and BMP5.

153. The BMP10pro polypeptide or BMP10pro-Fc fusion protein of claim 151 or 152, wherein the wherein the polypeptide or inhibits activity of one or more of BMP10, BMP9, BMP6, BMP3b, and BMP5 in a cell-based assay.

154. A pharmaceutical preparation comprising the BMP10pro polypeptide or BMP10pro-Fc fusion protein of any one of claims 131-153 and a pharmaceutically acceptable carrier.

155. The pharmaceutical preparation of claim 154, wherein the preparation is substantially pyrogen free.

156. An isolated and/or recombinant polynucleotide, comprising a coding sequence for the BMP10pro polypeptide or BMP10pro-Fc fusion protein of any one of claims 131-153.

157. The isolated and/or recombinant polynucleotide of claim 156, wherein the polynucleotide comprises a nucleic acid sequence selected from the group consisting of:

158. A recombinant polynucleotide, comprising a promoter sequence operably linked to the polynucleotide of claim 156 or 157.

159. A vector comprising the isolated and/or recombinant polynucleotide of any one of claims 156-158.

160. A cell comprising the isolated and/or recombinant polynucleotide or vector of any one of claims 156-159.

161. The cell of claim 160, wherein the cell is a mammalian cell.

162. The cell of claim 161, wherein the cell is a CHO cell.

163. A method of making a BMP10pro polypeptide, comprising: culturing a cell under conditions suitable for expression of the BMP10pro polypeptide, wherein the cell comprises the isolated and/or recombinant polynucleotide or vector of any one of claims 156-159.

164. The method of claim 163, wherein the method further comprises a step of recovering the expressed BMP10pro polypeptide.

165. The method of claim 163 or 164, wherein the cell is a CHO cell.

166. The method of any one of claims 163-165, wherein the BMP10pro polypeptide is expressed using a tissue plasminogen activator signal sequence.
